SEQUENCE LISTING (1428 sequences)

SEQ ID NO: 1 - protein, 9 aa, Homo sapiens
VVGARGVGK

SEQ ID NO: 2 - protein, 10 aa, Homo sapiens
EYKLVVVGAR

SEQ ID NO: 3 - protein, 10 aa, Homo sapiens
VVVGARGVGK

SEQ ID NO: 4 - protein, 9 aa, Homo sapiens
ARGVGKSAL

SEQ ID NO: 5 - protein, 10 aa, Homo sapiens
VVVGAVGVGK

SEQ ID NO: 6 - protein, 10 aa, Homo sapiens
SMLTGPPARV

SEQ ID NO: 7 - protein, 10 aa, Homo sapiens
LLANGRMPTV

SEQ ID NO: 8 - protein, 62 aa, Homo sapiens
MTEYKLVVVGACGVGKSALTIQLIQNHFVDEYDPTIEDSYRKQVVIDGETCLLDILDTAGQE

SEQ ID NO: 9 - protein, 62 aa, Homo sapiens
MTEYKLVVVGADGVGKSALTIQLIQNHFVDEYDPTIEDSYRKQVVIDGETCLLDILDTAGQE

SEQ ID NO: 10 - protein, 62 aa, Homo sapiens
MTEYKLVVVGAVGVGKSALTIQLIQNHFVDEYDPTIEDSYRKQVVIDGETCLLDILDTAGQE

SEQ ID NO: 11 - protein, 101 aa, Homo sapiens
AGGVGKSALTIQLIQNHFVDEYDPTIEDSYRKQVVIDGETCLLDILDTAGHEEYSAMRDQYMRTGEGFLCVFAINNTKSFEDIHHYREQIKRVKDSEDVPM

SEQ ID NO: 12 - protein, 101 aa, Homo sapiens
AGGVGKSALTIQLIQNHFVDEYDPTIEDSYRKQVVIDGETCLLDILDTAGLEEYSAMRDQYMRTGEGFLCVFAINNTKSFEDIHHYREQIKRVKDSEDVPM

SEQ ID NO: 13 - protein, 101 aa, Homo sapiens
AGGVGKSALTIQLIQNHFVDEYDPTIEDSYRKQVVIDGETCLLDILDTAGKEEYSAMRDQYMRTGEGFLCVFAINNSKSFADINLYREQIKRVKDSDDVPM

SEQ ID NO: 14 - protein, 101 aa, Homo sapiens
AGGVGKSALTIQLIQNHFVDEYDPTIEDSYRKQVVIDGETCLLDILDTAGREEYSAMRDQYMRTGEGFLCVFAINNSKSFADINLYREQIKRVKDSDDVPM

SEQ ID NO: 15 - protein, 101 aa, Homo sapiens
MIKEGSMSEDEFIEEAKVMMNLSHEKLVQLYGVCTKQRPIFIITEYMANGSLLNYLREMRHRFQTQQLLEMCKDVCEAMEYLESKQFLHRDLAARNCLVND

SEQ ID NO: 16 - protein, 101 aa, Homo sapiens
SLNITSLGLRSLKEISDGDVIISGNKNLCYANTINWKKLFGTSGQKTKIIRNRGENSCKATGQVCHALCSPEGCWGPEPRDCVSCRNVSRGRECVDKCNLL

SEQ ID NO: 17 - protein, 101 aa, Homo sapiens
IPVAIKELREATSPKANKEILDEAYVMASVDNPHVCRLLGICLTSTVQLIMQLMPFGCLLDYVREHKDNIGSQYLLNWCVQIAKGMNYLEDRRLVHRDLAA

SEQ ID NO: 18 - protein, 101 aa, Homo sapiens
VADGLITTLHYPAPKRNKPTVYGVSPNYDKWEMERTDITMKHKLGGGQYGKVYEGVWKKYSLTVAVKTLKEDTMEVEEFLKEAAVMKEIKHPNLVQLLGVC

SEQ ID NO: 19 - protein, 101 aa, Homo sapiens
VADGLITTLHYPAPKRNKPTVYGVSPNYDKWEMERTDITMKHKLGGGQYGVVYEGVWKKYSLTVAVKTLKEDTMEVEEFLKEAAVMKEIKHPNLVQLLGVC

SEQ ID NO: 20 - protein, 101 aa, Homo sapiens
LLGVCTREPPFYIITEFMTYGNLLDYLRECNRQEVNAVVLLYMATQISSATEYLEKKNFIHRDLAARNCLVGENHLVKVADFGLSRLMTGDTYTAHAGAKF

SEQ ID NO: 21 - protein, 101 aa, Homo sapiens
SLTVAVKTLKEDTMEVEEFLKEAAVMKEIKHPNLVQLLGVCTREPPFYIIIEFMTYGNLLDYLRECNRQEVNAVVLLYMATQISSAMEYLEKKNFIHRDLA

SEQ ID NO: 22 - protein, 101 aa, Homo sapiens
STVADGLITTLHYPAPKRNKPTVYGVSPNYDKWEMERTDITMKHKLGGGQHGEVYEGVWKKYSLTVAVKTLKEDTMEVEEFLKEAAVMKEIKHPNLVQLLG

SEQ ID NO: 23 - protein, 101 aa, Homo sapiens
SSLAMLDLLHVARDIACGCQYLEENHFIHRDIAARNCLLTCPGPGRVAKIADFGMARDIYRASYYRKGGCAMLPVKWMPPEAFMEGIFTSKTDTWSFGVLL

SEQ ID NO: 24 - protein, 101 aa, Homo sapiens
QVAVKTLPEVCSEQDELDFLMEALIISKFNHQNIVRCIGVSLQSLPRFILMELMAGGDLKSFLRETRPRPSQPSSLAMLDLLHVARDIACGCQYLEENHFI

SEQ ID NO: 25 - protein, 101 aa, Homo sapiens
MIKLIDIARQTAQGMDYLHAKSIIHRDLKSNNIFLHEDLTVKIGDFGLATEKSRWSGSHQFEQLSGSILWMAPEVIRMQDKNPYSFQSDVYAFGIVLYELM

SEQ ID NO: 26 - protein, 93 aa, Homo sapiens
MGFGDLKSPAGLQVLNDYLADKSYIEGYVPSQADVAVFEAVSGPPPADLCHALRWYNHIKSYEKEKASLPGVKKALGKYGPADVEDTTGSGAT

SEQ ID NO: 27 - protein, 101 aa, Homo sapiens
ERCEVVMGNLEIVLTGHNADLSFLQWIREVTGYVLVAMNEFSTLPLPNLRMVRGTQVYDGKFAIFVMLNYNTNSSHALRQLRLTQLTEILSGGVYIEKNDK

SEQ ID NO: 28 - protein, 101 aa, Homo sapiens
HLMAKAGLTLQQQHQRLAQLLLILSHIRHMSNKGMEHLYSMKCKNVVPLYGLLLEMLDAHRLHAPTSRGGASVEETDQSHLATAGSTSSHSLQKYYITGEA

SEQ ID NO: 29 - protein, 101 aa, Homo sapiens
NQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSH

SEQ ID NO: 30 - protein, 101 aa, Homo sapiens
IHLMAKAGLTLQQQHQRLAQLLLILSHIRHMSNKGMEHLYSMKCKNVVPLCDLLLEMLDAHRLHAPTSRGGASVEETDQSHLATAGSTSSHSLQKYYITGE

SEQ ID NO: 31 - protein, 101 aa, Homo sapiens
IHLMAKAGLTLQQQHQRLAQLLLILSHIRHMSNKGMEHLYSMKCKNVVPLNDLLLEMLDAHRLHAPTSRGGASVEETDQSHLATAGSTSSHSLQKYYITGE

SEQ ID NO: 32 - protein, 101 aa, Homo sapiens
IHLMAKAGLTLQQQHQRLAQLLLILSHIRHMSNKGMEHLYSMKCKNVVPLSDLLLEMLDAHRLHAPTSRGGASVEETDQSHLATAGSTSSHSLQKYYITGE

SEQ ID NO: 33 - protein, 101 aa, Homo sapiens
HRIGGIKLRHQQWSLVMESVVPSDRGNYTCVVENKFGSIRQTYTLDVLERCPHRPILQAGLPANQTAVLGSDVEFHCKVYSDAQPHIQWLKHVEVNGSKVG

SEQ ID NO: 34 - protein, 101 aa, Homo sapiens
AVKLSDSRIALKSGYGKYLGINSDELVGHSDAIGPREQWEPVFQNGKMALSASNSCFIRCNEAGDIEAKSKTAGEEEMIKIRSCAEKETKKKDDIPEEDKG

SEQ ID NO: 35 - protein, 101 aa, Homo sapiens
GSGAFGTVYKGIWIPDGENVKIPVAIKVLRENTSPKANKEILDEAYVMAGLGSPYVSRLLGICLTSTVQLVTQLMPYGCLLDHVRENRGRLGSQDLLNWCM

SEQ ID NO: 36 - protein, 101 aa, Homo sapiens
RVEEFKLKQMWKSPNGTIRNILGGTVFREAIICKNIPRLVSGWVKPIIIGHHAYGDQYRATDFVVPGPGKVEITYTPSDGTQKVTYLVHNFEEGGGVAMGM

SEQ ID NO: 37 - protein, 101 aa, Homo sapiens
RVEEFKLKQMWKSPNGTIRNILGGTVFREAIICKNIPRLVSGWVKPIIIGCHAYGDQYRATDFVVPGPGKVEITYTPSDGTQKVTYLVHNFEEGGGVAMGM

SEQ ID NO: 38 - protein, 101 aa, Homo sapiens
RVEEFKLKQMWKSPNGTIRNILGGTVFREAIICKNIPRLVSGWVKPIIIGGHAYGDQYRATDFVVPGPGKVEITYTPSDGTQKVTYLVHNFEEGGGVAMGM

SEQ ID NO: 39 - protein, 101 aa, Homo sapiens
RVEEFKLKQMWKSPNGTIRNILGGTVFREAIICKNIPRLVSGWVKPIIIGSHAYGDQYRATDFVVPGPGKVEITYTPSDGTQKVTYLVHNFEEGGGVAMGM

SEQ ID NO: 40 - protein, 101 aa, Homo sapiens
VAVKMLKPSAHLTEREALMSELKVLSYLGNHMNIVNLLGACTIGGPTLVIIEYCCYGDLLNFLRRKRDSFICSKQEDHAEAALYKNLLHSKESSCSDSTNE

SEQ ID NO: 41 - protein, 101 aa, Homo sapiens
VEATAYGLIKSDAAMTVAVKMLKPSAHLTEREALMSELKVLSYLGNHMNIANLLGACTIGGPTLVITEYCCYGDLLNFLRRKRDSFICSKQEDHAEAALYK

SEQ ID NO: 42 - protein, 101 aa, Homo sapiens
ISELGAGNGGVVFKVSHKPSGLVMARKLIHLEIKPAIRNQIIRELQVLHESNSPYIVGFYGAFYSDGEISICMEHMDGGSLDQVLKKAGRIPEQILGKVSI

SEQ ID NO: 43 - protein, 101 aa, Homo sapiens
LGAGNGGVVFKVSHKPSGLVMARKLIHLEIKPAIRNQIIRELQVLHECNSLYIVGFYGAFYSDGEISICMEHMDGGSLDQVLKKAGRIPEQILGKVSIAVI

SEQ ID NO: 44 - protein, 89 aa, Homo sapiens
MPLNVSFTNRNYDLDYDSVQPYFYCDEEENFYQQQQQSDLQPPAPSEDIWKKFELLPTPPLSPSRRSGLCSPSYVAVTPFSLRGDNDGG

SEQ ID NO: 45 - protein, 101 aa, Homo sapiens
FTNRNYDLDYDSVQPYFYCDEEENFYQQQQQSELQPPAPSEDIWKKFELLSTPPLSPSRRSGLCSPSYVAVTPFSLRGDNDGGGGSFSTADQLEMVTELLG

SEQ ID NO: 46 - protein, 101 aa, Homo sapiens
TNRNYDLDYDSVQPYFYCDEEENFYQQQQQSELQPPAPSEDIWKKFELLPIPPLSPSRRSGLCSPSYVAVTPFSLRGDNDGGGGSFSTADQLEMVTELLGG

SEQ ID NO: 47 - protein, 101 aa, Homo sapiens
VAVKMLKPTARSSEKQALMSELKIMTHLGPHLNIVNLLGACTKSGPIYIIIEYCFYGDLVNYLHKNRDSFLSHHPEKPKKELDIFGLNPADESTRSYVILS

SEQ ID NO: 48 - protein, 101 aa, Homo sapiens
IEEHANWSVSREAGFSYSHAGLSNRLARDNELRENDKEQLKAISTRDPLSKITEQEKDFLWSHRHYCVTIPEILPKLLLSVKWNSRDEVAQMYCLVKDWPP

SEQ ID NO: 49 - protein, 101 aa, Homo sapiens
HANWSVSREAGFSYSHAGLSNRLARDNELRENDKEQLKAISTRDPLSEITKQEKDFLWSHRHYCVTIPEILPKLLLSVKWNSRDEVAQMYCLVKDWPPIKP

SEQ ID NO: 50 - protein, 72 aa, Homo sapiens
LFINLFSMMLGSGMPELQSFDDIAYIRKTLALDKTEQEALEYFMKQMNDARHGGWTTKMDWIFHTIKQHALN

SEQ ID NO: 51 - protein, 101 aa, Homo sapiens
QRGGVITDEEETSKKIADQLDNIVDMREYDVPYHIRLSIDIETTKLPLKFRDAETDQIMMISYMIDGQGYLITNREIVSEDIEDFEFTPKPEYEGPFCVFN

SEQ ID NO: 52 - protein, 101 aa, Homo sapiens
KFNCRVAQYPFEDHNPPQLELIKPFCEDLDQWLSEDDNHVAAIHCKAGKGQTGVMICAYLLHRGKFLKAQEALDFYGEVRTRDKKGVTIPSQRRYVYYYSY

SEQ ID NO: 53 - protein, 79 aa, Homo sapiens
MQAIKCVVVGDGAVGKTCLLISYTTNAFSGEYIPTVFDNYSANVMVDGKPVNLGLWDTAGQEDYDRLRPLSYPQTVGET

SEQ ID NO: 54 - protein, 101 aa, Homo sapiens
IRVEGNLRVEYLDDRNTFRHSVVVPYEPPEVGSDCTTIHYNYMCNSSCMGSMNRRPILTIITLEDSSGNLLGRNSFEVRVCACPGRDRRTEEENLRKKGEP

SEQ ID NO: 55 - protein, 101 aa, Homo sapiens
TYSPALNKMFCQLAKTCPVQLWVDSTPPPGTRVRAMAIYKQSQHMTEVVRHCPHHERCSDSDGLAPPQHLIRVEGNLRVEYLDDRNTFRHSVVVPYEPPEV

SEQ ID NO: 56 - protein, 101 aa, Homo sapiens
EGNLRVEYLDDRNTFRHSVVVPYEPPEVGSDCTTIHYNYMCNSSCMGGMNQRPILTIITLEDSSGNLLGRNSFEVRVCACPGRDRRTEEENLRKKGEPHHE

SEQ ID NO: 57 - protein, 101 aa, Homo sapiens
EGNLRVEYLDDRNTFRHSVVVPYEPPEVGSDCTTIHYNYMCNSSCMGGMNWRPILTIITLEDSSGNLLGRNSFEVRVCACPGRDRRTEEENLRKKGEPHHE

SEQ ID NO: 58 - protein, 101 aa, Homo sapiens
PEVGSDCTTIHYNYMCNSSCMGGMNRRPILTIITLEDSSGNLLGRNSFEVCVCACPGRDRRTEEENLRKKGEPHHELPPGSTKRALPNNTSSSPQPKKKPL

SEQ ID NO: 59 - protein, 53 aa, Homo sapiens
GVEPCYGDKDKRRHCFATWKNISGSIEIVKQGCWLDDINCYDRTDCVEKKRQP

SEQ ID NO: 60 - protein, 103 aa, Homo sapiens
GVEPCYGDKDKRRHCFATWKNISGSIEIVKQGCWLDDINCYDRTDCVEKKTALKYIFVAVRAICVMKSFLIFRRWKSHSPLQIQLHLSHPITTSCSIPWCHLC

SEQ ID NO: 61 - protein, 118 aa, Homo sapiens
TAEAVNVAIAAPPSEGEANAELCRYLSKVLELRKSDVVLDKVGLALFFFFFETKSCSVAQAGVQWRSLGSLQPPPPGFKLFSCLSFLSSWDYRRMPPCLANFCIFNRDGVSPCWSGWS

SEQ ID NO: 62 - protein, 71 aa, Homo sapiens
LSVIIFFFVYIWHWALPLILNNHHICLMSSIILDCNSVRQSIMSVCFFFFSVIFSTRCLTDSRYPNICWFK

SEQ ID NO: 63 - protein, 60 aa, Homo sapiens
LSVIIFFFVYIWHWALPLILNNHHICLMSSIILDCNSVRQSIMSVCFFFFCYILNTMFDR

SEQ ID NO: 64 - protein, 106 aa, Homo sapiens
VLVLSCDLITDVALHEVVDLFRAYDASLAMLMRKGQDSIEPVPGQKGKKKQWSSVTSLEWTAQERGCSSWLMKQTWMKSWSLRDPSYRSILEYVSTRVLWMPTSTV

SEQ ID NO: 65 - protein, 112 aa, Homo sapiens
SIQVMRAQMNQIQSVEGQPLARRPRATGRTKRCQPRDVTKKTCNSNDGKKREWEKRKQILGGGGKYKEYFLKRILIRKAMTVLAGDKKGLGRFMRCVQSETKAVSLQLPLGR

SEQ ID NO: 66 - protein, 57 aa, Homo sapiens
LDFLGEFATDIRTHGVHMVLNHQGRPSGDAFIQMKSADRAFMAAQKCHKKKHEGQIC

SEQ ID NO: 67 - protein, 51 aa, Homo sapiens
LDFLGEFATDIRTHGVHMVLNHQGRPSGDAFIQMKSADRAFMAAQKCHKKT

SEQ ID NO: 68 - protein, 75 aa, Homo sapiens
GALCKDGRFRSDIGEFEWKLKEGHKKIYGKQSMVDEVSGKVLEMDISKKKHYNRKISIKKLNRMKVPLMKLITRV

SEQ ID NO: 69 - protein, 58 aa, Homo sapiens
RERAQLLEEQEKTLTSKLQEQARVLKERCQGESTQLQNEIQKLQKTLKKKPRDICRIS

SEQ ID NO: 70 - protein, 53 aa, Homo sapiens
VNTLKEGKRLPCPPNCPDEVYQLMRKCWEFQPSNRTSFQNLIEGFEALLKTSN

SEQ ID NO: 71 - protein, 65 aa, Homo sapiens
CRPVTPSCKELADLMTRCMNYDPNQRPFFRAIMRDINKLEEQNPDIVSEKNQQLKWTPHILKSAS

SEQ ID NO: 72 - protein, 69 aa, Homo sapiens
DDHDVLSFLTFQLTEPGKEPPTPDKEISEKEKEKYQEEFEHFQQELDKKKRGIPEGPPRPPRAACGGNI

SEQ ID NO: 73 - protein, 71 aa, Homo sapiens
DDHDVLSFLTFQLTEPGKEPPTPDKEISEKEKEKYQEEFEHFQQELDKKKRNSRRATPTSKGSLRRKYLRV

SEQ ID NO: 74 - protein, 68 aa, Homo sapiens
TKSTLIGEDVNPLIKLDDAVNVDEIMTDTSTSYLLCISENKENVRDKKKGQHFYWHCGSAACHRRGCV

SEQ ID NO: 75 - protein, 81 aa, Homo sapiens
LYTKSTLIGEDVNPLIKLDDAVNVDEIMTDTSTSYLLCISENKENVRDKKRATFLLALWECSLPQARLCLIVSRTLLLVQS

SEQ ID NO: 76 - protein, 58 aa, Homo sapiens
LPPPKLTDPRLLYIGFLGYCSGLIDNLIRRRPIATAGLHRQLLYITAFFFCWILSCKT

SEQ ID NO: 77 - protein, 56 aa, Homo sapiens
SLPPPKLTDPRLLYIGFLGYCSGLIDNLIRRRPIATAGLHRQLLYITAFFLLDIIL

SEQ ID NO: 78 - protein, 112 aa, Homo sapiens
NQSGGAGEDCQIFSTPGHPKMIYSSSNLKTPSKLCSGSKSHDVQEVLKKKTGSNEVTTRYEEKKTGSVRKANRMPKDVNIQVRKKQKHETRRKSKYNEDFERAWREDLTIKR

SEQ ID NO: 79 - protein, 23 aa, Homo sapiens
MAPVKKLVVKGGKKKEASSEVHS

SEQ ID NO: 80 - protein, 18 aa, Homo sapiens
MAPVKKLVVKGGKKRSKF

SEQ ID NO: 81 - protein, 51 aa, Homo sapiens
MPSHQGAEQQQQQHHVFISQVVTEKEFLSRSDQLQQAVQSQGFINYCQKKN

SEQ ID NO: 82 - protein, 65 aa, Homo sapiens
MPSHQGAEQQQQQHHVFISQVVTEKEFLSRSDQLQQAVQSQGFINYCQKKLMLLRLNLRKMCGPF

SEQ ID NO: 83 - protein, 79 aa, Homo sapiens
AEVFEKEQSICAAEEQPAEDGQGETNKNRTKGGWQQKSKGPKKTAKSKKKETFKKKTYTCAITTVKATETKAGKWSRWE

SEQ ID NO: 84 - protein, 53 aa, Homo sapiens
MAEVFEKEQSICAAEEQPAEDGQGETNKNRTKGGWQQKSKGPKKTAKSKKRNL

SEQ ID NO: 85 - protein, 71 aa, Homo sapiens
NIMEIRQLPSSHALEAKLSRMSYPVKEQESILKTVGKLTATQVAKISFFFALCGFWQICHIKKHFQTHKLL

SEQ ID NO: 86 - protein, 68 aa, Homo sapiens
YEKKKYYDKNAIAITNISSSDAPLQPLVSSPSLQAAVDKNKLEKEKEKKKGREKERKGARKAGKTTYS

SEQ ID NO: 87 - protein, 97 aa, Homo sapiens
KYEKKKYYDKNAIAITNISSSDAPLQPLVSSPSLQAAVDKNKLEKEKEKKRKRKREKRSQKSRQNHLQLKSCRRKISNWSLKKVPALKKLRSPLWIF

SEQ ID NO: 88 - protein, 65 aa, Homo sapiens
IYQDAYRAEWQVYKEEISRFKEQLTPSQIMSLEKEIMDKHLKRKAMTKKKRVNTAWKTKKTSFSL

SEQ ID NO: 89 - protein, 51 aa, Homo sapiens
IYQDAYRAEWQVYKEEISRFKEQLTPSQIMSLEKEIMDKHLKRKAMTKKKS

SEQ ID NO: 90 - protein, 52 aa, Homo sapiens
KPQEVCVAVWRKNDENITLETVCHDPKLPYHDFILEDAASPKCIMKEKKKAW

SEQ ID NO: 91 - protein, 84 aa, Homo sapiens
EKPQEVCVAVWRKNDENITLETVCHDPKLPYHDFILEDAASPKCIMKEKKSLVRLSSCVPVALMSAMTTSSSQKNITPAILTCC

SEQ ID NO: 92 - protein, 74 aa, Homo sapiens
VPSKYQFLCSDHFTPDSLDIRWGIRYLKQTAVPTIFSLPEDNQGKDPSKKNPRRKTWKMRKKYAQKPSQKNHLY

SEQ ID NO: 93 - protein, 88 aa, Homo sapiens
GTTEEMKYVLGQLVGLNSPNSILKAAKTLYEHYSGGESHNSSSSKTFEKKGEKNDLQLFVMSDTTYKIYWTVILLNPCGNLHLKTTSL

SEQ ID NO: 94 - protein, 55 aa, Homo sapiens
QQLIRETLISWLQAQMLNPQPEKTFIRNKAAQVFALLFVTEYLTKWPKFFLTFSQ

SEQ ID NO: 95 - protein, 93 aa, Homo sapiens
AKFQQCHSTLEPNPADCRVLVYLQNQPGTKLLNFLQERNLPPKVVLRHPKVHLNTMFRRPHSCLADVLLSVHLIVLRVVRLPAPFRVNHAVEW

SEQ ID NO: 96 - protein, 53 aa, Homo sapiens
APVIFQIALDKPCHQAEVKHLHHLLKQLKPSEKYLKIKHLLLKRERVDLSKLQ

SEQ ID NO: 97 - protein, 33 aa, Homo sapiens
MLQFRGSRFFQMLILYYILPRKVLQMDFLVHPA

SEQ ID NO: 98 - protein, 179 aa, Homo sapiens
ALGPHSRISCLPTQTRGCILLAATPRSSSSSSSNDMIPMAISSPPKAPLLAAPSPASRLQCINSNSRITSGQWMAHMALLPSGTKGRCTACHTALGRGSLSSSSCPQPSPSLPASNKLPSLPLSKMYTTSMAMPILPLPQLLLSADQQAAPRTNFHSSLAETVSLHPLAPMPSKTCHHK

SEQ ID NO: 99 - protein, 79 aa, Homo sapiens
AHQGFPAAKESRVIQLSLLSLLIPPLTCLASEALPRPLLALPPVLLSLAQDHSRLLQCQATRCHLGHPVASRTASCILP

SEQ ID NO: 100 - protein, 222 aa, Homo sapiens
PILAATGTSVRTAARTWVPRAAIRVPDPAAVPDDHAGPGAECHGRPLLYTADSSLWTTRPQRVWSTGPDSILQPAKSSPSAAAATLLPATTVPDPSCPTFVSAAATVSTTTAPVLSASILPAAIPASTSAVPGSIPLPAVDDTAAPPEPAPLLTATGSVSLPAAATSAASTLDALPAGCVSSAPVSAVPANCLFPAALPSTAGAISRFIWVSGILSPLNDLQ

SEQ ID NO: 101 - protein, 185 aa, Homo sapiens
PCRAGRRVPWAASLIHSRFLLMDNKAPAGMVNRARLHITTSKVLTLSSSSHPTPSNHRPRPLMPNLRISSSHSLNHHSSSPLSLHTPSSHPSLHISSPRLHTPPSSRRHSSTPRASPPTHSHRLSLLTSSSNLSSQHPRRSPSRLRILSPSLSSPSKLPIPSSASLHRRSYLKIHLGLRHPQPPQ

SEQ ID NO: 102 - protein, 58 aa, Homo sapiens
RTNPTVRMRPHCVPFWTGRILLPSAASVCPIPFEACHLCQAMTLRCPNTQGCCSSWAS

SEQ ID NO: 103 - protein, 90 aa, Homo sapiens
TNQALPKIEVICRGTPRCPSTVPPSPAQPYLRVSLPEDRYTQAWAPTSRTPWGAMVPRGVSMAHKVATPGSQTIMPCPMPTTPVQAWLEA

SEQ ID NO: 104 - protein, 41 aa, Homo sapiens
RMERELKKWSIQTCLSARTGLSISCTTLNSPPLKKMSMPAV

SEQ ID NO: 105 - protein, 35 aa, Homo sapiens
LCSRYSLFLAWRLSSVLQRFRFTHVIQQRMESQIS

SEQ ID NO: 106 - protein, 39 aa, Homo sapiens
RSACVTVKGPLASVGRHSLSKQDCKFLPFWGFLEEFLLC

SEQ ID NO: 107 - protein, 97 aa, Homo sapiens
IQWGTTTAPRPIRPPFLESKQNCSHFPTPLLASEDRRETGLFLPSAAQKMKKAHFLKTWFRSNPTKTKKARFSTASLAKELTHPLLVSLLLKEKQDG

SEQ ID NO: 108 - protein, 73 aa, Homo sapiens
PTDPFLGLRLGLHLQKVFHQSHAEYSGAPPPPPAPSGLRFWNPSRIAHISQLLSWPQKTEERLGYSSHQLPRK

SEQ ID NO: 109 - protein, 45 aa, Homo sapiens
FCCSCCFFGGERWSKSPYCPQRMTPGTTFITMMKKEAEKRTRTLT

SEQ ID NO: 110 - protein, 95 aa, Homo sapiens
WRRNCKAPVSLRKSVQTPARSSPARPDRTRRLPSLGVPGQPWALGAAASRRCCCCCRSPLGSARSRSPATLALTPRATRSRCPGATWREAASWAE

SEQ ID NO: 111 - protein, 113 aa, Homo sapiens
PGRPLQTHVLPEPHLALQPLQPHADHAHADAPAIQPVLWTTPPLQHGHRHGLEPCSMLTGPPARVPAVPFDLHFCRSSIMKPKRDGYMFLKAESKIMFATLQRSSLWCLCSNH

SEQ ID NO: 112 - protein, 57 aa, Homo sapiens
PRPRRCTRHPACPLDHTTPPAWSPPWVRALLDAHRAPSESPCSPFRLAFLQEQYHEA

SEQ ID NO: 113 - protein, 494 aa, Homo sapiens
TRRCHCCPHLRSHPCPHHLRNHPRPHHLRHHACHHHLRNCPHPHFLRHCTCPGRWRNRPSLRRLRSLLCLPHLNHHLFLHWRSRPCLHRKSHPHLLHLRRLYPHHLKHRPCPHHLKNLLCPRHLRNCPLPRHLKHLACLHHLRSHPCPLHLKSHPCLHHRRHLVCSHHLKSLLCPLHLRSLPFPHHLRHHACPHHLRTRLCPHHLKNHLCPPHLRYRAYPPCLWCHACLHRLRNLPCPHRLRSLPRPLHLRLHASPHHLRTPPHPHHLRTHLLPHHRRTRSCPCRWRSHPCCHYLRSRNSAPGPRGRTCHPGLRSRTCPPGLRSHTYLRRLRSHTCPPSLRSHAYALCLRSHTCPPRLRDHICPLSLRNCTCPPRLRSRTCLLCLRSHACPPNLRNHTCPPSLRSHACPPGLRNRICPLSLRSHPCPLGLKSPLRSQANALHLRSCPCSLPLGNHPYLPCLESQPCLSLGNHLCPLCPRSCRCPHLGSHPCRLS

SEQ ID NO: 114 - protein, 53 aa, Homo sapiens
SWKGTNWCNDMCIFITSGQIFKGTRGPRFLWGSKDQRQKGSNYSQSEALCVLL

SEQ ID NO: 115 - protein, 9 aa, Homo sapiens
KRTKCFTFG

SEQ ID NO: 116 - protein, 27 aa, Homo sapiens
PIFIQTLLLWDFLQKDLKAYTGTILMM

SEQ ID NO: 117 - protein, 22 aa, Homo sapiens
QKMILTKQIKTKPTDTFLQILR

SEQ ID NO: 118 - protein, 43 aa, Homo sapiens
GFWIQSIKTITRYTIFVLKDIMTPPNLIAELHNILLKTITHHS

SEQ ID NO: 119 - protein, 40 aa, Homo sapiens
NYSNVQWRNLQSSVCGLPAKGEDIFLQFRTHTTGRQVHVL

SEQ ID NO: 120 - protein, 44 aa, Homo sapiens
YQSRVLPQTEQDAKKGQNVSLLGKYILHTRTRGNLRKSRKWKSM

SEQ ID NO: 121 - protein, 85 aa, Homo sapiens
SSQNARGCSPRGPCTSSSYTGGPCTSPLLAPVIFCPFPENLPGQLRFPSGLLAFWDSQVCDLHVLPCPQQDVLPTGQDLPCAAVG

SEQ ID NO: 122 - protein, 72 aa, Homo sapiens
GAAPTMSAAQIAMVWPLLSILSEWKEICVWSIWMTETLFDIVWWCPMSRLRLALTVPPSTTTTCVTVPAWAA

SEQ ID NO: 123 - protein, 97 aa, Homo sapiens
TGGPSSPSSHWKTPVVIYWDGTALRCVFVPVLGETGAQRKRISARKGSLTTSCPQGALSEHCPTTPAPLPSQRRNHWMENISPFRSVGVSASRCSES

SEQ ID NO: 124 - protein, 31 aa, Homo sapiens
FHTPARHPRPRHGHLQAVTAHDGGCEALPPP

SEQ ID NO: 125 - protein, 78 aa, Homo sapiens
CCPRTILNNGSLKTQVQMKLPECQRLLPPWPLHQQLLHRRPLHQPPPGPCHLLSLPRKPTRAATVSVWASCILGQPSL

SEQ ID NO: 126 - protein, 28 aa, Homo sapiens
VRKHFQTYGNYFLKTTFCPPCRPKQWMI

SEQ ID NO: 127 - protein, 46 aa, Homo sapiens
LARTPLPSTRCFANWPRPALCSCGLIPHPRPAPASAPWPSTSSHST

SEQ ID NO: 128 - protein, 81 aa, Homo sapiens
ELQETGHRQVALRRSGRPPKCAERPGAADTGAHCTSTDGRLKISVETYTVSSQLLMVLMSLDLDTGLVPSLVSKCLILRVK

SEQ ID NO: 129 - protein, 10 aa, Homo sapiens
KSDASRLSGA

SEQ ID NO: 130 - protein, 29 aa, Homo sapiens
RTAYFCQYHTASVYSERAMPPGCPEPSQA

SEQ ID NO: 131 - protein, 91 aa, Homo sapiens
TRASPPRSSSAIAVRASCCPYGSTSTASRSPTQRCRLARAAASTATEVTFGSSEMQGHTMGFWLTKLNYLCHLSMLTDSLFLPISHCQCIL

SEQ ID NO: 132 - protein, 31 aa, Homo sapiens
SSLRITGDWTSSGRSTKIWKTTQMCRKTWSG

SEQ ID NO: 133 - protein, 104 aa, Homo sapiens
RRRRGGVGRRGVRPGRVRPGGTGRRGGDGGRAAAARAALGELARALPGHLLQSQSARRAARMAQLRRRAAALPNAAAWHGPPHPQLPRSPLALQRCRDTRWASG

SEQ ID NO: 134 - protein, 66 aa, Homo sapiens
SCKVFFKRAAEGKQKYLCASRNDCTIDKFRRKNCPSCRLRKCYEAGMTLGEKFRVGNCKHLKMTRP

SEQ ID NO: 135 - protein, 49 aa, Homo sapiens
MAGAPPPASLPPCSLISDCCASNQRDSVGVGPSEPGNNIKICNESASRK

SEQ ID NO: 136 - protein, 61 aa, Homo sapiens
HGWRPFLPVRARSRWNRRLDVTVANGRSWKYGWSLLRVPQVNGIQVLNVSLKSSSNVISYE

SEQ ID NO: 137 - protein, 65 aa, Homo sapiens
RLKEVFQTKIQEFRKACYTLTGYQIDITTENQYRLTSLYAEHPGDCLIFKLRVPGSSVLVTVPGL

SEQ ID NO: 138 - protein, 107 aa, Homo sapiens
AEVLKVIRQSAGQKTTCGQGLEGPWERPPPLDESERDGGSEDQVEDPALSALLLRPRPPRPEVGAHQDEQAAQGADPRLGAQPACRGLPGLLTVPQPEPLLAPPSAA

SEQ ID NO: 139 - protein, 100 aa, Homo sapiens
ERAEWRENIREQQKKCFRSFSLTSVELQMLTNSCVKLQTVHSIPLTINKEEALQRPVASDFEPQGLSEAARWNSKENLLAGPSENDPNLFVALYDFVASG

SEQ ID NO: 140 - protein, 101 aa, Homo sapiens
ELQMLTNSCVKLQTVHSIPLTINKEDDESPGLYGFLNVIVHSATGFKQSSKALQRPVASDFEPQGLSEAARWNSKENLLAGPSENDPNLFVALYDFVASGD

SEQ ID NO: 141 - protein, 101 aa, Homo sapiens
ISNSWDAHLGLGACGEAEGLGVQGAEEEEEEEEEEEEEGAGVPACPPKGPELFPLIFPAEPAQASGPYVEIIEQPKQRGMRFRYKCEGRSAGSIPGERSTD

SEQ ID NO: 142 - protein, 100 aa, Homo sapiens
LQRLDGMGCLEFDEERAQQEDALAQQAFEEARRRTREFEDRDRSHREEMEVHELEKSKRALETQMEEMKTQLEELEDELQATEDAKLRLEVNMQALKGQF

SEQ ID NO: 143 - protein, 100 aa, Homo sapiens
KGSFPENLRHLKNTMETIDWKVFESWMHHWLLFEMSRHSLEQKPTDAPPKAGVPNKPGIPKLLEGSKNSIQWEKAEDNGCRITYYILEIRKSTSNNLQNQ

SEQ ID NO: 144 - protein, 80 aa, Homo sapiens
MRPSGTAGAALLALLAALCPASRALEEKKGNYVVTDHGSCVRACGADSYEMEEDGVRKCKKCEGPCRKVCNGIGIGEFKD

SEQ ID NO: 145 - protein, 100 aa, Homo sapiens
LPQPPICTIDVYMIMVKCWMIDADSRPKFRELIIEFSKMARDPQRYLVIQLQDKFEHLKMIQQEEIRKLEEEKKQLEGEIIDFYKMKAASEALQTQLSTD

SEQ ID NO: 146 - protein, 101 aa, Homo sapiens
SWENSDDSRNKLSKIPSTPKLIPKVTKTADKHKDVIINQAKMSTREKNSQVYRRKHQELQAMQMELQSPEYKLSKLRTSTIMTDYNPNYCFAGKTSSISDL

SEQ ID NO: 147 - protein, 100 aa, Homo sapiens
EGHRMDKPANCTHDLYMIMRECWHAAPSQRPTFKQLVEDLDRVLTVTSTDVKATQEENRELRSRCEELHGKNLELGKIMDRFEEVVYQAMEEVQKQKELS

SEQ ID NO: 148 - protein, 107 aa, Homo sapiens
RDNTLLLRRVELFSLSRQVARESTYLSSLKGSRLHPEELGGPPLKKLKQEATSKSQIMSLWGLVSKMPPEKVQRLYVDFPQHLRHLLGDWLESQPWEFLVGSDAFCC

SEQ ID NO: 149 - protein, 83 aa, Homo sapiens
MSREMQDVDLAEVKPLVEKGETITGLLQEFDVQEALSVVSEDQSLFECAYGTPHLAKTEMTASSSSDYGQTSKMSPRVPQQDW

SEQ ID NO: 150 - protein, 101 aa, Homo sapiens
VLDMHGFLRQALCRLRQEEPQSLQAAVRTDGFDEFKVRLQDLSSCITQGKAIETQSSSSEEIVPSPPSPPPLPRIYKPCFVCQDKSSGYHYGVSACEGCKG

SEQ ID NO: 151 - protein, 101 aa, Homo sapiens
RSSPEQPRPSTSKAVSPPHLDGPPSPRSPVIGSEVFLPNSNHVASGAGEAAIETQSSSSEEIVPSPPSPPPLPRIYKPCFVCQDKSSGYHYGVSACEGCKG

SEQ ID NO: 152 - protein, 101 aa, Homo sapiens
VARFNDLRFVGRSGRGKSFTLTITVFTNPPQVATYHRAIKITVDGPREPRNRTEKHSTMPDSPVDVKTQSRLTPPTMPPPPTTQGAPRTSSFTPTTLTNGT

SEQ ID NO: 153 - protein, 55 aa, Homo sapiens
MALNSEALSVVSEDQSLFECAYGTPHLAKTEMTASSSSDYGQTSKMSPRVPQQDW

SEQ ID NO: 154 - protein, 10 aa, Homo sapiens
KLVVVGACGV

SEQ ID NO: 155 - protein, 9 aa, Homo sapiens
LVVVGACGV

SEQ ID NO: 156 - protein, 9 aa, Homo sapiens
VVGACGVGK

SEQ ID NO: 157 - protein, 10 aa, Homo sapiens
VVVGACGVGK

SEQ ID NO: 158 - protein, 9 aa, Homo sapiens
VVGADGVGK

SEQ ID NO: 159 - protein, 10 aa, Homo sapiens
VVVGADGVGK

SEQ ID NO: 160 - protein, 10 aa, Homo sapiens
KLVVVGADGV

SEQ ID NO: 161 - protein, 9 aa, Homo sapiens
LVVVGADGV

SEQ ID NO: 162 - protein, 10 aa, Homo sapiens
KLVVVGAVGV

SEQ ID NO: 163 - protein, 9 aa, Homo sapiens
LVVVGAVGV

SEQ ID NO: 164 - protein, 9 aa, Homo sapiens
VVGAVGVGK

SEQ ID NO: 165 - protein, 10 aa, Homo sapiens
ILDTAGHEEY

SEQ ID NO: 166 - protein, 10 aa, Homo sapiens
ILDTAGLEEY

SEQ ID NO: 167 - protein, 10 aa, Homo sapiens
LLDILDTAGL

SEQ ID NO: 168 - protein, 10 aa, Homo sapiens
ILDTAGKEEY

SEQ ID NO: 169 - protein, 10 aa, Homo sapiens
ILDTAGREEY

SEQ ID NO: 170 - protein, 9 aa, Homo sapiens
EYMANGSLL

SEQ ID NO: 171 - protein, 9 aa, Homo sapiens
MANGSLLNY

SEQ ID NO: 172 - protein, 10 aa, Homo sapiens
MANGSLLNYL

SEQ ID NO: 173 - protein, 9 aa, Homo sapiens
SLLNYLREM

SEQ ID NO: 174 - protein, 9 aa, Homo sapiens
YMANGSLLN

SEQ ID NO: 175 - protein, 10 aa, Homo sapiens
YMANGSLLNY

SEQ ID NO: 176 - protein, 11 aa, Homo sapiens
IIRNRGENSCK

SEQ ID NO: 177 - protein, 10 aa, Homo sapiens
CLTSTVQLIM

SEQ ID NO: 178 - protein, 9 aa, Homo sapiens
IMQLMPFGC

SEQ ID NO: 179 - protein, 10 aa, Homo sapiens
IMQLMPFGCL

SEQ ID NO: 180 - protein, 9 aa, Homo sapiens
LIMQLMPFG

SEQ ID NO: 181 - protein, 10 aa, Homo sapiens
LIMQLMPFGC

SEQ ID NO: 182 - protein, 9 aa, Homo sapiens
LTSTVQLIM

SEQ ID NO: 183 - protein, 9 aa, Homo sapiens
MQLMPFGCL

SEQ ID NO: 184 - protein, 10 aa, Homo sapiens
MQLMPFGCLL

SEQ ID NO: 185 - protein, 9 aa, Homo sapiens
QLIMQLMPF

SEQ ID NO: 186 - protein, 10 aa, Homo sapiens
QLIMQLMPFG

SEQ ID NO: 187 - protein, 9 aa, Homo sapiens
STVQLIMQL

SEQ ID NO: 188 - protein, 10 aa, Homo sapiens
VQLIMQLMPF

SEQ ID NO: 189 - protein, 9 aa, Homo sapiens
GQYGKVYEG

SEQ ID NO: 190 - protein, 10 aa, Homo sapiens
GQYGKVYEGV

SEQ ID NO: 191 - protein, 9 aa, Homo sapiens
KLGGGQYGK

SEQ ID NO: 192 - protein, 10 aa, Homo sapiens
KLGGGQYGKV

SEQ ID NO: 193 - protein, 9 aa, Homo sapiens
KVYEGVWKK

SEQ ID NO: 194 - protein, 10 aa, Homo sapiens
KVYEGVWKKY

SEQ ID NO: 195 - protein, 9 aa, Homo sapiens
QYGKVYEGV

SEQ ID NO: 196 - protein, 10 aa, Homo sapiens
QYGKVYEGVW

SEQ ID NO: 197 - protein, 9 aa, Homo sapiens
GQYGVVYEG

SEQ ID NO: 198 - protein, 10 aa, Homo sapiens
GQYGVVYEGV

SEQ ID NO: 199 - protein, 9 aa, Homo sapiens
KLGGGQYGV

SEQ ID NO: 200 - protein, 10 aa, Homo sapiens
KLGGGQYGVV

SEQ ID NO: 201 - protein, 9 aa, Homo sapiens
QYGVVYEGV

SEQ ID NO: 202 - protein, 10 aa, Homo sapiens
QYGVVYEGVW

SEQ ID NO: 203 - protein, 9 aa, Homo sapiens
VVYEGVWKK

SEQ ID NO: 204 - protein, 10 aa, Homo sapiens
VVYEGVWKKY

SEQ ID NO: 205 - protein, 10 aa, Homo sapiens
ATQISSATEY

SEQ ID NO: 206 - protein, 10 aa, Homo sapiens
ISSATEYLEK

SEQ ID NO: 207 - protein, 9 aa, Homo sapiens
SSATEYLEK

SEQ ID NO: 208 - protein, 10 aa, Homo sapiens
TQISSATEYL

SEQ ID NO: 209 - protein, 10 aa, Homo sapiens
YMATQISSAT

SEQ ID NO: 210 - protein, 10 aa, Homo sapiens
FYIIIEFMTY

SEQ ID NO: 211 - protein, 10 aa, Homo sapiens
IIEFMTYGNL

SEQ ID NO: 212 - protein, 9 aa, Homo sapiens
IIIEFMTYG

SEQ ID NO: 213 - protein, 10 aa, Homo sapiens
IIIEFMTYGN

SEQ ID NO: 214 - protein, 10 aa, Homo sapiens
YIIIEFMTYG

SEQ ID NO: 215 - protein, 10 aa, Homo sapiens
GQHGEVYEGV

SEQ ID NO: 216 - protein, 10 aa, Homo sapiens
KLGGGQHGEV

SEQ ID NO: 217 - protein, 9 aa, Homo sapiens
KIADFGMAR

SEQ ID NO: 218 - protein, 10 aa, Homo sapiens
RVAKIADFGM

SEQ ID NO: 219 - protein, 10 aa, Homo sapiens
FILMELMAGG

SEQ ID NO: 220 - protein, 9 aa, Homo sapiens
ILMELMAGG

SEQ ID NO: 221 - protein, 10 aa, Homo sapiens
ILMELMAGGD

SEQ ID NO: 222 - protein, 10 aa, Homo sapiens
LMELMAGGDL

SEQ ID NO: 223 - protein, 9 aa, Homo sapiens
LPRFILMEL

SEQ ID NO: 224 - protein, 10 aa, Homo sapiens
LPRFILMELM

SEQ ID NO: 225 - protein, 10 aa, Homo sapiens
LQSLPRFILM

SEQ ID NO: 226 - protein, 10 aa, Homo sapiens
SLPRFILMEL

SEQ ID NO: 227 - protein, 9 aa, Homo sapiens
LATEKSRWS

SEQ ID NO: 228 - protein, 10 aa, Homo sapiens
LATEKSRWSG

SEQ ID NO: 229 - protein, 11 aa, Homo sapiens
GPPPADLCHAL

SEQ ID NO: 230 - protein, 9 aa, Homo sapiens
GLLLEMLDA

SEQ ID NO: 231 - protein, 9 aa, Homo sapiens
LYGLLLEML

SEQ ID NO: 232 - protein, 9 aa, Homo sapiens
NVVPLYGLL

SEQ ID NO: 233 - protein, 9 aa, Homo sapiens
PLYGLLLEM

SEQ ID NO: 234 - protein, 10 aa, Homo sapiens
PLYGLLLEML

SEQ ID NO: 235 - protein, 10 aa, Homo sapiens
VPLYGLLLEM

SEQ ID NO: 236 - protein, 9 aa, Homo sapiens
VVPLYGLLL

SEQ ID NO: 237 - protein, 9 aa, Homo sapiens
FLPSTLKSL

SEQ ID NO: 238 - protein, 9 aa, Homo sapiens
GVYTFLPST

SEQ ID NO: 239 - protein, 10 aa, Homo sapiens
GVYTFLPSTL

SEQ ID NO: 240 - protein, 10 aa, Homo sapiens
TFLPSTLKSL

SEQ ID NO: 241 - protein, 9 aa, Homo sapiens
VYTFLPSTL

SEQ ID NO: 242 - protein, 9 aa, Homo sapiens
YTFLPSTLK

SEQ ID NO: 243 - protein, 9 aa, Homo sapiens
NVVPLCDLL

SEQ ID NO: 244 - protein, 10 aa, Homo sapiens
NVVPLCDLLL

SEQ ID NO: 245 - protein, 9 aa, Homo sapiens
PLCDLLLEM

SEQ ID NO: 246 - protein, 10 aa, Homo sapiens
PLCDLLLEML

SEQ ID NO: 247 - protein, 10 aa, Homo sapiens
VPLCDLLLEM

SEQ ID NO: 248 - protein, 9 aa, Homo sapiens
VVPLCDLLL

SEQ ID NO: 249 - protein, 9 aa, Homo sapiens
NVVPLNDLL

SEQ ID NO: 250 - protein, 10 aa, Homo sapiens
NVVPLNDLLL

SEQ ID NO: 251 - protein, 9 aa, Homo sapiens
PLNDLLLEM

SEQ ID NO: 252 - protein, 10 aa, Homo sapiens
PLNDLLLEML

SEQ ID NO: 253 - protein, 10 aa, Homo sapiens
VPLNDLLLEM

SEQ ID NO: 254 - protein, 9 aa, Homo sapiens
NVVPLSDLL

SEQ ID NO: 255 - protein, 10 aa, Homo sapiens
NVVPLSDLLL

SEQ ID NO: 256 - protein, 9 aa, Homo sapiens
PLSDLLLEM

SEQ ID NO: 257 - protein, 10 aa, Homo sapiens
PLSDLLLEML

SEQ ID NO: 258 - protein, 10 aa, Homo sapiens
VPLSDLLLEM

SEQ ID NO: 259 - protein, 9 aa, Homo sapiens
VVPLSDLLL

SEQ ID NO: 260 - protein, 10 aa, Homo sapiens
VLERCPHRPI

SEQ ID NO: 261 - protein, 9 aa, Homo sapiens
YTLDVLERC

SEQ ID NO: 262 - protein, 9 aa, Homo sapiens
FQNGKMALS

SEQ ID NO: 263 - protein, 10 aa, Homo sapiens
VMAGLGSPYV

SEQ ID NO: 264 - protein, 9 aa, Homo sapiens
KPIIIGHHA

SEQ ID NO: 265 - protein, 9 aa, Homo sapiens
KPIIIGCHA

SEQ ID NO: 266 - protein, 9 aa, Homo sapiens
KPIIIGGHA

SEQ ID NO: 267 - protein, 9 aa, Homo sapiens
KPIIIGSHA

SEQ ID NO: 268 - protein, 10 aa, Homo sapiens
IIEYCCYGDL

SEQ ID NO: 269 - protein, 10 aa, Homo sapiens
TIGGPTLVII

SEQ ID NO: 270 - protein, 9 aa, Homo sapiens
VIIEYCCYG

SEQ ID NO: 271 - protein, 10 aa, Homo sapiens
HMNIANLLGA

SEQ ID NO: 272 - protein, 10 aa, Homo sapiens
IANLLGACTI

SEQ ID NO: 273 - protein, 9 aa, Homo sapiens
MNIANLLGA

SEQ ID NO: 274 - protein, 9 aa, Homo sapiens
YLGNHMNIA

SEQ ID NO: 275 - protein, 10 aa, Homo sapiens
YLGNHMNIAN

SEQ ID NO: 276 - protein, 9 aa, Homo sapiens
VLHESNSPY

SEQ ID NO: 277 - protein, 10 aa, Homo sapiens
VLHESNSPYI

SEQ ID NO: 278 - protein, 10 aa, Homo sapiens
LQVLHECNSL

SEQ ID NO: 279 - protein, 10 aa, Homo sapiens
LYIVGFYGAF

SEQ ID NO: 280 - protein, 9 aa, Homo sapiens
NSLYIVGFY

SEQ ID NO: 281 - protein, 9 aa, Homo sapiens
QVLHECNSL

SEQ ID NO: 282 - protein, 9 aa, Homo sapiens
SLYIVGFYG

SEQ ID NO: 283 - protein, 10 aa, Homo sapiens
SLYIVGFYGA

SEQ ID NO: 284 - protein, 9 aa, Homo sapiens
VLHECNSLY

SEQ ID NO: 285 - protein, 10 aa, Homo sapiens
VLHECNSLYI

SEQ ID NO: 286 - protein, 10 aa, Homo sapiens
FYQQQQQSDL

SEQ ID NO: 287 - protein, 10 aa, Homo sapiens
QQQSDLQPPA

SEQ ID NO: 288 - protein, 9 aa, Homo sapiens
QQSDLQPPA

SEQ ID NO: 289 - protein, 9 aa, Homo sapiens
YQQQQQSDL

SEQ ID NO: 290 - protein, 9 aa, Homo sapiens
FELLSTPPL

SEQ ID NO: 291 - protein, 10 aa, Homo sapiens
LLSTPPLSPS

SEQ ID NO: 292 - protein, 9 aa, Homo sapiens
FELLPIPPL

SEQ ID NO: 293 - protein, 10 aa, Homo sapiens
IWKKFELLPI

SEQ ID NO: 294 - protein, 10 aa, Homo sapiens
LLPIPPLSPS

SEQ ID NO: 295 - protein, 9 aa, Homo sapiens
LPIPPLSPS

SEQ ID NO: 296 - protein, 10 aa, Homo sapiens
IIEYCFYGDL

SEQ ID NO: 297 - protein, 9 aa, Homo sapiens
IIIEYCFYG

SEQ ID NO: 298 - protein, 9 aa, Homo sapiens
IYIIIEYCF

SEQ ID NO: 299 - protein, 10 aa, Homo sapiens
IYIIIEYCFY

SEQ ID NO: 300 - protein, 10 aa, Homo sapiens
YIIIEYCFYG

SEQ ID NO: 301 - protein, 10 aa, Homo sapiens
KITEQEKDFL

SEQ ID NO: 302 - protein, 11 aa, Homo sapiens
STRDPLSEITK

SEQ ID NO: 303 - protein, 8 aa, Homo sapiens
DPLSEITK

SEQ ID NO: 304 - protein, 10 aa, Homo sapiens
LPLKFRDAET

SEQ ID NO: 305 - protein, 10 aa, Homo sapiens
QTGVMICAYL

SEQ ID NO: 306 - protein, 10 aa, Homo sapiens
AFSGEYIPTV

SEQ ID NO: 307 - protein, 9 aa, Homo sapiens
SMNRRPILT

SEQ ID NO: 308 - protein, 10 aa, Homo sapiens
YMCNSSCMGS

SEQ ID NO: 309 - protein, 9 aa, Homo sapiens
GMNQRPILT

SEQ ID NO: 310 - protein, 9 aa, Homo sapiens
GMNWRPILT

SEQ ID NO: 311 - protein, 10 aa, Homo sapiens
LLGRNSFEVC

SEQ ID NO: 312 - protein, 9 aa, Homo sapiens
ALKYIFVAV

SEQ ID NO: 313 - protein, 10 aa, Homo sapiens
ALKYIFVAVR

SEQ ID NO: 314 - protein, 9 aa, Homo sapiens
AVRAICVMK

SEQ ID NO: 315 - protein, 10 aa, Homo sapiens
AVRAICVMKS

SEQ ID NO: 316 - protein, 9 aa, Homo sapiens
CVEKKTALK

SEQ ID NO: 317 - protein, 10 aa, Homo sapiens
CVEKKTALKY

SEQ ID NO: 318 - protein, 9 aa, Homo sapiens
CVMKSFLIF

SEQ ID NO: 319 - protein, 10 aa, Homo sapiens
CVMKSFLIFR

SEQ ID NO: 320 - protein, 9 aa, Homo sapiens
FLIFRRWKS

SEQ ID NO: 321 - protein, 10 aa, Homo sapiens
FRRWKSHSPL

SEQ ID NO: 322 - protein, 9 aa, Homo sapiens
FVAVRAICV

SEQ ID NO: 323 - protein, 10 aa, Homo sapiens
FVAVRAICVM

SEQ ID NO: 324 - protein, 9 aa, Homo sapiens
IQLHLSHPI

SEQ ID NO: 325 - protein, 10 aa, Homo sapiens
KSFLIFRRWK

SEQ ID NO: 326 - protein, 9 aa, Homo sapiens
KTALKYIFV

SEQ ID NO: 327 - protein, 10 aa, Homo sapiens
KYIFVAVRAI

SEQ ID NO: 328 - protein, 10 aa, Homo sapiens
RWKSHSPLQI

SEQ ID NO: 329 - protein, 10 aa, Homo sapiens
TALKYIFVAV

SEQ ID NO: 330 - protein, 10 aa, Homo sapiens
VAVRAICVMK

SEQ ID NO: 331 - protein, 9 aa, Homo sapiens
VMKSFLIFR

SEQ ID NO: 332 - protein, 10 aa, Homo sapiens
VMKSFLIFRR

SEQ ID NO: 333 - protein, 9 aa, Homo sapiens
YIFVAVRAI

SEQ ID NO: 334 - protein, 9 aa, Homo sapiens
ALFFFFFET

SEQ ID NO: 335 - protein, 10 aa, Homo sapiens
ALFFFFFETK

SEQ ID NO: 336 - protein, 10 aa, Homo sapiens
AQAGVQWRSL

SEQ ID NO: 337 - protein, 10 aa, Homo sapiens
CLANFCIFNR

SEQ ID NO: 338 - protein, 10 aa, Homo sapiens
CLSFLSSWDY

SEQ ID NO: 339 - protein, 9 aa, Homo sapiens
FFETKSCSV

SEQ ID NO: 340 - protein, 10 aa, Homo sapiens
FFFETKSCSV

SEQ ID NO: 341 - protein, 10 aa, Homo sapiens
FKLFSCLSFL

SEQ ID NO: 342 - protein, 10 aa, Homo sapiens
FLSSWDYRRM

SEQ ID NO: 343 - protein, 10 aa, Homo sapiens
GFKLFSCLSF

SEQ ID NO: 344 - protein, 9 aa, Homo sapiens
KLFSCLSFL

SEQ ID NO: 345 - protein, 10 aa, Homo sapiens
KLFSCLSFLS

SEQ ID NO: 346 - protein, 10 aa, Homo sapiens
LALFFFFFET

SEQ ID NO: 347 - protein, 9 aa, Homo sapiens
LFFFFFETK

SEQ ID NO: 348 - protein, 9 aa, Homo sapiens
LSFLSSWDY

SEQ ID NO: 349 - protein, 10 aa, Homo sapiens
LSFLSSWDYR

SEQ ID NO: 350 - protein, 9 aa, Homo sapiens
RMPPCLANF

SEQ ID NO: 351 - protein, 10 aa, Homo sapiens
RRMPPCLANF

SEQ ID NO: 352 - protein, 10 aa, Homo sapiens
SLQPPPPGFK

SEQ ID NO: 353 - protein, 9 aa, Homo sapiens
VQWRSLGSL

SEQ ID NO: 354 - protein, 9 aa, Homo sapiens
FFFSVIFST

SEQ ID NO: 355 - protein, 10 aa, Homo sapiens
MSVCFFFFSV

SEQ ID NO: 356 - protein, 9 aa, Homo sapiens
SVCFFFFSV

SEQ ID NO: 357 - protein, 10 aa, Homo sapiens
SVCFFFFSVI

SEQ ID NO: 358 - protein, 10 aa, Homo sapiens
FFCYILNTMF

SEQ ID NO: 359 - protein, 10 aa, Homo sapiens
MSVCFFFFCY

SEQ ID NO: 360 - protein, 10 aa, Homo sapiens
SVCFFFFCYI

SEQ ID NO: 361 - protein, 9 aa, Homo sapiens
KQWSSVTSL

SEQ ID NO: 362 - protein, 9 aa, Homo sapiens
VLWMPTSTV

SEQ ID NO: 363 - protein, 9 aa, Homo sapiens
ILIRKAMTV

SEQ ID NO: 364 - protein, 8 aa, Homo sapiens
RMKVPLMK

SEQ ID NO: 365 - protein, 9 aa, Homo sapiens
TLKKKPRDI

SEQ ID NO: 366 - protein, 10 aa, Homo sapiens
LIEGFEALLK

SEQ ID NO: 367 - protein, 9 aa, Homo sapiens
QQLKWTPHI

SEQ ID NO: 368 - protein, 10 aa, Homo sapiens
QLKWTPHILK

SEQ ID NO: 369 - protein, 10 aa, Homo sapiens
IVSEKNQQLK

SEQ ID NO: 370 - protein, 11 aa, Homo sapiens
NQQLKWTPHIL

SEQ ID NO: 371 - protein, 10 aa, Homo sapiens
NQQLKWTPHI

SEQ ID NO: 372 - protein, 9 aa, Homo sapiens
QLKWTPHIL

SEQ ID NO: 373 - protein, 10 aa, Homo sapiens
GPPRPPRAAC

SEQ ID NO: 374 - protein, 9 aa, Homo sapiens
PPRPPRAAC

SEQ ID NO: 375 - protein, 9 aa, Homo sapiens
SLRRKYLRV

SEQ ID NO: 376 - protein, 10 aa, Homo sapiens
SAACHRRGCV

SEQ ID NO: 377 - protein, 10 aa, Homo sapiens
ALWECSLPQA

SEQ ID NO: 378 - protein, 9 aa, Homo sapiens
CLIVSRTLL

SEQ ID NO: 379 - protein, 10 aa, Homo sapiens
CLIVSRTLLL

SEQ ID NO: 380 - protein, 9 aa, Homo sapiens
FLLALWECS

SEQ ID NO: 381 - protein, 10 aa, Homo sapiens
FLLALWECSL

SEQ ID NO: 382 - protein, 9 aa, Homo sapiens
IVSRTLLLV

SEQ ID NO: 383 - protein, 9 aa, Homo sapiens
LIVSRTLLL

SEQ ID NO: 384 - protein, 10 aa, Homo sapiens
LIVSRTLLLV

SEQ ID NO: 385 - protein, 9 aa, Homo sapiens
LLALWECSL

SEQ ID NO: 386 - protein, 9 aa, Homo sapiens
LPQARLCLI

SEQ ID NO: 387 - protein, 10 aa, Homo sapiens
LPQARLCLIV

SEQ ID NO: 388 - protein, 10 aa, Homo sapiens
NVRDKKRATF

SEQ ID NO: 389 - protein, 10 aa, Homo sapiens
SLPQARLCLI

SEQ ID NO: 390 - protein, 9 aa, Homo sapiens
FFCWILSCK

SEQ ID NO: 391 - protein, 10 aa, Homo sapiens
FFFCWILSCK

SEQ ID NO: 392 - protein, 9 aa, Homo sapiens
ITAFFFCWI

SEQ ID NO: 393 - protein, 10 aa, Homo sapiens
LYITAFFFCW

SEQ ID NO: 394 - protein, 10 aa, Homo sapiens
YITAFFFCWI

SEQ ID NO: 395 - protein, 9 aa, Homo sapiens
ITAFFLLDI

SEQ ID NO: 396 - protein, 9 aa, Homo sapiens
LLYITAFFL

SEQ ID NO: 397 - protein, 10 aa, Homo sapiens
LLYITAFFLL

SEQ ID NO: 398 - protein, 9 aa, Homo sapiens
LYITAFFLL

SEQ ID NO: 399 - protein, 10 aa, Homo sapiens
LYITAFFLLD

SEQ ID NO: 400 - protein, 10 aa, Homo sapiens
YITAFFLLDI

SEQ ID NO: 401 - protein, 9 aa, Homo sapiens
GSNEVTTRY

SEQ ID NO: 402 - protein, 9 aa, Homo sapiens
MPKDVNIQV

SEQ ID NO: 403 - protein, 10 aa, Homo sapiens
TGSNEVTTRY

SEQ ID NO: 404 - protein, 10 aa, Homo sapiens
KKLMLLRLNL

SEQ ID NO: 405 - protein, 9 aa, Homo sapiens
KLMLLRLNL

SEQ ID NO: 406 - protein, 10 aa, Homo sapiens
KLMLLRLNLR

SEQ ID NO: 407 - protein, 9 aa, Homo sapiens
LLRLNLRKM

SEQ ID NO: 408 - protein, 8 aa, Homo sapiens
LMLLRLNL

SEQ ID NO: 409 - protein, 10 aa, Homo sapiens
LMLLRLNLRK

SEQ ID NO: 410 - protein, 10 aa, Homo sapiens
LNLRKMCGPF

SEQ ID NO: 411 - protein, 9 aa, Homo sapiens
MLLRLNLRK

SEQ ID NO: 412 - protein, 10 aa, Homo sapiens
MLLRLNLRKM

SEQ ID NO: 413 - protein, 9 aa, Homo sapiens
NLRKMCGPF

SEQ ID NO: 414 - protein, 10 aa, Homo sapiens
NYCQKKLMLL

SEQ ID NO: 415 - protein, 9 aa, Homo sapiens
YCQKKLMLL

SEQ ID NO: 416 - protein, 10 aa, Homo sapiens
FKKKTYTCAI

SEQ ID NO: 417 - protein, 10 aa, Homo sapiens
ITTVKATETK

SEQ ID NO: 418 - protein, 9 aa, Homo sapiens
KSKKKETFK

SEQ ID NO: 419 - protein, 10 aa, Homo sapiens
KSKKKETFKK

SEQ ID NO: 420 - protein, 10 aa, Homo sapiens
KTYTCAITTV

SEQ ID NO: 421 - protein, 9 aa, Homo sapiens
TFKKKTYTC

SEQ ID NO: 422 - protein, 9 aa, Homo sapiens
TYTCAITTV

SEQ ID NO: 423 - protein, 10 aa, Homo sapiens
TYTCAITTVK

SEQ ID NO: 424 - protein, 9 aa, Homo sapiens
YTCAITTVK

SEQ ID NO: 425 - protein, 9 aa, Homo sapiens
TAKSKKRNL

SEQ ID NO: 426 - protein, 9 aa, Homo sapiens
FALCGFWQI

SEQ ID NO: 427 - protein, 8 aa, Homo sapiens
LKKLRSPL

SEQ ID NO: 428 - protein, 8 aa, Homo sapiens
SLKKVPAL

SEQ ID NO: 429 - protein, 10 aa, Homo sapiens
RKISNWSLKK

SEQ ID NO: 430 - protein, 11 aa, Homo sapiens
VPALKKLRSPL

SEQ ID NO: 431 - protein, 10 aa, Homo sapiens
KRVNTAWKTK

SEQ ID NO: 432 - protein, 10 aa, Homo sapiens
MTKKKRVNTA

SEQ ID NO: 433 - protein, 9 aa, Homo sapiens
RVNTAWKTK

SEQ ID NO: 434 - protein, 10 aa, Homo sapiens
RVNTAWKTKK

SEQ ID NO: 435 - protein, 9 aa, Homo sapiens
TKKKRVNTA

SEQ ID NO: 436 - protein, 10 aa, Homo sapiens
WKTKKTSFSL

SEQ ID NO: 437 - protein, 9 aa, Homo sapiens
ALMSAMTTS

SEQ ID NO: 438 - protein, 9 aa, Homo sapiens
AMTTSSSQK

SEQ ID NO: 439 - protein, 10 aa, Homo sapiens
AMTTSSSQKN

SEQ ID NO: 440 - protein, 9 aa, Homo sapiens
CIMKEKKSL

SEQ ID NO: 441 - protein, 10 aa, Homo sapiens
CIMKEKKSLV

SEQ ID NO: 442 - protein, 8 aa, Homo sapiens
IMKEKKSL

SEQ ID NO: 443 - protein, 9 aa, Homo sapiens
IMKEKKSLV

SEQ ID NO: 444 - protein, 10 aa, Homo sapiens
KSLVRLSSCV

SEQ ID NO: 445 - protein, 10 aa, Homo sapiens
LVRLSSCVPV

SEQ ID NO: 446 - protein, 9 aa, Homo sapiens
RLSSCVPVA

SEQ ID NO: 447 - protein, 10 aa, Homo sapiens
RLSSCVPVAL

SEQ ID NO: 448 - protein, 10 aa, Homo sapiens
SAMTTSSSQK

SEQ ID NO: 449 - protein, 9 aa, Homo sapiens
SLVRLSSCV

SEQ ID NO: 450 - protein, 9 aa, Homo sapiens
VPVALMSAM

SEQ ID NO: 451 - protein, 10 aa, Homo sapiens
VRLSSCVPVA

SEQ ID NO: 452 - protein, 9 aa, Homo sapiens
KMRKKYAQK

SEQ ID NO: 453 - protein, 9 aa, Homo sapiens
FVMSDTTYK

SEQ ID NO: 454 - protein, 10 aa, Homo sapiens
FVMSDTTYKI

SEQ ID NO: 455 - protein, 9 aa, Homo sapiens
KTFEKKGEK

SEQ ID NO: 456 - protein, 10 aa, Homo sapiens
LFVMSDTTYK

SEQ ID NO: 457 - protein, 9 aa, Homo sapiens
MSDTTYKIY

SEQ ID NO: 458 - protein, 9 aa, Homo sapiens
VMSDTTYKI

SEQ ID NO: 459 - protein, 10 aa, Homo sapiens
VMSDTTYKIY

SEQ ID NO: 460 - protein, 10 aa, Homo sapiens
YLTKWPKFFL

SEQ ID NO: 461 - protein, 9 aa, Homo sapiens
FLQERNLPP

SEQ ID NO: 462 - protein, 9 aa, Homo sapiens
FRRPHSCLA

SEQ ID NO: 463 - protein, 9 aa, Homo sapiens
LIVLRVVRL

SEQ ID NO: 464 - protein, 9 aa, Homo sapiens
LLSVHLIVL

SEQ ID NO: 465 - protein, 9 aa, Homo sapiens
EVKHLHHLL

SEQ ID NO: 466 - protein, 10 aa, Homo sapiens
HLHHLLKQLK

SEQ ID NO: 467 - protein, 9 aa, Homo sapiens
HLLLKRERV

SEQ ID NO: 468 - protein, 9 aa, Homo sapiens
KIKHLLLKR

SEQ ID NO: 469 - protein, 9 aa, Homo sapiens
KPSEKYLKI

SEQ ID NO: 470 - protein, 9 aa, Homo sapiens
KYLKIKHLL

SEQ ID NO: 471 - protein, 10 aa, Homo sapiens
KYLKIKHLLL

SEQ ID NO: 472 - protein, 10 aa, Homo sapiens
LLKQLKPSEK

SEQ ID NO: 473 - protein, 9 aa, Homo sapiens
LLKRERVDL

SEQ ID NO: 474 - protein, 10 aa, Homo sapiens
LLLKRERVDL

SEQ ID NO: 475 - protein, 10 aa, Homo sapiens
QLKPSEKYLK

SEQ ID NO: 476 - protein, 9 aa, Homo sapiens
YLKIKHLLL

SEQ ID NO: 477 - protein, 10 aa, Homo sapiens
YLKIKHLLLK

SEQ ID NO: 478 - protein, 9 aa, Homo sapiens
ILPRKVLQM

SEQ ID NO: 479 - protein, 9 aa, Homo sapiens
KVLQMDFLV

SEQ ID NO: 480 - protein, 10 aa, Homo sapiens
LPRKVLQMDF

SEQ ID NO: 481 - protein, 10 aa, Homo sapiens
LQMDFLVHPA

SEQ ID NO: 482 - protein, 9 aa, Homo sapiens
QMDFLVHPA

SEQ ID NO: 483 - protein, 10 aa, Homo sapiens
YILPRKVLQM

SEQ ID NO: 484 - protein, 10 aa, Homo sapiens
APSPASRLQC

SEQ ID NO: 485 - protein, 10 aa, Homo sapiens
HPLAPMPSKT

SEQ ID NO: 486 - protein, 9 aa, Homo sapiens
ILPLPQLLL

SEQ ID NO: 487 - protein, 10 aa, Homo sapiens
LPTQTRGCIL

SEQ ID NO: 488 - protein, 10 aa, Homo sapiens
RISCLPTQTR

SEQ ID NO: 489 - protein, 9 aa, Homo sapiens
SLAETVSLH

SEQ ID NO: 490 - protein, 9 aa, Homo sapiens
TPRSSSSSS

SEQ ID NO: 491 - protein, 10 aa, Homo sapiens
TPRSSSSSSS

SEQ ID NO: 492 - protein, 9 aa, Homo sapiens
ALPPVLLSL

SEQ ID NO: 493 - protein, 10 aa, Homo sapiens
ALPPVLLSLA

SEQ ID NO: 494 - protein, 9 aa, Homo sapiens
ALPRPLLAL

SEQ ID NO: 495 - protein, 9 aa, Homo sapiens
ASRTASCIL

SEQ ID NO: 496 - protein, 10 aa, Homo sapiens
EALPRPLLAL

SEQ ID NO: 497 - protein, 9 aa, Homo sapiens
HLGHPVASR

SEQ ID NO: 498 - protein, 9 aa, Homo sapiens
HPVASRTAS

SEQ ID NO: 499 - protein, 10 aa, Homo sapiens
HPVASRTASC

SEQ ID NO: 500 - protein, 10 aa, Homo sapiens
IIQLSLLSLL

SEQ ID NO: 501 - protein, 9 aa, Homo sapiens
IQLSLLSLL

SEQ ID NO: 502 - protein, 10 aa, Homo sapiens
IQLSLLSLLI

SEQ ID NO: 503 - protein, 9 aa, Homo sapiens
LLALPPVLL

SEQ ID NO: 504 - protein, 9 aa, Homo sapiens
LLIPPLTCL

SEQ ID NO: 505 - protein, 10 aa, Homo sapiens
LLIPPLTCLA

SEQ ID NO: 506 - protein, 9 aa, Homo sapiens
LLSLLIPPL

SEQ ID NO: 507 - protein, 10 aa, Homo sapiens
LLSLLIPPLT

SEQ ID NO: 508 - protein, 10 aa, Homo sapiens
LPRPLLALPP

SEQ ID NO: 509 - protein, 9 aa, Homo sapiens
QLSLLSLLI

SEQ ID NO: 510 - protein, 9 aa, Homo sapiens
RLLQCQATR

SEQ ID NO: 511 - protein, 9 aa, Homo sapiens
RPLLALPPV

SEQ ID NO: 512 - protein, 10 aa, Homo sapiens
RPLLALPPVL

SEQ ID NO: 513 - protein, 9 aa, Homo sapiens
SLAQDHSRL

SEQ ID NO: 514 - protein, 10 aa, Homo sapiens
SLAQDHSRLL

SEQ ID NO: 515 - protein, 10 aa, Homo sapiens
SLLIPPLTCL

SEQ ID NO: 516 - protein, 9 aa, Homo sapiens
SLLSLLIPP

SEQ ID NO: 517 - protein, 10 aa, Homo sapiens
SLLSLLIPPL

SEQ ID NO: 518 - protein, 10 aa, Homo sapiens
AAATSAASTL

SEQ ID NO: 519 - protein, 10 aa, Homo sapiens
AAIPASTSAV

SEQ ID NO: 520 - protein, 9 aa, Homo sapiens
AIPASTSAV

SEQ ID NO: 521 - protein, 10 aa, Homo sapiens
ALPAGCVSSA

SEQ ID NO: 522 - protein, 10 aa, Homo sapiens
APLLTATGSV

SEQ ID NO: 523 - protein, 9 aa, Homo sapiens
APVLSASIL

SEQ ID NO: 524 - protein, 9 aa, Homo sapiens
ATLLPATTV

SEQ ID NO: 525 - protein, 10 aa, Homo sapiens
ATVSTTTAPV

SEQ ID NO: 526 - protein, 10 aa, Homo sapiens
AVPANCLFPA

SEQ ID NO: 527 - protein, 10 aa, Homo sapiens
CLFPAALPST

SEQ ID NO: 528 - protein, 9 aa, Homo sapiens
CPTFVSAAA

SEQ ID NO: 529 - protein, 9 aa, Homo sapiens
FPAALPSTA

SEQ ID NO: 530 - protein, 10 aa, Homo sapiens
FPAALPSTAG

SEQ ID NO: 531 - protein, 9 aa, Homo sapiens
GAECHGRPL

SEQ ID NO: 532 - protein, 9 aa, Homo sapiens
GAISRFIWV

SEQ ID NO: 533 - protein, 10 aa, Homo sapiens
ILPAAIPAST

SEQ ID NO: 534 - protein, 10 aa, Homo sapiens
IWVSGILSPL

SEQ ID NO: 535 - protein, 10 aa, Homo sapiens
LLTATGSVSL

SEQ ID NO: 536 - protein, 9 aa, Homo sapiens
LLYTADSSL

SEQ ID NO: 537 - protein, 9 aa, Homo sapiens
LPAAATSAA

SEQ ID NO: 538 - protein, 10 aa, Homo sapiens
LPAAATSAAS

SEQ ID NO: 539 - protein, 9 aa, Homo sapiens
LPAAIPAST

SEQ ID NO: 540 - protein, 9 aa, Homo sapiens
LPAGCVSSA

SEQ ID NO: 541 - protein, 10 aa, Homo sapiens
LPAGCVSSAP

SEQ ID NO: 542 - protein, 9 aa, Homo sapiens
LYTADSSLW

SEQ ID NO: 543 - protein, 9 aa, Homo sapiens
QPAKSSPSA

SEQ ID NO: 544 - protein, 10 aa, Homo sapiens
QPAKSSPSAA

SEQ ID NO: 545 - protein, 9 aa, Homo sapiens
RFIWVSGIL

SEQ ID NO: 546 - protein, 9 aa, Homo sapiens
RPQRVWSTG

SEQ ID NO: 547 - protein, 10 aa, Homo sapiens
RVWSTGPDSI

SEQ ID NO: 548 - protein, 9 aa, Homo sapiens
SAVPGSIPL

SEQ ID NO: 549 - protein, 9 aa, Homo sapiens
SILPAAIPA

SEQ ID NO: 550 - protein, 9 aa, Homo sapiens
SLPAAATSA

SEQ ID NO: 551 - protein, 10 aa, Homo sapiens
SLPAAATSAA

SEQ ID NO: 552 - protein, 9 aa, Homo sapiens
SLWTTRPQR

SEQ ID NO: 553 - protein, 10 aa, Homo sapiens
SLWTTRPQRV

SEQ ID NO: 554 - protein, 9 aa, Homo sapiens
SPSAAAATL

SEQ ID NO: 555 - protein, 10 aa, Homo sapiens
SPSAAAATLL

SEQ ID NO: 556 - protein, 10 aa, Homo sapiens
TLDALPAGCV

SEQ ID NO: 557 - protein, 9 aa, Homo sapiens
TVSTTTAPV

SEQ ID NO: 558 - protein, 9 aa, Homo sapiens
VLSASILPA

SEQ ID NO: 559 - protein, 10 aa, Homo sapiens
VLSASILPAA

SEQ ID NO: 560 - protein, 9 aa, Homo sapiens
VPANCLFPA

SEQ ID NO: 561 - protein, 10 aa, Homo sapiens
VPANCLFPAA

SEQ ID NO: 562 - protein, 9 aa, Homo sapiens
VPDPSCPTF

SEQ ID NO: 563 - protein, 9 aa, Homo sapiens
VPGSIPLPA

SEQ ID NO: 564 - protein, 10 aa, Homo sapiens
VPGSIPLPAV

SEQ ID NO: 565 - protein, 9 aa, Homo sapiens
WVSGILSPL

SEQ ID NO: 566 - protein, 10 aa, Homo sapiens
YTADSSLWTT

SEQ ID NO: 567 - protein, 9 aa, Homo sapiens
APAGMVNRA

SEQ ID NO: 568 - protein, 9 aa, Homo sapiens
ASLHRRSYL

SEQ ID NO: 569 - protein, 10 aa, Homo sapiens
ASLHRRSYLK

SEQ ID NO: 570 - protein, 10 aa, Homo sapiens
FLLMDNKAPA

SEQ ID NO: 571 - protein, 9 aa, Homo sapiens
HPRRSPSRL

SEQ ID NO: 572 - protein, 9 aa, Homo sapiens
HPSLHISSP

SEQ ID NO: 573 - protein, 10 aa, Homo sapiens
HRRSYLKIHL

SEQ ID NO: 574 - protein, 10 aa, Homo sapiens
HSRFLLMDNK

SEQ ID NO: 575 - protein, 10 aa, Homo sapiens
KLPIPSSASL

SEQ ID NO: 576 - protein, 10 aa, Homo sapiens
KVLTLSSSSH

SEQ ID NO: 577 - protein, 9 aa, Homo sapiens
LIHSRFLLM

SEQ ID NO: 578 - protein, 9 aa, Homo sapiens
LLMDNKAPA

SEQ ID NO: 579 - protein, 10 aa, Homo sapiens
LMDNKAPAGM

SEQ ID NO: 580 - protein, 9 aa, Homo sapiens
LPIPSSASL

SEQ ID NO: 581 - protein, 9 aa, Homo sapiens
MPNLRISSS

SEQ ID NO: 582 - protein, 10 aa, Homo sapiens
MPNLRISSSH

SEQ ID NO: 583 - protein, 10 aa, Homo sapiens
NLRISSSHSL

SEQ ID NO: 584 - protein, 10 aa, Homo sapiens
PPTHSHRLSL

SEQ ID NO: 585 - protein, 10 aa, Homo sapiens
RAGRRVPWAA

SEQ ID NO: 586 - protein, 10 aa, Homo sapiens
RARLHITTSK

SEQ ID NO: 587 - protein, 10 aa, Homo sapiens
RISSSHSLNH

SEQ ID NO: 588 - protein, 9 aa, Homo sapiens
RLHTPPSSR

SEQ ID NO: 589 - protein, 10 aa, Homo sapiens
RLHTPPSSRR

SEQ ID NO: 590 - protein, 9 aa, Homo sapiens
RLRILSPSL

SEQ ID NO: 591 - protein, 9 aa, Homo sapiens
RPLMPNLRI

SEQ ID NO: 592 - protein, 9 aa, Homo sapiens
RPRPLMPNL

SEQ ID NO: 593 - protein, 10 aa, Homo sapiens
SASLHRRSYL

SEQ ID NO: 594 - protein, 9 aa, Homo sapiens
SLHISSPRL

SEQ ID NO: 595 - protein, 9 aa, Homo sapiens
SLHRRSYLK

SEQ ID NO: 596 - protein, 10 aa, Homo sapiens
SLHRRSYLKI

SEQ ID NO: 597 - protein, 9 aa, Homo sapiens
SLIHSRFLL

SEQ ID NO: 598 - protein, 10 aa, Homo sapiens
SLIHSRFLLM

SEQ ID NO: 599 - protein, 9 aa, Homo sapiens
SLLTSSSNL

SEQ ID NO: 600 - protein, 10 aa, Homo sapiens
SLNHHSSSPL

SEQ ID NO: 601 - protein, 10 aa, Homo sapiens
SLSSPSKLPI

SEQ ID NO: 602 - protein, 9 aa, Homo sapiens
SPLSLHTPS

SEQ ID NO: 603 - protein, 10 aa, Homo sapiens
SPLSLHTPSS

SEQ ID NO: 604 - protein, 9 aa, Homo sapiens
SPPTHSHRL

SEQ ID NO: 605 - protein, 9 aa, Homo sapiens
SPRLHTPPS

SEQ ID NO: 606 - protein, 10 aa, Homo sapiens
SPRLHTPPSS

SEQ ID NO: 607 - protein, 10 aa, Homo sapiens
SPSLSSPSKL

SEQ ID NO: 608 - protein, 9 aa, Homo sapiens
SYLKIHLGL

SEQ ID NO: 609 - protein, 10 aa, Homo sapiens
TPSNHRPRPL

SEQ ID NO: 610 - protein, 10 aa, Homo sapiens
TPSSHPSLHI

SEQ ID NO: 611 - protein, 10 aa, Homo sapiens
CVPFWTGRIL

SEQ ID NO: 612 - protein, 11 aa, Homo sapiens
HCVPFWTGRIL

SEQ ID NO: 613 - protein, 9 aa, Homo sapiens
ILLPSAASV

SEQ ID NO: 614 - protein, 10 aa, Homo sapiens
ILLPSAASVC

SEQ ID NO: 615 - protein, 11 aa, Homo sapiens
LLPSAASVCPI

SEQ ID NO: 616 - protein, 10 aa, Homo sapiens
LPSAASVCPI

SEQ ID NO: 617 - protein, 8 aa, Homo sapiens
MRPHCVPF

SEQ ID NO: 618 - protein, 10 aa, Homo sapiens
RILLPSAASV

SEQ ID NO: 619 - protein, 9 aa, Homo sapiens
RMRPHCVPF

SEQ ID NO: 620 - protein, 10 aa, Homo sapiens
RMRPHCVPFW

SEQ ID NO: 621 - protein, 9 aa, Homo sapiens
RTNPTVRMR

SEQ ID NO: 622 - protein, 9 aa, Homo sapiens
SVCPIPFEA

SEQ ID NO: 623 - protein, 9 aa, Homo sapiens
TVRMRPHCV

SEQ ID NO: 624 - protein, 11 aa, Homo sapiens
TVRMRPHCVPF

SEQ ID NO: 625 - protein, 9 aa, Homo sapiens
VPFWTGRIL

SEQ ID NO: 626 - protein, 10 aa, Homo sapiens
VPFWTGRILL

SEQ ID NO: 627 - protein, 10 aa, Homo sapiens
VRMRPHCVPF

SEQ ID NO: 628 - protein, 9 aa, Homo sapiens
AMVPRGVSM

SEQ ID NO: 629 - protein, 10 aa, Homo sapiens
AMVPRGVSMA

SEQ ID NO: 630 - protein, 10 aa, Homo sapiens
AWAPTSRTPW

SEQ ID NO: 631 - protein, 10 aa, Homo sapiens
CPMPTTPVQA

SEQ ID NO: 632 - protein, 10 aa, Homo sapiens
CPSTVPPSPA

SEQ ID NO: 633 - protein, 10 aa, Homo sapiens
GAMVPRGVSM

SEQ ID NO: 634 - protein, 10 aa, Homo sapiens
MPCPMPTTPV

SEQ ID NO: 635 - protein, 9 aa, Homo sapiens
MPTTPVQAW

SEQ ID NO: 636 - protein, 10 aa, Homo sapiens
MPTTPVQAWL

SEQ ID NO: 637 - protein, 10 aa, Homo sapiens
SLPEDRYTQA

SEQ ID NO: 638 - protein, 9 aa, Homo sapiens
SPAQPYLRV

SEQ ID NO: 639 - protein, 10 aa, Homo sapiens
SPAQPYLRVS

SEQ ID NO: 640 - protein, 9 aa, Homo sapiens
TIMPCPMPT

SEQ ID NO: 641 - protein, 9 aa, Homo sapiens
TPVQAWLEA

SEQ ID NO: 642 - protein, 9 aa, Homo sapiens
TSRTPWGAM

SEQ ID NO: 643 - protein, 10 aa, Homo sapiens
VPPSPAQPYL

SEQ ID NO: 644 - protein, 9 aa, Homo sapiens
VPRGVSMAH

SEQ ID NO: 645 - protein, 10 aa, Homo sapiens
CLSARTGLSI

SEQ ID NO: 646 - protein, 10 aa, Homo sapiens
CTTLNSPPLK

SEQ ID NO: 647 - protein, 9 aa, Homo sapiens
GLSISCTTL

SEQ ID NO: 648 - protein, 9 aa, Homo sapiens
SPPLKKMSM

SEQ ID NO: 649 - protein, 9 aa, Homo sapiens
TLNSPPLKK

SEQ ID NO: 650 - protein, 9 aa, Homo sapiens
TTLNSPPLK

SEQ ID NO: 651 - protein, 10 aa, Homo sapiens
TTLNSPPLKK

SEQ ID NO: 652 - protein, 9 aa, Homo sapiens
LQRFRFTHV

SEQ ID NO: 653 - protein, 10 aa, Homo sapiens
LQRFRFTHVI

SEQ ID NO: 654 - protein, 9 aa, Homo sapiens
RLSSVLQRF

SEQ ID NO: 655 - protein, 10 aa, Homo sapiens
RLSSVLQRFR

SEQ ID NO: 656 - protein, 10 aa, Homo sapiens
VLQRFRFTHV

SEQ ID NO: 657 - protein, 10 aa, Homo sapiens
ASVGRHSLSK

SEQ ID NO: 658 - protein, 9 aa, Homo sapiens
KFLPFWGFL

SEQ ID NO: 659 - protein, 9 aa, Homo sapiens
LASVGRHSL

SEQ ID NO: 660 - protein, 10 aa, Homo sapiens
LPFWGFLEEF

SEQ ID NO: 661 - protein, 9 aa, Homo sapiens
PFWGFLEEF

SEQ ID NO: 662 - protein, 9 aa, Homo sapiens
SVGRHSLSK

SEQ ID NO: 663 - protein, 9 aa, Homo sapiens
APRPIRPPF

SEQ ID NO: 664 - protein, 10 aa, Homo sapiens
APRPIRPPFL

SEQ ID NO: 665 - protein, 10 aa, Homo sapiens
AQKMKKAHFL

SEQ ID NO: 666 - protein, 9 aa, Homo sapiens
FLPSAAQKM

SEQ ID NO: 667 - protein, 10 aa, Homo sapiens
GLFLPSAAQK

SEQ ID NO: 668 - protein, 9 aa, Homo sapiens
HPLLVSLLL

SEQ ID NO: 669 - protein, 10 aa, Homo sapiens
KAHFLKTWFR

SEQ ID NO: 670 - protein, 9 aa, Homo sapiens
KARFSTASL

SEQ ID NO: 671 - protein, 9 aa, Homo sapiens
KMKKAHFLK

SEQ ID NO: 672 - protein, 10 aa, Homo sapiens
KTWFRSNPTK

SEQ ID NO: 673 - protein, 9 aa, Homo sapiens
LAKELTHPL

SEQ ID NO: 674 - protein, 10 aa, Homo sapiens
LAKELTHPLL

SEQ ID NO: 675 - protein, 10 aa, Homo sapiens
NPTKTKKARF

SEQ ID NO: 676 - protein, 9 aa, Homo sapiens
QKMKKAHFL

SEQ ID NO: 677 - protein, 9 aa, Homo sapiens
RFSTASLAK

SEQ ID NO: 678 - protein, 10 aa, Homo sapiens
RPIRPPFLES

SEQ ID NO: 679 - protein, 9 aa, Homo sapiens
RSNPTKTKK

SEQ ID NO: 680 - protein, 10 aa, Homo sapiens
SLAKELTHPL

SEQ ID NO: 681 - protein, 9 aa, Homo sapiens
TKKARFSTA

SEQ ID NO: 682 - protein, 9 aa, Homo sapiens
GLRFWNPSR

SEQ ID NO: 683 - protein, 10 aa, Homo sapiens
ISQLLSWPQK

SEQ ID NO: 684 - protein, 9 aa, Homo sapiens
RIAHISQLL

SEQ ID NO: 685 - protein, 9 aa, Homo sapiens
RLGYSSHQL

SEQ ID NO: 686 - protein, 9 aa, Homo sapiens
SQLLSWPQK

SEQ ID NO: 687 - protein, 9 aa, Homo sapiens
SRIAHISQL

SEQ ID NO: 688 - protein, 9 aa, Homo sapiens
WPQKTEERL

SEQ ID NO: 689 - protein, 9 aa, Homo sapiens
YSSHQLPRK

SEQ ID NO: 690 - protein, 10 aa, Homo sapiens
CPQRMTPGTT

SEQ ID NO: 691 - protein, 9 aa, Homo sapiens
EAEKRTRTL

SEQ ID NO: 692 - protein, 9 aa, Homo sapiens
GTTFITMMK

SEQ ID NO: 693 - protein, 10 aa, Homo sapiens
GTTFITMMKK

SEQ ID NO: 694 - protein, 10 aa, Homo sapiens
ITMMKKEAEK

SEQ ID NO: 695 - protein, 9 aa, Homo sapiens
RMTPGTTFI

SEQ ID NO: 696 - protein, 9 aa, Homo sapiens
SPYCPQRMT

SEQ ID NO: 697 - protein, 9 aa, Homo sapiens
TMMKKEAEK

SEQ ID NO: 698 - protein, 9 aa, Homo sapiens
TPGTTFITM

SEQ ID NO: 699 - protein, 10 aa, Homo sapiens
TPGTTFITMM

SEQ ID NO: 700 - protein, 9 aa, Homo sapiens
TTFITMMKK

SEQ ID NO: 701 - protein, 9 aa, Homo sapiens
CPGATWREA

SEQ ID NO: 702 - protein, 10 aa, Homo sapiens
CPGATWREAA

SEQ ID NO: 703 - protein, 10 aa, Homo sapiens
RSRCPGATWR

SEQ ID NO: 704 - protein, 9 aa, Homo sapiens
TPRATRSRC

SEQ ID NO: 705 - protein, 10 aa, Homo sapiens
HVLPEPHLAL

SEQ ID NO: 706 - protein, 10 aa, Homo sapiens
RPLQTHVLPE

SEQ ID NO: 707 - protein, 10 aa, Homo sapiens
VLWTTPPLQH

SEQ ID NO: 708 - protein, 10 aa, Homo sapiens
APSESPCSPF

SEQ ID NO: 709 - protein, 10 aa, Homo sapiens
CPLDHTTPPA

SEQ ID NO: 710 - protein, 9 aa, Homo sapiens
FLQEQYHEA

SEQ ID NO: 711 - protein, 10 aa, Homo sapiens
RLAFLQEQYH

SEQ ID NO: 712 - protein, 10 aa, Homo sapiens
SPCSPFRLAF

SEQ ID NO: 713 - protein, 9 aa, Homo sapiens
SPPWVRALL

SEQ ID NO: 714 - protein, 10 aa, Homo sapiens
YPACPLDHTT

SEQ ID NO: 715 - protein, 9 aa, Homo sapiens
ALHLRSCPC

SEQ ID NO: 716 - protein, 9 aa, Homo sapiens
CLHHRRHLV

SEQ ID NO: 717 - protein, 10 aa, Homo sapiens
CLHHRRHLVC

SEQ ID NO: 718 - protein, 10 aa, Homo sapiens
CLHRKSHPHL

SEQ ID NO: 719 - protein, 9 aa, Homo sapiens
CLRSHACPP

SEQ ID NO: 720 - protein, 9 aa, Homo sapiens
CLRSHTCPP

SEQ ID NO: 721 - protein, 9 aa, Homo sapiens
CLWCHACLH

SEQ ID NO: 722 - protein, 9 aa, Homo sapiens
CPHHLKNHL

SEQ ID NO: 723 - protein, 9 aa, Homo sapiens
CPHHLKNLL

SEQ ID NO: 724 - protein, 9 aa, Homo sapiens
CPHHLRTRL

SEQ ID NO: 725 - protein, 10 aa, Homo sapiens
CPLHLRSLPF

SEQ ID NO: 726 - protein, 10 aa, Homo sapiens
CPLPRHLKHL

SEQ ID NO: 727 - protein, 10 aa, Homo sapiens
CPLSLRSHPC

SEQ ID NO: 728 - protein, 10 aa, Homo sapiens
CPRHLRNCPL

SEQ ID NO: 729 - protein, 9 aa, Homo sapiens
FPHHLRHHA

SEQ ID NO: 730 - protein, 10 aa, Homo sapiens
FPHHLRHHAC

SEQ ID NO: 731 - protein, 9 aa, Homo sapiens
GLRSRTCPP

SEQ ID NO: 732 - protein, 10 aa, Homo sapiens
HACLHRLRNL

SEQ ID NO: 733 - protein, 9 aa, Homo sapiens
HLACLHHLR

SEQ ID NO: 734 - protein, 9 aa, Homo sapiens
HLCPPHLRY

SEQ ID NO: 735 - protein, 10 aa, Homo sapiens
HLCPPHLRYR

SEQ ID NO: 736 - protein, 9 aa, Homo sapiens
HLKHLACLH

SEQ ID NO: 737 - protein, 9 aa, Homo sapiens
HLKHRPCPH

SEQ ID NO: 738 - protein, 9 aa, Homo sapiens
HLKNHLCPP

SEQ ID NO: 739 - protein, 9 aa, Homo sapiens
HLKSHPCLH

SEQ ID NO: 740 - protein, 9 aa, Homo sapiens
HLKSLLCPL

SEQ ID NO: 741 - protein, 9 aa, Homo sapiens
HLLHLRRLY

SEQ ID NO: 742 - protein, 9 aa, Homo sapiens
HLRNCPLPR

SEQ ID NO: 743 - protein, 10 aa, Homo sapiens
HLRNCPLPRH

SEQ ID NO: 744 - protein, 10 aa, Homo sapiens
HLRRLYPHHL

SEQ ID NO: 745 - protein, 9 aa, Homo sapiens
HLRSHPCPL

SEQ ID NO: 746 - protein, 10 aa, Homo sapiens
HLRSHPCPLH

SEQ ID NO: 747 - protein, 9 aa, Homo sapiens
HLRSLPFPH

SEQ ID NO: 748 - protein, 9 aa, Homo sapiens
HLRTRLCPH

SEQ ID NO: 749 - protein, 9 aa, Homo sapiens
HLVCSHHLK

SEQ ID NO: 750 - protein, 10 aa, Homo sapiens
HPCLHHRRHL

SEQ ID NO: 751 - protein, 9 aa, Homo sapiens
HPGLRSRTC

SEQ ID NO: 752 - protein, 10 aa, Homo sapiens
HPHLLHLRRL

SEQ ID NO: 753 - protein, 9 aa, Homo sapiens
HRKSHPHLL

SEQ ID NO: 754 - protein, 9 aa, Homo sapiens
HRRTRSCPC

SEQ ID NO: 755 - protein, 10 aa, Homo sapiens
KSHPHLLHLR

SEQ ID NO: 756 - protein, 10 aa, Homo sapiens
KSLLCPLHLR

SEQ ID NO: 757 - protein, 10 aa, Homo sapiens
LLCPLHLRSL

SEQ ID NO: 758 - protein, 10 aa, Homo sapiens
LLHLRRLYPH

SEQ ID NO: 759 - protein, 9 aa, Homo sapiens
LPRHLKHLA

SEQ ID NO: 760 - protein, 10 aa, Homo sapiens
LPRHLKHLAC

SEQ ID NO: 761 - protein, 9 aa, Homo sapiens
LRRLRSHTC

SEQ ID NO: 762 - protein, 9 aa, Homo sapiens
LRRLYPHHL

SEQ ID NO: 763 - protein, 10 aa, Homo sapiens
LVCSHHLKSL

SEQ ID NO: 764 - protein, 10 aa, Homo sapiens
NLRNHTCPPS

SEQ ID NO: 765 - protein, 9 aa, Homo sapiens
PLHLRSLPF

SEQ ID NO: 766 - protein, 10 aa, Homo sapiens
RLCPHHLKNH

SEQ ID NO: 767 - protein, 9 aa, Homo sapiens
RLYPHHLKH

SEQ ID NO: 768 - protein, 10 aa, Homo sapiens
RLYPHHLKHR

SEQ ID NO: 769 - protein, 10 aa, Homo sapiens
RPCPHHLKNL

SEQ ID NO: 770 - protein, 10 aa, Homo sapiens
RSHPCPLHLK

SEQ ID NO: 771 - protein, 10 aa, Homo sapiens
RSLPFPHHLR

SEQ ID NO: 772 - protein, 9 aa, Homo sapiens
RTRLCPHHL

SEQ ID NO: 773 - protein, 10 aa, Homo sapiens
RTRLCPHHLK

SEQ ID NO: 774 - protein, 9 aa, Homo sapiens
SLLCPLHLR

SEQ ID NO: 775 - protein, 9 aa, Homo sapiens
SLRSHACPP

SEQ ID NO: 776 - protein, 9 aa, Homo sapiens
SPLRSQANA

SEQ ID NO: 777 - protein, 9 aa, Homo sapiens
YLRRLRSHT

SEQ ID NO: 778 - protein, 10 aa, Homo sapiens
YPHHLKHRPC

SEQ ID NO: 779 - protein, 9 aa, Homo sapiens
FITSGQIFK

SEQ ID NO: 780 - protein, 9 aa, Homo sapiens
IFITSGQIF

SEQ ID NO: 781 - protein, 9 aa, Homo sapiens
SQSEALCVL

SEQ ID NO: 782 - protein, 10 aa, Homo sapiens
SQSEALCVLL

SEQ ID NO: 783 - protein, 9 aa, Homo sapiens
AYTGTILMM

SEQ ID NO: 784 - protein, 10 aa, Homo sapiens
DLKAYTGTIL

SEQ ID NO: 785 - protein, 9 aa, Homo sapiens
ILTKQIKTK

SEQ ID NO: 786 - protein, 9 aa, Homo sapiens
KMILTKQIK

SEQ ID NO: 787 - protein, 9 aa, Homo sapiens
KPTDTFLQI

SEQ ID NO: 788 - protein, 10 aa, Homo sapiens
KPTDTFLQIL

SEQ ID NO: 789 - protein, 10 aa, Homo sapiens
MILTKQIKTK

SEQ ID NO: 790 - protein, 10 aa, Homo sapiens
ITRYTIFVLK

SEQ ID NO: 791 - protein, 9 aa, Homo sapiens
LIAELHNIL

SEQ ID NO: 792 - protein, 10 aa, Homo sapiens
LIAELHNILL

SEQ ID NO: 793 - protein, 10 aa, Homo sapiens
MTPPNLIAEL

SEQ ID NO: 794 - protein, 9 aa, Homo sapiens
NLIAELHNI

SEQ ID NO: 795 - protein, 10 aa, Homo sapiens
NLIAELHNIL

SEQ ID NO: 796 - protein, 10 aa, Homo sapiens
RYTIFVLKDI

SEQ ID NO: 797 - protein, 10 aa, Homo sapiens
TITRYTIFVL

SEQ ID NO: 798 - protein, 9 aa, Homo sapiens
TPPNLIAEL

SEQ ID NO: 799 - protein, 9 aa, Homo sapiens
FLQFRTHTT

SEQ ID NO: 800 - protein, 10 aa, Homo sapiens
LPAKGEDIFL

SEQ ID NO: 801 - protein, 10 aa, Homo sapiens
LQFRTHTTGR

SEQ ID NO: 802 - protein, 9 aa, Homo sapiens
NLQSSVCGL

SEQ ID NO: 803 - protein, 9 aa, Homo sapiens
SSVCGLPAK

SEQ ID NO: 804 - protein, 10 aa, Homo sapiens
VQWRNLQSSV

SEQ ID NO: 805 - protein, 9 aa, Homo sapiens
GQNVSLLGK

SEQ ID NO: 806 - protein, 10 aa, Homo sapiens
HTRTRGNLRK

SEQ ID NO: 807 - protein, 10 aa, Homo sapiens
ILHTRTRGNL

SEQ ID NO: 808 - protein, 10 aa, Homo sapiens
KGQNVSLLGK

SEQ ID NO: 809 - protein, 9 aa, Homo sapiens
LLGKYILHT

SEQ ID NO: 810 - protein, 10 aa, Homo sapiens
LRKSRKWKSM

SEQ ID NO: 811 - protein, 9 aa, Homo sapiens
SLLGKYILH

SEQ ID NO: 812 - protein, 10 aa, Homo sapiens
SLLGKYILHT

SEQ ID NO: 813 - protein, 9 aa, Homo sapiens
CTSPLLAPV

SEQ ID NO: 814 - protein, 9 aa, Homo sapiens
FPENLPGQL

SEQ ID NO: 815 - protein, 10 aa, Homo sapiens
GLLAFWDSQV

SEQ ID NO: 816 - protein, 9 aa, Homo sapiens
IFCPFPENL

SEQ ID NO: 817 - protein, 9 aa, Homo sapiens
LLAFWDSQV

SEQ ID NO: 818 - protein, 9 aa, Homo sapiens
LLAPVIFCP

SEQ ID NO: 819 - protein, 10 aa, Homo sapiens
LLAPVIFCPF

SEQ ID NO: 820 - protein, 9 aa, Homo sapiens
LPCPQQDVL

SEQ ID NO: 821 - protein, 9 aa, Homo sapiens
RFPSGLLAF

SEQ ID NO: 822 - protein, 10 aa, Homo sapiens
RFPSGLLAFW

SEQ ID NO: 823 - protein, 9 aa, Homo sapiens
SPLLAPVIF

SEQ ID NO: 824 - protein, 9 aa, Homo sapiens
SPRGPCTSS

SEQ ID NO: 825 - protein, 10 aa, Homo sapiens
SPRGPCTSSS

SEQ ID NO: 826 - protein, 9 aa, Homo sapiens
SQVCDLHVL

SEQ ID NO: 827 - protein, 10 aa, Homo sapiens
VIFCPFPENL

SEQ ID NO: 828 - protein, 9 aa, Homo sapiens
AMVWPLLSI

SEQ ID NO: 829 - protein, 10 aa, Homo sapiens
AMVWPLLSIL

SEQ ID NO: 830 - protein, 9 aa, Homo sapiens
AQIAMVWPL

SEQ ID NO: 831 - protein, 10 aa, Homo sapiens
AQIAMVWPLL

SEQ ID NO: 832 - protein, 9 aa, Homo sapiens
CPMSRLRLA

SEQ ID NO: 833 - protein, 10 aa, Homo sapiens
CPMSRLRLAL

SEQ ID NO: 834 - protein, 10 aa, Homo sapiens
IAMVWPLLSI

SEQ ID NO: 835 - protein, 10 aa, Homo sapiens
ILSEWKEICV

SEQ ID NO: 836 - protein, 9 aa, Homo sapiens
IVWWCPMSR

SEQ ID NO: 837 - protein, 10 aa, Homo sapiens
IVWWCPMSRL

SEQ ID NO: 838 - protein, 10 aa, Homo sapiens
IWMTETLFDI

SEQ ID NO: 839 - protein, 9 aa, Homo sapiens
LLSILSEWK

SEQ ID NO: 840 - protein, 9 aa, Homo sapiens
MSAAQIAMV

SEQ ID NO: 841 - protein, 9 aa, Homo sapiens
MSRLRLALT

SEQ ID NO: 842 - protein, 10 aa, Homo sapiens
MSRLRLALTV

SEQ ID NO: 843 - protein, 9 aa, Homo sapiens
MVWPLLSIL

SEQ ID NO: 844 - protein, 10 aa, Homo sapiens
RLALTVPPST

SEQ ID NO: 845 - protein, 9 aa, Homo sapiens
TLFDIVWWC

SEQ ID NO: 846 - protein, 10 aa, Homo sapiens
TLFDIVWWCP

SEQ ID NO: 847 - protein, 10 aa, Homo sapiens
TMSAAQIAMV

SEQ ID NO: 848 - protein, 10 aa, Homo sapiens
VWSIWMTETL

SEQ ID NO: 849 - protein, 9 aa, Homo sapiens
WMTETLFDI

SEQ ID NO: 850 - protein, 10 aa, Homo sapiens
WMTETLFDIV

SEQ ID NO: 851 - protein, 9 aa, Homo sapiens
ALRCVFVPV

SEQ ID NO: 852 - protein, 10 aa, Homo sapiens
ALRCVFVPVL

SEQ ID NO: 853 - protein, 9 aa, Homo sapiens
ALSEHCPTT

SEQ ID NO: 854 - protein, 10 aa, Homo sapiens
AQRKRISARK

SEQ ID NO: 855 - protein, 9 aa, Homo sapiens
GAQRKRISA

SEQ ID NO: 856 - protein, 9 aa, Homo sapiens
HWMENISPF

SEQ ID NO: 857 - protein, 9 aa, Homo sapiens
LPSQRRNHW

SEQ ID NO: 858 - protein, 10 aa, Homo sapiens
LPSQRRNHWM

SEQ ID NO: 859 - protein, 10 aa, Homo sapiens
NISPFRSVGV

SEQ ID NO: 860 - protein, 9 aa, Homo sapiens
RISARKGSL

SEQ ID NO: 861 - protein, 10 aa, Homo sapiens
SPFRSVGVSA

SEQ ID NO: 862 - protein, 10 aa, Homo sapiens
SPSSHWKTPV

SEQ ID NO: 863 - protein, 10 aa, Homo sapiens
TALRCVFVPV

SEQ ID NO: 864 - protein, 9 aa, Homo sapiens
VIYWDGTAL

SEQ ID NO: 865 - protein, 10 aa, Homo sapiens
VIYWDGTALR

SEQ ID NO: 866 - protein, 10 aa, Homo sapiens
VLGETGAQRK

SEQ ID NO: 867 - protein, 9 aa, Homo sapiens
HPRPRHGHL

SEQ ID NO: 868 - protein, 10 aa, Homo sapiens
HPRPRHGHLQ

SEQ ID NO: 869 - protein, 9 aa, Homo sapiens
RPRHGHLQA

SEQ ID NO: 870 - protein, 10 aa, Homo sapiens
RPRHGHLQAV

SEQ ID NO: 871 - protein, 10 aa, Homo sapiens
GSLKTQVQMK

SEQ ID NO: 872 - protein, 10 aa, Homo sapiens
PPGPCHLLSL

SEQ ID NO: 873 - protein, 10 aa, Homo sapiens
RTILNNGSLK

SEQ ID NO: 874 - protein, 9 aa, Homo sapiens
SLKTQVQMK

SEQ ID NO: 875 - protein, 10 aa, Homo sapiens
SLKTQVQMKL

SEQ ID NO: 876 - protein, 9 aa, Homo sapiens
TILNNGSLK

SEQ ID NO: 877 - protein, 10 aa, Homo sapiens
CPPCRPKQWM

SEQ ID NO: 878 - protein, 10 aa, Homo sapiens
TTFCPPCRPK

SEQ ID NO: 879 - protein, 10 aa, Homo sapiens
CFANWPRPAL

SEQ ID NO: 880 - protein, 9 aa, Homo sapiens
FANWPRPAL

SEQ ID NO: 881 - protein, 9 aa, Homo sapiens
GLIPHPRPA

SEQ ID NO: 882 - protein, 9 aa, Homo sapiens
HPRPAPASA

SEQ ID NO: 883 - protein, 10 aa, Homo sapiens
HPRPAPASAP

SEQ ID NO: 884 - protein, 9 aa, Homo sapiens
IPHPRPAPA

SEQ ID NO: 885 - protein, 10 aa, Homo sapiens
IPHPRPAPAS

SEQ ID NO: 886 - protein, 9 aa, Homo sapiens
RPALCSCGL

SEQ ID NO: 887 - protein, 10 aa, Homo sapiens
RPALCSCGLI

SEQ ID NO: 888 - protein, 9 aa, Homo sapiens
TPLPSTRCF

SEQ ID NO: 889 - protein, 9 aa, Homo sapiens
WPRPALCSC

SEQ ID NO: 890 - protein, 10 aa, Homo sapiens
WPRPALCSCG

SEQ ID NO: 891 - protein, 10 aa, Homo sapiens
ALRRSGRPPK

SEQ ID NO: 892 - protein, 9 aa, Homo sapiens
GLVPSLVSK

SEQ ID NO: 893 - protein, 9 aa, Homo sapiens
KISVETYTV

SEQ ID NO: 894 - protein, 10 aa, Homo sapiens
LLMVLMSLDL

SEQ ID NO: 895 - protein, 10 aa, Homo sapiens
LMSLDLDTGL

SEQ ID NO: 896 - protein, 9 aa, Homo sapiens
LMVLMSLDL

SEQ ID NO: 897 - protein, 10 aa, Homo sapiens
LVSKCLILRV

SEQ ID NO: 898 - protein, 9 aa, Homo sapiens
QLLMVLMSL

SEQ ID NO: 899 - protein, 9 aa, Homo sapiens
RPGAADTGA

SEQ ID NO: 900 - protein, 10 aa, Homo sapiens
RPGAADTGAH

SEQ ID NO: 901 - protein, 9 aa, Homo sapiens
SLDLDTGLV

SEQ ID NO: 902 - protein, 9 aa, Homo sapiens
SLVSKCLIL

SEQ ID NO: 903 - protein, 10 aa, Homo sapiens
SQLLMVLMSL

SEQ ID NO: 904 - protein, 9 aa, Homo sapiens
TVSSQLLMV

SEQ ID NO: 905 - protein, 9 aa, Homo sapiens
TYTVSSQLL

SEQ ID NO: 906 - protein, 10 aa, Homo sapiens
TYTVSSQLLM

SEQ ID NO: 907 - protein, 9 aa, Homo sapiens
VLMSLDLDT

SEQ ID NO: 908 - protein, 9 aa, Homo sapiens
VPSLVSKCL

SEQ ID NO: 909 - protein, 10 aa, Homo sapiens
VSKCLILRVK

SEQ ID NO: 910 - protein, 9 aa, Homo sapiens
YTVSSQLLM

SEQ ID NO: 911 - protein, 10 aa, Homo sapiens
YTVSSQLLMV

SEQ ID NO: 912 - protein, 9 aa, Homo sapiens
FCQYHTASV

SEQ ID NO: 913 - protein, 9 aa, Homo sapiens
CPYGSTSTA

SEQ ID NO: 914 - protein, 10 aa, Homo sapiens
CPYGSTSTAS

SEQ ID NO: 915 - protein, 10 aa, Homo sapiens
LARAAASTAT

SEQ ID NO: 916 - protein, 9 aa, Homo sapiens
MLTDSLFLP

SEQ ID NO: 917 - protein, 10 aa, Homo sapiens
PPRSSSAIAV

SEQ ID NO: 918 - protein, 10 aa, Homo sapiens
RAAASTATEV

SEQ ID NO: 919 - protein, 9 aa, Homo sapiens
SPPRSSSAI

SEQ ID NO: 920 - protein, 10 aa, Homo sapiens
SPPRSSSAIA

SEQ ID NO: 921 - protein, 9 aa, Homo sapiens
SPTQRCRLA

SEQ ID NO: 922 - protein, 9 aa, Homo sapiens
TQRCRLARA

SEQ ID NO: 923 - protein, 10 aa, Homo sapiens
TQRCRLARAA

SEQ ID NO: 924 - protein, 10 aa, Homo sapiens
KIWKTTQMCR

SEQ ID NO: 925 - protein, 9 aa, Homo sapiens
WTSSGRSTK

SEQ ID NO: 926 - protein, 9 aa, Homo sapiens
ALGELARAL

SEQ ID NO: 927 - protein, 9 aa, Homo sapiens
AQLRRRAAA

SEQ ID NO: 928 - protein, 10 aa, Homo sapiens
AQLRRRAAAL

SEQ ID NO: 929 - protein, 10 aa, Homo sapiens
ARRAARMAQL

SEQ ID NO: 930 - protein, 9 aa, Homo sapiens
HPQLPRSPL

SEQ ID NO: 931 - protein, 10 aa, Homo sapiens
HPQLPRSPLA

SEQ ID NO: 932 - protein, 9 aa, Homo sapiens
LARALPGHL

SEQ ID NO: 933 - protein, 10 aa, Homo sapiens
LARALPGHLL

SEQ ID NO: 934 - protein, 9 aa, Homo sapiens
MAQLRRRAA

SEQ ID NO: 935 - protein, 10 aa, Homo sapiens
MAQLRRRAAA

SEQ ID NO: 936 - protein, 9 aa, Homo sapiens
QLRRRAAAL

SEQ ID NO: 937 - protein, 10 aa, Homo sapiens
RAAALPNAAA

SEQ ID NO: 938 - protein, 10 aa, Homo sapiens
RMAQLRRRAA

SEQ ID NO: 939 - protein, 10 aa, Homo sapiens
SQSARRAARM

SEQ ID NO: 940 - protein, 10 aa, Homo sapiens
GMTLGEKFRV

SEQ ID NO: 941 - protein, 9 aa, Homo sapiens
RVGNCKHLK

SEQ ID NO: 942 - protein, 9 aa, Homo sapiens
GPSEPGNNI

SEQ ID NO: 943 - protein, 10 aa, Homo sapiens
KICNESASRK

SEQ ID NO: 944 - protein, 10 aa, Homo sapiens
GIQVLNVSLK

SEQ ID NO: 945 - protein, 9 aa, Homo sapiens
IQVLNVSLK

SEQ ID NO: 946 - protein, 9 aa, Homo sapiens
KSSSNVISY

SEQ ID NO: 947 - protein, 9 aa, Homo sapiens
KYGWSLLRV

SEQ ID NO: 948 - protein, 9 aa, Homo sapiens
RSWKYGWSL

SEQ ID NO: 949 - protein, 9 aa, Homo sapiens
SLKSSSNVI

SEQ ID NO: 950 - protein, 9 aa, Homo sapiens
SWKYGWSLL

SEQ ID NO: 951 - protein, 9 aa, Homo sapiens
TVANGRSWK

SEQ ID NO: 952 - protein, 9 aa, Homo sapiens
VPQVNGIQV

SEQ ID NO: 953 - protein, 10 aa, Homo sapiens
VPQVNGIQVL

SEQ ID NO: 954 - protein, 10 aa, Homo sapiens
VTVANGRSWK

SEQ ID NO: 955 - protein, 9 aa, Homo sapiens
WSLLRVPQV

SEQ ID NO: 956 - protein, 10 aa, Homo sapiens
HPGDCLIFKL

SEQ ID NO: 957 - protein, 9 aa, Homo sapiens
KLRVPGSSV

SEQ ID NO: 958 - protein, 10 aa, Homo sapiens
KLRVPGSSVL

SEQ ID NO: 959 - protein, 9 aa, Homo sapiens
RVPGSSVLV

SEQ ID NO: 960 - protein, 9 aa, Homo sapiens
SVLVTVPGL

SEQ ID NO: 961 - protein, 10 aa, Homo sapiens
VPGSSVLVTV

SEQ ID NO: 962 - protein, 10 aa, Homo sapiens
ALLLRPRPPR

SEQ ID NO: 963 - protein, 10 aa, Homo sapiens
ALSALLLRPR

SEQ ID NO: 964 - protein, 9 aa, Homo sapiens
LTINKEEAL

SEQ ID NO: 965 - protein, 9 aa, Homo sapiens
IVHSATGFK

SEQ ID NO: 966 - protein, 9 aa, Homo sapiens
ATGFKQSSK

SEQ ID NO: 967 - protein, 9 aa, Homo sapiens
ELFPLIFPA

SEQ ID NO: 968 - protein, 9 aa, Homo sapiens
KGPELFPLI

SEQ ID NO: 969 - protein, 10 aa, Homo sapiens
KGPELFPLIF

SEQ ID NO: 970 - protein, 11 aa, Homo sapiens
KPTDAPPKAGV

SEQ ID NO: 971 - protein, 10 aa, Homo sapiens
ALEEKKGNYV

SEQ ID NO: 972 - protein, 10 aa, Homo sapiens
IQLQDKFEHL

SEQ ID NO: 973 - protein, 9 aa, Homo sapiens
QLQDKFEHL

SEQ ID NO: 974 - protein, 10 aa, Homo sapiens
QLQDKFEHLK

SEQ ID NO: 975 - protein, 10 aa, Homo sapiens
YLVIQLQDKF

SEQ ID NO: 976 - protein, 10 aa, Homo sapiens
QVYRRKHQEL

SEQ ID NO: 977 - protein, 9 aa, Homo sapiens
STREKNSQV

SEQ ID NO: 978 - protein, 9 aa, Homo sapiens
VYRRKHQEL

SEQ ID NO: 979 - protein, 9 aa, Homo sapiens
VLTVTSTDV

SEQ ID NO: 980 - protein, 10 aa, Homo sapiens
VLTVTSTDVK

SEQ ID NO: 981 - protein, 9 aa, Homo sapiens
IMSLWGLVS

SEQ ID NO: 982 - protein, 10 aa, Homo sapiens
IMSLWGLVSK

SEQ ID NO: 983 - protein, 9 aa, Homo sapiens
KLKQEATSK

SEQ ID NO: 984 - protein, 9 aa, Homo sapiens
QIMSLWGLV

SEQ ID NO: 985 - protein, 9 aa, Homo sapiens
SQIMSLWGL

SEQ ID NO: 986 - protein, 10 aa, Homo sapiens
SQIMSLWGLV

SEQ ID NO: 987 - protein, 9 aa, Homo sapiens
TSKSQIMSL

SEQ ID NO: 988 - protein, 10 aa, Homo sapiens
LLQEFDVQEA

SEQ ID NO: 989 - protein, 10 aa, Homo sapiens
LQEFDVQEAL

SEQ ID NO: 990 - protein, 9 aa, Homo sapiens
GPREPRNRT

SEQ ID NO: 991 - protein, 10 aa, Homo sapiens
RNRTEKHSTM

SEQ ID NO: 992 - protein, 9 aa, Homo sapiens
ALNSEALSV

SEQ ID NO: 993 - protein, 10 aa, Homo sapiens
ALNSEALSVV

SEQ ID NO: 994 - protein, 10 aa, Homo sapiens
MALNSEALSV

SEQ ID NO: 995 - protein, 67 aa, Homo sapiens
MSDVAIVKEGWLHKRGKYIKTWRPRYFLLKNDGTFIGYKERPQDVDQREAPLNNFSVAQCQLMKTER

SEQ ID NO: 996 - protein, 101 aa, Homo sapiens
TMLVLEGSGNLVLYTGVVRVGKVFIPGLPAPSLTMSNTMPRPSTPLDGVSAPKPLSKLLGSLDEVVLLSPVPELRDSSKLHDSLYNEDCTFQQLGTYIHSI

SEQ ID NO: 997 - protein, 90 aa, Homo sapiens
MREPIYMHSTMVFLPWELHTKKGPSPPEQFMAVKLSDSRTALKSGYGKYLGINSDELVGHSDAIGPREQWEPVFQNGKMALLASNSCFIR

SEQ ID NO: 998 - protein, 101 aa, Homo sapiens
AMPNQAQMRILKETELRKVKVLGSGAFGTVYKGIWIPDGENVKIPVAIKVSRENTSPKANKEILDEAYVMAGVGSPYVSRLLGICLTSTVQLVTQLMPYGC

SEQ ID NO: 999 - protein, 101 aa, Homo sapiens
AVCKSKKSWQARHTGIKIVQQIAILMGCAILPHLRSLVEIIEHGLVDEQQEVRTISALAIAALAEAATPYGIESFDSVLKPLWKGIRQHRGKGLAAFLKAI

SEQ ID NO: 1000 - protein, 101 aa, Homo sapiens
YLSLYLLLVSCPKSEVRAKFKFSILNAKGEETKAMESQRAYRFVQGKDWGLKKFIRRDFLLDEANGLLPDDKLTLFCEVSVVQDSVNISGQNTMNMVKVPE

SEQ ID NO: 1001 - protein, 101 aa, Homo sapiens
YLSLYLLLVSCPKSEVRAKFKFSILNAKGEETKAMESQRAYRFVQGKDWGVKKFIRRDFLLDEANGLLPDDKLTLFCEVSVVQDSVNISGQNTMNMVKVPE

SEQ ID NO: 1002 - protein, 101 aa, Homo sapiens
PEVGSDCTTIHYNYMCNSSCMGGMNRRPILTIITLEDSSGNLLGRNSFEVHVCACPGRDRRTEEENLRKKGEPHHELPPGSTKRALPNNTSSSPQPKKKPL

SEQ ID NO: 1003 - protein, 101 aa, Homo sapiens
TEVVRRCPHHERCSDSDGLAPPQHLIRVEGNLRVEYLDDRNTFRHSVVVPCEPPEVGSDCTTIHYNYMCNSSCMGGMNRRPILTIITLEDSSGNLLGRNSF

SEQ ID NO: 1004 - protein, 54 aa, Homo sapiens
YNFDKNYKDWQSAVECIAVLDVLLCLANYSRGGDGPMCRPVILLPEDTPPLLRA

SEQ ID NO: 1005 - protein, 10 aa, Homo sapiens
KYIKTWRPRY

SEQ ID NO: 1006 - protein, 9 aa, Homo sapiens
WLHKRGKYI

SEQ ID NO: 1007 - protein, 10 aa, Homo sapiens
WLHKRGKYIK

SEQ ID NO: 1008 - protein, 9 aa, Homo sapiens
APKPLSKLL

SEQ ID NO: 1009 - protein, 10 aa, Homo sapiens
GVSAPKPLSK

SEQ ID NO: 1010 - protein, 9 aa, Homo sapiens
VSAPKPLSK

SEQ ID NO: 1011 - protein, 9 aa, Homo sapiens
CPHRPILQA

SEQ ID NO: 1012 - protein, 9 aa, Homo sapiens
KLSDSRTAL

SEQ ID NO: 1013 - protein, 10 aa, Homo sapiens
KLSDSRTALK

SEQ ID NO: 1014 - protein, 9 aa, Homo sapiens
LSDSRTALK

SEQ ID NO: 1015 - protein, 10 aa, Homo sapiens
RTALKSGYGK

SEQ ID NO: 1016 - protein, 9 aa, Homo sapiens
TALKSGYGK

SEQ ID NO: 1017 - protein, 9 aa, Homo sapiens
ALSASNSCF

SEQ ID NO: 1018 - protein, 10 aa, Homo sapiens
ALSASNSCFI

SEQ ID NO: 1019 - protein, 10 aa, Homo sapiens
FQNGKMALSA

SEQ ID NO: 1020 - protein, 10 aa, Homo sapiens
KVSRENTSPK

SEQ ID NO: 1021 - protein, 10 aa, Homo sapiens
KPIIIGGHAY

SEQ ID NO: 1022 - protein, 10 aa, Homo sapiens
AISTRDPLSK

SEQ ID NO: 1023 - protein, 9 aa, Homo sapiens
QTGVMICAY

SEQ ID NO: 1024 - protein, 9 aa, Homo sapiens
FSGEYIPTV

SEQ ID NO: 1025 - protein, 9 aa, Homo sapiens
TTNAFSGEY

SEQ ID NO: 1026 - protein, 10 aa, Homo sapiens
YTTNAFSGEY

SEQ ID NO: 1027 - protein, 9 aa, Homo sapiens
GLVDEQQEV

SEQ ID NO: 1028 - protein, 9 aa, Homo sapiens
FVQGKDWGL

SEQ ID NO: 1029 - protein, 9 aa, Homo sapiens
FVQGKDWGV

SEQ ID NO: 1030 - protein, 10 aa, Homo sapiens
CMGSMNRRPI

SEQ ID NO: 1031 - protein, 9 aa, Homo sapiens
GSMNRRPIL

SEQ ID NO: 1032 - protein, 9 aa, Homo sapiens
MGSMNRRPI

SEQ ID NO: 1033 - protein, 10 aa, Homo sapiens
MGSMNRRPIL

SEQ ID NO: 1034 - protein, 10 aa, Homo sapiens
SMNRRPILTI

SEQ ID NO: 1035 - protein, 10 aa, Homo sapiens
CMGGMNQRPI

SEQ ID NO: 1036 - protein, 10 aa, Homo sapiens
GMNQRPILTI

SEQ ID NO: 1037 - protein, 9 aa, Homo sapiens
NQRPILTII

SEQ ID NO: 1038 - protein, 10 aa, Homo sapiens
CMGGMNWRPI

SEQ ID NO: 1039 - protein, 10 aa, Homo sapiens
GMNWRPILTI

SEQ ID NO: 1040 - protein, 9 aa, Homo sapiens
MNWRPILTI

SEQ ID NO: 1041 - protein, 10 aa, Homo sapiens
MNWRPILTII

SEQ ID NO: 1042 - protein, 9 aa, Homo sapiens
NSFEVCVCA

SEQ ID NO: 1043 - protein, 9 aa, Homo sapiens
NSFEVHVCA

SEQ ID NO: 1044 - protein, 9 aa, Homo sapiens
VVPCEPPEV

SEQ ID NO: 1045 - protein, 10 aa, Homo sapiens
VVVPCEPPEV

SEQ ID NO: 1046 - protein, 10 aa, Homo sapiens
ILLPEDTPPL

SEQ ID NO: 1047 - protein, 9 aa, Homo sapiens
LLPEDTPPL

SEQ ID NO: 1048 - protein, 9 aa, Homo sapiens
APFRVNHAV

SEQ ID NO: 1049 - protein, 9 aa, Homo sapiens
CLADVLLSV

SEQ ID NO: 1050 - protein, 10 aa, Homo sapiens
FLQERNLPPK

SEQ ID NO: 1051 - protein, 10 aa, Homo sapiens
HLIVLRVVRL

SEQ ID NO: 1052 - protein, 9 aa, Homo sapiens
HPKVHLNTM

SEQ ID NO: 1053 - protein, 10 aa, Homo sapiens
HPKVHLNTMF

SEQ ID NO: 1054 - protein, 9 aa, Homo sapiens
KVHLNTMFR

SEQ ID NO: 1055 - protein, 10 aa, Homo sapiens
KVHLNTMFRR

SEQ ID NO: 1056 - protein, 10 aa, Homo sapiens
LPAPFRVNHA

SEQ ID NO: 1057 - protein, 9 aa, Homo sapiens
MFRRPHSCL

SEQ ID NO: 1058 - protein, 10 aa, Homo sapiens
MFRRPHSCLA

SEQ ID NO: 1059 - protein, 10 aa, Homo sapiens
NTMFRRPHSC

SEQ ID NO: 1060 - protein, 9 aa, Homo sapiens
RPHSCLADV

SEQ ID NO: 1061 - protein, 10 aa, Homo sapiens
RPHSCLADVL

SEQ ID NO: 1062 - protein, 10 aa, Homo sapiens
RVVRLPAPFR

SEQ ID NO: 1063 - protein, 9 aa, Homo sapiens
SVHLIVLRV

SEQ ID NO: 1064 - protein, 9 aa, Homo sapiens
TMFRRPHSC

SEQ ID NO: 1065 - protein, 10 aa, Homo sapiens
TMFRRPHSCL

SEQ ID NO: 1066 - protein, 9 aa, Homo sapiens
VLLSVHLIV

SEQ ID NO: 1067 - protein, 10 aa, Homo sapiens
VLLSVHLIVL

SEQ ID NO: 1068 - protein, 9 aa, Homo sapiens
VLRVVRLPA

SEQ ID NO: 1069 - protein, 9 aa, Homo sapiens
VVRLPAPFR

SEQ ID NO: 1070 - protein, 9 aa, Homo sapiens
LTSSSREWK

SEQ ID NO: 1071 - protein, 10 aa, Homo sapiens
LLTSSSREWK

SEQ ID NO: 1072 - protein, 10 aa, Homo sapiens
YPAYSKDSGL

SEQ ID NO: 1073 - protein, 9 aa, Homo sapiens
EWKVKFPEL

SEQ ID NO: 1074 - protein, 9 aa, Homo sapiens
KFPELLCVW

SEQ ID NO: 1075 - protein, 9 aa, Homo sapiens
FLKAESKIM

SEQ ID NO: 1076 - protein, 9 aa, Homo sapiens
LQHGHRHGL

SEQ ID NO: 1077 - protein, 9 aa, Homo sapiens
EPHLALQPL

SEQ ID NO: 1078 - protein, 9 aa, Homo sapiens
FATLQRSSL

SEQ ID NO: 1079 - protein, 10 aa, Homo sapiens
MFATLQRSSL

SEQ ID NO: 1080 - protein, 9 aa, Homo sapiens
MFLKAESKI

SEQ ID NO: 1081 - protein, 9 aa, Homo sapiens
MLTGPPARV

SEQ ID NO: 1082 - protein, 9 aa, Homo sapiens
GPPARVPAV

SEQ ID NO: 1083 - protein, 9 aa, Homo sapiens
ALQPLQPHA

SEQ ID NO: 1084 - protein, 9 aa, Homo sapiens
VLPEPHLAL

SEQ ID NO: 1085 - protein, 9 aa, Homo sapiens
YMFLKAESK

SEQ ID NO: 1086 - protein, 10 aa, Homo sapiens
VPAVPFDLHF

SEQ ID NO: 1087 - protein, 9 aa, Homo sapiens
AIQPVLWTT

SEQ ID NO: 1088 - protein, 10 aa, Homo sapiens
TLQRSSLWCL

SEQ ID NO: 1089 - protein, 9 aa, Homo sapiens
KIMFATLQR

SEQ ID NO: 1090 - protein, 10 aa, Homo sapiens
QPVLWTTPPL

SEQ ID NO: 1091 - protein, 9 aa, Homo sapiens
ESKIMFATL

SEQ ID NO: 1092 - protein, 10 aa, Homo sapiens
IMKPKRDGYM

SEQ ID NO: 1093 - protein, 9 aa, Homo sapiens
KPKRDGYMF

SEQ ID NO: 1094 - protein, 10 aa, Homo sapiens
FLKAESKIMF

SEQ ID NO: 1095 - protein, 10 aa, Homo sapiens
KPKRDGYMFL

SEQ ID NO: 1096 - protein, 9 aa, Homo sapiens
LHFCRSSIM

SEQ ID NO: 1097 - protein, 10 aa, Homo sapiens
YMFLKAESKI

SEQ ID NO: 1098 - protein, 9 aa, Homo sapiens
VMAYVMAGV

SEQ ID NO: 1099 - protein, 10 aa, Homo sapiens
YVMAYVMAGV

SEQ ID NO: 1100 - protein, 9 aa, Homo sapiens
YVMAYVMAG

SEQ ID NO: 1101 - protein, 9 aa, Homo sapiens
SLMPAHWSI

SEQ ID NO: 1102 - protein, 10 aa, Homo sapiens
LSLMPAHWSI

SEQ ID NO: 1103 - protein, 9 aa, Homo sapiens
FQMQPLSLM

SEQ ID NO: 1104 - protein, 10 aa, Homo sapiens
LLQVTQTSFA

SEQ ID NO: 1105 - protein, 9 aa, Homo sapiens
RLWHLLLQV

SEQ ID NO: 1106 - protein, 10 aa, Homo sapiens
LQVTQTSFAL

SEQ ID NO: 1107 - protein, 10 aa, Homo sapiens
RLWHLLLQVT

SEQ ID NO: 1108 - protein, 9 aa, Homo sapiens
ALAPTLTHM

SEQ ID NO: 1109 - protein, 10 aa, Homo sapiens
ALAPTLTHML

SEQ ID NO: 1110 - protein, 9 aa, Homo sapiens
SLGNHLCPL

SEQ ID NO: 1111 - protein, 9 aa, Homo sapiens
TQLARFFPI

SEQ ID NO: 1112 - protein, 10 aa, Homo sapiens
KSRQNHLQLK

SEQ ID NO: 1113 - protein, 9 aa, Homo sapiens
KSRQNHLQL

SEQ ID NO: 1114 - protein, 9 aa, Homo sapiens
KLRSPLWIF

SEQ ID NO: 1115 - protein, 9 aa, Homo sapiens
KISNWSLKK

SEQ ID NO: 1116 - protein, 9 aa, Homo sapiens
SLKKVPALK

SEQ ID NO: 1117 - protein, 10 aa, Homo sapiens
WSLKKVPALK

SEQ ID NO: 1118 - protein, 10 aa, Homo sapiens
KISNWSLKKV

SEQ ID NO: 1119 - protein, 9 aa, Homo sapiens
SQKSRQNHL

SEQ ID NO: 1120 - protein, 9 aa, Homo sapiens
ALKKLRSPL

SEQ ID NO: 1121 - protein, 9 aa, Homo sapiens
WSLKKVPAL

SEQ ID NO: 1122 - protein, 10 aa, Homo sapiens
HLQLKSCRRK

SEQ ID NO: 1123 - protein, 10 aa, Homo sapiens
SLKKVPALKK

SEQ ID NO: 1124 - protein, 10 aa, Homo sapiens
LPRKPTRAAT

SEQ ID NO: 1125 - protein, 9 aa, Homo sapiens
LPRKPTRAA

SEQ ID NO: 1126 - protein, 10 aa, Homo sapiens
KPTRAATVSV

SEQ ID NO: 1127 - protein, 9 aa, Homo sapiens
RVQNQGHLL

SEQ ID NO: 1128 - protein, 10 aa, Homo sapiens
MLTDSLFLPI

SEQ ID NO: 1129 - protein, 9 aa, Homo sapiens
SMLTDSLFL

SEQ ID NO: 1130 - protein, 4 aa, Homo sapiens
AVEW

SEQ ID NO: 1131 - protein, 4 aa, Homo sapiens
VHPA

SEQ ID NO: 1132 - protein, 4 aa, Homo sapiens
MPAV

SEQ ID NO: 1133 - protein, 4 aa, Homo sapiens
SIRH

SEQ ID NO: 1134 - protein, 4 aa, Homo sapiens
SQIS

SEQ ID NO: 1135 - protein, 4 aa, Homo sapiens
CSNH

SEQ ID NO: 1136 - protein, 4 aa, Homo sapiens
YHEA

SEQ ID NO: 1137 - protein, 4 aa, Homo sapiens
YVMA

SEQ ID NO: 1138 - protein, 4 aa, Homo sapiens
LSPH

SEQ ID NO: 1139 - protein, 4 aa, Homo sapiens
CRLS

SEQ ID NO: 1140 - protein, 4 aa, Homo sapiens
RHTP

SEQ ID NO: 1141 - protein, 4 aa, Homo sapiens
AAVG

SEQ ID NO: 1142 - protein, 4 aa, Homo sapiens
AWAA

SEQ ID NO: 1143 - protein, 4 aa, Homo sapiens
CSES

SEQ ID NO: 1144 - protein, 4 aa, Homo sapiens
QPSL

SEQ ID NO: 1145 - protein, 4 aa, Homo sapiens
SHST

SEQ ID NO: 1146 - protein, 4 aa, Homo sapiens
QCIL

SEQ ID NO: 1147 - protein, 8 aa, Homo sapiens
DGVGKSAL

SEQ ID NO: 1148 - protein, 8 aa, Homo sapiens
VGVGKSAL

SEQ ID NO: 1149 - protein, 8 aa, Homo sapiens
CGVGKSAL

SEQ ID NO: 1150 - protein, 11 aa, Homo sapiens
DTAGHEEYSAM

SEQ ID NO: 1151 - protein, 8 aa, Homo sapiens
GHEEYSAM

SEQ ID NO: 1152 - protein, 8 aa, Homo sapiens
DTAGHEEY

SEQ ID NO: 1153 - protein, 9 aa, Homo sapiens
ILDTAGHEE

SEQ ID NO: 1154 - protein, 9 aa, Homo sapiens
AGHEEYSAM

SEQ ID NO: 1155 - protein, 9 aa, Homo sapiens
HEEYSAMRD

SEQ ID NO: 1156 - protein, 9 aa, Homo sapiens
TAGHEEYSA

SEQ ID NO: 1157 - protein, 9 aa, Homo sapiens
DTAGHEEYS

SEQ ID NO: 1158 - protein, 12 aa, Homo sapiens
DTAGHEEYSAMR

SEQ ID NO: 1159 - protein, 9 aa, Homo sapiens
LDTAGHEEY

SEQ ID NO: 1160 - protein, 10 aa, Homo sapiens
LLDILDTAGH

SEQ ID NO: 1161 - protein, 10 aa, Homo sapiens
TAGHEEYSAM

SEQ ID NO: 1162 - protein, 9 aa, Homo sapiens
DILDTAGHE

SEQ ID NO: 1163 - protein, 11 aa, Homo sapiens
DILDTAGHEEY

SEQ ID NO: 1164 - protein, 8 aa, Homo sapiens
DILDTAGH

SEQ ID NO: 1165 - protein, 8 aa, Homo sapiens
ILDTAGHE

SEQ ID NO: 1166 - protein, 10 aa, Homo sapiens
DTAGHEEYSA

SEQ ID NO: 1167 - protein, 11 aa, Homo sapiens
DTAGREEYSAM

SEQ ID NO: 1168 - protein, 8 aa, Homo sapiens
DILDTAGR

SEQ ID NO: 1169 - protein, 8 aa, Homo sapiens
DTAGREEY

SEQ ID NO: 1170 - protein, 11 aa, Homo sapiens
CLLDILDTAGR

SEQ ID NO: 1171 - protein, 9 aa, Homo sapiens
REEYSAMRD

SEQ ID NO: 1172 - protein, 9 aa, Homo sapiens
GREEYSAMR

SEQ ID NO: 1173 - protein, 9 aa, Homo sapiens
ILDTAGREE

SEQ ID NO: 1174 - protein, 9 aa, Homo sapiens
AGREEYSAM

SEQ ID NO: 1175 - protein, 9 aa, Homo sapiens
DILDTAGRE

SEQ ID NO: 1176 - protein, 8 aa, Homo sapiens
GREEYSAM

SEQ ID NO: 1177 - protein, 9 aa, Homo sapiens
TAGREEYSA

SEQ ID NO: 1178 - protein, 9 aa, Homo sapiens
DTAGREEYS

SEQ ID NO: 1179 - protein, 8 aa, Homo sapiens
ILDTAGRE

SEQ ID NO: 1180 - protein, 12 aa, Homo sapiens
DTAGREEYSAMR

SEQ ID NO: 1181 - protein, 10 aa, Homo sapiens
LLDILDTAGR

SEQ ID NO: 1182 - protein, 10 aa, Homo sapiens
TAGREEYSAM

SEQ ID NO: 1183 - protein, 11 aa, Homo sapiens
DILDTAGREEY

SEQ ID NO: 1184 - protein, 10 aa, Homo sapiens
AGREEYSAMR

SEQ ID NO: 1185 - protein, 9 aa, Homo sapiens
LDILDTAGR

SEQ ID NO: 1186 - protein, 9 aa, Homo sapiens
LDTAGREEY

SEQ ID NO: 1187 - protein, 11 aa, Homo sapiens
REEYSAMRDQY

SEQ ID NO: 1188 - protein, 11 aa, Homo sapiens
DTAGKEEYSAM

SEQ ID NO: 1189 - protein, 11 aa, Homo sapiens
CLLDILDTAGK

SEQ ID NO: 1190 - protein, 8 aa, Homo sapiens
DTAGKEEY

SEQ ID NO: 1191 - protein, 9 aa, Homo sapiens
AGKEEYSAM

SEQ ID NO: 1192 - protein, 9 aa, Homo sapiens
DILDTAGKE

SEQ ID NO: 1193 - protein, 9 aa, Homo sapiens
KEEYSAMRD

SEQ ID NO: 1194 - protein, 8 aa, Homo sapiens
GKEEYSAM

SEQ ID NO: 1195 - protein, 9 aa, Homo sapiens
ILDTAGKEE

SEQ ID NO: 1196 - protein, 9 aa, Homo sapiens
DTAGKEEYS

SEQ ID NO: 1197 - protein, 9 aa, Homo sapiens
LDTAGKEEY

SEQ ID NO: 1198 - protein, 9 aa, Homo sapiens
TAGKEEYSA

SEQ ID NO: 1199 - protein, 8 aa, Homo sapiens
DILDTAGK

SEQ ID NO: 1200 - protein, 12 aa, Homo sapiens
DTAGKEEYSAMR

SEQ ID NO: 1201 - protein, 10 aa, Homo sapiens
LLDILDTAGK

SEQ ID NO: 1202 - protein, 8 aa, Homo sapiens
ILDTAGKE

SEQ ID NO: 1203 - protein, 10 aa, Homo sapiens
AGKEEYSAMR

SEQ ID NO: 1204 - protein, 10 aa, Homo sapiens
TAGKEEYSAM

SEQ ID NO: 1205 - protein, 12 aa, Homo sapiens
GLEEYSAMRDQY

SEQ ID NO: 1206 - protein, 8 aa, Homo sapiens
DTAGLEEY

SEQ ID NO: 1207 - protein, 11 aa, Homo sapiens
DTAGLEEYSAM

SEQ ID NO: 1208 - protein, 9 aa, Homo sapiens
ILDTAGLEE

SEQ ID NO: 1209 - protein, 11 aa, Homo sapiens
CLLDILDTAGL

SEQ ID NO: 1210 - protein, 8 aa, Homo sapiens
DILDTAGL

SEQ ID NO: 1211 - protein, 11 aa, Homo sapiens
DILDTAGLEEY

SEQ ID NO: 1212 - protein, 9 aa, Homo sapiens
DTAGLEEYS

SEQ ID NO: 1213 - protein, 9 aa, Homo sapiens
GLEEYSAMR

SEQ ID NO: 1214 - protein, 8 aa, Homo sapiens
ILDTAGLE

SEQ ID NO: 1215 - protein, 9 aa, Homo sapiens
LDTAGLEEY

SEQ ID NO: 1216 - protein, 12 aa, Homo sapiens
DTAGLEEYSAMR

SEQ ID NO: 1217 - protein, 9 aa, Homo sapiens
AGLEEYSAM

SEQ ID NO: 1218 - protein, 10 aa, Homo sapiens
DTAGLEEYSA

SEQ ID NO: 1219 - protein, 9 aa, Homo sapiens
LDILDTAGL

SEQ ID NO: 1220 - protein, 10 aa, Homo sapiens
AGLEEYSAMR

SEQ ID NO: 1221 - protein, 9 aa, Homo sapiens
LEEYSAMRD

SEQ ID NO: 1222 - protein, 11 aa, Homo sapiens
LLDILDTAGLE

SEQ ID NO: 1223 - protein, 9 aa, Homo sapiens
DILDTAGLE

SEQ ID NO: 1224 - protein, 9 aa, Homo sapiens
TAGLEEYSA

SEQ ID NO: 1225 - protein, 8 aa, Homo sapiens
GLEEYSAM

SEQ ID NO: 1226 - protein, 9 aa, Homo sapiens
AAGVGKSAL

SEQ ID NO: 1227 - protein, 10 aa, Homo sapiens
VVVGAAGVGK

SEQ ID NO: 1228 - protein, 9 aa, Homo sapiens
VVGAAGVGK

SEQ ID NO: 1229 - protein, 11 aa, Homo sapiens
TEYKLVVVGAA

SEQ ID NO: 1230 - protein, 10 aa, Homo sapiens
GAAGVGKSAL

SEQ ID NO: 1231 - protein, 9 aa, Homo sapiens
LVVVGAAGV

SEQ ID NO: 1232 - protein, 9 aa, Homo sapiens
GAAGVGKSA

SEQ ID NO: 1233 - protein, 10 aa, Homo sapiens
KLVVVGAAGV

SEQ ID NO: 1234 - protein, 8 aa, Homo sapiens
AGVGKSAL

SEQ ID NO: 1235 - protein, 8 aa, Homo sapiens
VVVGAAGV

SEQ ID NO: 1236 - protein, 9 aa, Homo sapiens
YKLVVVGAA

SEQ ID NO: 1237 - protein, 8 aa, Homo sapiens
AAGVGKSA

SEQ ID NO: 1238 - protein, 10 aa, Homo sapiens
VGAAGVGKSA

SEQ ID NO: 1239 - protein, 10 aa, Homo sapiens
AGVGKSALTI

SEQ ID NO: 1240 - protein, 9 aa, Homo sapiens
ACGVGKSAL

SEQ ID NO: 1241 - protein, 10 aa, Homo sapiens
GACGVGKSAL

SEQ ID NO: 1242 - protein, 9 aa, Homo sapiens
YKLVVVGAC

SEQ ID NO: 1243 - protein, 9 aa, Homo sapiens
GACGVGKSA

SEQ ID NO: 1244 - protein, 10 aa, Homo sapiens
GADGVGKSAL

SEQ ID NO: 1245 - protein, 9 aa, Homo sapiens
ADGVGKSAL

SEQ ID NO: 1246 - protein, 9 aa, Homo sapiens
GADGVGKSA

SEQ ID NO: 1247 - protein, 8 aa, Homo sapiens
GADGVGKS

SEQ ID NO: 1248 - protein, 10 aa, Homo sapiens
VGADGVGKSA

SEQ ID NO: 1249 - protein, 8 aa, Homo sapiens
VGADGVGK

SEQ ID NO: 1250 - protein, 9 aa, Homo sapiens
YKLVVVGAD

SEQ ID NO: 1251 - protein, 10 aa, Homo sapiens
DGVGKSALTI

SEQ ID NO: 1252 - protein, 12 aa, Homo sapiens
KLVVVGADGVGK

SEQ ID NO: 1253 - protein, 9 aa, Homo sapiens
GARGVGKSA

SEQ ID NO: 1254 - protein, 8 aa, Homo sapiens
RGVGKSAL

SEQ ID NO: 1255 - protein, 10 aa, Homo sapiens
GARGVGKSAL

SEQ ID NO: 1256 - protein, 10 aa, Homo sapiens
RGVGKSALTI

SEQ ID NO: 1257 - protein, 9 aa, Homo sapiens
LVVVGARGV

SEQ ID NO: 1258 - protein, 10 aa, Homo sapiens
KLVVVGARGV

SEQ ID NO: 1259 - protein, 9 aa, Homo sapiens
YKLVVVGAR

SEQ ID NO: 1260 - protein, 8 aa, Homo sapiens
KLVVVGAR

SEQ ID NO: 1261 - protein, 8 aa, Homo sapiens
VVVGARGV

SEQ ID NO: 1262 - protein, 12 aa, Homo sapiens
KLVVVGARGVGK

SEQ ID NO: 1263 - protein, 10 aa, Homo sapiens
VVVGASGVGK

SEQ ID NO: 1264 - protein, 9 aa, Homo sapiens
VVGASGVGK

SEQ ID NO: 1265 - protein, 9 aa, Homo sapiens
ASGVGKSAL

SEQ ID NO: 1266 - protein, 10 aa, Homo sapiens
GASGVGKSAL

SEQ ID NO: 1267 - protein, 8 aa, Homo sapiens
SGVGKSAL

SEQ ID NO: 1268 - protein, 9 aa, Homo sapiens
GASGVGKSA

SEQ ID NO: 1269 - protein, 10 aa, Homo sapiens
KLVVVGASGV

SEQ ID NO: 1270 - protein, 9 aa, Homo sapiens
LVVVGASGV

SEQ ID NO: 1271 - protein, 8 aa, Homo sapiens
ASGVGKSA

SEQ ID NO: 1272 - protein, 10 aa, Homo sapiens
SGVGKSALTI

SEQ ID NO: 1273 - protein, 10 aa, Homo sapiens
VGASGVGKSA

SEQ ID NO: 1274 - protein, 9 aa, Homo sapiens
KLVVVGASG

SEQ ID NO: 1275 - protein, 9 aa, Homo sapiens
AVGVGKSAL

SEQ ID NO: 1276 - protein, 10 aa, Homo sapiens
GAVGVGKSAL

SEQ ID NO: 1277 - protein, 8 aa, Homo sapiens
VVVGAVGV

SEQ ID NO: 1278 - protein, 9 aa, Homo sapiens
GAVGVGKSA

SEQ ID NO: 1279 - protein, 10 aa, Homo sapiens
VGVGKSALTI

SEQ ID NO: 1280 - protein, 8 aa, Homo sapiens
AVGVGKSA

SEQ ID NO: 1281 - protein, 9 aa, Homo sapiens
YKLVVVGAV

SEQ ID NO: 1282 - protein, 11 aa, Homo sapiens
LVVVGAVGVGK

SEQ ID NO: 1283 - protein, 12 aa, Homo sapiens
KLVVVGAVGVGK

SEQ ID NO: 1284 - protein, 10 aa, Homo sapiens
VVVGAGCVGK

SEQ ID NO: 1285 - protein, 9 aa, Homo sapiens
VVGAGCVGK

SEQ ID NO: 1286 - protein, 9 aa, Homo sapiens
AGCVGKSAL

SEQ ID NO: 1287 - protein, 9 aa, Homo sapiens
CVGKSALTI

SEQ ID NO: 1288 - protein, 10 aa, Homo sapiens
GCVGKSALTI

SEQ ID NO: 1289 - protein, 9 aa, Homo sapiens
KLVVVGAGC

SEQ ID NO: 1290 - protein, 8 aa, Homo sapiens
GCVGKSAL

SEQ ID NO: 1291 - protein, 9 aa, Homo sapiens
AGDVGKSAL

SEQ ID NO: 1292 - protein, 9 aa, Homo sapiens
VVGAGDVGK

SEQ ID NO: 1293 - protein, 10 aa, Homo sapiens
VVVGAGDVGK

SEQ ID NO: 1294 - protein, 9 aa, Homo sapiens
GAGDVGKSA

SEQ ID NO: 1295 - protein, 10 aa, Homo sapiens
GAGDVGKSAL

SEQ ID NO: 1296 - protein, 9 aa, Homo sapiens
DVGKSALTI

SEQ ID NO: 1297 - protein, 8 aa, Homo sapiens
GDVGKSAL

SEQ ID NO: 1298 - protein, 48 aa, Homo sapiens
AQAKAVCSQESRDVLCELSDHHNHTLEEECQWGPCLQCLWALLQASQY

SEQ ID NO: 1299 - protein, 9 aa, Homo sapiens
CLQCLWALL

SEQ ID NO: 1300 - protein, 10 aa, Homo sapiens
CQWGPCLQCL

SEQ ID NO: 1301 - protein, 9 aa, Homo sapiens
QWGPCLQCL

SEQ ID NO: 1302 - protein, 10 aa, Homo sapiens
QWGPCLQCLW

SEQ ID NO: 1303 - protein, 9 aa, Homo sapiens
AIQPVLWTT

SEQ ID NO: 1304 - protein, 9 aa, Homo sapiens
ALQPLQPHA

SEQ ID NO: 1305 - protein, 10 aa, Homo sapiens
DLHFCRSSIM

SEQ ID NO: 1306 - protein, 9 aa, Homo sapiens
EPHLALQPL

SEQ ID NO: 1307 - protein, 9 aa, Homo sapiens
ESKIMFATL

SEQ ID NO: 1308 - protein, 9 aa, Homo sapiens
FATLQRSSL

SEQ ID NO: 1309 - protein, 9 aa, Homo sapiens
FLKAESKIM

SEQ ID NO: 1310 - protein, 10 aa, Homo sapiens
FLKAESKIMF

SEQ ID NO: 1311 - protein, 9 aa, Homo sapiens
GPPARVPAV

SEQ ID NO: 1312 - protein, 10 aa, Homo sapiens
IMKPKRDGYM

SEQ ID NO: 1313 - protein, 9 aa, Homo sapiens
KIMFATLQR

SEQ ID NO: 1314 - protein, 9 aa, Homo sapiens
KPKRDGYMF

SEQ ID NO: 1315 - protein, 10 aa, Homo sapiens
KPKRDGYMFL

SEQ ID NO: 1316 - protein, 9 aa, Homo sapiens
LHFCRSSIM

SEQ ID NO: 1317 - protein, 9 aa, Homo sapiens
LQHGHRHGL

SEQ ID NO: 1318 - protein, 10 aa, Homo sapiens
MFATLQRSSL

SEQ ID NO: 1319 - protein, 9 aa, Homo sapiens
MFLKAESKI

SEQ ID NO: 1320 - protein, 9 aa, Homo sapiens
MLTGPPARV

SEQ ID NO: 1321 - protein, 10 aa, Homo sapiens
QPVLWTTPPL

SEQ ID NO: 1322 - protein, 10 aa, Homo sapiens
SMLTGPPARV

SEQ ID NO: 1323 - protein, 10 aa, Homo sapiens
TLQRSSLWCL

SEQ ID NO: 1324 - protein, 9 aa, Homo sapiens
VLPEPHLAL

SEQ ID NO: 1325 - protein, 10 aa, Homo sapiens
VPAVPFDLHF

SEQ ID NO: 1326 - protein, 9 aa, Homo sapiens
YMFLKAESK

SEQ ID NO: 1327 - protein, 10 aa, Homo sapiens
YMFLKAESKI

SEQ ID NO: 1328 - protein, 10 aa, Homo sapiens
FTCQIPGIYY

SEQ ID NO: 1329 - protein, 8 aa, Homo sapiens
GSDGKPGY

SEQ ID NO: 1330 - protein, 8 aa, Homo sapiens
NAESNGLY

SEQ ID NO: 1331 - protein, 9 aa, Homo sapiens
LTENDQVWL

SEQ ID NO: 1332 - protein, 9 aa, Homo sapiens
GTHVWVGLY

SEQ ID NO: 1333 - protein, 9 aa, Homo sapiens
TYDEYTKGY

SEQ ID NO: 1334 - protein, 10 aa, Homo sapiens
YTYDEYTKGY

SEQ ID NO: 1335 - protein, 9 aa, Homo sapiens
FTCQIPGIY

SEQ ID NO: 1336 - protein, 12 aa, Homo sapiens
NAESNGLYSSEY

SEQ ID NO: 1337 - protein, 9 aa, Homo sapiens
YLDQASGSA

SEQ ID NO: 1338 - protein, 9 aa, Homo sapiens
FLLLVSLNL

SEQ ID NO: 1339 - protein, 10 aa, Homo sapiens
FLLLVSLNLV

SEQ ID NO: 1340 - protein, 9 aa, Homo sapiens
GLYKNGTPV

SEQ ID NO: 1341 - protein, 11 aa, Homo sapiens
GLDGPKGNPGL

SEQ ID NO: 1342 - protein, 9 aa, Homo sapiens
LLLVSLNLV

SEQ ID NO: 1343 - protein, 10 aa, Homo sapiens
SLSGTPLVSA

SEQ ID NO: 1344 - protein, 8 aa, Homo sapiens
GLYSSEYV

SEQ ID NO: 1345 - protein, 8 aa, Homo sapiens
SLSGTPLV

SEQ ID NO: 1346 - protein, 9 aa, Homo sapiens
MLPQIPFLL

SEQ ID NO: 1347 - protein, 10 aa, Homo sapiens
GLPGPPGPSA

SEQ ID NO: 1348 - protein, 9 aa, Homo sapiens
SAFTVILSK

SEQ ID NO: 1349 - protein, 9 aa, Homo sapiens
AVMPEGFIK

SEQ ID NO: 1350 - protein, 11 aa, Homo sapiens
GLYKNGTPVMY

SEQ ID NO: 1351 - protein, 10 aa, Homo sapiens
AIGTPIPFDK

SEQ ID NO: 1352 - protein, 9 aa, Homo sapiens
GLPGGPGAK

SEQ ID NO: 1353 - protein, 9 aa, Homo sapiens
ILYNRQQHY

SEQ ID NO: 1354 - protein, 11 aa, Homo sapiens
AGPPGPPGFGK

SEQ ID NO: 1355 - protein, 9 aa, Homo sapiens
GIPGFPGSK

SEQ ID NO: 1356 - protein, 10 aa, Homo sapiens
GTHVWVGLYK

SEQ ID NO: 1357 - protein, 9 aa, Homo sapiens
GVPGQPGIK

SEQ ID NO: 1358 - protein, 10 aa, Homo sapiens
VSAFTVILSK

SEQ ID NO: 1359 - protein, 10 aa, Homo sapiens
AVMPEGFIKA

SEQ ID NO: 1360 - protein, 9 aa, Homo sapiens
SSFSGFLVA

SEQ ID NO: 1361 - protein, 11 aa, Homo sapiens
PVSAFTVILSK

SEQ ID NO: 1362 - protein, 9 aa, Homo sapiens
GVPGMNGQK

SEQ ID NO: 1363 - protein, 11 aa, Homo sapiens
AYPAIGTPIPF

SEQ ID NO: 1364 - protein, 9 aa, Homo sapiens
IGPPGIPGF

SEQ ID NO: 1365 - protein, 9 aa, Homo sapiens
HYDPRTGIF

SEQ ID NO: 1366 - protein, 10 aa, Homo sapiens
EYVHSSFSGF

SEQ ID NO: 1367 - protein, 9 aa, Homo sapiens
AGPPGPPGF

SEQ ID NO: 1368 - protein, 9 aa, Homo sapiens
YYFSYHVHV

SEQ ID NO: 1369 - protein, 9 aa, Homo sapiens
AYPAIGTPI

SEQ ID NO: 1370 - protein, 9 aa, Homo sapiens
PLPNTKTQF

SEQ ID NO: 1371 - protein, 9 aa, Homo sapiens
CQIPGIYYF

SEQ ID NO: 1372 - protein, 9 aa, Homo sapiens
RPSLSGTPL

SEQ ID NO: 1373 - protein, 9 aa, Homo sapiens
LPQIPFLLL

SEQ ID NO: 1374 - protein, 9 aa, Homo sapiens
IPFLLLVSL

SEQ ID NO: 1375 - protein, 10 aa, Homo sapiens
LPGPPGPSAV

SEQ ID NO: 1376 - protein, 11 aa, Homo sapiens
GPIGPPGIPGF

SEQ ID NO: 1377 - protein, 9 aa, Homo sapiens
IPGPAGISV

SEQ ID NO: 1378 - protein, 10 aa, Homo sapiens
YPAIGTPIPF

SEQ ID NO: 1379 - protein, 10 aa, Homo sapiens
SPGPPGPAGI

SEQ ID NO: 1380 - protein, 9 aa, Homo sapiens
LPGPPGPSA

SEQ ID NO: 1381 - protein, 9 aa, Homo sapiens
SPGPPGPAG

SEQ ID NO: 1382 - protein, 9 aa, Homo sapiens
TIKSKGIAV

SEQ ID NO: 1383 - protein, 9 aa, Homo sapiens
HVHVKGTHV

SEQ ID NO: 1384 - protein, 8 aa, Homo sapiens
LPNTKTQF

SEQ ID NO: 1385 - protein, 8 aa, Homo sapiens
LPQIPFLL

SEQ ID NO: 1386 - protein, 8 aa, Homo sapiens
PFLLLVSL

SEQ ID NO: 1387 - protein, 8 aa, Homo sapiens
SLNLVHGV

SEQ ID NO: 1388 - protein, 8 aa, Homo sapiens
TGMPVSAF

SEQ ID NO: 1389 - protein, 9 aa, Homo sapiens
TPIPFDKIL

SEQ ID NO: 1390 - protein, 9 aa, Homo sapiens
IMKPKRDGY

SEQ ID NO: 1391 - protein, 10 aa, Homo sapiens
SIMKPKRDGY

SEQ ID NO: 1392 - protein, 10 aa, Homo sapiens
AESKIMFATL

SEQ ID NO: 1393 - protein, 9 aa, Homo sapiens
AESKIMFAT

SEQ ID NO: 1394 - protein, 9 aa, Homo sapiens
SLQCVSLHL

SEQ ID NO: 1395 - protein, 9 aa, Homo sapiens
LVLSIALSV

SEQ ID NO: 1396 - protein, 9 aa, Homo sapiens
VILGVHLSV

SEQ ID NO: 1397 - protein, 9 aa, Homo sapiens
VLAPQESSV

SEQ ID NO: 1398 - protein, 10 aa, Homo sapiens
SLQCVSLHLL

SEQ ID NO: 1399 - protein, 9 aa, Homo sapiens
MLLRLSEPA

SEQ ID NO: 1400 - protein, 9 aa, Homo sapiens
LTMPALPMV

SEQ ID NO: 1401 - protein, 10 aa, Homo sapiens
FLTLSVTWIA

SEQ ID NO: 1402 - protein, 9 aa, Homo sapiens
KLQCVDLHV

SEQ ID NO: 1403 - protein, 10 aa, Homo sapiens
FLTPKKLQCV

SEQ ID NO: 1404 - protein, 10 aa, Homo sapiens
FLRPGDDSTL

SEQ ID NO: 1405 - protein, 9 aa, Homo sapiens
FLGYLILGV

SEQ ID NO: 1406 - protein, 9 aa, Homo sapiens
LLANDLMLI

SEQ ID NO: 1407 - protein, 9 aa, Homo sapiens
FQNSYTIGL

SEQ ID NO: 1408 - protein, 9 aa, Homo sapiens
MLIKLDESV

SEQ ID NO: 1409 - protein, 9 aa, Homo sapiens
VLQCVNVSV

SEQ ID NO: 1410 - protein, 10 aa, Homo sapiens
ILNDTGCHYV

SEQ ID NO: 1411 - protein, 10 aa, Homo sapiens
FQYPEFSIEL

SEQ ID NO: 1412 - protein, 9 aa, Homo sapiens
ILGKYQLNV

SEQ ID NO: 1413 - protein, 10 aa, Homo sapiens
LLGNSVNFTV

SEQ ID NO: 1414 - protein, 9 aa, Homo sapiens
VLDCCISLL

SEQ ID NO: 1415 - protein, 9 aa, Homo sapiens
ILGSFELQL

SEQ ID NO: 1416 - protein, 9 aa, Homo sapiens
RLIWLVKMV

SEQ ID NO: 1417 - protein, 11 aa, Homo sapiens
VLLGNSVNFTV

SEQ ID NO: 1418 - protein, 9 aa, Homo sapiens
TLAIPLTDV

SEQ ID NO: 1419 - protein, 4 aa, Homo sapiens
DEAH

SEQ ID NO: 1420 - protein, 10 aa, Homo sapiens
  MISC_FEATURE (1)..(1): May or may not be present
VVVGADGVGK

SEQ ID NO: 1421 - protein, 10 aa, Homo sapiens
  MISC_FEATURE (10)..(10): May or may not be present
GADGVGKSAL

SEQ ID NO: 1422 - protein, 10 aa, Homo sapiens
  MISC_FEATURE (1)..(1): May or may not be present
VVVGAVGVGK

SEQ ID NO: 1423 - protein, 10 aa, Homo sapiens
  MISC_FEATURE (1)..(1): May or may not be present
GAVGVGKSAL

SEQ ID NO: 1424 - protein, 10 aa, Homo sapiens
  MISC_FEATURE (1)..(1): May or may not be present
VVVGACGVGK

SEQ ID NO: 1425 - protein, 10 aa, Homo sapiens
  MISC_FEATURE (1)..(1): May or may not be present
VVVGARGVGK

SEQ ID NO: 1426 - protein, 9 aa, Homo sapiens
  MISC_FEATURE (1)..(1): May or may not be present
ARGVGKSAL

SEQ ID NO: 1427 - protein, 15 aa, Homo sapiens
TEYKLVVVGAVGVGK

SEQ ID NO: 1428 - protein, 20 aa, Homo sapiens
TEYKLVVVGAVGVGKSALTI

(19) United States
(12) Patent Application Publication
(10) Pub. No.: US 2022/0282217 A1
(43) Pub. Date: Sep. 8, 2022

(54) COMPOSITIONS AND METHODS FOR PREPARING T CELL COMPOSITIONS AND USES THEREOF

(71) Applicant: BIONTECH US INC., Cambridge, MA (US)

(72) Inventors: Robert ANG, Waban, MA (US); Vikram JUNEJA, Newton, MA (US); Richard GAYNOR, Carmel, IN (US)

(21) Appl. No.: 17/599,468

(22) PCT Filed: Mar. 30, 2020
(86) PCT No.: PCT/US20/25796

§ 371 Date: Sep. 28, 2021

Related U.S. Application Data
(60) Provisional application No. 62/827,018, filed on Mar. 30, 2019.

(51) Int. Cl. C12N 5/0783; A61K 35/17

(52) U.S. Cl. CPC C12N 5/0638; C12N 5/0637; A61K 35/17; C12N 2501/26

(57) ABSTRACT

Provided herein are compositions and methods for preparing T cell compositions and uses thereof, including methods for treating cancer in a subject in need thereof by administering T cells induced with peptides comprising an epitope sequence from a library of epitope sequences, wherein each epitope sequence in the library is matched to a protein encoded by an HLA allele and binds to a protein encoded by an HLA allele of the subject, is immunogenic according to an immunogenic assay, is presented by antigen presenting cells according to a mass spectrometry assay, and stimulates T cells to be cytotoxic according to a cytotoxicity assay.

### CROSS REFERENCE

This application claims the benefit of U.S. Provisional Application No. 62/827,018, filed on Mar. 30, 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 15, 2020, is named 50401-744(Generic)_SL.txt and is 313,317 bytes in size.

### BACKGROUND

Adoptive immunotherapy or adoptive cellular therapy with lymphocytes (ACT) is the transfer of gene modified T lymphocytes to a subject for the therapy of disease. Adoptive immunotherapy has yet to realize its potential for treating a wide variety of diseases including cancer, infectious disease, autoimmune disease, inflammatory disease, and immunodeficiency. However, most, if not all adoptive immunotherapy strategies require T cell activation and expansion steps to generate a clinically effective, therapeutic dose of T cells. Existing strategies of obtaining patient cells, and ex vivo activation, expansion and recovery of effective number of cells for ACT is a prolonged, cumbersome and an inherently complex process—and poses a serious challenge. Accordingly, there remains a need for developing compositions and methods for expansion and induction of antigen specific T cells with a favorable phenotype and function and within a shorter time span.

### SUMMARY

Provided herein is a method for treating cancer in a subject in need thereof comprising: selecting at least one epitope sequence from a library of epitope sequences, wherein each epitope sequence in the library is matched to a protein encoded by an HLA allele of the subject; and contacting a T cell from the subject or an allogeneic T cell with one or more peptides comprising the at least one selected epitope sequence, wherein each of the at least one selected epitope sequence is pre-validated to satisfy at least three of the following criteria: binds to a protein encoded by an HLA allele of the subject, is immunogenic according to an immunogenicity assay, is presented by antigen presenting cells (APCs) according to a mass spectrometry assay, and stimulates T cells to be cytotoxic according to a cytotoxicity assay.

In some embodiments, the at least one selected epitope sequence comprises a mutation and the method comprises identifying cancer cells of the subject to encode the epitope with the mutation; the at least one selected epitope sequence is within a protein overexpressed by cancer cells of the subject and the method comprises identifying cancer cells of the subject to overexpress the protein containing the epitope; or the at least one epitope sequence comprises a protein expressed by a cell in a tumor microenvironment.

In some embodiments, one or more of the least one selected epitope sequence comprises an epitope that is not expressed by cancer cells of the subject.

In some embodiments, the epitope that is not expressed by cancer cells of the subject is expressed by cells in a tumor microenvironment of the subject.

In some embodiments, an epitope that binds to a protein encoded by an HLA allele of the subject binds to an MHC molecule encoded by the HLA allele with an affinity of 500 nM or less according to a binding assay.

In some embodiments, an epitope that binds to a protein encoded by an HLA allele of the subject is predicted to bind to an MHC molecule encoded by the HLA allele with an affinity of 500 nM or less using an MHC epitope prediction program implemented on a computer.

In some embodiments, the MHC epitope prediction program implemented on a computer is NetMHCpan In some embodiments, the MHC epitope prediction program implemented on a computer is NetMHCpan version 4.0.

In some embodiments, the epitope that is presented by antigen presenting cells (APCs) according to a mass spectrometry assay are detected by mass spectrometry after elution from the APCs with a mass accuracy of the detected peptide to be less than 15 Da, 10 Da or 5 Da, or less than 10,000 or 5,000 parts per million (ppm).

In some embodiments, the epitope that is immunogenic according to an immunogenicity assay is immunogenic according to a multimer assay or a functional assay.

In some embodiments, the multimer assay comprises flow cytometry analysis.

In some embodiments, the multimer assay comprises detecting T cells bound to a peptide-MHC multimer comprising the at least one selected epitope sequence and the matched HLA allele, wherein the T cells have been stimulated with APCs comprising a peptide containing the at least one selected epitope sequence.

In some embodiments, epitope is immunogenic according to the multimer assay when (i) at least 10 T cells that have been stimulated with APCs comprising a peptide containing the at least one selected epitope sequence are detected, (ii) the detected T cells make up at least 0.1% or 0.01% or 0.005% of the CD8⁺ cells analyzed, and (iii) the percentage of detected T cells of CD8+ T cells is higher than the percentage of detected T cells of CD8+ T cells detected in a control sample.

In some embodiments, the epitope is immunogenic according to the multimer assay when at least 10 T cells that have been stimulated with APCs comprising a peptide containing the at least one selected epitope sequence are detected in at least one out of six stimulations from the same starting sample.

In some embodiments, the control sample comprises T cells that have been stimulated with APCs that (i) do not comprise a peptide containing the at least one selected epitope sequence, (ii) comprise a peptide derived from a different protein than the at least one selected epitope sequence, or (iii) comprise a peptide with a random sequence.

In some embodiments, the T cells have been stimulated with APCs comprising a peptide containing the at least one selected epitope sequence for at least 3, 4, 5, 6, 7, 8, 9, 10, 11, 12, 13, 14, 15, 16, 7, 18, 19, 20 or more days.

In some embodiments, antigen-specific T cells have been expanded at least 5-fold, 10-fold, 20, fold, 50-fold, 100-fold, 500-fold or 1,000-fold or more in the presence of APCs comprising a peptide containing the at least one selected epitope sequence.

In some embodiments, the functional assay comprises an immunoassay.

In some embodiments, the functional assay comprises detecting T cells with intracellular staining of IFNγ or TNFα or cell surface expression of CD107a and/or CD107b, wherein the T cells have been stimulated with APCs comprising a peptide containing the at least one selected epitope sequence

In some embodiments, the epitope is immunogenic according to the functional assay when (i) at least 10 T cells that have been stimulated with APCs comprising a peptide containing the at least one selected epitope sequence are detected, (ii) the detected T cells make up at least 0.1% or 0.01% or 0.005% of the CD8⁺ or the CD4⁺ cells analyzed, and (iii) the percentage of detected T cells of CD8+ or CD4⁺ T cells is higher than the percentage of detected T cells of CD8+ or CD4⁺ T cells detected in a control sample.

In some embodiments, the T cells stimulated to be cytotoxic according to the cytotoxicity assay are T cells that have been stimulated with APCs comprising a peptide containing the at least one selected epitope sequence that kill cells presenting the epitope.

In some embodiments, a number of cells presenting the epitope that are killed by the T cells is at least 1.1, 2, 3, 4, 5, 6, 7, 8, 9, 10, 15, 20, 25, 30, 50, 100, 500, or 1,000 fold higher than a number of cells that do not present the epitope that are killed by the T cells.

In some embodiments, a number of cells presenting the epitope that are killed by the T cells is at least 1.1, 2, 3, 4, 5, 6, 7, 8, 9, 10, 15, 20, 25, 30, 50, 100, 500, or 1,000 fold higher than a number of cells presenting the epitope killed by T cells that have been stimulated with APCs that (i) do not comprise a peptide containing the at least one selected epitope sequence, (ii) comprise a peptide derived from a different protein than the at least one selected epitope sequence, or (iii) comprise a peptide with a random sequence.

In some embodiments, a number of cells presenting a mutant epitope that are killed by the T cells is at least 1.1, 2, 3, 4, 5, 6, 7, 8, 9, 10, 15, 20, 25, 30, 50, 100, 500, or 1,000 fold higher than a number of cells presenting a corresponding wild-type epitope that are killed by the T cells.

In some embodiments, the T cells stimulated to be cytotoxic according to the cytotoxicity assay are T cells stimulated to be specifically cytotoxic according to the cytotoxicity assay.

In some embodiments, the method comprises selecting the subject using a circulating tumor DNA assay.

In some embodiments, the method comprises selecting the subject using a gene panel.

In some embodiments, the T cell is from a biological sample from the subject.

In some embodiments, the T cell is from an apheresis or a leukopheresis sample from the subject.

In some embodiments, the T cell is an allogeneic T cell.

In some embodiments, each of the at least one selected epitope sequence is pre-validated to satisfy each of the following criteria: binds to a protein encoded by an HLA allele of the subject, is immunogenic according to an immunogenicity assay, is presented by antigen presenting cells (APCs) according to a mass spectrometry assay, and stimulates T cells to be cytotoxic according to a cytotoxicity assay.

In some embodiments, at least one of the one or more peptides is a synthesized peptide or a peptide expressed from a nucleic acid sequence.

In some embodiments, the method comprises identifying a protein encoded by an HLA allele of the subject or identifying an HLA allele in the genome of the subject.

In some embodiments, the at least one selected epitope sequence is selected from one or more epitope sequences of Table 1A-1F, Table 2A-2C, Table 3, Table 4A-4M, Table 5, Table 6, Table 7, Table 8, Table 11, Table 12, Table 13 and Table 14.

In some embodiments, the method comprises expanding the T cell contacted with the one or more peptides in vitro or ex vivo to obtain a population of T cells specific to the at least one selected epitope sequence in complex with an MEC protein.

In some embodiments, the method further comprises administering the population of T cells to the subject.

In some embodiments, a protein comprising the at least one selected epitope sequence is expressed by a cancer cell of the subject.

In some embodiments, a protein comprising the at least one selected epitope sequences is expressed by cells in the tumor microenvironment of the subject.

In some embodiments, one or more of the at least one selected epitope sequence comprises a mutation.

In some embodiments, one or more of the at least one selected epitope sequence comprises a tumor specific mutation.

In some embodiments, one or more of the at least one selected epitope sequence is from a protein overexpressed by a cancer cell of the subject.

In some embodiments, one or more of the at least one selected epitope sequence comprises a driver mutation.

In some embodiments, one or more of the at least one selected epitope sequence comprises a drug resistance mutation.

In some embodiments, one or more of the at least one selected epitope sequence is from a tissue-specific protein.

In some embodiments, one or more of the at least one selected epitope sequence is from a cancer testes protein.

In some embodiments, one or more of the at least one selected epitope sequence is a viral epitope.

In some embodiments, one or more of the at least one selected epitope sequence is a minor histocompatibility epitope.

In some embodiments, one or more of the at least one selected epitope sequence is from a RAS protein.

In some embodiments, one or more of the at least one selected epitope sequence is from a GATA3 protein.

In some embodiments, one or more of the at least one selected epitope sequence is from a EGFR protein.

In some embodiments, one or more of the at least one selected epitope sequence is from a BTK protein.

In some embodiments, one or more of the at least one selected epitope sequence is from a p53 protein.

In some embodiments, one or more of the at least one selected epitope sequence is from aTMPRSS2::ERG fusion polypeptide.

In some embodiments, one or more of the at least one selected epitope sequence is from a Myc protein.

In some embodiments, at least one of the at least one selected epitope sequence is from a protein encoded by a gene selected from the group consisting of ANKRD30A, COL10A1, CTCFL, PPIAL4G, POTEE, DLL3, MMP13, SSX1, DCAF4L2, MAGEA4, MAGEA11, MAGEC2, MAGEA12, PRAME, CLDN6, EPYC, KLK3, KLK2, KLK4, TGM4, POTEG, RLN1, POTEH, SLC45A2, TSPAN10, PAGES, CSAG1, PRDM7, TG, TSHR, RSPH6A, SCXB, HIST1H4K, ALPPL2, PRM2, PRM1, TNP1, LELP1, HMGB4, AKAP4, CETN1, UBQLN3, ACTL7A, ACTL9, ACTRT2, PGK2, C2orf53, KIF2B, ADAD1, SPATA8, CCDC70, TPD52L3, ACTL7B, DMRTB1, SYCN, CELA2A, CELA2B, PNLIPRP1, CTRC, AMY2A, SERPINI2, RBPJL, AQP12A, IAPP, KIRREL2, G6PC2, AQP12B, CYP11B1, CYP11B2, STAR, CYP11A1, and MC2R.

In some embodiments, at least one of the at least one selected epitope sequence is from a tissue-specific protein that has an expression level in a target tissue of the subject that is at least 2 fold more than an expression level of the tissue-specific protein in each tissue of a plurality of non-target tissues that are different than the target tissue.

In some embodiments, contacting a T cell from the subject or an allogeneic T cell with one or more peptides comprising the at least one selected epitope sequence comprises contacting the T cell with APCs presenting the epitope.

In some embodiments, the APCs presenting the epitope comprises one or more peptides comprising the at least one selected epitope sequence or a polynucleic acid that encodes one or more peptides comprising the at least one selected epitope sequence.

In some embodiments, the method comprises depleting CD14+ cells and CD25+ cells from a population of immune cells comprising antigen presenting cells (APCs) and T cells, thereby forming a CD14/CD25 depleted population of immune cells comprising a first population of APCs and T cells.

In some embodiments, the population of immune cells is from a biological sample from the subject.

In some embodiments, the method further comprises (b) incubating the CD14/CD25 depleted population of immune cells comprising a first population of APCs and T cells for a first time period in the presence of FMS-like tyrosine kinase 3 receptor ligand (FLT3L), and (A) a polypeptide comprising the at least one selected epitope sequence, or (B) a polynucleotide encoding the polypeptide; thereby forming a population of cells comprising stimulated T cells.

In some embodiments, the method further comprises (c) expanding the population of cells comprising stimulated T cells, thereby forming an expanded population of cells comprising tumor antigen-specific T cells, wherein the tumor antigen-specific T cells comprise T cells that are specific to a complex comprising (i) the at least one selected epitope sequence and (ii) an MEC protein expressed by the cancer cells or APCs of the subject.

In some embodiments, the T cells are expanded in less than 28 days.

In some embodiments, the fraction of CD8+ tumor antigen-specific T cells of the total number of CD8+ T cells in the expanded population of cells comprising tumor antigen specific T cells is at least two-fold higher than the fraction of CD8+ tumor antigen-specific T cells of the total number of CD8+ T cells in the biological sample.

In some embodiments, the fraction of CD4+ tumor antigen-specific T cells of the total number of CD4+ T cells in the expanded population of cells comprising tumor antigen specific T cells is at least two-fold higher than the fraction of CD4+ tumor antigen-specific T cells of the total number of CD4+ T cells in the biological sample.

In some embodiments, at least 0.1% of the CD8+ T cells in the expanded population of cells comprising tumor antigen specific T cells are CD8+ tumor antigen-specific T cells derived from naïve CD8+ T cells.

In some embodiments, at least 0.1% of the CD4+ T cells in the expanded population of cells comprising tumor antigen specific T cells are CD4+ tumor antigen-specific T cells derived from naïve CD4+ T cells.

In some embodiments, expanding comprises contacting the population of cells comprising stimulated T cells with a second population of mature APCs, wherein the second population of mature APCs have been incubated with FLT3L and present the at least one selected epitope sequence; and expanding the population of cells comprising stimulated T cells for a second time period, thereby forming an expanded population of T cells.

In some embodiments, the second population of mature APCs have been incubated with FLT3L for at least 1 day prior to contacting the population of cells comprising stimulated T cells with the second population of mature APCs.

In some embodiments, expanding further comprises (C) contacting the expanded population of T cells with a third population of mature APCs, wherein the third population of mature APCs (i) have been incubated with FLT3L and (ii) present the at least one selected epitope sequence; and (D) expanding the expanded population of T cells for a third time period, thereby forming the expanded population of cells comprising tumor antigen-specific T cells.

In some embodiments, the third population of mature APCs have been incubated with FLT3L for at least 1 day prior to contacting the expanded population of T cells with the third population of mature APCs.

In some embodiments, the biological sample is a peripheral blood sample, a leukapheresis sample or an apheresis sample.

In some embodiments, the method further comprises harvesting the expanded population of cells comprising tumor antigen-specific T cells, cryopreserving the expanded population of cells comprising tumor antigen-specific T cells or preparing a pharmaceutical composition containing the expanded population of cells comprising tumor antigen-specific T cells.

In some embodiments, incubating comprises incubating the CD14/CD25 depleted population of immune cells comprising a first population of APCs and T cells for a first time period in the presence of FLT3L and an RNA encoding the polypeptide.

In some embodiments, the method further comprises administering a pharmaceutical composition comprising the expanded population of cells comprising tumor antigen specific T cells to a human subject with cancer.

In some embodiments, the human subject with cancer is the human subject from which the biological sample was obtained.

In some embodiments, the polypeptide is from 8 to 50 amino acids in length.

In some embodiments, the polypeptide comprises at least two of the selected epitope sequence, each expressed by cancer cells of a human subject with cancer.

In some embodiments, depleting CD14+ cells and CD25+ cells from the population of immune cells comprising a first population of APCs and T cells comprises contacting the population of immune cells comprising a first population of APCs and T cells with a CD14 binding agent and a CD25 binding agent.

In some embodiments, depleting further comprising depleting CD19+ cells from the population of immune cells comprising a first population of APCs and T cells.

In some embodiments, depleting further comprising depleting CD11b+ cells from the population of immune cells comprising a first population of APCs and T cells.

In some embodiments, the method comprises generating cancer cell nucleic acids from a first biological sample comprising cancer cells obtained from a subject and generating non-cancer cell nucleic acids from a second biological sample comprising non-cancer cells obtained from the same subject.

In some embodiments, the protein encoded by an HLA allele of the subject is a protein encoded by an HLA allele selected from the group consisting of HLA-A01:01, HLA-A02:01, HLA-A03:01, HLA-A11:01, HLA-A24:01, HLA-A30:01, HLA-A31:01, HLA-A32:01, HLA-A33:01, HLA-A68:01, HLA-B07:02, HLA-B08:01, HLA-B15:01, HLA-B44:03, HLA-007:01 and HLA-007:02.

In some embodiments, the method comprises identifying one or two or more different proteins that comprise the at least one selected epitope sequence and that are expressed by cancer cells of the subject

In some embodiments, the method comprises identifying one or two or more different proteins that comprise the at least one selected epitope sequence and that are expressed by cancer cells of the subject by measuring levels of RNA encoding the one or two or more different proteins in the cancer cells.

In some embodiments, one or more of the at least one selected epitope sequence has a length of from 8 to 12 amino acids.

In some embodiments, one or more of the at least one selected epitope sequence has a length of from 13-25 amino acids.

In some embodiments, the method comprises isolating genomic DNA or RNA from cancer cells and non-cancer cells of the subject.

In some embodiments, one or more of the at least one selected epitope sequence comprises a point mutation or a sequence encoded by a point mutation.

In some embodiments, one or more of the at least one selected epitope sequence comprises a sequence encoded by a neoORF mutation.

In some embodiments, one or more of the at least one selected epitope sequence comprises a sequence encoded by a gene fusion mutation.

In some embodiments, one or more of the at least one selected epitope sequence comprises a sequence encoded by an indel mutation.

In some embodiments, one or more of the at least one selected epitope sequence comprises a sequence encoded by a splice site mutation.

In some embodiments, at least two of the at least one selected epitope sequence are from a same protein.

In some embodiments, at least two of the at least one selected epitope sequence comprise an overlapping sequence.

In some embodiments, at least two of the at least one selected epitope sequence are from different proteins.

In some embodiments, the one or more peptides comprise at least 2, 3, 4, 5, 6, 7, 8, 9, or 10 or more peptides.

In some embodiments, cancer cells of the subject are cancer cells of a solid cancer.

In some embodiments, cancer cells of the subject are cancer cells of a leukemia or a lymphoma.

In some embodiments, the mutation is a mutation that occur in a plurality of cancer patients.

In some embodiments, the MEC is a Class I MEC.

In some embodiments, the MEC is a Class II MEC.

In some embodiments, the T cell is a CD8 T cell.

In some embodiments, the T cell is a CD4 T cell.

In some embodiments, the T cell is a cytotoxic T cell.

In some embodiments, the T cell is a memory T cell.

In some embodiments, the T cell is a naive T cell.

In some embodiments, the method further comprises selecting one or more subpopulation of cells from an expanded population of T cells prior to administering to the subject.

In some embodiments, eliciting an immune response in the T cell culture comprises inducing IL2 production from the T cell culture upon contact with the peptide.

In some embodiments, eliciting an immune response in the T cell culture comprises inducing a cytokine production from the T cell culture upon contact with the peptide, wherein the cytokine is an Interferon gamma (IFN-γ), Tumor Necrosis Factor (TNF) alpha (α) and/or beta (β) or a combination thereof.

In some embodiments, eliciting an immune response in the T cell culture comprises inducing the T cell culture to kill a cell expressing the peptide.

In some embodiments, eliciting an immune response in the T cell culture comprises detecting an expression of a Fas ligand, granzyme, perforins, IFN, TNF, or a combination thereof in the T cell culture.

In some embodiments, the one or more peptides comprising the at least one selected epitope sequence is purified.

In some embodiments, the one or more peptides comprising the at least one selected epitope sequence is lyophilized.

In some embodiments, the one or more peptides comprising the at least one selected epitope sequence is in a solution.

In some embodiments, the one or more peptides comprising the at least one selected epitope sequence is present in a storage condition such that the integrity of the peptide is ≥99%.

In some embodiments, the method comprises stimulating T cells to be cytotoxic against cells loaded with the at least one selected epitope sequences according to a cytotoxicity assay.

In some embodiments, the method comprises stimulating T cells to be cytotoxic against cancer cells expressing a protein comprising the at least one selected epitope sequences according to a cytotoxicity assay.

In some embodiments, the method comprises stimulating T cells to be cytotoxic against a cancer associated cell expressing a protein comprising the at least one selected epitope sequences according to a cytotoxicity assay.

In some embodiments, the at least one selected epitope is expressed by a cancer cell, and an additional selected epitope is expressed by a cancer associated cell.

In some embodiments, the additional selected epitope is expressed on a cancer associated fibroblast cell.

In some embodiments, the additional selected epitope is selected from Table 8.

Also provided herein is a pharmaceutical composition comprising a T cell produced by a method provided herein.

Also provided herein is a library of polypeptides comprising epitope sequences or polynucleotides encoding the polypeptides, wherein each epitope sequence in the library is matched to a protein encoded by an HLA allele; and wherein each epitope sequence in the library is pre-validated to satisfy at least three of the following criteria: binds to a protein encoded by an HLA allele of a subject with cancer to be treated, is immunogenic according to an immunogenic assay, is presented by antigen presenting cells (APCs) according to a mass spectrometry assay, and/or stimulates T cells to be cytotoxic according to a cytotoxicity assay.

Also provided herein is a method of treating cancer in a subject comprising administering to the subject (i) a polypeptide comprising a G12R RAS epitope, or (ii) a polynucleotide encoding the polypeptide; wherein: (a) the G12R RAS epitope is vvgaRgvgk (SEQ ID NO: 1) and the subject expresses a protein encoded by an HLA-A03:01 allele; (b) the G12R RAS epitope is eyklvvvgaR (SEQ ID NO: 2) and the subject expresses a protein encoded by an HLA-A33:03 allele; (c) the G12R RAS epitope is vvvgaRgvgk (SEQ ID NO: 3) and the subject expresses a protein encoded by an HLA-A11:01 allele; or (d) the G12R RAS epitope is aRgvgksal (SEQ ID NO: 4) and the subject expresses a protein encoded by an HLA-allele selected from the group consisting of HLA-C07:02, HLA-B39:01 and HLA-C07:01.

### BRIEF DESCRIPTION OF THE DRAWINGS

FIG. 1A is schematic of an exemplary method provided herein to prime, activate and expand antigen-specific T cells.

FIG. 1B is schematic of an exemplary method provided herein to prime, activate and expand antigen-specific T cells.

FIG. 2 is schematic of an exemplary method for offline characterization of shared epitopes.

FIG. 3A depicts data illustrating that in silico epitope prediction identified multiple neoantigens derived from RAS G12D mutations that are presented according to mass spectrometry. Figure discloses SEQ ID NOS 1420, 1421, 1147, 1245, and 1247, respectively, in order of appearance.

FIG. 3B depicts data illustrating that in silico epitope prediction identified multiple neoantigens derived from RAS G12V mutations that are presented according to mass spectrometry. Figure discloses SEQ ID NOS 1422, 1423, 162, 163, and 1148, respectively, in order of appearance.

FIG. 3C depicts data illustrating that in silico epitope prediction identified multiple neoantigens derived from RAS G12C mutations that are presented according to mass spectrometry. Figure discloses SEQ ID NO: 1424.

FIG. 3D depicts data illustrating that in silico epitope prediction identified multiple neoantigens derived from RAS G12R mutations that are presented according to mass spectrometry. Figure discloses SEQ ID NOS 1425, 1426, 1253, and 2, respectively, in order of appearance.

FIG. 4A depicts data illustrating that presentation of shared neoantigen epitopes can be directly confirmed by mass spectrometry and that RAS neoantigens are targetable in defined patient populations.

FIG. 4B shows head-to-toe plot of MS/MS spectra for the endogenously processed mutant RAS peptide epitope VVVGAVGVGK (SEQ ID NO: 5) (top) and its corresponding heavy peptide (bottom). 293T cells were lentivirally transduced with both a polypeptide containing the RAS^{G12V} mutant peptide and an HLA-A*03:01 gene.

FIG. 4C shows head-to-toe plot of MS/MS spectra for the endogenously processed mutant RAS peptide epitope VVVGAVGVGK (SEQ ID NO: 5) (top) and its corresponding heavy peptide (bottom). SW620 cells that naturally express the RAS^{G12V} mutant were transduced with a lentiviral vector encoding an HLA-A*03:01 gene.

FIG. 4D shows head-to-toe plot of MS/MS spectra for the endogenously processed mutant RAS peptide epitope VVVGAVGVGK (SEQ ID NO: 5) (top) and its corresponding heavy peptide (bottom). NCI-H441 cells naturally expressing both the RAS^{G12V} mutation and the HLA-A*03:01 gene were used for this experiment.

FIG. 4E shows head-to-toe plot of MS/MS spectra for the endogenously processed GATA3 neoORF peptide epitope SMLTGPPARV (SEQ ID NO: 6). Endogenous peptide spectrum is shown in the top panel and corresponding light synthetic spectrum is shown in the bottom panels.

FIG. 5 depicts data illustrating that an exemplary method provided herein to prime, activate and expand antigen-specific T cells induces de novo CD8 T cell responses against RAS G12 neoantigens on HLA-A11:01 and HLA-A03:01.

FIG. 6 depicts data illustrating that an exemplary method provided herein to prime, activate and expand antigen-specific T cells induces multiple de novo CD8 T cell responses against RAS G12V neoantigen on HLA-A11:01. As indicated in the pie charts, the frequency of individual T cell clones induced against RAS G12V neoantigen on HLA-A11:01 in 3 independent healthy donors is depicted.

FIG. 7 depicts data illustrating that RAS^{G12V}-activated T cells generated ex vivo can kill target cells. A375 target cells expressing GFP were loaded with 2 μM RAS^{G12V} antigen, wild-type RAS antigen, or no peptide as control GFP+ cells. RAS^{G12V}-specific CD8 T cells (effector cells) were incubated with control cells or target cells in a 0.05:1 ratio. In presence of the effector cells, target cells were lysed and depleted more readily that control cells which present either RAS' antigen or no antigen. Graph of specific cell killing as normalized by target cell growth with no peptide is shown in the left diagram. Representative images are shown on the right.

FIG. 8 depicts data illustrating that an exemplary method provided herein to prime, activate and expand RAS G12V-specific T cells with RAS G12V neoantigens on HLA-11:01, but not the corresponding wild-type antigens, induces T cells to become cytotoxic using the indicated effector:target cell ratios and increasing peptide concentration.

FIG. 9 depicts data illustrating that an exemplary method provided herein to prime, activate and expand antigen-specific T cells with one round (1× stimulated) or two rounds (2× stimulated) of FLT3L-treated PBMCs presenting an epitope with the RAS^{G12V} mutation induces T cells to become cytotoxic as measured by AnnexinV positive cells over time after co culturing these T cells with SW620 cells (naturally express the RAS^{G12V} mutant) that were transduced with a lentiviral vector encoding an HLA-A*11:01 gene.

FIG. 10 depicts a graph of AnnexinV positive cells over time after co-culturing NCI-H441 cells naturally expressing both the RAS^{G12V} mutation and the HLA-A*03:01 gene with T cells that had been primed and activated and expanded with a peptide containing an epitope with the RAS^{G12V} mutation at the indicated effector:target cell ratio.

FIG. 11A depicts a graph of IL-2 concentration (pg/mL) vs RAS-G12V wild-type or mutant peptide loaded target cells (A375-A11:01) after incubation in the presence of Jurkat cells transduced with a TCR that binds to the RAS-G12V epitope bound to an MHC encoded by the HLA-A11:01 allele.

FIG. 11B depicts graphs of AnnexinV positive cells over time after co culturing TCR-transduced PBMCs with 5,000 SNGM cells with natural G12V and HLA-A11:01 across a range of effector:target cell ratios.

FIG. 11C depicts a graph of IL-2 concentration (pg/mL) vs RAS-G12V wild-type or mutant peptide loaded target cells (A375-A03:01) after incubation in the presence of Jurkat cells transduced with a TCR that binds to RAS-G12V bound to an MHC encoded by the HLA-A03:01 allele.

FIG. 11D depicts a graph of AnnexinV positive cells over time (top) after co-culturing TCR-transduced PBMCs with cells with natural G12V and HLA-A03:01 using an effector:target cell ratio of 0.75:1 and a graph of IFNγ concentration (pg/mL) after 24 hours of coculturing TCR-transduced PBMCs with cells with natural G12V and HLA-A03:01 using an effector:target cell ratio of 0.75:1.

FIG. 12A depicts a graph of IL-2 concentration (pg/mL) vs FLT3L-treated PBMCs contacted with increasing amounts of the indicated RAS-G12V mutant peptides after being co-cultured with Jurkat cells transduced with a TCR that binds to the underlined RAS-G12V epitope bound to an MHC encoded by the HLA-A11:01 allele. Figure discloses SEQ ID NOS 164, 1427, and 1428, respectively, in order of appearance.

FIG. 12B depicts data illustrating the immunogenicity of the indicated RAS-G12V mutant peptides from FIG. 12A both in vitro using PBMCs from healthy donors (top) and in vivo using HLA-A11:01 transgenic mice immunized with the peptides (bottom).

FIG. 13 depicts data illustrating that an exemplary method provided herein to prime, activate and expand antigen-specific T cells induces de novo CD8 T cell responses against RAS G12V neoantigen on HLA-02:01.

FIG. 14 depicts data illustrating that an exemplary method provided herein to prime, activate and expand antigen-specific T cells induces de novo CD8 T cell responses against RAS G12 neoantigens on HLA-A68:01.

FIG. 15 depicts data illustrating that an exemplary method provided herein to prime, activate and expand antigen-specific T cells induces de novo CD8 T cell responses against RAS G12 neoantigens on HLA-B07:02

FIG. 16 depicts data illustrating that an exemplary method provided herein to prime, activate and expand antigen-specific T cells induces de novo CD8 T cell responses against RAS G12 neoantigens on HLA-B08:01.

FIG. 17 depicts data illustrating that an exemplary method provided herein to prime, activate and expand antigen-specific T cells induces de novo CD8 T cell responses against RAS G12D neoantigen on HLA-008:02.

FIG. 18 depicts data illustrating that an exemplary method provided herein to prime, activate and expand antigen-specific T cells induces de novo CD4 T cell responses against RAS neoantigens.

FIG. 19A depicts data illustrating flow cytometry data demonstrating that enrichment procedures can be used prior to further expansion of antigen-specific T cells. Cells upregulating 4-1BB were enriched using Magnetic-Assisted Cell Separation (MACS; Miltenyi). T cells that were stained by multimers were enriched by MACS on day 14 of stimulation. This approach was able to enrich for multiple antigen-specific T cell populations.

FIG. 19B depicts an exemplary bar graph quantifying the results in FIG. 19A.

FIG. 20 illustrates a summary of experiments illustrating that predicted GATA3 neoORF epitopes have strong affinity (<500 nM), long stability (>0.5 hr) and/or can be detected by mass spectrometry analysis of epitopes eluted from HLA molecules from cells expressing the GATA3 neoORF. Figure discloses SEQ ID NOS 1081, 6, 1088, 1097, 1089, 1085, 1089, 1078, 1093, 1095, 1082, 1079, 1091, 1075, 1078, 1097, 1092, 1079, 1094, and 1096, respectively, in order of appearance.

FIG. 21 depicts data illustrating that an exemplary method provided herein to prime, activate and expand antigen-specific T cells induces de novo CD8 T cell responses against GATA3 neoORF neoantigens on HLA-A02:01, HLA-A03:01, HLA-A11:01, HLA-B07:02 and HLA-B08:01. Figure discloses SEQ ID NOS 1081, 1089, 1089, 1095, and 1091, respectively, in order of appearance.

FIG. 22 depicts data illustrating GATA3 neoORF epitope-activated T cells generated ex vivo can kill target cells. 293T target cells expressing GFP were loaded with 2 μM GATA3 neoORF antigen or left unloaded as control GFP+ cells. GATA3-neoORF-specific CD8 T cells (effector cells) were incubated with control cells or target cells in a 1:10 ratio. In presence of the effector cells, target cells were lysed and depleted more readily that control cells which do present GATA3 neoantigen. Graph of GFP+ cells over 100 hours is shown in the top diagram. Images of the control (bottom left image) and target GFP+ cells (bottom right image) in the presence of GATA3 neoantigen activated CD8 cells are shown.

FIG. 23 depicts a graph of a comparison of Caspase-3 positive fraction of live target cells in GATA3 neoantigen transduced HEK 293T cells versus non-transduced HEK 293T cells. Two different GATA3 induced healthy donor PBMCs were co-cultured with GATA3 neoantigen transduced HEK 293T cells or non-transduced HEK 293T cells as a negative control group.

FIG. 24 depicts flow cytometry data illustrating induction of antigen-specific CD4+ T cells with GATA3 neoORF specific peptide after 20 days in culture, including two stimulations. Antigen-specific T cells are detected by increase in IFNγ and/or TNFα after incubation with GATA3 neoORF peptides (right) relative to no peptides (left)

FIG. 25A depicts a schematic diagram of steps followed through discovery and validation of peptides presented in prostate cancer cell lines or prostate tissue from human donors, and generating validated peptides for a curated validated peptide library.

FIG. 25B depicts data illustrating generation of epitope specific CD8T cells in vitro. The peptides were predicted using T cell epitope prediction software in proteins specific to prostate cancer. Figure discloses SEQ ID NOS 1403, 1405, and 7, respectively, in order of appearance.

FIG. 25C depicts data illustrating KLK4 epitope-activated T cells generated ex vivo are immunogenic and kill target cells. 293T target cells expressing GFP were loaded with 2 μM KLK4 antigen (LLANGRMPTV (SEQ ID NO: 7)) or left unloaded as control GFP+ cells. KLK4 specific CD8 T cells (effector cells) were incubated with control cells or target cells in a 1:10 ratio. In presence of the effector cells, target cell growth was controlled more readily than control cells which do not express KLK4. Also shown is a graph of GFP+ cells over 100 hours (bottom). Images of the control (bottom left image) and target GFP+ cells (bottom right image) in the presence of KLK4 activated CD8 cells are shown.

FIG. 26 depicts data illustrating that an exemplary method provided herein to prime, activate and expand antigen-specific T cells induces de novo CD8 T cell responses against a BTK C481S neoantigen on HLA-02:01.

FIG. 27 depicts data illustrating that an exemplary method provided herein to prime, activate and expand antigen-specific T cells induces de novo CD8 T cell responses against EGFR T790M neoantigens on HLA-02:01.

FIG. 28A depicts a schematic of an exemplary method provided herein for application of T cell therapies.

FIG. 28B depicts a schematic of an exemplary method provided herein for application of T cell therapies.

FIG. 29 depicts a schematic of an exemplary method for in silico T cell epitope prediction. PPV was determined for a given n number of hits and 5,000 decoys, what fraction of the n top-ranked peptides were hits.

FIG. 30 depicts a schematic of allelic coverage of the MHC ligandome using in silico epitope prediction.

FIG. 31 depicts a schematic comparing in silico T cell epitope prediction models.

FIG. 32 depicts a schematic illustrating identification and validation of immunogenic peptides using in silico T cell epitope prediction and an exemplary method provided herein to prime, activate and expand antigen-specific T cells.

FIG. 33 depicts data illustrating that an exemplary method provided herein to prime, activate and expand antigen-specific T cells can induce and expand multiple neoantigen CD8+ T cell populations. The data shown is representative data from sample from a melanoma patient.

FIG. 34 depicts data illustrating that an exemplary method provided herein to prime, activate and expand antigen-specific T cells generated three CD4+ populations in the same patient. The data shown is representative data from sample from a melanoma patient.

FIG. 35 depicts data illustrating that an exemplary method provided herein to prime, activate and expand antigen-specific T cells repeatedly demonstrates T cell inductions across melanoma patient samples.

FIG. 36 depicts representative data from a melanoma patient sample illustrating that an exemplary method provided herein to prime, activate and expand antigen-specific T cells induces T cells highly specific for mutant epitopes.

FIG. 37 depicts representative data from a melanoma patient sample illustrating that an exemplary method provided herein to prime, activate and expand antigen-specific T cells induces T cells that are highly functional.

FIG. 38 depicts data illustrating that an exemplary method provided herein to prime, activate and expand antigen-specific T cells induces CD8+ T cells can kill tumor cells.

### DETAILED DESCRIPTION

Although many epitopes have the potential to bind to an MEC molecule, few are capable of binding to an MEC molecule when tested experimentally. Although many epitopes also have the potential to potential to be presented by an MEC molecule that can, for example, be detected by mass spectrometry, only a select number of these epitopes can be presented and detected by mass spectrometry. Although many epitopes also have the potential to be immunogenic, when tested experimentally many of these epitopes are not immunogenic, despite being demonstrated to be presented by antigen presenting cells. Many epitopes also have the potential to activate T cells to become cytotoxic; however, many epitopes that have been demonstrated to be presented by antigen presenting cells and/or to be immunogenic are still not capable of activating T cells to become cytotoxic.

Provided herein are antigens containing T cell epitopes that have been identified and validated as binding to one or more MEC molecules, presented by the one or more MEC molecules, being immunogenic and capable of activating T cells to become cytotoxic. The validated antigens and polynucleotides encoding these antigens can be used in preparing antigen specific T cells for therapeutic uses. In some embodiments, the validated antigens and polynucleotides encoding these antigens can be pre-manufactured and stored for use in a method of manufacturing T cells for therapeutic uses. For example, the validated antigens and polynucleotides encoding these antigens can be pre-manufactured or manufactured quickly to prepare therapeutic T cell compositions for patients quickly. Using validated antigens with T cell epitopes, immunogens such as peptides having HLA binding activity or RNA encoding such peptides can be manufactured. Multiple immunogens can be identified, validated and pre-manufactured in a library. In some embodiments, peptides can be manufactured in a scale suitable for storage, archiving and use for pharmacological intervention on a suitable patient at a suitable time.

Some, if not all cancers have antigens that are potential targets for immunotherapy. Each peptide antigen may be presented for T cell activation on an antigen presenting cells in association with a specific HLA-encoded MEC molecule. On the other hand, provided herein is a potentially universal approach, where particular epitopes are pre-identified and pre-validated for particular HLAs, and these epitopes can be pre-manufactured for a cell therapy manufacturing process. For example, a number of KRAS epitopes with G12, G13 and Q61 mutations can be identified using a reliable T cell epitope presentation prediction model (see, e.g., PCT/US2018/017849, filed Feb. 12, 2018, and PCT/US2019/068084 filed Dec. 20, 2019, each of which are incorporated by reference in their entirety), with validation of immunogenicity of these epitopes, processing and presentation using mass spectrometry of these epitopes, and ability to generate cytotoxic T cells with TCRs against these epitopes and MHCs encoded by different HLAs. Each epitope is validated with its specific amino acid sequence and relevant HLA. Once these epitopes are validated, a library can be created containing pre-manufactured immunogens, such as peptides containing the epitopes or RNA encoding peptides containing these epitopes.

The antigens can be non-mutated antigens or mutated antigens. For example, the antigens can be tumor-associated antigens, mutated antigens, tissue-specific antigens or neoantigens. In some embodiments, the antigens are tumor-associated antigens. In some embodiments, the antigens are mutated antigens. In some embodiments, the antigens are tissue-specific antigens. In some embodiments, the antigens are neoantigens. Neoantigens are found in the cancer or the tumor in a subject and is not evident in the germline or expressed in the healthy tissue of the subject. Therefore, for a gene mutation in cancer to satisfy the criteria of generating a neoantigen, the gene mutation in the cancer must be a non-silent mutation that translates into an altered protein product. The altered protein product contains an amino acid sequence with a mutation that can be a mutated epitope for a T cell. The mutated epitope has the potential to bind to an MEC molecule. The mutated epitope also has the potential to be presented by an MEC molecule that can, for example, be detected by mass spectrometry. Furthermore, the mutated epitope has the potential to be immunogenic. Additionally, the mutated epitope has the potential to activate T cells to become cytotoxic.

Provided herein is a method for treating cancer in a subject in need thereof comprising selecting at least one epitope sequence from a library of epitope sequences, wherein each epitope sequence in the library is matched to a protein encoded by an HLA allele of the subject; and contacting a T cell from the subject or an allogeneic T cell with one or more peptides comprising the at least one selected epitope sequence, wherein each of the at least one selected epitope sequence is pre-validated to satisfy at least two or three or four of the following criteria binds to a protein encoded by an HLA allele of the subject, is immunogenic according to an immunogenicity assay, is presented by antigen presenting cells (APCs) according to a mass spectrometry assay, and stimulates T cells to be cytotoxic according to a cytotoxicity assay. In some embodiments, the method further comprises administering the population of T cells to the subject.

In some embodiments, the at least one selected epitope sequence comprises a mutation and the method comprises identifying cancer cells of the subject to encode the epitope with the mutation; the at least one selected epitope sequence is within a protein overexpressed by cancer cells of the subject and the method comprises identifying cancer cells of the subject to overexpress the protein containing the epitope; or the at least one epitope sequence comprises a protein expressed by a cell in a tumor microenvironment. In some embodiments, one or more of the least one selected epitope sequence comprises an epitope that is not expressed by cancer cells of the subject. In some embodiments, the epitope that is not expressed by cancer cells of the subject is expressed by cells in a tumor microenvironment of the subject. In some embodiments, the method comprises selecting the subject using a circulating tumor DNA assay. In some embodiments, the method comprises selecting the subject using a gene panel.

In some embodiments, the T cell is from a biological sample from the subject. In some embodiments, the T cell is from an apheresis or a leukopheresis sample from the subject. In some embodiments, the T cell is an allogeneic T cell.

In some embodiments, each of the at least one selected epitope sequence is pre-validated to satisfy one or more or each of the following criteria: binds to a protein encoded by an HLA allele of the subject, is immunogenic according to an immunogenicity assay, is presented by antigen presenting cells (APCs) according to a mass spectrometry assay, and stimulates T cells to be cytotoxic according to a cytotoxicity assay.

In some embodiments, an epitope that binds to a protein encoded by an HLA allele of the subject binds to an MHC molecule encoded by the HLA allele with an affinity of 500 nM or less according to a binding assay. For example, an epitope that binds to a protein encoded by an HLA allele of the subject can bind to an MHC molecule encoded by the HLA allele with an affinity of 400 nM, 300 nM, 200 nM, 150 nM, 100 nM, 75 nM, 50 nM, or 25 nM or less according to a binding assay. In some embodiments, an epitope that binds to a protein encoded by an HLA allele of the subject is predicted to bind to an MHC molecule encoded by the HLA allele with an affinity of 500 nM or less using an MHC epitope prediction program implemented on a computer. For example, an epitope that binds to a protein encoded by an HLA allele of the subject can be predicted to bind to an MHC molecule encoded by the HLA allele with an affinity of 400 nM, 300 nM, 200 nM, 150 nM, 100 nM, 75 nM, 50 nM, or 25 nM or less using an MHC epitope prediction program implemented on a computer. In some embodiments, the MHC epitope prediction program implemented on a computer is NetMHCpan. In some embodiments, the MHC epitope prediction program implemented on a computer is NetMHCpan version 4.0.

In some embodiments, the epitope that is presented by antigen presenting cells (APCs) according to a mass spectrometry assay is detected by mass spectrometry after elution from the APCs with a mass accuracy of the detected peptide to be less than 15 Da. For example, the epitope that is presented by antigen presenting cells (APCs) according to a mass spectrometry assay can be detected by mass spectrometry after elution from the APCs with a mass accuracy of the detected peptide to be less than 14 Da, 13 Da, 12 Da, 11 Da, 10 Da, 9 Da, 8 Da, 7 Da, 6 Da, 5 Da, 4 Da, 3 Da, 2 Da, or 1 Da. In some embodiments, the epitope that is presented by antigen presenting cells (APCs) according to a mass spectrometry assay is detected by mass spectrometry after elution from the APCs with a mass accuracy of the detected peptide to be less than 10,000 parts per million (ppm). For example, the epitope that is presented by antigen presenting cells (APCs) according to a mass spectrometry assay can be detected by mass spectrometry after elution from the APCs with a mass accuracy of the detected peptide to be less than 7,500 ppm; 5,000 ppm; 2,500 ppm; 1,000 ppm; 900 ppm; 800 ppm; 700 ppm; 600 ppm; 500 ppm; 400 ppm; 300 ppm; 200 ppm or 100 ppm.

In some embodiments, the epitope that is immunogenic according to an immunogenicity assay is immunogenic according to a multimer assay. In some embodiments, the multimer assay comprises flow cytometry analysis. In some embodiments, the multimer assay comprises detecting T cells bound to a peptide-MHC multimer comprising the at least one selected epitope sequence and the matched HLA allele, wherein the T cells have been stimulated with APCs comprising a peptide containing the at least one selected epitope sequence. In some embodiments, an epitope is immunogenic according to the multimer assay when (i) at least 10 T cells that have been stimulated with APCs comprising a peptide containing the at least one selected epitope sequence are detected, (ii) the detected T cells make up at least 0.005% of the CD8⁺ cells analyzed, and (iii) the percentage of detected T cells of CD8+ T cells is higher than the percentage of detected T cells of CD8+ T cells detected in a control sample. For example, an epitope can be immunogenic according to the multimer assay when (i) at least 10, 15, 20, 30, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected, (ii) the detected T cells make up at least 0.005% of the CD8+ cells analyzed, and (iii) the percentage of detected T cells of CD8+ T cells is higher than the percentage of detected T cells of CD8+ T cells detected in a control sample. For example, an epitope can be immunogenic according to the multimer assay when (i) at least 10 T cells that have been stimulated with APCs comprising a peptide containing the at least one selected epitope sequence are detected, (ii) the detected T cells make up at least 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, or 10% of the CD8⁺ cells analyzed, and (iii) the percentage of detected T cells of CD8+ T cells is higher than the percentage of detected T cells of CD8+ T cells detected in a control sample. For example, an epitope can be immunogenic according to the multimer assay when (i) at least 10, 15, 20, 30, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected, (ii) the detected T cells make up at least 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, or 10% of the CD8+ cells analyzed, and (iii) the percentage of detected T cells of CD8+ T cells is higher than the percentage of detected T cells of CD8+ T cells detected in a control sample.

In some embodiments, the epitope is immunogenic according to the multimer assay when at least 10 T cells that have been stimulated with APCs comprising a peptide containing the at least one selected epitope sequence are detected in at least one out of six stimulations from the same starting sample. For example, the epitope can be immunogenic according to the multimer assay when at least 10, 15, 20, 30, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected in at least one out of six stimulations from the same starting sample. For example, the epitope can be immunogenic according to the multimer assay when at least 10 T cells that have been stimulated with APCs comprising a peptide containing the at least one selected epitope sequence are detected in at least 2 out of 6, 7, 8, 9, 10, 11 or 12 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5 or 6 out of 6 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5, 6 or 7 out of 7 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5, 6, 7 or 8 out of 8 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5, 6, 7, 8 or 9 out of 9 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5, 6, 7, 8, 9 or 10 out of 10 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5, 6, 7, 8, 9, 10 or 11 out of 11 stimulations from the same starting sample. For example, the epitope can be immunogenic according to the multimer assay when at least 10 T cells that have been stimulated with APCs comprising a peptide containing the at least one selected epitope sequence are detected in at least 2, 3, 4, 5, 6, 7, 8, 9, 10, 11 or 12 out of 12 stimulations from the same starting sample. For example, the epitope can be immunogenic according to the multimer assay when at least 10 T cells that have been stimulated with APCs comprising a peptide containing the at least one selected epitope sequence are detected in at least 3 out of 6, 7, 8, 9, 10, 11, 12, 13, 14, 15, 16, 17 or 18 stimulations from the same starting sample. For example, the epitope can be immunogenic according to the multimer assay when at least 10 T cells that have been stimulated with APCs comprising a peptide containing the at least one selected epitope sequence are detected in at least 4 out of 6, 7, 8, 9, 10, 11, 12, 13, 14, 15, 16, 17, 18, 19, 20, 21, 22, 23 or 24 stimulations from the same starting sample. For example, the epitope can be immunogenic according to the multimer assay when at least 10, 15, 20, 30, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected in at least one out of six stimulations from the same starting sample. For example, the epitope can be immunogenic according to the multimer assay when at least 10, 15, 20, 30, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected in at least 2 out of 6, 7, 8, 9, 10, 11 or 12 stimulations from the same starting sample or in at least 3 out of 6, 7, 8, 9, 10, 11, 12, 13, 14, 15, 16, 17 or 18 stimulations from the same starting sample or in at least 4 out of 6, 7, 8, 9, 10, 11, 12, 13, 14, 15, 16, 17, 18, 19, 20, 21, 22, 23 or 24 stimulations from the same starting sample. In some embodiments, the control sample comprises T cells that have been stimulated with APCs that (i) do not comprise a peptide containing the at least one selected epitope sequence, (ii) comprise a peptide derived from a different protein than the at least one selected epitope sequence, or (iii) comprise a peptide with a random sequence. In some embodiments, the T cells have been stimulated with APCs comprising a peptide containing the at least one selected epitope sequence for at least 3, 4, 5, 6, 7, 8, 9, 10, 11, 12, 13, 14, 15, 16, 7, 18, 19, 20 or more days. In some embodiments, antigen-specific T cells have been expanded at least 5-fold, 10-fold, 20, fold, 50-fold, 100-fold, 500-fold or 1,000-fold or more in the presence of APCs comprising a peptide containing the at least one selected epitope sequence.

In some embodiments, the epitope that is immunogenic according to an immunogenicity assay is immunogenic according to a functional assay. In some embodiments, the functional assay comprises an immunoassay. In some embodiments, the functional assay comprises detecting T cells with intracellular staining of IFNγ or TNFα or cell surface expression of CD107a and/or CD107b, wherein the T cells have been stimulated with APCs comprising a peptide containing the at least one selected epitope sequence In some embodiments, the epitope is immunogenic according to the functional assay when (i) at least 10 T cells that have been stimulated with APCs comprising a peptide containing the at least one selected epitope sequence are detected, (ii) the detected T cells make up at least 0.005% of the CD8⁺ or the CD4⁺ cells analyzed, and (iii) the percentage of detected T cells of CD8+ or CD4⁺ T cells is higher than the percentage of detected T cells of CD8+ or CD4⁺ T cells detected in a control sample. For example the epitope can be immunogenic according to the functional assay when (i) at least 10, 15, 20, 30, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected, (ii) the detected T cells make up at least 0.005% of the CD8⁺ or the CD4⁺ cells analyzed, and (iii) the percentage of detected T cells of CD8+ or CD4⁺ T cells is higher than the percentage of detected T cells of CD8+ or CD4⁺ T cells detected in a control sample. For example the epitope can be immunogenic according to the functional assay when (i) at least 10 T cells that have been stimulated with APCs comprising a peptide containing the at least one selected epitope sequence are detected, (ii) the detected T cells make up at least 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, or 10% of the CD8⁺ or the CD4⁺ cells analyzed, and (iii) the percentage of detected T cells of CD8+ or CD4⁺ T cells is higher than the percentage of detected T cells of CD8+ or CD4⁺ T cells detected in a control sample. For example the epitope can be immunogenic according to the functional assay when (i) at least 10, 15, 20, 30, 40, 50, 60, 70, 80, 90, 100, 150, 200, 250, 300, 400, 500, 600, 700, 800, 900 or more T cells that have been stimulated with APCs comprising a peptide containing the at least one selected epitope sequence are detected, (ii) the detected T cells make up at least 0.01%, 0.05%, 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, or 10% of the CD8⁺ or the CD4⁺ cells analyzed, and (iii) the percentage of detected T cells of CD8+ or CD4⁺ T cells is higher than the percentage of detected T cells of CD8+ or CD4⁺ T cells detected in a control sample.

In some embodiments, the T cells stimulated to be cytotoxic according to the cytotoxicity assay are T cells that have been stimulated with APCs comprising a peptide containing the at least one selected epitope sequence that kill cells presenting the epitope. In some embodiments, a number of cells presenting the epitope that are killed by the T cells is at least 1.1, 2, 3, 4, 5, 6, 7, 8, 9, 10, 15, 20, 25, 30, 50, 100, 500, or 1,000 fold higher than a number of cells that do not present the epitope that are killed by the T cells. In some embodiments, a number of cells presenting the epitope that are killed by the T cells is at least 1.1, 2, 3, 4, 5, 6, 7, 8, 9, 10, 15, 20, 25, 30, 50, 100, 500, or 1,000 fold higher than a number of cells presenting the epitope killed by T cells that have been stimulated with APCs that (i) do not comprise a peptide containing the at least one selected epitope sequence, (ii) comprise a peptide derived from a different protein than the at least one selected epitope sequence, or (iii) comprise a peptide with a random sequence In some embodiments, a number of cells presenting a mutant epitope that are killed by the T cells is at least 1.1, 2, 3, 4, 5, 6, 7, 8, 9, 10, 15, 20, 25, 30, 50, 100, 500, or 1,000 fold higher than a number of cells presenting a corresponding wild-type epitope that are killed by the T cells. In some embodiments, the T cells stimulated to be cytotoxic according to the cytotoxicity assay are T cells stimulated to be specifically cytotoxic according to the cytotoxicity assay.

In some embodiments, at least one of the one or more peptides is a synthesized peptide or a peptide expressed from a nucleic acid sequence.

In some embodiments, the method comprises identifying a protein encoded by an HLA allele of the subject or identifying an HLA allele in the genome of the subject.

In some embodiments, the at least one selected epitope sequence is selected from one or more epitope sequences of Table 1-8 and 11-14.

In some embodiments, the method comprises expanding the T cell contacted with the one or more peptides in vitro or ex vivo to obtain a population of T cells specific to the at least one selected epitope sequence in complex with an MEC protein.

In some embodiments, a protein comprising the at least one selected epitope sequence is expressed by a cancer cell of the subject. In some embodiments, a protein comprising the at least one selected epitope sequences is expressed by cells in the tumor microenvironment of the subject.

In some embodiments, one or more of the at least one selected epitope sequence comprises a mutation. In some embodiments, one or more of the at least one selected epitope sequence comprises a tumor specific mutation. In some embodiments, one or more of the at least one selected epitope sequence is from a protein overexpressed by a cancer cell of the subject. In some embodiments, one or more of the at least one selected epitope sequence comprises a driver mutation. In some embodiments, one or more of the at least one selected epitope sequence comprises a drug resistance mutation. In some embodiments, one or more of the at least one selected epitope sequence is from a tissue-specific protein. In some embodiments, one or more of the at least one selected epitope sequence is from a cancer testes protein. In some embodiments, one or more of the at least one selected epitope sequence is a viral epitope. In some embodiments, one or more of the at least one selected epitope sequence is a minor histocompatibility epitope. In some embodiments, one or more of the at least one selected epitope sequence is from a RAS protein. In some embodiments, one or more of the at least one selected epitope sequence is from a GATA3 protein. In some embodiments, one or more of the at least one selected epitope sequence is from a EGFR protein. In some embodiments, one or more of the at least one selected epitope sequence is from a BTK protein. In some embodiments, one or more of the at least one selected epitope sequence is from a p53 protein. In some embodiments, one or more of the at least one selected epitope sequence is from aTMPRSS2::ERG fusion polypeptide. In some embodiments, one or more of the at least one selected epitope sequence is from a Myc protein. In some embodiments, at least one of the at least one selected epitope sequence is from a protein encoded by a gene selected from the group consisting of ANKRD30A, COL10A1, CTCFL, PPIAL4G, POTEE, DLL3, MMP13, SSX1, DCAF4L2, MAGEA4, MAGEA11, MAGEC2, MAGEA12, PRAME, CLDN6, EPYC, KLK3, KLK2, KLK4, TGM4, POTEG, RLN1, POTEH, SLC45A2, TSPAN10, PAGES, CSAG1, PRDM7, TG, TSHR, RSPH6A, SCXB, HIST1H4K, ALPPL2, PRM2, PRM1, TNP1, LELP1, HMGB4, AKAP4, CETN1, UBQLN3, ACTL7A, ACTL9, ACTRT2, PGK2, C2orf53, KIF2B, ADAD1, SPATA8, CCDC70, TPD52L3, ACTL7B, DMRTB1, SYCN, CELA2A, CELA2B, PNLIPRP1, CTRC, AMY2A, SERPINI2, RBPJL, AQP12A, IAPP, KIRREL2, G6PC2, AQP12B, CYP11B1, CYP11B2, STAR, CYP11A1, and MC2R.

In some embodiments, at least one of the at least one selected epitope sequence is from a tissue-specific protein that has an expression level in a target tissue of the subject that is at least 2 fold more than an expression level of the tissue-specific protein in each tissue of a plurality of non-target tissues that are different than the target tissue.

In some embodiments, contacting a T cell from the subject or an allogeneic T cell with one or more peptides comprising the at least one selected epitope sequence comprises contacting the T cell with APCs presenting the epitope.

In some embodiments, the APCs presenting the epitope comprises one or more peptides comprising the at least one selected epitope sequence or a polynucleic acid that encodes one or more peptides comprising the at least one selected epitope sequence. In some embodiments, the polypeptide comprises at least two of the selected epitope sequence, each expressed by cancer cells of a human subject with cancer.

In some embodiments, the method comprises depleting CD14+ cells and CD25+ cells from a population of immune cells comprising antigen presenting cells (APCs) and T cells, thereby forming a CD14/CD25 depleted population of immune cells comprising a first population of APCs and T cells. In some embodiments, the population of immune cells is from a biological sample from the subject. In some embodiments, the method further comprises incubating the CD14/CD25 depleted population of immune cells comprising a first population of APCs and T cells for a first time period in the presence of FMS-like tyrosine kinase 3 receptor ligand (FLT3L), and a polypeptide comprising the at least one selected epitope sequence, or a polynucleotide encoding the polypeptide; thereby forming a population of cells comprising stimulated T cells. In some embodiments, the method further comprises expanding the population of cells comprising stimulated T cells, thereby forming an expanded population of cells comprising tumor antigen-specific T cells, wherein the tumor antigen-specific T cells comprise T cells that are specific to a complex comprising the at least one selected epitope sequence and an MHC protein expressed by the cancer cells or APCs of the subject. In some embodiments, expanding is performed in less than 28 days. In some embodiments, incubating comprises incubating the CD14/CD25 depleted population of immune cells comprising a first population of APCs and T cells for a first time period in the presence of FLT3L and an RNA encoding the polypeptide. In some embodiments, depleting CD14+ cells and CD25+ cells from the population of immune cells comprising a first population of APCs and T cells comprises contacting the population of immune cells comprising a first population of APCs and T cells with a CD14 binding agent and a CD25 binding agent. In some embodiments, depleting further comprising depleting CD19+ cells from the population of immune cells comprising a first population of APCs and T cells. In some embodiments, depleting further comprising depleting CD11b+ cells from the population of immune cells comprising a first population of APCs and T cells.

In some embodiments, the method further comprises administering a pharmaceutical composition comprising the expanded population of cells comprising tumor antigen specific T cells to a human subject with cancer. In some embodiments, the human subject with cancer is the human subject from which the biological sample was obtained.

In some embodiments, the fraction of CD8+ tumor antigen-specific T cells of the total number of CD8+ T cells in the expanded population of cells comprising tumor antigen specific T cells is at least two-fold higher than the fraction of CD8+ tumor antigen-specific T cells of the total number of CD8+ T cells in the biological sample. In some embodiments, the fraction of CD4+ tumor antigen-specific T cells of the total number of CD4+ T cells in the expanded population of cells comprising tumor antigen specific T cells is at least two-fold higher than the fraction of CD4+ tumor antigen-specific T cells of the total number of CD4+ T cells in the biological sample. In some embodiments, at least 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, or 10% of the CD8+ T cells in the expanded population of cells comprising tumor antigen specific T cells are CD8+ tumor antigen-specific T cells derived from naïve CD8+ T cells. In some embodiments, at least 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, or 10% of the CD8+ T cells in the expanded population of cells comprising tumor antigen specific T cells are CD8+ tumor antigen-specific T cells derived from memory CD8+ T cells. In some embodiments, at least 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, or 10% of the CD4+ T cells in the expanded population of cells comprising tumor antigen specific T cells are CD4+ tumor antigen-specific T cells derived from naïve CD4+ T cells. In some embodiments, at least 0.1%, 0.2%, 0.3%, 0.4%, 0.5%, 0.6%, 0.7%, 0.8%, 0.9%, 1%, 1.5%, 2%, 3%, 4%, 5%, 6%, 7%, 8%, 9%, or 10% of the CD4+ T cells in the expanded population of cells comprising tumor antigen specific T cells are CD4+ tumor antigen-specific T cells derived from memory CD4+ T cells.

In some embodiments, expanding comprises contacting the population of cells comprising stimulated T cells with a second population of mature APCs, wherein the second population of mature APCs have been incubated with FLT3L and present the at least one selected epitope sequence; and expanding the population of cells comprising stimulated T cells for a second time period, thereby forming an expanded population of T cells. In some embodiments, the second population of mature APCs has been incubated with FLT3L for at least 1 day prior to contacting the population of cells comprising stimulated T cells with the second population of mature APCs. In some embodiments, expanding further comprises contacting the expanded population of T cells with a third population of mature APCs, wherein the third population of mature APCs have been incubated with FLT3L and present the at least one selected epitope sequence; and expanding the expanded population of T cells for a third time period, thereby forming the expanded population of cells comprising tumor antigen-specific T cells. In some embodiments, the third population of mature APCs has been incubated with FLT3L for at least 1 day prior to contacting the expanded population of T cells with the third population of mature APCs. In some embodiments, the biological sample is a peripheral blood sample, a leukapheresis sample or an apheresis sample.

In some embodiments, the method further comprises harvesting the expanded population of cells comprising tumor antigen-specific T cells, cryopreserving the expanded population of cells comprising tumor antigen-specific T cells or preparing a pharmaceutical composition containing the expanded population of cells comprising tumor antigen-specific T cells.

In some embodiments, the method comprises generating cancer cell nucleic acids from a first biological sample comprising cancer cells obtained from a subject and generating non-cancer cell nucleic acids from a second biological sample comprising non-cancer cells obtained from the same subject.

In some embodiments, the protein encoded by an HLA allele of the subject is a protein encoded by an HLA allele selected from the group consisting of HLA-A01:01, HLA-A02:01, HLA-A03:01, HLA-A11:01, HLA-A24:01, HLA-A30:01, HLA-A31:01, HLA-A32:01, HLA-A33:01, HLA-A68:01, HLA-B07:02, HLA-B08:01, HLA-B15:01, HLA-B44:03, HLA-007:01 and HLA-007:02.

In some embodiments, the method comprises identifying one or two or more different proteins that comprise the at least one selected epitope sequence and that are expressed by cancer cells of the subject. In some embodiments, the method comprises identifying one or two or more different proteins that comprise the at least one selected epitope sequence and that are expressed by cancer cells of the subject by measuring levels of RNA encoding the one or two or more different proteins in the cancer cells. In some embodiments, the method comprises isolating genomic DNA or RNA from cancer cells and non-cancer cells of the subject.

In some embodiments, one or more of the at least one selected epitope sequence comprises a point mutation or a sequence encoded by a point mutation. In some embodiments, one or more of the at least one selected epitope sequence comprises a sequence encoded by a neoORF mutation. In some embodiments, one or more of the at least one selected epitope sequence comprises a sequence encoded by a gene fusion mutation. In some embodiments, one or more of the at least one selected epitope sequence comprises a sequence encoded by an indel mutation. In some embodiments, one or more of the at least one selected epitope sequence comprises a sequence encoded by a splice site mutation. In some embodiments, at least two of the at least one selected epitope sequence are from a same protein. In some embodiments, at least two of the at least one selected epitope sequence comprise an overlapping sequence. In some embodiments, at least two of the at least one selected epitope sequence are from different proteins. In some embodiments, the one or more peptides comprise at least 2, 3, 4, 5, 6, 7, 8, 9, or 10 or more peptides.

In some embodiments, cancer cells of the subject are cancer cells of a solid cancer. In some embodiments, cancer cells of the subject are cancer cells of a leukemia or a lymphoma.

In some embodiments, the mutation is a mutation that occurs in a plurality of cancer patients.

In some embodiments, the MEC is a Class I MEC. In some embodiments, the MHC is a Class II MHC.

In some embodiments, the T cell is a CD8 T cell. In some embodiments, the T cell is a CD4 T cell. In some embodiments, the T cell is a cytotoxic T cell. In some embodiments, the T cell t is a memory T cell. In some embodiments, the T cell is a naive T cell.

In some embodiments, the method further comprises selecting one or more subpopulation of cells from an expanded population of T cells prior to administering to the subject.

In some embodiments, eliciting an elicit an immune response in the T cell culture comprises inducing IL2 production from the T cell culture upon contact with the peptide. In some embodiments, eliciting an immune response in the T cell culture comprises inducing a cytokine production from the T cell culture upon contact with the peptide, wherein the cytokine is an Interferon gamma (IFN-γ), Tumor Necrosis Factor (TNF) alpha (α) and/or beta (β) or a combination thereof. In some embodiments, eliciting an immune response in the T cell culture comprises inducing the T cell culture to kill a cell expressing the peptide. In some embodiments, eliciting an immune response in the T cell culture comprises detecting an expression of a Fas ligand, granzyme, perforins, IFN, TNF, or a combination thereof in the T cell culture.

In some embodiments, the one or more peptides comprising the at least one selected epitope sequence is purified. In some embodiments, the one or more peptides comprising the at least one selected epitope sequence is lyophilized. In some embodiments, the one or more peptides comprising the at least one selected epitope sequence is in a solution. In some embodiments, the one or more peptides comprising the at least one selected epitope sequence is present in a storage condition such that the integrity of the peptide is ≥99%.

In some embodiments, the method comprises stimulating T cells to be cytotoxic against cells loaded with the at least one selected epitope sequences according to a cytotoxicity assay. In some embodiments, the method comprises stimulating T cells to be cytotoxic against cancer cells expressing a protein comprising the at least one selected epitope sequences according to a cytotoxicity assay. In some embodiments, the method comprises stimulating T cells to be cytotoxic against a cancer associated cell expressing a protein comprising the at least one selected epitope sequences according to a cytotoxicity assay.

In some embodiments, the at least one selected epitope is expressed by a cancer cell, and an additional selected epitope is expressed by a cancer associated cell. In some embodiments, the additional selected epitope is expressed on a cancer associated fibroblast cell. In some embodiments, the additional selected epitope is selected from Table 8.

In some embodiments, a method provided herein is a method for treating cancer in a subject in need thereof comprising: selecting at least one epitope sequence from a library of epitope sequences, wherein each epitope sequence in the library is matched to a protein encoded by an HLA allele; and contacting a T cell from the subject or an allogeneic T cell with one or more peptides comprising the at least one selected epitope sequence, wherein each of the at least one selected epitope sequences; binds to a protein encoded by an HLA allele of the subject; is immunogenic according to an immunogenic assay; is presented by antigen presenting cells (APCs) according to a mass spectrometry assay, and stimulates T cells to be cytotoxic according to a cytotoxicity assay.

In some embodiments, the method comprises selecting the subject using a circulating tumor DNA assay. In some embodiments, the method comprises selecting the subject using a gene panel.

In some embodiments, the T cell is from a biological sample from the subject. In some embodiments, the T cell is from an apheresis or a leukopheresis sample from the subject.

In some embodiments, at least one of the one or more peptides a synthesized peptide or a peptide expressed from a nucleic acid sequence.

In some embodiments, the method comprises identifying a protein encoded by an HLA allele of the subject or identifying an HLA allele in the genome of the subject. In some embodiments, the method comprises identifying a protein encoded by an HLA allele of the subject that is expressed by the subject. In some embodiments, the method comprises contacting a T cell from the subject with one or more peptides selected from one or more peptides of a table provided herein. In some embodiments, the method comprises contacting a T cell from the subject with one or more peptides comprising an epitope selected from an epitope of a table provided herein. In some embodiments, the method further comprises expanding in vitro or ex vivo the T cell contacted with the one or more peptides to obtain a population of T cells. In some embodiments, the method further comprises administering the population of T cells to the subject at a dose and a time interval such that the cancer is reduced or eliminated.

In some embodiments, at least one of the one or more peptides is expressed by a cancer cell of the subject. In some embodiments, at least one of the epitopes of the one or more peptides comprises a mutation.

In some embodiments, at least one of the epitopes of the one or more peptides comprises a tumor specific mutation. In some embodiments, at least one of the epitopes of the one or more peptides is from a protein overexpressed by a cancer cell of the subject. In some embodiments, at least one of the epitopes of the one or more peptides is from a protein encoded by a gene selected from the group consisting of ANKRD30A, COL10A1, CTCFL, PPIAL4G, POTEE, DLL3, MMP13, SSX1, DCAF4L2, MAGEA4, MAGEA11, MAGEC2, MAGEA12, PRAME, CLDN6, EPYC, KLK3, KLK2, KLK4, TGM4, POTEG, RLN1, POTEH, SLC45A2, TSPAN10, PAGES, CSAG1, PRDM7, TG, TSHR, RSPH6A, SCXB, HIST1H4K, ALPPL2, PRM2, PRM1, TNP1, LELP1, HMGB4, AKAP4, CETN1, UBQLN3, ACTL7A, ACTL9, ACTRT2, PGK2, C2orf53, KIF2B, ADAD1, SPATA8, CCDC70, TPD52L3, ACTL7B, DMRTB1, SYCN, CELA2A, CELA2B, PNLIPRP1, CTRC, AMY2A, SERPINI2, RBPJL, AQP12A, LAPP, KIRREL2, G6PC2, AQP12B, CYP11B1, CYP11B2, STAR, CYP11A1, and MC2R.

In some embodiments, at least one of the one or more peptides is from a protein encoded by a tissue-specific antigen epitope gene that has an expression level in a target tissue of the subject that is at least 2 fold more than an expression level of the tissue-specific antigen gene in each tissue of a plurality of non-target tissues that are different than the target tissue.

In some embodiments, the method comprises: incubating one or more antigen presenting cell (APC) preparations with a population of immune cells from a biological sample depleted of cells expressing CD14 and CD25 for one or more separate time periods; incubating one or more APC preparations with a population of immune cells from a biological sample for one or more separate time periods, wherein the one or more APCs comprise one or more FMS-like tyrosine kinase 3 receptor ligand (FLT3L)-stimulated APCs; or incubating FLT3L and at least one peptide with a population of immune cells from a biological sample, wherein the FLT3L is incubated with the population of immune cells for a first time period and wherein the at least one peptide is incubated with the population of immune cells for a first peptide stimulation time period, thereby obtaining a first stimulated T cell sample, wherein the population of immune cells comprises at least one T cell and at least one APC; wherein at least one antigen specific memory T cell is expanded, or at least one antigen specific naïve T cell is induced.

In some embodiments, the method comprises incubating a population of immune cells from a biological sample with one or more APC preparations for one or more separate time periods of less than 28 days from incubating the population of immune cells with a first APC preparation of the one or more APC preparations. In some embodiments, the method comprises incubating a population of immune cells from a biological sample with 3 or less APC preparations for 3 or less separate time periods. In some embodiments, the method comprises incubating a population of immune cells from a biological sample with 2 or less APC preparations for 2 or less separate time periods. In some embodiments, the method comprises incubating a population of immune cells from a biological sample with one or more APC preparations for one or more separate time periods of less than 28 days from incubating the population of immune cells with a first APC preparation of the one or more APC preparations. In some embodiments, the total period of preparation of T cells stimulated with an antigen by incubating a population of immune cells from a biological sample with one or more APC preparations for one or more separate time periods is less than 28 days.

In some embodiments, at least two of the one or more APC preparations comprise a FLT3L-stimulated APC. In some embodiments, at least three of the one or more APC preparations comprise a FLT3L-stimulated APC. In some embodiments, incubating comprises incubating a first APC preparation of the APC preparations to the T cells for more than 7 days. In some embodiments, an APC of the APC preparations comprises an APC loaded with one or more antigen peptides comprising one or more of the at least one antigen peptide sequence. In some embodiments, an APC of the APC preparations is an autologous APC or an allogenic APC. In some embodiments, an APC of the APC preparations comprises a dendritic cell (DC). In some embodiments, the DC is a CD141⁺ DC. In some embodiments, the method comprises depleting cells expressing CD14 and CD25 from the biological sample, thereby obtaining the population of immune cells from a biological sample depleted of cells expressing CD14 and CD25. In some embodiments, the method further comprises depleting cells expressing CD19. In some embodiments, the method further comprises depleting cells expressing CD11b. In some embodiments, depleting cells expressing CD14 and CD25 comprises binding a CD14 or CD25 binding agent to an APC of the one or more APC preparations. In some embodiments, the method further comprises administering one or more of the at least one antigen specific T cell to a subject.

In some embodiments, incubating comprises incubating a first APC preparation of the one or more APC preparations to the T cells for more than 7, 8, 9, 10, 11, 12, 13, 14, 15, 16, 17, 18, 19, or 20 days. In some embodiments, the method comprises incubating at least one of the one or more of the APC preparations with a first medium comprising at least one cytokine or growth factor for a first time period. In some embodiments, the method comprises incubating at least one of the one or more of the APC preparations with a second medium comprising one or more cytokines or growth factors for a third time period, thereby obtaining a matured APC. In some embodiments, the method further comprises removing the one or more cytokines or growth factors of the second medium after the third time period. In some embodiments, an APC of the APC preparations is stimulated with one or more cytokines or growth factors. In some embodiments, the one or more cytokines or growth factors comprise GM-CSF, IL-4, FLT3L, TNF-α, IL-1β, PGE1, IL-6, IL-7, IFN-α, R848, LPS, ss-rna40, poly I:C, or a combination thereof.

In some embodiments, the antigen is a neoantigen, a tumor associated antigen, a viral antigen, a minor histocompatibility antigen or a combination thereof.

In some embodiments, the method is performed ex vivo.

In some embodiments, wherein the method comprises incubating the population of immune cells from a biological sample depleted of cells expressing CD14 and CD25 with FLT3L for a first time period. In some embodiments, the method comprises incubating at least one peptide with the population of immune cells from a biological sample depleted of cells expressing CD14 and CD25 for a second time period, thereby obtaining a first matured APC peptide loaded sample. In some embodiments, the method comprises depleting cells expressing CD14, cells expressing CD19 and cells expressing CD25 from the population of immune cells. In some embodiments, the method comprises depleting cells expressing CD14, cells expressing CD11b and cells expressing CD25 from the population of immune cells. In some embodiments, the method comprises depleting cells expressing CD14, cells expressing CD11b, cells expressing CD19 and cells expressing CD25. In some embodiments, the method comprises depleting at least CD14, CD11b, CD19 and CD25. In some embodiments, the method comprises depleting cells expressing at least one of CD14, CD11b, CD19 and CD25, and at least a fifth cell type expressing a fifth cell surface marker. In some embodiments, the method comprises selectively depleting CD14 and CD25 expressing cells from the population of immune cells, and any one or more of CD19, CD11b expressing cells, from the population of immune cells, at a first incubation period, at a second incubation period, and/or at a third incubation period.

In some embodiments of the method described herein, contacting a T cell from the subject or an allogeneic T cell with one or more peptides comprising the at least one selected epitope sequence comprises contacting the T cell with APCs presenting the epitope.

In some embodiments of the method described herein, the APCs presenting the epitope comprises one or more peptides comprising the at least one selected epitope sequence or a polynucleic acid that encodes one or more peptides comprising the at least one selected epitope sequence.

In some embodiments, the method comprises depleting CD14+ cells and CD25+ cells from a population of immune cells comprising antigen presenting cells (APCs) and T cells, thereby forming a CD14/CD25 depleted population of immune cells comprising a first population of APCs and T cells. In some embodiments, the population of immune cells is from a biological sample from the subject. In some embodiments of the method described herein, the method further comprises incubating the CD14/CD25 depleted population of immune cells comprising a first population of APCs and T cells for a first time period in the presence of FMS-like tyrosine kinase 3 receptor ligand (FLT3L), and a polypeptide comprising the at least one selected epitope sequences, or a polynucleotide encoding the polypeptide; thereby forming a population of cells comprising stimulated T cells. In some embodiments, the method further comprises expanding the population of cells comprising stimulated T cells, thereby forming an expanded population of cells comprising tumor antigen-specific T cells, wherein the tumor antigen-specific T cells comprise T cells that are specific to a complex comprising the at least one selected epitope sequences and an MEC protein expressed by the cancer cells or APCs of the subject.

In some embodiments of the method described herein, expanding comprises contacting the population of cells comprising stimulated T cells with a second population of mature APCs, wherein the second population of mature APCs have been incubated with FLT3L and present the at least one selected epitope sequence and expanding the population of cells comprising stimulated T cells for a second time period, thereby forming an expanded population of T cells. In some embodiments, the second population of mature APCs has been incubated with FLT3L for at least 1 day prior to contacting the population of cells comprising stimulated T cells with the second population of mature APCs. In some embodiments, the expanding further comprises contacting the expanded population of T cells with a third population of mature APCs, wherein the third population of mature APCs have been incubated with FLT3L and present the at least one selected epitope sequence; and expanding the expanded population of T cells for a third time period, thereby forming the expanded population of cells comprising tumor antigen-specific T cells. In some embodiments, the third population of mature APCs has been incubated with FLT3L for at least 1 day prior to contacting the expanded population of T cells with the third population of mature APCs. In some embodiments of the method described herein, the method further comprises harvesting the expanded population of cells comprising tumor antigen-specific T cells, cryopreserving the expanded population of cells comprising tumor antigen-specific T cells or preparing a pharmaceutical composition containing the expanded population of cells comprising tumor antigen-specific T cells. In some embodiments, the incubating comprises incubating the CD14/CD25 depleted population of immune cells comprising a first population of APCs and T cells for a first time period in the presence of FLT3L and an RNA encoding the polypeptide.

In some embodiments, the method further comprises administering a pharmaceutical composition comprising the expanded population of cells comprising tumor antigen specific T cells to a human subject with cancer. In some embodiments, the human subject with cancer is the human subject from which the biological sample was obtained. In some embodiments, the polypeptide is from 8 to 50 amino acids in length. In some embodiments, the polypeptide comprises at least two of the selected epitope sequence, each expressed by cancer cells of a human subject with cancer.

In some embodiments, depleting CD14+ cells and CD25+ cells from the population of immune cells comprising a first population of APCs and T cells comprises contacting the population of immune cells comprising a first population of APCs and T cells with a CD14 binding agent and a CD25 binding agent. In some embodiments, depleting further comprising depleting CD19+ cells from the population of immune cells comprising a first population of APCs and T cells. In some embodiments, the method further comprises contacting the population of immune cells with a CD19 binding agent. In some embodiments, depleting further comprising depleting CD11b+ cells from the population of immune cells comprising a first population of APCs and T cells. In some embodiments, the method further comprises contacting the population of immune cells with a CD11b binding agent.

In some embodiments, the method comprises incubating the first matured APC peptide loaded sample with at least one T cell for a third time period, thereby obtaining a stimulated T cell sample. In some embodiments, the method comprises incubating a T cell of a first stimulated T cell sample with a FLT3L-stimulated APC of a matured APC sample for a fourth time period, FLT3L and a second APC peptide loaded sample of a matured APC sample for a fourth time period or FLT3L and a FLT3L-stimulated APC of a matured APC sample for a fourth time period, thereby obtaining a stimulated T cell sample. In some embodiments, the method comprises incubating a T cell of a second stimulated T cell sample with a FLT3L-stimulated APC of a matured APC sample for a fifth time period, FLT3L and a third APC peptide loaded sample of a matured APC sample for a fifth time period, or FLT3L and a third APC peptide loaded sample of a matured APC sample for a fifth time period, thereby obtaining a stimulated T cell sample.

In some embodiments, the one or more separate time periods, the 3 or less separate time periods, the first time period, the second time period, the third time period, the fourth time period, or the fifth time period is at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 37 hours, at least 38 hours, at least 39 hours, or at least 40 hours.

In some embodiments, the one or more separate time periods, the 3 or less separate time periods, the first time period, the second time period, the third time period, the fourth time period, or the fifth time period is from 1 to 4 hours, from 1 to 3 hours, from 1 to 2 hours, from 4 to 40 hours, from 7 to 40 hours, from 4 to 35 hours, from 4 to 32 hours, from 7 to 35 hours or from 7 to 32 hours.

In some embodiments, the population of immune cells comprises the APC or at least one of the one or more APC preparations. In some embodiments, the population of immune cells does not comprise the APC and/or the population of immune cells does not comprise one of the one or more APC preparations.

In some embodiments, the method comprises incubating FLT3L and at least one peptide with a population of immune cells from a biological sample, wherein the FLT3L is incubated with the population of immune cells for a first time period and wherein the at least one peptide is incubated with the population of immune cells for a first peptide stimulation time period, thereby obtaining a first stimulated T cell sample, wherein the population of immune cells comprises at least one T cell and at least one APC. In some embodiments, the method comprises incubating FLT3L and at least one peptide with at least one APC, wherein the FLT3L is incubated with the at least one APC for a second time period and wherein the at least one peptide is incubated with the at least one APC for a second peptide stimulation time period, thereby obtaining a first matured APC peptide loaded sample; and incubating the first matured APC peptide loaded sample with the first stimulated T cell sample, thereby obtaining a second stimulated T cell sample. In some embodiments, the method comprises incubating FLT3L and at least one peptide with at least one APC, wherein the FLT3L is incubated with the at least one APC for a third time period and wherein the at least one peptide is incubated with the at least one APC for a third peptide stimulation time period, thereby obtaining a second matured APC peptide loaded sample; and incubating the second matured APC peptide loaded sample with the second stimulated T cell sample, thereby obtaining a third stimulated T cell sample.

In some embodiments, the method further comprises isolating the first stimulated T cell from the stimulated T cell sample. In some embodiments, isolating as described in the preceding sentence comprises enriching a stimulated T cell from a population of immune cells that have been contacted with the at least one APC incubated with the at least one peptide. In some embodiments, the enriching comprises determining expression of one or more cell markers of at least one the stimulated T cell and isolating the stimulated T cell expressing the one or more cell markers. In some embodiments the cell surface markers may be but not limited to one or more of TNF-α, IFN-γ, LAMP-1, 4-1BB, IL-2, IL-17A, Granzyme B, PD-1, CD25, CD69, TIM3, LAG3, CTLA-4, CD62L, CD45RA, CD45RO, FoxP3, or any combination thereof. In some embodiments, the one or more cell markers comprise a cytokine.

In some embodiments, the method comprises administering at least one T cell of a first or a second or a third stimulated T cell sample to a subject in need thereof.

In some embodiments, the method comprises: obtaining a biological sample from a subject comprising at least one antigen presenting cell (APC); enriching cells expressing CD14 from the biological sample, thereby obtaining a CD14⁺ cell enriched sample; incubating the CD14⁺ cell enriched sample with at least one cytokine or growth factor for a first time period; incubating at least one peptide with the CD14⁺ cell enriched sample of for a second time period, thereby obtaining an APC peptide loaded sample; incubating the APC peptide loaded sample with one or more cytokines or growth factors for a third time period, thereby obtaining a matured APC sample; incubating APCs of the matured APC sample with a CD14 and CD25 depleted sample comprising T cells for a fourth time period; incubating the T cells with APCs of a matured APC sample for a fifth time period; incubating the T cells with APCs of a matured APC sample for a sixth time period; and administering at least one T cell of the T cells to a subject in need thereof.

In some embodiments, the method comprises: obtaining a biological sample from a subject comprising at least one APC and at least one T cell; depleting cells expressing CD14 and CD25 from the biological sample, thereby obtaining a CD14 and CD25 cell depleted sample; incubating the CD14 and CD25 cell depleted sample with FLT3L for a first time period; incubating at least one peptide with the CD14 and CD25 cell depleted sample of for a second time period, thereby obtaining an APC peptide loaded sample; incubating the APC peptide loaded sample with the at least one T cell for a third time period, thereby obtaining a first stimulated T cell sample; incubating a T cell of the first stimulated T cell sample with an APC of a matured APC sample for a fourth time period, thereby obtaining a second stimulated T cell sample; optionally, incubating a T cell of the second stimulated T cell sample with an APC of a matured APC sample for a fifth time period, thereby obtaining a third stimulated T cell sample; administering at least one T cell of the first, the second or the third stimulated T cell sample to a subject in need thereof.

In some embodiments, the method comprises: obtaining a biological sample from a subject comprising at least one APC and at least one T cell; depleting cells expressing CD14 and CD25 from the biological sample, thereby obtaining a CD14 and CD25 cell depleted sample; incubating the CD14 and CD25 cell depleted sample with FLT3L for a first time period; incubating at least one peptide with the CD14 and CD25 cell depleted sample of for a second time period, thereby obtaining an APC peptide loaded sample; incubating the APC peptide loaded sample with the at least one T cell for a third time period, thereby obtaining a first stimulated T cell sample; optionally, incubating a T cell of the first stimulated T cell sample with a FLT3L-stimulated APC of a matured APC sample for a fourth time period, thereby obtaining a second stimulated T cell sample; optionally, incubating a T cell of the second stimulated T cell sample with a FLT3L-stimulated APC of a matured APC sample for a fifth time period, thereby obtaining a third stimulated T cell sample; administering at least one T cell of the first, the second or the third stimulated T cell sample to a subject in need thereof.

In some embodiments, the method comprises: obtaining a biological sample from a subject comprising at least one APC and at least one T cell; depleting cells expressing CD14 and CD25 from the biological sample, thereby obtaining a CD14 and CD25 cell depleted sample; incubating the CD14 and CD25 cell depleted sample with FLT3L for a first time period; incubating at least one peptide with the CD14 and CD25 cell depleted sample of for a second time period, thereby obtaining a first APC peptide loaded sample; incubating the first APC peptide loaded sample with the at least one T cell for a third time period, thereby obtaining a first stimulated T cell sample; optionally, incubating a T cell of the first stimulated T cell sample with FLT3L and a second APC peptide loaded sample of a matured APC sample for a fourth time period, thereby obtaining a second stimulated T cell sample; optionally, incubating a T cell of the second stimulated T cell sample with FLT3L and a third APC peptide loaded sample of a matured APC sample for a fifth time period, thereby obtaining a third stimulated T cell sample; administering at least one T cell of the first, the second or the third stimulated T cell sample to a subject in need thereof.

In some embodiments, the method comprises: obtaining a biological sample from a subject comprising at least one APC and at least one T cell; depleting cells expressing CD14 and CD25 from the biological sample, thereby obtaining a CD14 and CD25 cell depleted sample; incubating the CD14 and CD25 cell depleted sample with FLT3L for a first time period; incubating at least one peptide with the CD14 and CD25 cell depleted sample of for a second time period, thereby obtaining a first APC peptide loaded sample; incubating the first APC peptide loaded sample with the at least one T cell for a third time period, thereby obtaining a first stimulated T cell sample; optionally, incubating a T cell of the first stimulated T cell sample with FLT3L and a FLT3L-stimulated APC of a matured APC sample for a fourth time period, thereby obtaining a second stimulated T cell sample; optionally, incubating a T cell of the second stimulated T cell sample with FLT3L and a FLT3L-stimulated APC of a matured APC sample for a fifth time period, thereby obtaining a third stimulated T cell sample; administering at least one T cell of the first, the second or the third stimulated T cell sample to a subject in need thereof.

In some embodiments, the method comprises: incubating FLT3L and at least one peptide with a population of immune cells from a biological sample, wherein the FLT3L is incubated with the population of immune cells for a first time period and wherein the at least one peptide is incubated with the population of immune cells for a first peptide stimulation time period, thereby obtaining a first stimulated T cell sample, wherein the population of immune cells comprises at least one T cell and at least one APC; optionally, incubating FLT3L and at least one peptide with at least one APC, wherein the FLT3L is incubated with the at least one APC for a second time period and wherein the at least one peptide is incubated with the at least one APC for a second peptide stimulation time period, thereby obtaining a first matured APC peptide loaded sample; and incubating the first matured APC peptide loaded sample with the first stimulated T cell sample, thereby obtaining a second stimulated T cell sample; optionally, incubating FLT3L and at least one peptide with at least one APC, wherein the FLT3L is incubated with the at least one APC for a third time period and wherein the at least one peptide is incubated with the at least one APC for a third peptide stimulation time period, thereby obtaining a second matured APC peptide loaded sample; and incubating the second matured APC peptide loaded sample with the second stimulated T cell sample, thereby obtaining a third stimulated T cell sample; and administering at least one T cell of the first stimulated T cell sample, the second stimulated T cell sample or the third stimulated T cell sample to a subject in need thereof.

In some embodiments, the method comprises generating cancer cell nucleic acids from a first biological sample comprising cancer cells obtained from a subject and generating non-cancer cell nucleic acids from a second biological sample comprising non-cancer cells obtained from the same subject.

In some embodiments, the method comprises sequencing cancer cell nucleic acids by whole genome sequencing or whole exome sequencing, thereby obtaining a first plurality of nucleic acid sequences comprising cancer cell nucleic acid sequences; and sequencing non-cancer cell nucleic acids by whole genome sequencing or whole exome sequencing, thereby obtaining a second plurality of nucleic acid sequences comprising non-cancer cell nucleic acid sequences. In some embodiments, the method comprises identifying a plurality of cancer specific nucleic acid sequences from a first plurality of nucleic acid sequences that are unique to cancer cells of the subject and that do not include nucleic acid sequences from a second plurality of nucleic acid sequences from non-cancer cells of the subject.

In some embodiments, the method further comprises selecting one or more subpopulation of cells from the expanded population of T cells prior to administering to the subject. In some embodiments, the selecting one or more subpopulation is performed by cell sorting based on expression of one or more cell surface markers provided herein. In some embodiments, the activated T cells may be sorted based on cell surface markers including but not limited to any one or more of the following: CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, CD45RO, CCR7, FLT3LG, IL-6 and others.

In some embodiments, the method further comprises depleting one or more cells in the subject prior to administering the population of T cells.

In some embodiments, the one or more subpopulation of cells expressing a cell surface marker provided herein.

In some embodiments, the amino acid sequence of a peptide provided herein is validated by peptide sequencing. In some embodiments, the amino acid sequence a peptide provided herein is validated by mass spectrometry.

Also provided herein is a pharmaceutical composition comprising a T cell produced by expanding the T cell in the presence of an antigen presenting cell presenting one or more epitope sequence of any of Tables 1-8 and 11-14.

Also provided herein is library of polypeptides comprising epitope sequences or polynucleotides encoding the polypeptides, wherein each epitope sequence in the library is matched to a protein encoded by an HLA allele; and wherein each epitope sequence in the library is pre-validated to satisfy at least two or three or four of the following criteria: binds to a protein encoded by an HLA allele of a subject with cancer to be treated, is immunogenic according to an immunogenic assay, is presented by antigen presenting cells (APCs) according to a mass spectrometry assay, and stimulates T cells to be cytotoxic according to a cytotoxicity assay. In some embodiments, the library comprises one or two or more peptide sequences comprising an epitope sequence of any of Tables 1-8 and 11-14.

The peptides and polynucleotides provided herein can be for preparing antigen-specific T cells and include recombinant peptides and polynucleotides and synthetic peptides comprising epitopes, such as a tumor-specific neoepitopes, that have been identified and validated as binding to one or more MEC molecules, presented by the one or more MEC molecules, being immunogenic and/or capable of activating T cells to become cytotoxic. The peptides can be prepared for use in a method to prime T cells ex vivo. The peptides can be prepared for use in a method to activate T cells ex vivo. The peptides can be prepared for use in a method to expand antigen-specific T cells. The peptides can be prepared for use in a method to induce de novo CD8 T cell responses ex vivo. The peptides can be prepared for use in a method to induce de novo CD4 T cell responses ex vivo. The peptides can be prepared for use in a method to stimulate memory CD8 T cell responses ex vivo. The peptides can be prepared for use in a method to stimulate memory CD4 T cell responses ex vivo. The T cells can be obtained from a human subject. The T cells can be allogeneic T cells. The T cells can be T cell lines.

The epitopes can comprise at least 8 contiguous amino acids of an amino acid sequence encoded by the genome of a cancer cell. The epitopes can comprise from 8-12 contiguous amino acids of an amino acid sequence encoded by the genome of a cancer cell. The epitopes can comprise from 13-25 contiguous amino acids of an amino acid sequence encoded by the genome of a cancer cell. The epitopes can comprise from 8-50 contiguous amino acids of an amino acid sequence encoded by the genome of a cancer cell. In some embodiments, an epitope is from about 8 and about 30 amino acids in length. In some embodiments, an epitope is from about 8 to about 25 amino acids in length. In some embodiments, an epitope is from about 15 to about 24 amino acids in length. In some embodiments, an epitope is from about 9 to about 15 amino acids in length. In some embodiments, an epitope is 8 amino acids in length. In some embodiments, an epitope is 9 amino acids in length. In some embodiments, an epitope is 10 amino acids in length.

In some embodiments, a peptide containing an epitope is at most 500, at most 250, at most 150, at most 125, or at most 100 amino acids in length In some embodiments, a peptide containing an epitope is at least 8, at least 50, at least 100, at least 200, or at least 300 amino acids in length. In some embodiments, a peptide containing an epitope is from about 8 to about 500 amino acids in length. In some embodiments, a peptide containing an epitope is from about 8 to about 100 amino acids in length. In some embodiments, a peptide containing an epitope is from about 8 to about 50 amino acids in length. In some embodiments, a peptide containing an epitope is from about 15 to about 35 amino acids in length. In some embodiments, a peptide containing an epitope is from about 8 and about 15 amino acids in length. In some embodiments, a peptide containing an epitope is from about 8 and about 11 amino acids in length. In some embodiments, a peptide containing an epitope is 9 or 10 amino acids in length. In some embodiments, a peptide containing an epitope is from about 8 and about 30 amino acids in length. In some embodiments, a peptide containing an epitope is from about 8 to about 25 amino acids in length. In some embodiments, a peptide containing an epitope is from about 15 to about 24 amino acids in length. In some embodiments, a peptide containing an epitope is from about 9 to about 15 amino acids in length.

In some embodiments, a peptide containing an epitope has a total length of at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 150, at least 200, at least 250, at least 300, at least 350, at least 400, at least 450, or at least 500 amino acids. In some embodiments, a peptide containing an epitope has a total length of at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 40, at most 50, at most 60, at most 70, at most 80, at most 90, at most 100, at most 150, at most 200, at most 250, at most 300, at most 350, at most 400, at most 450, or at most 500 amino acids. In some embodiments, a peptide containing an epitope comprises a first neoepitope peptide linked to at least a second neoepitope.

In some embodiments, a peptide contains a validated epitope from one or more of: ABL1, AC011997, ACVR2A, AFP, AKT1, ALK, ALPPL2, ANAPC1, APC, ARID1A, AR, AR-v7, ASCL2, β2M, BRAF, BTK, C15ORF40, CDH1, CLDN6, CNOT1, CT45A5, CTAG1B, DCT, DKK4, EEF1B2, EEF1DP3, EGFR, EIF2B3, env, EPHB2, ERBB3, ESR1, ESRP1, FAM111B, FGFR3, FRG1B, GAGE1, GAGE10, GATA3, GBP3, HER2, IDH1, JAK1, KIT, KRAS, LMAN1, MABEB16, MAGEA1, MAGEA10, MAGEA4, MAGEA8, MAGEB17, MAGEB4, MAGEC1, MEK, MLANA, MLL2, MMP13, MSH3, MSH6, MYC, NDUFC2, NRAS, PAGE2, PAGES, PDGFRa, PIK3CA, PMEL, pol protein, POLE, PTEN, RAC1, RBM27, RNF43, RPL22, RUNX1, SEC31A, SEC63, SF3B1, SLC35F5, SLC45A2, SMAP1, SMAP1, SPOP, TFAM, TGFBR2, THAP5, TP53, TTK, TYR, UBR5, VHL, XPOT an EEF1DP3:FRY fusion polypeptide, an EGFR:SEPT14 fusion polypeptide, an EGFRVIII deletion polypeptide, an EML4:ALK fusion polypeptide, an NDRG1:ERG fusion polypeptide, an AC011997.1:LRRC69 fusion polypeptide, a RUNX1(ex5)-RUNX1T1fusion polypeptide, a TMPRSS2:ERG fusion polypeptide, a NAB:STAT6 fusion polypeptide, a NDRG1:ERG fusion polypeptide, a PML:RARA fusion polypeptide, a PPP1R1B:STARD3 fusion polypeptide, a MAD1L1:MAFK fusion polypeptide, a FGFR3:TAC fusion polypeptide, a FGFR3:TACC3 fusion polypeptide, a BCR:ABL fusion polypeptide, a C11orf95:RELA fusion polypeptide, a CBFB:MYH11 fusion polypeptide, a CBFB:MYH11 fusion polypeptide, a CD74:ROS1 fusion polypeptide, a CD74:ROS1 fusion polypeptide, ERVE-4: protease, ERVE-4: reverse transcriptase, ERVE-4: reverse transcriptase, ERVE-4: unknown, ERVH-2 matrix protein, ERVH-2: gag, ERVH-2: retroviral matrix, ERVH48-1: coat protein, ERVH48-1: syncytin, ERVI-1 envelope protein, ERVK-5 gag, ERVK-5 env, ERVK-5 pol, EBV A73, EBV BALF3, EBV BALF4, EBV BALF5, EBV BARF0, EBV LF2, EBV RPMS1, HPV-16, HPV-16 E7, and HPV-16 E6. In some embodiments, a neoepitope contains a mutation due to a mutational event in β2M, BTK, EGFR, GATA3, KRAS, MLL2, a TMPRSS2:ERG fusion polypeptide, or TP53 or Myc.

In some embodiments, an epitope binds a major histocompatibility complex (MEC) class I molecule. In some embodiments, an epitope binds an MEC class I molecule with a binding affinity of about 500 nM or less. In some embodiments an epitope binds an MEC class I molecule with a binding affinity of about 250 nM or less. In some embodiments, an epitope binds an MEC class I molecule with a binding affinity of about 150 nM or less. In some embodiments, an epitope binds an MEC class I molecule with a binding affinity of about 50 nM or less.

In some embodiments, an epitope binds an binds MEC class I molecule and a peptide containing the class I epitope binds to an MEC class II molecule.

In some embodiments, an epitope binds an MEC class II molecule. In some embodiments, an epitope binds to human leukocyte antigen (HLA)-A, -B, -C, -DP, -DQ, or -DR. In some embodiments, an epitope binds an MEC class II molecule with a binding affinity of 1000 nM or less. In some embodiments, an epitope binds MEC class II with a binding affinity of 500 nM or less. In some embodiments an epitope binds an MEC class II molecule with a binding affinity of about 250 nM or less. In some embodiments, an epitope binds an MEC class II molecule with a binding affinity of about 150 nM or less. In some embodiments, an epitope binds an MEC class II molecule with a binding affinity of about 50 nM or less.

In some embodiments, a peptide containing a validated epitope further comprises one or more amino acids flanking the C-terminus of the epitope. In some embodiments, a peptide containing a validated epitope further comprises one or more amino acids flanking the N-terminus of the epitope. In some embodiments, a peptide containing a validated epitope further comprises one or more amino acids flanking the C-terminus of the epitope and one or more amino acids flanking the N-terminus of the epitope. In some embodiments, the flanking amino acids are not native flanking amino acids. In some embodiments, a first epitope used in a method described herein binds an MEC class I molecule and a second epitope binds an MHC class II molecule. In some embodiments, a peptide containing a validated epitope further comprises a modification which increases in vivo half-life of the peptide. In some embodiments, a peptide containing a validated epitope further comprises a modification which increases cellular targeting by the peptide. In some embodiments, a peptide containing a validated epitope further comprises a modification which increases cellular uptake of the peptide. In some embodiments, a peptide containing a validated epitope further comprises a modification which increases peptide processing. In some embodiments, a peptide containing a validated epitope further comprises a modification which increases MHC affinity of the epitope. In some embodiments, a peptide containing a validated epitope further comprises a modification which increases MEC stability of the epitope. In some embodiments, a peptide containing a validated epitope further comprises a modification which increases presentation of the epitope by an MHC class I molecule, and/or an MHC class II molecule.

In some embodiments, sequencing methods are used to identify tumor specific mutations. Any suitable sequencing method can be used according to the invention, for example, Next Generation Sequencing (NGS) technologies. Third Generation Sequencing methods might substitute for the NGS technology in the future to speed up the sequencing step of the method. For clarification purposes: the terms “Next Generation Sequencing” or “NGS” in the context of the present invention mean all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks, for example, within 1-7 days or within less than 24 hours and allow, in principle, single cell sequencing approaches. Multiple NGS platforms which are commercially available or which are mentioned in the literature can be used in the context of the invention e.g. those described in detail in WO 2012/159643.

In some embodiments, a peptide containing a validated epitope is linked to the at least second peptide, such as by a poly-glycine or poly-serine linker. In some embodiments, the modification is conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids. In some embodiments, a peptide containing a validated epitope further comprises a modification which increases cellular targeting to specific organs, tissues, or cell types. In some embodiments, a peptide containing a validated epitope comprises an antigen presenting cell targeting moiety or marker. In some embodiments, the antigen presenting cells are dendritic cells. In some embodiments, the dendritic cells are targeted using DEC205, XCR1, CD197, CD80, CD86, CD123, CD209, CD273, CD283, CD289, CD184, CD85h, CD85j, CD85k, CD85d, CD85g, CD85a, CD141, CD11c, CD83, TSLP receptor, Clec9a, or CD1a marker. In some embodiments, the dendritic cells are targeted using the CD141, DEC205, Clec9a, or XCR1 marker. In some embodiments, the dendritic cells are autologous cells. In some embodiments, one or more of the dendritic cells are bound to a T cell.

In some embodiments, the method described herein comprises large scale manufacture of and storage of HLA-matched peptides corresponding to shared antigens for treatment of a cancer or a tumor.

In some embodiments, the method described herein comprises treatment methods, comprising administering to a subject with cancer antigen-specific T cell that are specific to a validated epitope selected from the HLA matched peptide repertoire presented in any of Tables 1-8 and 11-14. In some embodiments, epitope-specific T cells are administered to the patient by infusion. In some embodiments, the T cells are administered to the patient by direct intravenous injection. In some embodiments, the T cell is an autologous T cell. In some embodiments, the T cell is an allogeneic T cell.

The methods of the disclosure can be used to treat any type of cancer known in the art. In some embodiments, a method of treating cancer comprises treating breast cancer, prostate cancer, ovarian cancer, cervical cancer, skin cancer, pancreatic cancer, colorectal cancer, renal cancer, liver cancer, brain cancer, lung cancer, metastatic melanoma, thymoma, lymphoma, sarcoma, mesothelioma, renal cell carcinoma, stomach cancer, gastric cancer, ovarian cancer, NHL, leukemia, uterine cancer, colon cancer, bladder cancer, kidney cancer or endometrial cancer. In some embodiments, the cancer is selected from the group consisting of carcinoma, lymphoma, blastoma, sarcoma, leukemia, squamous cell cancer, lung cancer (including small cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, melanoma, endometrial or uterine carcinoma, salivary gland carcinoma, kidney or renal cancer, liver cancer, prostate cancer, vulval cancer, thyroid cancer, hepatic carcinoma, head and neck cancer, colorectal cancer, rectal cancer, soft-tissue sarcoma, Kaposi's sarcoma, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's macroglobulinemia), chronic lymphocytic leukemia (CLL), acute lymphoblastic leukemia (ALL), myeloma, Hairy cell leukemia, chronic myeloblasts leukemia, and post-transplant lymphoproliferative disorder (PTLD), abnormal vascular proliferation associated with phakomatoses, edema, Meigs' syndrome. Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is prostate cancer. In some embodiments, a cancer to be treated by the methods of treatment of the present disclosure is colorectal cancer. In some embodiments, a patient or population of patients to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma.

The pharmaceutical compositions provided herein may be used alone or in combination with conventional therapeutic regimens such as surgery, irradiation, chemotherapy and/or bone marrow transplantation (autologous, syngeneic, allogeneic or unrelated). In some embodiments, at least one or more chemotherapeutic agents may be administered in addition to the pharmaceutical composition comprising an immunogenic therapy. In some embodiments, the one or more chemotherapeutic agents may belong to different classes of chemotherapeutic agents. In practicing the methods of treatment or use provided herein, therapeutically-effective amounts of the pharmaceutical compositions can be administered to a subject having a disease or condition. A therapeutically-effective amount can vary widely depending on the severity of the disease, the age and relative health of the subject, the potency of the compounds used, and other factors.

In some embodiments, the methods for treatment include one or more rounds of leukapheresis prior to transplantation of T cells. The leukapheresis may include collection of peripheral blood mononuclear cells (PBMCs). Leukapheresis may include mobilizing the PBMCs prior to collection. Alternatively, non-mobilized PBMCs may be collected. A large volume of PBMCs may be collected from the subject in one round. Alternatively, the subject may undergo two or more rounds of leukapheresis. The volume of apheresis may be dependent on the number of cells required for transplant. For instance, 12-15 liters of non-mobilized PBMCs may be collected from a subject in one round. The number of PBMCs to be collected from a subject may be between 1×10⁸ to 5×10¹⁰ cells. The number of PBMCs to be collected from a subject may be 1×10⁸, 5×10⁸, 1×10⁹, 5×10⁹, 1×10¹⁰ or 5×10¹⁰ cells. The minimum number of PBMCs to be collected from a subject may be 1×10⁶/kg of the subject's weight. The minimum number of PBMCs to be collected from a subject may be 1×10⁶/kg, 5×10⁶/kg, 1×10⁷/kg, 5×10⁷/kg, 1×10⁸/kg, 5×10⁸/kg of the subject's weight.

A single infusion may comprise a dose between 1×10⁶ cells per square meter body surface of the subject (cells/m²) and 5×10⁹ cells/m². A single infusion may comprise between about 2.5×10⁶ to about 5×10⁹ cells/m². A single infusion may comprise between at least about 2.5×10⁶ cells/m². A single infusion may comprise between at most 5×10⁹ cells/m². A single infusion may comprise between 1×10⁶ to 2.5×10⁶, 1×10⁶ to 5×10⁶, 1×10⁶ to 7.5×10⁶, 1×10⁶ to 1×10⁷, 1×10⁶ to 5×10⁷, 1×10⁶ to 7.5×10⁷, 1×10⁶ to 1×10⁸, 1×10⁶ to 2.5×10⁸, 1×10⁶ to 5×10⁸, 1×10⁶ to 1×10⁹, 1×10⁶ to 5×10⁹, 2.5×10⁶ to 5×10⁶, 2.5×10⁶ to 7.5×10⁶, 2.5×10⁶ to 1×10⁷, 2.5×10⁶ to 5×10⁷, 2.5×10⁶ to 7.5×10⁷, 2.5×10⁶ to 1×10⁸, 2.5×10⁶ to 2.5×10⁸, 2.5×10⁶ to 5×10⁸, 2.5×10⁶ to 1×10⁹, 2.5×10⁶ to 5×10⁹, 5×10⁶ to 7.5×10⁶, 5×10⁶ to 1×10⁷, 5×10⁶ to 5×10⁷, 5×10⁶ to 7.5×10⁷, 5×10⁶ to 1×10⁸, 5×10⁶ to 2.5×10⁸, 5×10⁶ to 5×10⁸, 5×10⁶ to 1×10⁹, 5×10⁶ to 5×10⁹, 7.5×10⁶ to 1×10⁷, 7.5×10⁶ to 5×10⁷, 7.5×10⁶ to 7.5×10⁷, 7.5×10⁶ to 1×10⁸, 7.5×10⁶ to 2.5×10⁸, 7.5×10⁶ to 5×10⁸, 7.5×10⁶ to 1×10⁹, 7.5×10⁶ to 5×10⁹, 1×10⁷ to 5×10⁷, 1×10⁷ to 7.5×10⁷, 1×10⁷ to 1×10⁸, 1×10⁷ to 2.5×10⁸, 1×10⁷ to 5×10⁸, 1×10⁷ to 1×10⁹, 1×10⁷ to 5×10⁹, 5×10⁷ to 7.5×10⁷, 5×10⁷ to 1×10⁸, 5×10⁷ to 2.5×10⁸, 5×10⁷ to 5×10⁸, 5×10⁷ to 1×10⁹, 5×10⁷ to 5×10⁹, 7.5×10⁷ to 1×10⁸, 7.5×10⁷ to 2.5×10⁸, 7.5×10⁷ to 5×10⁸, 7.5×10⁷ to 1×10⁹, 7.5×10⁷ to 5×10⁹, 1×10⁸ to 2.5×10⁸, 1×10⁸ to 5×10⁸, 1×10⁸ to 1×10⁹, 1×10⁸ to 5×10⁹, 2.5×10⁸ to 5×10⁸, 2.5×10⁸ to 1×10⁹, 2.5×10⁸ to 5×10⁹, 5×10⁸ to 1×10⁹, 5×10⁸ to 5×10⁹, or 1×10⁹ to 5×10⁹ cells/m². A single infusion may comprise between 1×10⁶ cells/m², 2.5×10⁶ cells/m², 5×10⁶ cells/m², 7.5×10⁶ cells/m², 1×10⁷ cells/m², 5×10⁷ cells/m², 7.5×10⁷ cells/m², 1×10⁸ cells/m², 2.5×10⁸ cells/m², 5×10⁸ cells/m², 1×10⁹ cells/m², or 5×10⁹ cells/m².

The methods may include administering chemotherapy to a subject including lymphodepleting chemotherapy using high doses of myeloablative agents. In some embodiments, the methods include administering a preconditioning agent, such as a lymphodepleting or chemotherapeutic agent, such as cyclophosphamide, fludarabine, or combinations thereof, to a subject prior to the first or subsequent dose. For example, the subject may be administered a preconditioning agent at least 2 days prior, such as at least 3, 4, 5, 6, 7, 8, 9 or 10 days prior, to the first or subsequent dose. In some embodiments, the subject is administered a preconditioning agent no more than 10 days prior, such as no more than 9, 8, 7, 6, 5, 4, 3, or 2 days prior, to the first or subsequent dose.

In some embodiments, where the lymphodepleting agent comprises cyclophosphamide, the subject is administered between 0.3 grams per square meter of the body surface of the subject (g/m²) and 5 g/m² cyclophosphamide. In some cases, the amount of cyclophosphamide administered to a subject is about at least 0.3 g/m². In some cases, the amount of cyclophosphamide administered to a subject is about at most 5 g/m². In some cases, the amount of cyclophosphamide administered to a subject is about 0.3 g/m² to 0.4 g/m², 0.3 g/m² to 0.5 g/m², 0.3 g/m² to 0.6 g/m², 0.3 g/m² to 0.7 g/m², 0.3 g/m² to 0.8 g/m², 0.3 g/m² to 0.9 g/m², 0.3 g/m² to 1 g/m², 0.3 g/m² to 2 g/m², 0.3 g/m² to 3 g/m², 0.3 g/m² to 4 g/m², 0.3 g/m² to 5 g/m², 0.4 g/m² to 0.5 g/m², 0.4 g/m² to 0.6 g/m², 0.4 g/m² to 0.7 g/m², 0.4 g/m² to 0.8 g/m², 0.4 g/m² to 0.9 g/m², 0.4 g/m² to 1 g/m², 0.4 g/m² to 2 g/m², 0.4 g/m² to 3 g/m², 0.4 g/m² to 4 g/m², 0.4 g/m² to 5 g/m², 0.5 g/m² to 0.6 g/m², 0.5 g/m² to 0.7 g/m², 0.5 g/m² to 0.8 g/m², 0.5 g/m² to 0.9 g/m², 0.5 g/m² to 1 g/m², 0.5 g/m² to 2 g/m², 0.5 g/m² to 3 g/m², 0.5 g/m² to 4 g/m², 0.5 g/m² to 5 g/m², 0.6 g/m² to 0.7 g/m², 0.6 g/m² to 0.8 g/m², 0.6 g/m² to 0.9 g/m², 0.6 g/m² to 1 g/m², 0.6 g/m² to 2 g/m², 0.6 g/m² to 3 g/m², 0.6 g/m² to 4 g/m², 0.6 g/m² to 5 g/m², 0.7 g/m² to 0.8 g/m², 0.7 g/m² to 0.9 g/m², 0.7 g/m² to 1 g/m², 0.7 g/m² to 2 g/m², 0.7 g/m² to 3 g/m², 0.7 g/m² to 4 g/m², 0.7 g/m² to 5 g/m², 0.8 g/m² to 0.9 g/m², 0.8 g/m² to 1 g/m², 0.8 g/m² to 2 g/m², 0.8 g/m² to 3 g/m², 0.8 g/m² to 4 g/m², 0.8 g/m² to 5 g/m², 0.9 g/m² to 1 g/m², 0.9 g/m² to 2 g/m², 0.9 g/m² to 3 g/m², 0.9 g/m² to 4 g/m², 0.9 g/m² to 5 g/m², 1 g/m² to 2 g/m², 1 g/m² to 3 g/m², 1 g/m² to 4 g/m², 1 g/m² to 5 g/m², 2 g/m² to 3 g/m², 2 g/m² to 4 g/m², 2 g/m² to 5 g/m², 3 g/m² to 4 g/m², 3 g/m² to 5 g/m², or 4 g/m² to 5 g/m². In some cases, the amount of cyclophosphamide administered to a subject is about 0.3 g/m², 0.4 g/m², 0.5 g/m², 0.6 g/m², 0.7 g/m², 0.8 g/m², 0.9 g/m², 1 g/m², 2 g/m², 3 g/m², 4 g/m², or 5 g/m². In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide.

In some embodiments, where the lymphodepleting agent comprises fludarabine, the subject is administered fludarabine at a dose between or between about 1 milligrams per square meter of the body surface of the subject (mg/m²) and 100 mg/m². In some cases, the amount of fludarabine administered to a subject is about at least 1 mg/m². In some cases, the amount of fludarabine administered to a subject is about at most 100 mg/m². In some cases, the amount of fludarabine administered to a subject is about 1 mg/m² to 5 mg/m², 1 mg/m² to 10 mg/m², 1 mg/m² to 15 mg/m², 1 mg/m² to 20 mg/m², 1 mg/m² to 30 mg/m², 1 mg/m² to 40 mg/m², 1 mg/m² to 50 mg/m², 1 mg/m² to 70 mg/m², 1 mg/m² to 90 mg/m², 1 mg/m² to 100 mg/m², 5 mg/m² to 10 mg/m², 5 mg/m² to 15 mg/m², 5 mg/m² to 20 mg/m², 5 mg/m² to 30 mg/m², 5 mg/m² to 40 mg/m², 5 mg/m² to 50 mg/m², 5 mg/m² to 70 mg/m², 5 mg/m² to 90 mg/m², 5 mg/m² to 100 mg/m², 10 mg/m² to 15 mg/m², 10 mg/m² to 20 mg/m², 10 mg/m² to 30 mg/m², 10 mg/m² to 40 mg/m², 10 mg/m² to 50 mg/m², 10 mg/m² to 70 mg/m², 10 mg/m² to 90 mg/m², 10 mg/m² to 100 mg/m², 15 mg/m² to 20 mg/m², 15 mg/m² to 30 mg/m², 15 mg/m² to 40 mg/m², 15 mg/m² to 50 mg/m², 15 mg/m² to 70 mg/m², 15 mg/m² to 90 mg/m², 15 mg/m² to 100 mg/m², 20 mg/m² to 30 mg/m², 20 mg/m² to 40 mg/m², 20 mg/m² to 50 mg/m², 20 mg/m² to 70 mg/m², 20 mg/m² to 90 mg/m², 20 mg/m² to 100 mg/m², 30 mg/m² to 40 mg/m², 30 mg/m² to 50 mg/m², 30 mg/m² to 70 mg/m², 30 mg/m² to 90 mg/m², 30 mg/m² to 100 mg/m², 40 mg/m² to 50 mg/m², 40 mg/m² to 70 mg/m², 40 mg/m² to 90 mg/m², 40 mg/m² to 100 mg/m², 50 mg/m² to 70 mg/m², 50 mg/m² to 90 mg/m², 50 mg/m² to 100 mg/m², 70 mg/m² to 90 mg/m², 70 mg/m² to 100 mg/m², or 90 mg/m² to 100 mg/m². In some cases, the amount of fludarabine administered to a subject is about 1 mg/m², 5 mg/m², 10 mg/m², 15 mg/m², 20 mg/m², 30 mg/m², 40 mg/m², 50 mg/m², 70 mg/m², 90 mg/m², or 100 mg/m². In some embodiments, the fludarabine can be administered in a single dose or can be administered in a plurality of doses, such as given daily, every other day or every three days. For example, in some instances, the agent, e.g., fludarabine, is administered between or between about 1 and 5 times, such as between or between about 3 and 5 times. In some embodiments, such plurality of doses is administered in the same day, such as 1 to 5 times or 3 to 5 times daily.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 400 mg/m² of cyclophosphamide and one or more doses of 20 mg/m² fludarabine prior to the first or subsequent dose of T cells. In some examples, the subject is administered 500 mg/m² of cyclophosphamide and one or more doses of 25 mg/m² fludarabine prior to the first or subsequent dose of T cells. In some examples, the subject is administered 600 mg/m² of cyclophosphamide and one or more doses of 30 mg/m² fludarabine prior to the first or subsequent dose of T cells. In some examples, the subject is administered 700 mg/m² of cyclophosphamide and one or more doses of 35 mg/m² fludarabine prior to the first or subsequent dose of T cells. In some examples, the subject is administered 700 mg/m² of cyclophosphamide and one or more doses of 40 mg/m² fludarabine prior to the first or subsequent dose of T cells. In some examples, the subject is administered 800 mg/m² of cyclophosphamide and one or more doses of 45 mg/m² fludarabine prior to the first or subsequent dose of T cells.

Fludarabine and cyclophosphamide may be administered on alternative days. In some cases, fludarabine and cyclophosphamide may be administered concurrently. In some cases, an initial dose of fludarabine is followed by a dose of cyclophosphamide. In some cases, an initial dose of cyclophosphamide may be followed by an initial dose of fludarabine. In some examples, a treatment regimen may include treatment of a subject with an initial dose of fludarabine 10 days prior to the transplant, followed by treatment with an initial dose of cyclophosphamide administered 9 days prior to the cell transplant, concurrently with a second dose of fludarabine. In some examples, a treatment regimen may include treatment of a subject with an initial dose of fludarabine 8 days prior to the transplant, followed by treatment with an initial dose of cyclophosphamide administered 7 days prior to the transplant concurrently with a second dose of fludarabine.

In some embodiments, a peptide comprises an epitope sequence according to any one of Tables 1-8 and 11-14. In some embodiments, a peptide comprises an epitope sequence according to Table 1. In some embodiments, a peptide comprises an epitope sequence according to Table 2. In some embodiments, a peptide comprises an epitope sequence according to Table 3. In some embodiments, a peptide comprises an epitope sequence according to Table 4A. In some embodiments, a peptide comprises an epitope sequence according to Table 4B. In some embodiments, a peptide comprises an epitope sequence according to Table 4C. In some embodiments, a peptide comprises an epitope sequence according to Table 4D. In some embodiments, a peptide comprises an epitope sequence according to Table 4E. In some embodiments, a peptide comprises an epitope sequence according to Table 4F. In some embodiments, a peptide comprises an epitope sequence according to Table 4G. In some embodiments, a peptide comprises an epitope sequence according to Table 4H. In some embodiments, a peptide comprises an epitope sequence according to Table 41. In some embodiments, a peptide comprises an epitope sequence according to Table 4J. In some embodiments, a peptide comprises an epitope sequence according to Table 4K. In some embodiments, a peptide comprises an epitope sequence according to Table 4L. In some embodiments, a peptide comprises an epitope sequence according to Table 4M. In some embodiments, a peptide comprises an epitope sequence according to Table 5. In some embodiments, a peptide comprises an epitope sequence according to Table 6. In some embodiments, a peptide comprises an epitope sequence according to Table 7. In some embodiments, a peptide comprises an epitope sequence according to Table 8. In some embodiments, a peptide comprises an epitope sequence according to Table 11. In some embodiments, a peptide comprises an epitope sequence according to Table 12. In some embodiments, a peptide comprises an epitope sequence according to Table 13. In some embodiments, a peptide comprises an epitope sequence according to Table 14.

TABLE 1
TABLE 1A POINT MUTATION

| | Amino Acid | | Peptides (Binding HLA allele | Exemplary |
|---|---|---|---|---|
| Gene | Alteration | Mutation Sequence Context | example(s)) | Diseases |
| KRAS | G12C | MTEYKLVVVGACGVGKSA | KLVVVGACGV (SEQ ID | BRCA, CESC, |
| | | LTIQLIQNHFVDEYDPTIEDS | NO: 154)(A02.01) | CRC, HNSC, |
| | | YRKQVVIDGETCLLDILDT | LVVVGACGV (SEQ ID | LUAD, PAAD, |
| | | AGQE (SEQ ID NO: 8) | NO: 155)(A02.01) | UCEC |
| | | | VVGACGVGK (SEQ ID | |
| | | | NO: 156)(A03.01, A11.01) | |
| | | | VVVGACGVGK (SEQ ID | |
| | | | NO: 157)(A03.01) | |
| KRAS | G12D | MTEYKLVVVGADGVGKSA | VVGADGVGK (SEQ ID | BLCA, BRCA, |
| | | LTIQLIQNHFVDEYDPTIEDS | NO: 158)(A11.01) | CESC, CRC, |
| | | YRKQVVIDGETCLLDILDT | VVVGADGVGK (SEQ ID | GBM, HNSC, |
| | | AGQE (SEQ ID NO: 9) | NO: 159)(A11.01) | KIRP, LIHC, |
| | | | KLVVVGADGV (SEQ ID | LUAD, PAAD, |
| | | | NO: 160)(A02.01) | SKCM, UCEC |
| | | | LVVVGADGV (SEQ ID | |
| | | | NO: 161)(A02.01) | |
| KRAS | G12V | MTEYKLVVVGAVGVGKSA | KLVVVGAVGV (SEQ ID | BRCA, CESC, |
| | | LTIQLIQNHFVDEYDPTIEDS | NO: 162)(A02.01) | CRC, LUAD, |
| | | YRKQVVIDGETCLLDILDT | LVVVGAVGV (SEQ ID | PAAD, THCA, |
| | | AGQE (SEQ ID NO: 10) | NO: 163)(A02.01) | UCEC |
| | | | VVGAVGVGK (SEQ ID | |
| | | | NO: 164)(A03.01, A11.01) | |
| | | | VVVGAVGVGK (SEQ ID | |
| | | | NO: 5)(A03.01, A11.01) | |
| KRAS | Q61H | AGGVGKSALTIQLIQNHFV | ILDTAGHEEY (SEQ ID | CRC, LUSC, |
| | | DEYDPTIEDSYRKQVVIDGE | NO: 165)(A01.01) | PAAD, SKCM, |
| | | TCLLDILDTAGHEEYSAMR | | UCEC |
| | | DQYMRTGEGFLCVFAINNT | | |
| | | KSFEDIHHYREQIKRVKDSE | | |
| | | DVPM (SEQ ID NO: 11) | | |
| KRAS | Q61L | AGGVGKSALTIQLIQNHFV | ILDTAGLEEY (SEQ ID | CRC, GBM, |
| | | DEYDPTIEDSYRKQVVIDGE | NO: 166)(A01.01) | HNSC, LUAD, |
| | | TCLLDILDTAGLEEYSAMR | LLDILDTAGL (SEQ ID | SKCM, UCEC |
| | | DQYMRTGEGFLCVFAINNT | NO: 167)(A02.01) | |
| | | KSFEDIHHYREQIKRVKDSE | | |
| | | DVPM (SEQ ID NO: 12) | | |
| NRAS | Q61K | AGGVGKSALTIQLIQNHFV | ILDTAGKEEY (SEQ ID | BLCA, CRC, |
| | | DEYDPTIEDSYRKQVVIDGE | NO: 168)(A01.01) | LIHC, LUAD, |
| | | TCLLDILDTAGKEEYSAMR | | LUSC, SKCM, |
| | | DQYMRTGEGFLCVFAINNS | | THCA, UCEC |
| | | KSFADINLYREQIKRVKDSD | | |
| | | DVPM (SEQ ID NO: 13) | | |
| NRAS | Q61R | AGGVGKSALTIQLIQNHFV | ILDTAGREEY (SEQ ID | BLCA, CRC, |
| | | DEYDPTIEDSYRKQVVIDGE | NO: 169)(A01.01) | LUSC, PAAD, |
| | | TCLLDILDTAGREEYSAMR | | PRAD, SKCM, |
| | | DQYMRTGEGFLCVFAINNS | | THCA, UCEC |
| | | KSFADINLYREQIKRVKDSD | | |
| | | DVPM (SEQ ID NO: 14) | | |
| BTK | C481S | MIKEGSMSEDEFIEEAKVM | EYMANGSLL (SEQ ID NO: | CLL |
| | | MNLSHEKLVQLYGVCTKQ | 170)(A24.02) | |
| | | RPIFIITEYMANGSLLNYLR | MANGSLLNY (SEQ ID | |
| | | EMRHRFQTQQLLEMCKDV | NO: 171)(A01.01, A03.01, | |
| | | CEAMEYLESKQFLHRDLA | A11.01) | |
| | | ARNCLVND (SEQ ID NO: | MANGSLLNYL (SEQ ID | |
| | | 15) | NO: 172)(A02.01, B07.02, | |
| | | | B08.01) | |
| | | | SLLNYLREM (SEQ ID NO: | |
| | | | 173)(A02.01, B07.02, | |
| | | | B08.01) | |
| | | | YMANGSLLN (SEQ ID | |
| | | | NO: 174)(A02.01) | |
| | | | YMANGSLLNY (SEQ ID | |
| | | | NO: 175)(A01.01, A03.01, | |
| | | | A11.01) | |
| EGFR | S492R | SLNITSLGLRSLKEISDGDVI | IIRNRGENSCK (SEQ ID | CRC |
| | | ISGNKNLCYANTINWKKLF | NO: 176)(A03.01) | |
| | | GTSGQKTKIIRNRGENSCK | | |
| | | ATGQVCHALCSPEGCWGP | | |
| | | EPRDCVSCRNVSRGRECVD | | |
| | | KCNLL (SEQ ID NO: 16) | | |
| EGFR | T790M | IPVAIKELREATSPKANKEI | CLTSTVQLIM (SEQ ID | NSCLC, PRAD |
| | | LDEAYVMASVDNPHVCRL | NO: 177)(A01.01, A02.01) | |
| | | LGICLTSTVQLIMQLMPFGC | IMQLMPFGC (SEQ ID NO: | |
| | | LLDYVREHKDNIGSQYLLN | 178)(A02.01) | |
| | | WCVQIAKGMNYLEDRRLV | IMQLMPFGCL (SEQ ID | |
| | | HRDLAA (SEQ ID NO: 17) | NO: 179)(A02.01, A24.02, | |
| | | | B08.01) | |
| | | | LIMQLMPFG (SEQ ID NO: | |
| | | | 180)(A02.01) | |
| | | | LIMQLMPFGC (SEQ ID | |
| | | | NO: 181)(A02.01) | |
| | | | LTSTVQLIM (SEQ ID NO: | |
| | | | 182)(A01.01) | |
| | | | MQLMPFGCL (SEQ ID NO: | |
| | | | 183)(A02.01, B07.02, | |
| | | | B08.01) | |
| | | | MQLMPFGCLL (SEQ ID | |
| | | | NO: 184)(A02.01, A24.02, | |
| | | | B08.01) | |
| | | | QLIMQLMPF (SEQ ID NO: | |
| | | | 185)(A02.01, A24.02, | |
| | | | B08.01) | |
| | | | QLIMQLMPFG (SEQ ID | |
| | | | NO: 186)(A02.01) | |
| | | | STVQLIMQL (SEQ ID NO: | |
| | | | 187)(A02.01) | |
| | | | VQLIMQLMPF (SEQ ID | |
| | | | NO: 188)(A02.01, A24.02, | |
| | | | B08.01) | |
| ABL1 | E255K | VADGLITTLHYPAPKRNKP | GQYGKVYEG (SEQ ID | Chronic |
| | | TVYGVSPNYDKWEMERTD | NO: 189)(A02.01) | myeloid |
| | | ITMKHKLGGGQYGKVYEG | GQYGKVYEGV (SEQ ID | leukemia |
| | | VWKKYSLTVAVKTLKEDT | NO: 190)(A02.01) | (CML), Acute |
| | | MEVEEFLKEAAVMKEIKHP | KLGGGQYGK (SEQ ID | lymphocytic |
| | | NLVQLLGVC (SEQ ID NO: | NO: 191)(A03.01) | leukemia |
| | | 18) | KLGGGQYGKV (SEQ ID | (ALL), |
| | | | NO: 192)(A02.01) | Gastrointestinal |
| | | | KVYEGVWKK (SEQ ID | stromal tumors |
| | | | NO: 193)(A02.01, A03.01) | (GIST) |
| | | | KVYEGVWKKY (SEQ ID | |
| | | | NO: 194)(A03.01) | |
| | | | QYGKVYEGV (SEQ ID | |
| | | | NO: 195)(A24.02) | |
| | | | QYGKVYEGVW (SEQ ID | |
| | | | NO: 196)(A24.02) | |
| ABL1 | E255V | VADGLITTLHYPAPKRNKP | GQYGVVYEG (SEQ ID | Chronic |
| | | TVYGVSPNYDKWEMERTD | NO: 197)(A02.01) | myeloid |
| | | ITMKHKLGGGQYGVVYEG | GQYGVVYEGV (SEQ ID | leukemia |
| | | VWKKYSLTVAVKTLKEDT | NO: 198)(A02.01) | (CML), Acute |
| | | MEVEEFLKEAAVMKEIKHP | KLGGGQYGV (SEQ ID | lymphocytic |
| | | NLVQLLGVC (SEQ ID NO: | NO: 199)(A02.01) | leukemia |
| | | 19) | KLGGGQYGVV (SEQ ID | (ALL), |
| | | | NO: 200)(A02.01) | Gastrointestinal |
| | | | QYGVVYEGV (SEQ ID | stromal tumors |
| | | | NO: 201)(A24.02) | (GIST) |
| | | | QYGVVYEGVW (SEQ ID | |
| | | | NO: 202)(A24.02) | |
| | | | VVYEGVWKK (SEQ ID | |
| | | | NO: 203)(A02.01, A03.01) | |
| | | | VVYEGVWKKY (SEQ ID | |
| | | | NO: 204)(A03.01) | |
| ABL1 | M351T | LLGVCTREPPFYIITEFMTY | ATQISSATEY (SEQ ID NO: | Chronic |
| | | GNLLDYLRECNRQEVNAV | 205)(A01.01) | myeloid |
| | | VLLYMATQISSATEYLEKK | ISSATEYLEK (SEQ ID NO: | leukemia |
| | | NFIHRDLAARNCLVGENHL | 206)(A03.01) | (CML), Acute |
| | | VKVADFGLSRLMTGDTYT | SSATEYLEK (SEQ ID NO: | lymphocytic |
| | | AHAGAKF (SEQ ID NO: 20) | 207)(A03.01) | leukemia |
| | | | TQISSATEYL (SEQ ID NO: | (ALL), |
| | | | 208)(A02.01) | Gastrointestinal |
| | | | YMATQISSAT (SEQ ID | stromal tumors |
| | | | NO: 209)(A02.01) | (GIST) |
| ABL1 | T315I | SLTVAVKTLKEDTMEVEEF | FYIIIEFMTY (SEQ ID NO: | Chronic |
| | | LKEAAVMKEIKHPNLVQLL | 210)(A24.02) | myeloid |
| | | GVCTREPPFYIIIEFMTYGN | IIEFMTYGNL (SEQ ID NO: | leukemia |
| | | LLDYLRECNRQEVNAVVL | 211)(A02.01) | (CML), Acute |
| | | LYMATQISSAMEYLEKKNF | IIIEFMTYG (SEQ ID NO: | lymphocytic |
| | | IHRDLA (SEQ ID NO: 21) | 212)(A02.01) | leukemia |
| | | | IIIEFMTYGN (SEQ ID NO: | (ALL), |
| | | | 213)(A02.01) | Gastrointestinal |
| | | | YIIIEFMTYG (SEQ ID NO: | stromal tumors |
| | | | 214)(A02.01) | (GIST) |
| ABL1 | Y253H | STVADGLITTLHYPAPKRN | GQHGEVYEGV (SEQ ID | Chronic |
| | | KPTVYGVSPNYDKWEMER | NO: 215)(A02.01) | myeloid |
| | | TDITMKHKLGGGQHGEVY | KLGGGQHGEV (SEQ ID | leukemia |
| | | EGVWKKYSLTVAVKTLKE | NO: 216)(A02.01) | (CML), Acute |
| | | DTMEVEEFLKEAAVMKEIK | | lymphocytic |
| | | HPNLVQLLG (SEQ ID NO: | | leukemia |
| | | 22) | | (ALL), |
| | | | | Gastrointestinal |
| | | | | stromal tumors |
| | | | | (GIST) |
| ALK | G1269A | SSLAMLDLLHVARDIACGC | KIADFGMAR (SEQ ID NO: | NSCLC |
| | | QYLEENHFIHRDIAARNCL | 217)(A03.01) | |
| | | LTCPGPGRVAKIADFGMAR | RVAKIADFGM (SEQ ID | |
| | | DIYRASYYRKGGCAMLPV | NO: 218)(A02.01, B07.02) | |
| | | KWMPPEAFMEGIFTSKTDT | | |
| | | WSFGVLL (SEQ ID NO: 23) | | |
| ALK | L1196M | QVAVKTLPEVCSEQDELDF | FILMELMAGG (SEQ ID | NSCLC |
| | | LMEALIISKFNHQNIVRCIG | NO: 219)(A02.01) | |
| | | VSLQSLPRFILMELMAGGD | ILMELMAGG (SEQ ID NO: | |
| | | LKSFLRETRPRPSQPSSLAM | 220)(A02.01) | |
| | | LDLLHVARDIACGCQYLEE | ILMELMAGGD (SEQ ID | |
| | | NHFI (SEQ ID NO: 24) | NO: 221)(A02.01) | |
| | | | LMELMAGGDL (SEQ ID | |
| | | | NO: 222)(A02.01) | |
| | | | LPRFILMEL (SEQ ID NO: | |
| | | | 223)(B07.02, B08.01) | |
| | | | LPRFILMELM (SEQ ID | |
| | | | NO: 224)(B07.02) | |
| | | | LQSLPRFILM (SEQ ID NO: | |
| | | | 225)(A02.01, B08.01) | |
| | | | SLPRFILMEL (SEQ ID NO: | |
| | | | 226)(A02.01, A24.02, | |
| | | | B07.02, B08.01) | |
| BRAF | V600E | MIKLIDIARQTAQGMDYLH | LATEKSRWS (SEQ ID NO: | CRC, GBM, |
| | | AKSIIHRDLKSNNIFLHEDL | 227)(A02.01, B08.01) | KIRP, LUAD, |
| | | TVKIGDFGLATEKSRWSGS | LATEKSRWSG (SEQ ID | SKCM, THCA |
| | | HQFEQLSGSILWMAPEVIR | NO: 228)(A02.01, B08.01) | |
| | | MQDKNPYSFQSDVYAFGIV | | |
| | | LYELM (SEQ ID NO: 25) | | |
| EEF1B2 | S43G | MGFGDLKSPAGLQVLNDY | GPPPADLCHAL (SEQ ID | BLCA, KIRP, |
| | | LADKSYIEGYVPSQADVAV | NO: 229)(B07.02) | PRAD, SKCM |
| | | FEAVSGPPPADLCHALRWY | | |
| | | NHIKSYEKEKASLPGVKKA | | |
| | | LGKYGPADVEDTTGSGAT | | |
| | | (SEQ ID NO: 26) | | |
| ERBB3 | V104M | ERCEVVMGNLEIVLTGHNA | | CRC, Stomach |
| | | DLSFLQWIREVTGYVLVA | | Cancer |
| | | MNEFSTLPLPNLRMVRGTQ | | |
| | | VYDGKFAIFVMLNYNTNSS | | |
| | | HALRQLRLTQLTEILSGGV | | |
| | | YIEKNDK (SEQ ID NO: 27) | | |
| ESR1 | D538G | HLMAKAGLTLQQQHQRLA | GLLLEMLDA (SEQ ID NO: | Breast Cancer |
| | | QLLLILSHIRHMSNKGMEH | 230)(A02.01) | |
| | | LYSMKCKNVVPLYGLLLE | LYGLLLEML (SEQ ID NO: | |
| | | MLDAHRLHAPTSRGGASV | 231)(A24.02) | |
| | | EETDQSHLATAGSTSSHSL | NVVPLYGLL (SEQ ID NO: | |
| | | QKYYITGEA (SEQ ID NO: | 232)(A02.01) | |
| | | 28) | PLYGLLLEM (SEQ ID NO: | |
| | | | 233)(A02.01) | |
| | | | PLYGLLLEML (SEQ ID | |
| | | | NO: 234)(A02.01, A24.02) | |
| | | | VPLYGLLLEM (SEQ ID | |
| | | | NO: 235)(B07.02) | |
| | | | VVPLYGLLL (SEQ ID NO: | |
| | | | 236)(A02.01, A24.02) | |
| ESR1 | S463P | NQGKCVEGMVEIFDMLLA | FLPSTLKSL (SEQ ID NO: | Breast Cancer |
| | | TSSRFRMMNLQGEEFVCLK | 237)(A02.01, A24.02, | |
| | | SIILLNSGVYTFLPSTLKSLE | B08.01) | |
| | | EKDHIHRVLDKITDTLIHLM | GVYTFLPST (SEQ ID NO: | |
| | | AKAGLTLQQQHQRLAQLL | 238)(A02.01) | |
| | | LILSH (SEQ ID NO: 29) | GVYTFLPSTL (SEQ ID | |
| | | | NO. 239)(A02.01, A24.02) | |
| | | | TFLPSTLKSL (SEQ ID NO: | |
| | | | 240)(A24.02) | |
| | | | VYTFLPSTL (SEQ ID NO: | |
| | | | 241)(A24.02) | |
| | | | YTFLPSTLK (SEQ ID NO: | |
| | | | 242)(A03.01) | |
| ESR1 | Y537C | IHLMAKAGLTLQQQHQRL | NVVPLCDLL (SEQ ID NO: | Breast Cancer |
| | | AQLLLILSHIRHMSNKGME | 243)(A02.01) | |
| | | HLYSMKCKNVVPLCDLLL | NVVPLCDLLL (SEQ ID | |
| | | EMLDAHRLHAPTSRGGAS | NO: 244)(A02.01) | |
| | | VEETDQSHLATAGSTSSHS | PLCDLLLEM (SEQ ID NO: | |
| | | LQKYYITGE (SEQ ID NO: | 245)(A02. 01) | |
| | | 30) | PLCDLLLEML (SEQ ID | |
| | | | NO: 246)(A02.01) | |
| | | | VPLCDLLLEM (SEQ ID | |
| | | | NO: 247)(B07.02) | |
| | | | VVPLCDLLL (SEQ ID NO: | |
| | | | 248)(A02.01, A24.02) | |
| ESR1 | Y537N | IHLMAKAGLTLQQQHQRL | NVVPLNDLL (SEQ ID NO: | Breast Cancer |
| | | AQLLLILSHIRHMSNKGME | 249)(A02.01) | |
| | | HLYSMKCKNVVPLNDLLL | NVVPLNDLLL (SEQ ID | |
| | | EMLDAHRLHAPTSRGGAS | NO: 250)(A02.01) | |
| | | VEETDQSHLATAGSTSSHS | PLNDLLLEM (SEQ ID NO: | |
| | | LQKYYITGE (SEQ ID NO: | 251)(A02.01) | |
| | | 31) | PLNDLLLEML (SEQ ID | |
| | | | NO: 252)(A02.01) | |
| | | | VPLNDLLLEM (SEQ ID | |
| | | | NO: 253)(B07.02) | |
| ESR1 | Y537S | IHLMAKAGLTLQQQHQRL | NVVPLSDLL (SEQ ID NO: | Breast Cancer |
| | | AQLLLILSHIRHMSNKGME | 254)(A02.01) | |
| | | HLYSMKCKNVVPLSDLLLE | NVVPLSDLLL (SEQ ID | |
| | | MLDAHRLHAPTSRGGASV | NO: 255)(A02.01) | |
| | | EETDQSHLATAGSTSSHSL | PLSDLLLEM (SEQ ID NO: | |
| | | QKYYITGE (SEQ ID NO: 32) | 256)(A02.01) | |
| | | | PLSDLLLEML (SEQ ID | |
| | | | NO: 257)(A02.01) | |
| | | | VPLSDLLLEM (SEQ ID | |
| | | | NO: 258)(B07.02) | |
| | | | VVPLSDLLL (SEQ ID NO: | |
| | | | 259)(A02.01, A24.02) | |
| FGFR3 | S249C | HRIGGIKLRHQQWSLVMES | VLERCPHRPI (SEQ ID NO: | BLCA, HNSC, |
| | | VVPSDRGNYTCVVENKFGS | 260)(A02.01, B08.01) | KIRP, LUSC |
| | | IRQTYTLDVLERCPHRPILQ | YTLDVLERC (SEQ ID NO: | |
| | | AGLPANQTAVLGSDVEFHC | 261)(A02.01) | |
| | | KVYSDAQPHIQWLKHVEV | | |
| | | NGSKVG (SEQ ID NO: 33) | | |
| FRG1B | L52S | AVKLSDSRIALKSGYGKYL | FQNGKMALS (SEQ ID NO: | GBM, KIRP, |
| | | GINSDELVGHSDAIGPREQ | 262)(A02.01) | PRAD, SKCM |
| | | WEPVFQNGKMALSASNSC | | |
| | | FIRCNEAGDIEAKSKTAGEE | | |
| | | EMIKIRSCAEKETKKKDDIP | | |
| | | EEDKG (SEQ ID NO: 34) | | |
| HER2 | V777L | GSGAFGTVYKGIWIPDGEN | VMAGLGSPYV (SEQ ID | BRCA |
| | (Resistance) | VKIPVAIKVLRENTSPKAN | NO: 263)(A02.01, A03.01) | |
| | | KEILDEAYVMAGLGSPYVS | | |
| | | RLLGICLTSTVQLVTQLMP | | |
| | | YGCLLDHVRENRGRLGSQ | | |
| | | DLLNWCM (SEQ ID NO: 35) | | |
| IDH1 | R132H | RVEEFKLKQMWKSPNGTIR | KPIIIGHHA (SEQ ID NO: | BLCA, GBM, |
| | | NILGGTVFREAIICKNIPRLV | 264)(B07.02) | PRAD |
| | | SGWVKPIIIGHHAYGDQYR | | |
| | | ATDFVVPGPGKVEITYTPS | | |
| | | DGTQKVTYLVHNFEEGGG | | |
| | | VAMGM (SEQ ID NO: 36) | | |
| IDH1 | R132C | RVEEFKLKQMWKSPNGTIR | KPIIIGCHA (SEQ ID NO: | BLCA, GBM, |
| | | NILGGTVFREAIICKNIPRLV | 265)(B07.02) | PRAD |
| | | SGWVKPIIIGCHAYGDQYR | | |
| | | ATDFVVPGPGKVEITYTPS | | |
| | | DGTQKVTYLVHNFEEGGG | | |
| | | VAMGM (SEQ ID NO: 37) | | |
| IDH1 | R132G | RVEEFKLKQMWKSPNGTIR | KPIIIGGHA (SEQ ID NO: | BLCA, BRCA, |
| | | NILGGTVFREAIICKNIPRLV | 266)(B07.02) | CRC, GBM, |
| | | SGWVKPIIIGGHAYGDQYR | | HNSC, LUAD, |
| | | ATDFVVPGPGKVEITYTPS | | PAAD, PRAD, |
| | | DGTQKVTYLVHNFEEGGG | | UCEC |
| | | VAMGM (SEQ ID NO: 38) | | |
| IDH1 | R132S | RVEEFKLKQMWKSPNGTIR | KPIIIGSHA (SEQ ID NO: | BLCA, BRCA, |
| | | NILGGTVFREAIICKNIPRLV | 267)(B07.02) | GBM, HNSC, |
| | | SGWVKPIIIGSHAYGDQYR | | LIHC, LUAD, |
| | | ATDFVVPGPGKVEITYTPS | | LUSC, PAAD, |
| | | DGTQKVTYLVHNFEEGGG | | SKCM, UCEC |
| | | VAMGM (SEQ ID NO: 39) | | |
| KIT | T670I | VAVKMLKPSAHLTEREAL | IIEYCCYGDL (SEQ ID NO: | Gastrointestinal |
| | | MSELKVLSYLGNHMNIVN | 268)(A02.01) | stromal tumors |
| | | LLGACTIGGPTLVIIEYCCY | TIGGPTLVII (SEQ ID NO: | (GIST) |
| | | GDLLNFLRRKRDSFICSKQE | 269)(A02.01) | |
| | | DHAEAALYKNLLHSKESSC | VIIEYCCYG (SEQ ID NO: | |
| | | SDSTNE (SEQ ID NO: 40) | 270)(A02.01) | |
| KIT | V654A | VEATAYGLIKSDAAMTVA | HMNIANLLGA (SEQ ID | Gastrointestinal |
| | | VKMLKPSAHLTEREALMSE | NO: 271)(A02.01) | stromal tumors |
| | | LKVLSYLGNHMNIANLLG | IANLLGACTI (SEQ ID NO: | (GIST) |
| | | ACTIGGPTLVITEYCCYGDL | 272)(A02.01) | |
| | | LNFLRRKRDSFICSKQEDH | MNIANLLGA (SEQ ID NO: | |
| | | AEAALYK (SEQ ID NO: 41) | 273)(A02.01) | |
| | | | YLGNHMNIA (SEQ ID NO: | |
| | | | 274)(A02.01, B08.01) | |
| | | | YLGNHMNIAN (SEQ ID | |
| | | | NO: 275)(A02.01) | |
| MEK | C121S | ISELGAGNGGVVFKVSHKP | VLHESNSPY (SEQ ID NO: | Melanoma |
| | | SGLVMARKLIHLEIKPAIRN | 276)(A03.01) | |
| | | QIIRELQVLHESNSPYIVGF | VLHESNSPYI (SEQ ID NO: | |
| | | YGAFYSDGEISICMEHMDG | 277)(A02.01) | |
| | | GSLDQVLKKAGRIPEQILG | | |
| | | KVSI (SEQ ID NO: 42) | | |
| MEK | P124L | LGAGNGGVVFKVSHKPSG | LQVLHECNSL (SEQ ID | Melanoma |
| | | LVMARKLIHLEIKPAIRNQII | NO: 278)(A02.01, B08.01) | |
| | | RELQVLHECNSLYIVGFYG | LYIVGFYGAF (SEQ ID | |
| | | AFYSDGEISICMEHMDGGS | NO: 279)(A24.02) | |
| | | LDQVLKKAGRIPEQILGKV | NSLYIVGFY (SEQ ID NO: | |
| | | SIAVI (SEQ ID NO: 43) | 280)(A01.01) | |
| | | | QVLHECNSL (SEQ ID NO: | |
| | | | 281)(A02.01, B08.01) | |
| | | | SLYIVGFYG (SEQ ID NO: | |
| | | | 282)(A02.01) | |
| | | | SLYIVGFYGA (SEQ ID | |
| | | | NO: 283)(A02.01) | |
| | | | VLHECNSLY (SEQ ID NO: | |
| | | | 284)(A03.01) | |
| | | | VLHECNSLYI (SEQ ID | |
| | | | NO: 285)(A02.01, A03.01) | |
| MYC | E39D | MPLNVSFTNRNYDLDYDS | FYQQQQQSDL (SEQ ID | Lymphoid |
| | | VQPYFYCDEEENFYQQQQ | NO: 286)(A24.02) | Cancer; Burkitt |
| | | QSDLQPPAPSEDIWKKFELL | QQQSDLQPPA (SEQ ID | Lymphoma |
| | | PTPPLSPSRRSGLCSPSYVA | NO: 287)(A02.01) | |
| | | VTPFSLRGDNDGG (SEQ ID | QQSDLQPPA (SEQ ID NO: | |
| | | NO: 44) | 288)(A02.01) | |
| | | | YQQQQQSDL (SEQ ID NO: | |
| | | | 289)(A02.01, B08.01) | |
| MYC | P57S | FTNRNYDLDYDSVQPYFYC | FELLSTPPL (SEQ ID NO: | Lymphoid |
| | | DEEENFYQQQQQSELQPPA | 290)(A02.01, B08.01) | Cancer |
| | | PSEDIWKKFELLSTPPLSPS | LLSTPPLSPS (SEQ ID NO: | |
| | | RRSGLCSPSYVAVTPFSLRG | 291)(A02.01) | |
| | | DNDGGGGSFSTADQLEMV | | |
| | | TELLG (SEQ ID NO: 45) | | |
| MYC | T58I | TNRNYDLDYDSVQPYFYC | FELLPIPPL (SEQ ID NO: | Neuroblastoma |
| | | DEEENFYQQQQQSELQPPA | 292)(A02.01) | |
| | | PSEDIWKKFELLPIPPLSPSR | IWKKFELLPI (SEQ ID NO: | |
| | | RSGLCSPSYVAVTPFSLRG | 293)(A24.02) | |
| | | DNDGGGGSFSTADQLEMV | LLPIPPLSPS (SEQ ID NO: | |
| | | TELLGG (SEQ ID NO: 46) | 294)(A02.01, B07.02) | |
| | | | LPIPPLSPS (SEQ ID NO: | |
| | | | 295)(B07.02) | |
| PDGFRa | T674I | VAVKMLKPTARSSEKQAL | IIEYCFYGDL (SEQ ID NO: | Chronic |
| | | MSELKIMTHLGPHLNIVNL | 296)(A02.01) | Eosinophilic |
| | | LGACTKSGPIYIIIEYCFYGD | IIIEYCFYG (SEQ ID NO: | Leukemia |
| | | LVNYLHKNRDSFLSHHPEK | 297)(A02.01) | |
| | | PKKELDIFGLNPADESTRSY | IYIIIEYCF (SEQ ID NO: | |
| | | VILS (SEQ ID NO: 47) | 298)(A24.02) | |
| | | | IYIIIEYCFY (SEQ ID NO: | |
| | | | 299)(A24.02) | |
| | | | YIIIEYCFYG (SEQ ID NO: | |
| | | | 300)(A02.01) | |
| PIK3CA | E542K | IEEHANWSVSREAGFSYSH | KITEQEKDFL (SEQ ID NO: | BLCA, BRCA, |
| | | AGLSNRLARDNELRENDKE | 301)(A02.01) | CESC, CRC, |
| | | QLKAISTRDPLSKITEQEKD | | GBM, HNSC, |
| | | FLWSHRHYCVTIPEILPKLL | | KIRC, KIRP, |
| | | LSVKWNSRDEVAQMYCLV | | LIHC, LUAD, |
| | | KDWPP (SEQ ID NO: 48) | | LUSC, PRAD, |
| | | | | UCEC |
| PIK3CA | E545K | HANWSVSREAGFSYSHAG | STRDPLSEITK (SEQ ID | BLCA, BRCA, |
| | | LSNRLARDNELRENDKEQL | NO: 302)(A03.01) | CESC, CRC, |
| | | KAISTRDPLSEITKQEKDFL | DPLSEITK (SEQ ID NO: | GBM, HNSC, |
| | | WSHRHYCVTIPEILPKLLLS | 303)(A03.01) | KIRC, KIRP, |
| | | VKWNSRDEVAQMYCLVK | | LIHC, LUAD, |
| | | DWPPIKP (SEQ ID NO: 49) | | LUSC, PRAD, |
| | | | | SKCM, UCEC |
| PIK3CA | H1047R | LFINLFSMMLGSGMPELQS | | BRCA, CESC, |
| | | FDDIAYIRKTLALDKTEQE | | CRC, GBM, |
| | | ALEYFMKQMNDARHGGW | | HNSC, LIHC, |
| | | TTKMDWIFHTIKQHALN | | LUAD, LUSC, |
| | | (SEQ ID NO: 50) | | PRAD, UCEC |
| POLE | P286R | QRGGVITDEEETSKKIADQ | LPLKFRDAET (SEQ ID | Colorectal |
| | | LDNIVDMREYDVPYHIRLSI | NO: 304)(B07.02) | adenocarcinoma, |
| | | DIETTKLPLKFRDAETDQIM | | Uterine/ |
| | | MISYMIDGQGYLITNREIVS | | Endometrium |
| | | EDIEDFEFTPKPEYEGPFCV | | Adenocarcinoma; |
| | | FN (SEQ ID NO: 51) | | Colorectal |
| | | | | adenocarcinoma, |
| | | | | MSI+; |
| | | | | Uterine/ |
| | | | | Endometrium |
| | | | | Adenocarcinoma, |
| | | | | MSI+; |
| | | | | Endometrioid |
| | | | | carcinoma; |
| | | | | Endometrium |
| | | | | Serous |
| | | | | carcinoma; |
| | | | | Endometrium |
| | | | | Carcinosarcoma- |
| | | | | malignant |
| | | | | mesodermal |
| | | | | mixed tumor; |
| | | | | Glioma; |
| | | | | Astrocytoma; |
| | | | | GBM |
| PTEN | R130Q | KFNCRVAQYPFEDHNPPQL | QTGVMICAYL (SEQ ID | BRCA, CESC, |
| | | ELIKPFCEDLDQWLSEDDN | NO: 305)(A02.01) | CRC, GBM, |
| | | HVAAIHCKAGKGQTGVMI | | KIRC, LUSC, |
| | | CAYLLHRGKFLKAQEALDF | | UCEC |
| | | YGEVRTRDKKGVTIPSQRR | | |
| | | YVYYYSY (SEQ ID NO: 52) | | |
| RAC1 | P29S | MQAIKCVVVGDGAVGKTC | AFSGEYIPTV (SEQ ID NO: | Melanoma |
| | | LLISYTTNAFSGEYIPTVFD | 306)(A02.01, A24.02) | |
| | | NYSANVMVDGKPVNLGL | | |
| | | WDTAGQEDYDRLRPLSYP | | |
| | | QTVGET (SEQ ID NO: 53) | | |
| TP53 | G245S | IRVEGNLRVEYLDDRNTFR | SMNRRPILT (SEQ ID NO: | BLCA, BRCA, |
| | | HSVVVPYEPPEVGSDCTTIH | 307)(A02.01, B08.01) | CRC, GBM, |
| | | YNYMCNSSCMGSMNRRPI | YMCNSSCMGS (SEQ ID | HNSC, LUSC, |
| | | LTIITLEDSSGNLLGRNSFE | NO: 308)(A02.01) | PAAD, PRAD |
| | | VRVCACPGRDRRTEEENLR | | |
| | | KKGEP (SEQ ID NO: 54) | | |
| TP53 | R175H | TYSPALNKMFCQLAKTCPV | | BLCA, BRCA, |
| | | QLWVDSTPPPGTRVRAMAI | | CRC, GBM, |
| | | YKQSQHMTEVVRHCPHHE | | HNSC, LUAD, |
| | | RCSDSDGLAPPQHLIRVEG | | PAAD, PRAD, |
| | | NLRVEYLDDRNTFRHSVV | | UCEC |
| | | VPYEPPEV (SEQ ID NO: 55) | | |
| TP53 | R248Q | EGNLRVEYLDDRNTFRHSV | GMNQRPILT (SEQ ID NO: | BLCA, BRCA, |
| | | VVPYEPPEVGSDCTTIHYN | 309)(A02.01) | CRC, GBM, |
| | | YMCNSSCMGGMNQRPILTI | | HNSC, KIRC, |
| | | ITLEDSSGNLLGRNSFEVRV | | LIHC, LUSC, |
| | | CACPGRDRRTEEENLRKKG | | PAAD, PRAD, |
| | | EPHHE (SEQ ID NO: 56) | | UCEC |
| TP53 | R248W | EGNLRVEYLDDRNTFRHSV | GMNWRPILT (SEQ ID NO: | BLCA, BRCA, |
| | | VVPYEPPEVGSDCTTIHYN | 310)(A02.01) | CRC, GBM, |
| | | YMCNSSCMGGMNWRPILT | | HNSC, LIHC, |
| | | IITLEDSSGNLLGRNSFEVR | | LUSC, PAAD, |
| | | VCACPGRDRRTEEENLRKK | | SKCM, UCEC |
| | | GEPHHE (SEQ ID NO: 57) | | |
| TP53 | R273C | PEVGSDCTTIHYNYMCNSS | LLGRNSFEVC (SEQ ID | BLCA, BRCA, |
| | | CMGGMNRRPILTIITLEDSS | NO. 311)(A02.01) | CRC, GBM, |
| | | GNLLGRNSFEVCVCACPGR | | HNSC, LUSC, |
| | | DRRTEEENLRKKGEPHHEL | | PAAD, UCEC |
| | | PPGSTKRALPNNTSSSPQPK | | |
| | | KKPL (SEQ ID NO: 58) | | |

TABLE 1B
MSI-ASSOCIATED FRAMESHIFTS

| | | | | |
|---|---|---|---|---|
| ACVR2A | D96fs; | GVEPCYGDKDKRRHCFAT | | MSI+CRC, |
| | +1 | WKNISGSIEIVKQGCWLDDI | | MSI+ |
| | | NCYDRTDCVEKKRQP* | | Uterine/Endo- |
| | | (SEQ ID NO: 59) | | metrium Cancer, |
| | | | | MSI+ Stomach |
| | | | | Cancer, Lynch |
| | | | | syndrome |
| ACVR2A | D96fs; | GVEPCYGDKDKRRHCFAT | ALKYIFVAV (SEQ ID NO: | MSI+ CRC, |
| | −1 | WKNISGSIEIVKQGCWLDDI | 312) (A02.01, B08.01) | MSI+ |
| | | NCYDRTDCVEKKTALKYIF | ALKYIFVAVR (SEQ ID | Uterine/Endo- |
| | | VAVRAICVMKSFLIFRRWK | NO: 313) (A03.01) | metrium Cancer, |
| | | SHSPLQIQLHLSHPITTSCSI | AVRAICVMK (SEQ ID NO: | MST+ Stomach |
| | | PWCHLC* (SEQ ID NO: 60) | 314) (A03.01) | Cancer, Lynch |
| | | | AVRAICVMKS (SEQ ID | syndrome |
| | | | NO: 315) (A03.01) | |
| | | | CVEKKTALK (SEQ ID NO: | |
| | | | 316) (A03.01) | |
| | | | CVEKKTALKY (SEQ ID | |
| | | | NO: 317) (A01.01) | |
| | | | CVMKSFLIF (SEQ ID NO: | |
| | | | 318) (A24.02, B08.01) | |
| | | | CVMKSFLIFR (SEQ ID | |
| | | | NO: 319) (A03.01) | |
| | | | FLIFRRWKS (SEQ ID NO: | |
| | | | 320) (A02.01, B08.01) | |
| | | | FRRWKSHSPL (SEQ ID | |
| | | | NO: 321) (B08.01) | |
| | | | FVAVRAICV (SEQ ID NO: | |
| | | | 322) (A02.01, B08.01) | |
| | | | FVAVRAICVM (SEQ ID | |
| | | | NO: 323) (B08.01) | |
| | | | IQLEILSHPI (SEQ ID NO: | |
| | | | 324) (A02.01) | |
| | | | KSFLIFRRWK (SEQ ID | |
| | | | NO: 325) (A03.01) | |
| | | | KTALKYIFV (SEQ ID NO: | |
| | | | 326) (A02.01) | |
| | | | KYIFVAVRAI (SEQ ID NO: | |
| | | | 327) (A24.02) | |
| | | | RWKSHSPLQI (SEQ ID | |
| | | | NO: 328) (A24.02) | |
| | | | TALKYIFVAV (SEQ ID | |
| | | | NO: 329) (A02.01, B08.01) | |
| | | | VAVRAICVMK (SEQ ID | |
| | | | NO: 330) (A03.01) | |
| | | | VMKSFLIFR (SEQ ID NO: | |
| | | | 331) (A03.01) | |
| | | | VMKSFLIFRR (SEQ ID | |
| | | | NO: 332) (A03.01) | |
| | | | YIFVAVRAI (SEQ ID NO: | |
| | | | 333) (A02.01) | |
| C15ORF40 | L132fs; | TAEAVNVAIAAPPSEGEAN | ALFFFFFET (SEQ ID NO: | MSI+ CRC, |
| | +1 | AELCRYLSKVLELRKSDVV | 334) (A02.01) | MSI+ |
| | | LDKVGLALFFFFFETKSCSV | ALFFFFFETK (SEQ ID NO: | Uterine/Endo- |
| | | AQAGVQWRSLGSLQPPPPG | 335) (A03.01) | metrium Cancer, |
| | | FKLFSCLSFLSSWDYRRMP | AQAGVQWRSL (SEQ ID | MSI+ Stomach |
| | | PCLANFCIFNRDGVSPCWS | NO: 336) (A02.01) | Cancer, Lynch |
| | | GWS* (SEQ ID NO: 61) | CLANFCIFNR (SEQ ID NO: | syndrome |
| | | | 337) (A03.01) | |
| | | | CLSFLSSWDY (SEQ ID | |
| | | | NO: 338) (A01.01, A03.01) | |
| | | | FFETKSCSV (SEQ ID NO: | |
| | | | 339) (B08.01) | |
| | | | FFFETKSCSV (SEQ ID NO: | |
| | | | 340) (A02.01) | |
| | | | FKLFSCLSFL (SEQ ID NO: | |
| | | | 341) (A02.01) | |
| | | | FLSSWDYRRM (SEQ ID | |
| | | | NO. 342) (A02.01) | |
| | | | GFKLFSCLSF (SEQ ID NO: | |
| | | | 343) (A24.02) | |
| | | | KLFSCLSFL (SEQ ID NO: | |
| | | | 344) (A02.01, A03.01) | |
| | | | KLFSCLSFLS (SEQ ID NO: | |
| | | | 345) (A02.01, A03.01) | |
| | | | LALFFFFFET (SEQ ID NO: | |
| | | | 346) (A02.01) | |
| | | | LFFFFFETK (SEQ ID NO: | |
| | | | 347) (A03.01) | |
| | | | LSFLSSWDY (SEQ ID NO: | |
| | | | 348) (A01.01) | |
| | | | LSFLSSWDYR (SEQ ID | |
| | | | NO: 349) (A03.01) | |
| | | | RMPPCLANF (SEQ ID NO: | |
| | | | 350) (A24.02) | |
| | | | RRMPPCLANF (SEQ ID | |
| | | | NO: 351) (A24.02) | |
| | | | SLQPPPPGFK (SEQ ID NO: | |
| | | | 352) (A03.01) | |
| | | | VQWRSLGSL (SEQ ID NO: | |
| | | | 353) (A02.01) | |
| CNOT1 | L1544fs; | LSVIIFFFVYIWHWALPLIL | FFFSVIFST (SEQ ID NO: | MSI+ CRC, |
| | +1 | NNHHICLMSSIILDCNSVRQ | 354) (A02.01) | MSI+ |
| | | SIMSVCFFFFSVIFSTRCLTD | MSVCFFFFSV (SEQ ID | Uterine/Endo- |
| | | SRYPNICWFK* (SEQ ID NO: | NO: 355) (A02.01) | metrium Cancer, |
| | | 62) | SVCFFFFSV (SEQ ID NO: | MSI+ Stomach |
| | | | 356) (A02.01, B08.01) | Cancer, Lynch |
| | | | SVCFFFFSVI (SEQ ID NO: | syndrome |
| | | | 357) (A02.01) | |
| CNOT1 | L1544fs; | LSVIIFFFVYIWHWALPLIL | FFCYILNTMF (SEQ ID NO: | MSI+ CRC |
| | −1 | NNHHICLMSSIILDCNSVRQ | 358) (A24.02) | MSI+ |
| | | SIMSVCFFFFCYILNTMFDR* | MSVCFFFFCY (SEQ ID | Uterine/Endo- |
| | | (SEQ ID NO: 63) | NO: 359) (A01.01) | metrium Cancer, |
| | | | SVCFFFFCYI (SEQ ID NO: | MSI+ Stomach |
| | | | 360) (A02.01) | Cancer, Lynch |
| | | | | syndrome |
| EIF2B3 | A151fs; | VLVLSCDLITDVALHEVVD | KQWSSVTSL (SEQ ID NO: | MSI+ CRC, |
| | −1 | LFRAYDASLAMLMRKGQD | 361) (A02. 01) | MSI+ |
| | | SIEPVPGQKGKKKQWSSVT | VLWMPTSTV (SEQ ID NO: | Uterine/Endo- |
| | | SLEWTAQERGCSSWLMKQ | 362) (A02.01) | metrium Cancer, |
| | | TWMKSWSLRDPSYRSILEY | | MSI+ Stomach |
| | | VSTRVLWMPTSTV* (SEQ | | Cancer, Lynch |
| | | ID NO: 64) | | syndrome |
| EPHB2 | K1020fs; | SIQVMRAQMNQIQSVEGQP | ILIRKAMTV (SEQ ID | MSI+ CRC, |
| | −1 | LARRPRATGRTKRCQPRDV | NO. 363) (A02.01) | MSI+ |
| | | TKKTCNSNDGKKREWEKR | | Uterine/Endo- |
| | | KQILGGGGKYKEYFLKRILI | | metrium Cancer, |
| | | RKAMTVLAGDKKGLGRFM | | MSI+ Stomach |
| | | RCVQSETKAVSLQLPLGR* | | Cancer, Lynch |
| | | (SEQ ID NO: 65) | | syndrome |
| ESRP1 | N512fs; | LDFLGEFATDIRTHGVHMV | | MSI+ CRC, |
| | +1 | LNHQGRPSGDAFIQMKSAD | | MSI+ |
| | | RAFMAAQKCHKKKHEGQI | | Uterine/Endo- |
| | | C* (SEQ ID NO: 66) | | metrium Cancer, |
| | | | | MSI+ Stomach |
| | | | | Cancer, Lynch |
| | | | | syndrome |
| ESRP1 | N512fs; | LDFLGEFATDIRTHGVHMV | | MSI+ CRC, |
| | −1 | LNHQGRPSGDAFIQMKSAD | | MSI+ |
| | | RAFMAAQKCHKKT* (SEQ | | Uterine/Endo- |
| | | ID NO: 67) | | metrium Cancer, |
| | | | | MSI+ Stomach |
| | | | | Cancer, Lynch |
| | | | | syndrome |
| FAM111B | A273fs; | GALCKDGRFRSDIGEFEWK | RMKVPLMK (SEQ ID NO: | MSI+ CRC, |
| | −1 | LKEGHKKIYGKQSMVDEV | 364) (A03.01) | MSI+ |
| | | SGKVLEMDISKKKHYNRKI | | Uterine/Endo- |
| | | SIKKLNRMKVPLMKLITRV* | | metrium Cancer, |
| | | (SEQ ID NO: 68) | | MSI+ Stomach |
| | | | | Cancer, Lynch |
| | | | | syndrome |
| GBP3 | T585fs; | RERAQLLEEQEKTLTSKLQ | TLKKKPRDI (SEQ ID | MSI+ CRC, |
| | −1 | EQARVLKERCQGESTQLQN | NO: 365) (B08.01) | MSI+ |
| | | EIQKLQKTLKKKPRDICRIS* | | Uterine/Endo- |
| | | (SEQ ID NO: 69) | | metrium Cancer, |
| | | | | MSI+ Stomach |
| | | | | Cancer, Lynch |
| | | | | syndrome |
| JAK1 | P861fs; | VNTLKEGKRLPCPPNCPDE | LIEGFEALLK (SEQ ID NO: | MSI+ CRC, |
| | +1 | VYQLMRKCWEFQPSNRTS | 366) (A03.01) | MSI+ |
| | | FQNLIEGFEALLKTSN* | | Uterine/Endo- |
| | | (SEQ ID NO: 70) | | metrium Cancer, |
| | | | | MSI+ Stomach |
| | | | | Cancer, Lynch |
| | | | | syndrome |
| JAK1 | K860fs; | CRPVTPSCKELADLMTRCM | QQLKWTPHI (SEQ ID NO: | MSI+ CRC, |
| | −1 | NYDPNQRPFFRAIMRDINK | 367) (A02.01) | MSI+ |
| | | LEEQNPDIVSEKNQQLKWT | QLKWTPHILK (SEQ ID | Uterine/Endo- |
| | | PHILKSAS* | NO: 368) (A03.01) | metrium Cancer, |
| | | (SEQ ID NO: 71) | IVSEKNQQLK (SEQ ID | MSI+ Stomach |
| | | | NO: 369) (A03.01) | Cancer, Lynch |
| | | | QLKWTPHILK (SEQ ID | syndrome |
| | | | NO: 368) (A03.01) | |
| | | | QQLKWTPHI (SEQ ID NO: | |
| | | | 367) (A24.02) | |
| | | | NQQLKWTPHIL (SEQ ID | |
| | | | NO: 370) (B08.01) | |
| | | | NQQLKWTPHI (SEQ ID | |
| | | | NO: 371) (B08.01) | |
| | | | QLKWTPHIL (SEQ ID NO: | |
| | | | 372) (B08.01) | |
| LMAN1 | E305fs; | DDHDVLSFLTFQLTEPGKE | GPPRPPRAAC (SEQ ID | MSI+ CRC, |
| | +1 | PPTPDKEISEKEKEKYQEEF | NO: 373) (B07.02) | MSI+ |
| | | EHFQQELDKKKRGIPEGPP | PPRPPRAAC (SEQ ID NO: | Uterine/Endo- |
| | | RPPRAACGGNI* (SEQ ID | 374) (B07.02) | metrium Cancer, |
| | | NO: 72) | | MSI+ Stomach |
| | | | | Cancer, Lynch |
| | | | | syndrome |
| LMAN1 | E305fs; | DDHDVLSFLTFQLTEPGKE | SLRRKYLRV (SEQ ID NO: | MSI+ CRC, |
| | −1 | PTPDKEISEKEKEKYQEEF | 375) (B08.01) | MSI+ |
| | | EHFQQELDKKKRNSRRATP | | Uterine/Endo- |
| | | TSKGSLRRKYLRV* (SEQ | | metrium Cancer, |
| | | ID NO: 73) | | MSI+ Stomach |
| | | | | Cancer, Lynch |
| | | | | syndrome |
| MSH3 | N385fs; | TKSTLIGEDVNPLIKLDDAV | SAACHRRGCV (SEQ ID | MSI+ CRC, |
| | +1 | NVDEIMTDTSTSYLLCISEN | NO: 376) (B08.01) | MSI+ |
| | | KENVRDKKKGQHFYWHC | | Uterine/Endo- |
| | | GSAACHRRGCV* (SEQ ID | | metrium Cancer, |
| | | NO: 74) | | MSI+ Stomach |
| | | | | Cancer, Lynch |
| | | | | syndrome |
| MSH3 | K383fs; | LYTKSTLIGEDVNPLIKLDD | ALWECSLPQA (SEQ ID | MSI+ CRC, |
| | −1 | AVNVDEIMTDTSTSYLLCIS | NO: 377) (A02.01) | MSI+ |
| | | ENKENVRDKKRATFLLAL | CLIVSRTLL (SEQ ID NO: | Uterine/Endo- |
| | | WECSLPQARLCLIVSRTLLL | 378) (B08.01) | metrium Cancer, |
| | | VQS* (SEQ ID NO: 75) | CLIVSRTLLL (SEQ ID NO: | MSI+ Stomach |
| | | | 379) (A02.01, B08.01) | Cancer, Lynch |
| | | | FLLALWECS (SEQ ID NO: | syndrome |
| | | | 380) (A02.01) | |
| | | | FLLALWECSL (SEQ ID | |
| | | | NO: 381) (A02.01, B08.01) | |
| | | | IVSRTLLLV (SEQ ID NO: | |
| | | | 382) (A02.01) | |
| | | | LIVSRTLLL (SEQ ID NO: | |
| | | | 383) (A02.01, B08.01) | |
| | | | LIVSRTLLLV (SEQ ID NO: | |
| | | | 384) (A02.01) | |
| | | | LLALWECSL (SEQ ID NO: | |
| | | | 385) (A02.01, B08.01) | |
| | | | LPQARLCLI (SEQ ID NO: | |
| | | | 386) (B08.01, B07.02) | |
| | | | LPQARLCLIV (SEQ ID NO: | |
| | | | 387) (B08.01) | |
| | | | NVRDKKRATF (SEQ ID | |
| | | | NO: 388) (B08.01) | |
| | | | SLPQARLCLI (SEQ ID NO: | |
| | | | 389) (A02.01, B08.01) | |
| NDUFC2 | A70fs; | LPPPKLTDPRLLYIGFLGYC | FFCWILSCK (SEQ ID NO: | MSI+ CRC, |
| | +1 | SGLIDNLIRRRPIATAGLEIR | 390) (A03.01) | MSI+ |
| | | QLLYITAFFFCWILSCKT* | FFFCWILSCK (SEQ ID NO: | Uterine/Endo- |
| | | (SEQ ID NO: 76) | 391) (A03.01) | metrium Cancer, |
| | | | ITAFFFCWI (SEQ ID NO: | MSI+ Stomach |
| | | | 392) (A02.01) | Cancer, Lynch |
| | | | LYITAFFFCW (SEQ ID | syndrome |
| | | | NO: 393) (A24.02) | |
| | | | YITAFFFCWI (SEQ ID NO: | |
| | | | 394) (A02.01) | |
| NDUFC2 | F69fs; | SLPPPKLTDPRLLYIGFLGY | ITAFFLLDI (SEQ ID NO: | MSI+ CRC, |
| | −1 | CSGLIDNLIRRRPIATAGLH | 395) (A02.01) | MSI+ |
| | | RQLLYITAFFLLDIIL* (SEQ | LLYITAFFL (SEQ ID NO: | Uterine/Endo- |
| | | ID NO: 77) | 396) (A02.01, B08.01) | metrium Cancer, |
| | | | LLYITAFFLL (SEQ ID NO: | MSI+ Stomach |
| | | | 397) (A02.01, A24.02) | Cancer, Lynch |
| | | | LYITAFFLL (SEQ ID NO: | syndrome |
| | | | 398) (A24.02) | |
| | | | LYITAFFLLD (SEQ ID NO: | |
| | | | 399) (A24.02) | |
| | | | YITAFFLLDI (SEQ ID NO: | |
| | | | 400) (A02.01) | |
| RBM27 | Q817; | NQSGGAGEDCQIFSTPGHP | GSNEVTTRY (SEQ ID NO: | MSI+ CRC, |
| | +1 | KMIYSSSNLKTPSKLCSGSK | 401) (A01.01) | MSI+ |
| | | SHDVQEVLKKKTGSNEVTT | MPKDVNIQV (SEQ ID NO: | Uterine/Endo- |
| | | RYEEKKTGSVRKANRMPK | 402) (B07.02) | metrium Cancer, |
| | | DVNIQVRKKQKHETRRKS | TGSNEVTTRY (SEQ ID | MSI+ Stomach |
| | | KYNEDFERAWREDLTIKR* | NO: 403) (A01.01) | Cancer, Lynch |
| | | (SEQ ID NO: 78) | | syndrome |
| RPL22 | K16fs; | MAPVKKLVVKGGKKKEAS | | MSI+ CRC, |
| | +1 | SEVHS* (SEQ ID NO: 79) | | MSI+ |
| | | | | Uterine/Endo- |
| | | | | metrium Cancer, |
| | | | | MSI+ Stomach |
| | | | | Cancer, Lynch |
| | | | | syndrome |
| RPL22 | K15fs; | MAPVKKLVVKGGKKRSKF* | | MSI+ CRC, |
| | −1 | (SEQ ID NO: 80) | | MSI+ |
| | | | | Uterine/Endo- |
| | | | | metrium Cancer, |
| | | | | MSI+ Stomach |
| | | | | Cancer, Lynch |
| | | | | syndrome |
| SEC31A | I462fs; | MPSHQGAEQQQQQHHVFIS | | MSI+ CRC, |
| | +1 | QVVTEKEFLSRSDQLQQAV | | MSI+ |
| | | QSQGFINYCQKKN* (SEQ | | Uterine/Endo- |
| | | ID NO: 81) | | metrium Cancer, |
| | | | | MSI+ Stomach |
| | | | | Cancer, Lynch |
| | | | | syndrome |
| SEC31A | I462fs; | MPSHQGAEQQQQQHHVFIS | KKLMLLRLNL (SEQ ID | MSI+ CRC, |
| | −1 | QVVTEKEFLSRSDQLQQAV | NO: 404) (A02.01) | MSI+ |
| | | QSQGFINYCQKKLMLLRLN | KLMLLRLNL (SEQ ID NO: | Uterine/Endo- |
| | | LRKMCGPF* (SEQ ID NO: | 405) (A02.01, A03.01, | metrium Cancer, |
| | | 82) | B07.02, B08.01) | MSI+ Stomach |
| | | | KLMLLRLNLR (SEQ ID | Cancer, Lynch |
| | | | NO: 406) (A03.01) | syndrome |
| | | | LLRLNLRKM (SEQ ID NO: | |
| | | | 407) (B08.01) | |
| | | | LMLLRLNL (SEQ ID NO: | |
| | | | 408) (B08.01) | |
| | | | LMLLRLNLRK (SEQ ID | |
| | | | NO: 409) (A03.01) | |
| | | | LNLCGPF (SEQ ID | |
| | | | NO: 410) (B08.01) | |
| | | | MLLRLNLRK (SEQ ID NO: | |
| | | | 411) (A03.01) | |
| | | | MLLRLNLRKM (SEQ ID | |
| | | | NO: 412) (A02.01, A03.01, | |
| | | | B08.01) | |
| | | | NLRKMCGPF (SEQ ID NO: | |
| | | | 413) (B08.01) | |
| | | | NYCQKKLMLL (SEQ ID | |
| | | | NO: 414) (A24.02) | |
| | | | YCQKKLMLL (SEQ ID | |
| | | | NO: 415) (B08.01) | |
| SEC63 | K530fs; | AEVFEKEQSICAAEEQPAE | FKKKTYTCAI (SEQ ID | MSI+ CRC, |
| | +1 | DGQGETNKNRTKGGWQQ | NO: 416) (B08.01) | MSI+ |
| | | KSKGPKKTAKSKKKETFKK | ITTVKATETK (SEQ ID | Uterine/Endo- |
| | | KTYTCAITTVKATETKAGK | NO: 417) (A03.01) | metrium Cancer, |
| | | WSRWE* (SEQ ID NO: 83) | KSKKKETFK (SEQ ID NO: | MSI+ Stomach |
| | | | 418) (A03.01) | Cancer, Lynch |
| | | | KSKKKETFKK (SEQ ID | syndrome |
| | | | NO: 419) (A03.01) | |
| | | | KTYTCAITTV (SEQ ID | |
| | | | NO: 420) (A02.01, A24.02) | |
| | | | TFKKKTYTC (SEQ ID NO: | |
| | | | 421) (B08.01) | |
| | | | TYTCAITTV (SEQ ID NO: | |
| | | | 422) (A24.02) | |
| | | | TYTCAITTVK (SEQ ID | |
| | | | NO: 423) (A03.01) | |
| | | | YTCAITTVK (SEQ ID NO: | |
| | | | 424) (A03.01) | |
| SEC63 | K529fs; | MAEVFEKEQSICAAEEQPA | TAKSKKRNL (SEQ ID NO: | MSI+ CRC, |
| | −1 | EDGQGETNKNRTKGGWQQ | 425) (B08.01) | MSI+ |
| | | KSKGPKKTAKSKKRNL* | | Uterine/Endo- |
| | | (SEQ ID NO: 84) | | metrium Cancer, |
| | | | | MSI+ Stomach |
| | | | | Cancer, Lynch |
| | | | | syndrome |
| SLC35F5 | C248fs; | NIMEIRQLPSSHALEAKLSR | FALCGFWQI (SEQ ID NO: | MSI+ CRC, |
| | −1 | MSYPVKEQESILKTVGKLT | 426) (A02.01) | MSI+ |
| | | ATQVAKISFFFALCGFWQIC | | Uterine/Endo- |
| | | HIKKHFQTEIKLL* (SEQ ID | | metrium Cancer, |
| | | NO: 85) | | MSI+ Stomach |
| | | | | Cancer, Lynch |
| | | | | syndrome |
| SMAP1 | K172fs; | YEKKKYYDKNAIAITNISSS | | MSI+ CRC, |
| | +1 | DAPLQPLVSSPSLQAAVDK | | MSI+ |
| | | NKLEKEKEKKKGREKERK | | Uterine/Endo- |
| | | GARKAGKTTYS* (SEQ ID | | metrium Cancer, |
| | | NO: 86) | | MSI+ Stomach |
| | | | | Cancer, Lynch |
| | | | | syndrome |
| SMAP1 | K171fs; | KYEKKKYYDKNAIAITNISS | LKKLRSPL (SEQ ID NO: | MSI+ CRC, |
| | −1 | SDAPLQPLVSSPSLQAAVD | 427) (B08.01) | MSI+ |
| | | KNKLEKEKEKKRKRKREK | SLKKVPAL (SEQ ID NO: | Uterine/Endo- |
| | | RSQKSRQNEILQLKSCRRKI | 428) (B08.01) | metrium Cancer, |
| | | SNWSLKKVPALKKLRSPL | RKISNWSLKK (SEQ ID | MSI+ Stomach |
| | | WIF* (SEQ ID NO: 87) | NO: 429) (A03.01) | Cancer, Lynch |
| | | | VPALKKLRSPL (SEQ ID | syndrome |
| | | | NO: 430) (B07.02) | |
| TFAM | E148fs; | IYQDAYRAEWQVYKEEISR | KRVNTAWKTK (SEQ ID | MSI+ CRC, |
| | +1 | FKEQLTPSQIMSLEKEIMDK | NO: 431) (A03.01) | MSI+ |
| | | HLKRKAMTKKKRVNTAW | MTKKKRVNTA (SEQ ID | Uterine/Endo- |
| | | KTKKTSFSL* | NO: 432) (B08.01) | metrium Cancer, |
| | | (SEQ ID NO: 88) | RVNTAWKTK (SEQ ID | MSI+ Stomach |
| | | | NO: 433) (A03.01) | Cancer, Lynch |
| | | | RVNTAWKTKK (SEQ ID | syndrome |
| | | | NO: 434) (A03.01) | |
| | | | TKKKRVNTA (SEQ ID NO: | |
| | | | 435) (B08.01) | |
| | | | WKTKKTSFSL (SEQ ID | |
| | | | NO: 436) (B08.01) | |
| TFAM | E148fs; | IYQDAYRAEWQVYKEEISR | | MSI+CRC, |
| | −1 | FKEQLTPSQIMSLEKEIMDK | | MSI+ |
| | | HLKRKAMTKKKS* (SEQ ID | | Uterine/Endo- |
| | | NO: 89) | | metrium Cancer, |
| | | | | MSI+ Stomach |
| | | | | Cancer, Lynch |
| | | | | syndrome |
| TGFBR2 | P129fs; | KPQEVCVAVWRKNDENIT | | MSI+ CRC, |
| | +1 | LETVCHDPKLPYHDFILED | | MSI+ |
| | | AASPKCIMKEKKKAW* | | Uterine/Endo- |
| | | (SEQ ID NO: 90) | | metrium Cancer, |
| | | | | MSI+ Stomach |
| | | | | Cancer, Lynch |
| | | | | syndrome |
| TGFBR2 | K128fs; | EKPQEVCVAVWRKNDENI | ALMSAMTTS (SEQ ID NO: | MSI+ CRC, |
| | −1 | TLETVCHDPKLPYHDFILED | 437) (A02.01) | MSI+ |
| | | AASPKCIMKEKKSLVRLSS | AMTTSSSQK (SEQ ID NO: | Uterine/Endo- |
| | | CVPVALMSAMTTSSSQKNI | 438) (A03.01, A11.01) | metrium Cancer, |
| | | TPAILTCC* | AMTTSSSQKN (SEQ ID | MSI+ Stomach |
| | | (SEQ ID NO: 91) | NO: 439) (A03.01) | Cancer, Lynch |
| | | | CIMKEKKSL (SEQ ID NO: | syndrome |
| | | | 440) (B08.01) | |
| | | | CIMKEKKSLV (SEQ ID | |
| | | | NO: 441) (B08.01) | |
| | | | IMKEKKSL (SEQ ID NO: | |
| | | | 442) (B08.01) | |
| | | | IMKEKKSLV (SEQ ID NO: | |
| | | | 443) (B08.01) | |
| | | | KSLVRLSSCV (SEQ ID | |
| | | | NO: 444) (A02.01) | |
| | | | LVRLSSCVPV (SEQ ID | |
| | | | NO: 445) (A02.01) | |
| | | | RLSSCVPVA (SEQ ID NO: | |
| | | | 446) (A02.01, A03.01) | |
| | | | RLSSCVPVAL (SEQ ID | |
| | | | NO: 447) (A02.01) | |
| | | | SAMTTSSSQK (SEQ ID | |
| | | | NO: 448) (A03.01, A11.01) | |
| | | | SLVRLSSCV (SEQ ID NO: | |
| | | | 449) (A02.01) | |
| | | | VPVALMSAM (SEQ ID | |
| | | | NO: 450) (B07.02) | |
| | | | VRLSSCVPVA (SEQ ID | |
| | | | NO: 451) (A02.01) | |
| THAP5 | K99fs; | VPSKYQFLCSDEIFTPDSLDI | KMRKKYAQK (SEQ ID | MSI+ CRC, |
| | −1 | RWGIRYLKQTAVPTIFSLPE | NO: 452) (A03.01) | MSI+ |
| | | DNQGKDPSKKNPRRKTWK | | Uterine/Endo- |
| | | MRKKYAQKPSQKNHLY* | | metrium Cancer, |
| | | (SEQ ID NO: 92) | | MSI+ Stomach |
| | | | | Cancer, Lynch |
| | | | | syndrome |
| TTK | R854fs; | GTTEEMKYVLGQLVGLNS | FVMSDTTYK (SEQ ID NO: | MSI+ CRC, |
| | −1 | PNSILKAAKTLYEHYSGGE | 453) (A03.01) | MSI+ |
| | | SHNSSSSKTFEKKGEKNDL | FVMSDTTYKI (SEQ ID | Uterine/Endo- |
| | | QLFVMSDTTYKIYWTVILL | NO: 454) (A02.01) | metrium Cancer, |
| | | NPCGNLEILKTTSL* | KTFEKKGEK (SEQ ID NO: | MSI+ Stomach |
| | | (SEQ ID NO: 93) | 455) (A03.01) | Cancer, Lynch |
| | | | LFVMSDTTYK (SEQ ID | syndrome |
| | | | NO: 456) (A03.01) | |
| | | | MSDTTYKIY (SEQ ID NO: | |
| | | | 457) (A01.01) | |
| | | | VMSDTTYKI (SEQ ID NO: | |
| | | | 458) (A02.01) | |
| | | | VMSDTTYKIY (SEQ ID | |
| | | | NO: 459) (A01.01) | |
| XPOT | F126fs; | QQLIRETLISWLQAQMLNP | YLTKWPKFFL (SEQ ID | MSI+ CRC, |
| | −1 | QPEKTFIRNKAAQVFALLF | NO: 460) (A02.01) | MSI+ |
| | | VTEYLTKWPKFFLTFSQ* | | Uterine/Endo- |
| | | (SEQ ID NO: 94) | | metrium Cancer, |
| | | | | MSI+ Stomach |
| | | | | Cancer, Lynch |
| | | | | syndrome |

TABLE 1C
FRAMESHIFT

| | | | | |
|---|---|---|---|---|
| APC | V1352fs | AKFQQCHSTLEPNPADCRV | FLQERNLPP (SEQ ID NO: | CRC, LUAD, |
| | F1354fs | LVYLQNQPGTKLLNFLQER | 461)(A02.01) | UCEC, STAD |
| | Q1378fs | NLPPKVVLRHPKVHLNTMF | FRRPHSCLA (SEQ ID NO: | |
| | S1398fs | RRPHSCLADVLLSVHLIVL | 462)(B08. 01) | |
| | | RVVRLPAPFRVNHAVEW* | LIVLRVVRL (SEQ ID NO: | |
| | | (SEQ ID NO: 95) | 463)(B08.01) | |
| | | | LLSVHLIVL (SEQ ID NO: | |
| | | | 464)(A02.01, B08.01) | |
| APC | S1421fs | APVIFQIALDKPCHQAEVK | EVKHLHHLL (SEQ ID NO: | CRC, LUAD, |
| | R1435fs | HLHHLLKQLKPSEKYLKIK | 465)(B08.01) | UCEC, STAD |
| | T1438fs | HLLLKRERVDLSKLQ* | HLHHLLKQLK (SEQ ID | |
| | P1442fs | (SEQ ID NO: 96) | NO: 466)(A03.01) | |
| | P1443fs | | HLLLKRERV (SEQ ID NO: | |
| | V1452fs | | 467)(B08.01) | |
| | P1453fs | | KIKHLLLKR (SEQ ID NO: | |
| | K1462fs | | 468)(A03.01) | |
| | E1464fs | | KPSEKYLKI (SEQ ID NO: | |
| | | | 469)(B07.02) | |
| | | | KYLKIKHLL (SEQ ID NO: | |
| | | | 470)(A24.02) | |
| | | | KYLKIKHLLL (SEQ ID | |
| | | | NO: 471)(A24.02) | |
| | | | LLKQLKPSEK (SEQ ID | |
| | | | NO: 472)(A03.01) | |
| | | | LLKRERVDL (SEQ ID NO: | |
| | | | 473)(B08.01) | |
| | | | LLLKRERVDL (SEQ ID | |
| | | | NO: 474)(B08.01) | |
| | | | QLKPSEKYLK (SEQ ID | |
| | | | NO: 475)(A03.01) | |
| | | | YLKIKHLLL (SEQ ID NO: | |
| | | | 476)(A02.01, B08.01) | |
| | | | YLKIKHLLLK (SEQ ID | |
| | | | NO: 477)(A03.01) | |
| APC | T1487fs | MLQFRGSRFFQMLILYYILP | ILPRKVLQM (SEQ ID NO: | CRC, LUAD, |
| | H1490fs | RKVLQMDFLVHPA* (SEQ | 478)(B08.01) | UCEC, STAD |
| | L1488fs | ID NO: 97) | KVLQMDFLV (SEQ ID | |
| | | | NO: 479)(A02.01, A24.02) | |
| | | | LPRKVLQMDF (SEQ ID | |
| | | | NO: 480)(B07.02, B08.01) | |
| | | | LQMDFLVHPA (SEQ ID | |
| | | | NO: 481)(A02.01) | |
| | | | QMDFLVHPA (SEQ ID NO: | |
| | | | 482)(A02.01) | |
| | | | YILPRKVLQM (SEQ ID | |
| | | | NO: 483)(A02.01, B08.01) | |
| ARID1 | Q1306fs | ALGPHSRISCLPTQTRGCIL | APSPASRLQC (SEQ ID | STAD, UCEC, |
| A | S1316fs | LAATPRSSSSSSSNDMIPMA | NO: 484)(B07.02) | BLCA, BRCA, |
| | Y1324fs | ISSPPKAPLLAAPSPASRLQ | HPLAPMPSKT (SEQ ID | LUSC, CESC, |
| | T1348fs | CINSNSRITSGQWMAHMAL | NO: 485)(B07.02) | KIRC, UCS |
| | G1351fs | LPSGTKGRCTACHTALGRG | ILPLPQLLL (SEQ ID NO: | |
| | G1378fs | SLSSSSCPQPSPSLPASNKLP | 486)(A02.01) | |
| | P1467fs | SLPLSKMYTTSMAMPILPLP | LPTQTRGCIL (SEQ ID NO: | |
| | | QLLLSADQQAAPRTNFHSS | 487)(B07.02) | |
| | | LAETVSLHPLAPMPSKTCH | RISCLPTQTR (SEQ ID NO: | |
| | | HK* (SEQ ID NO: 98) | 488)(A03.01) | |
| | | | SLAETVSLH (SEQ ID NO: | |
| | | | 489)(A03.01) | |
| | | | TPRSSSSSS (SEQ ID NO: | |
| | | | 490)(B07.02) | |
| | | | TPRSSSSSSS (SEQ ID NO: | |
| | | | 491)(B07.02) | |
| ARID1 | S674fs | AHQGFPAAKESRVIQLSLLS | ALPPVLLSL (SEQ ID NO: | STAD, UCEC, |
| A | P725fs | LLIPPLTCLASEALPRPLLAL | 492)(A02.01) | BLCA, BRCA, |
| | R727fs | PPVLLSLAQDHSRLLQCQA | ALPPVLLSLA (SEQ ID | LUSC, CESC, |
| | I736fs | TRCHLGHPVASRTASCILP* | NO: 493)(A02.01) | KIRC, UCS |
| | | (SEQ ID NO: 99) | ALPRPLLAL (SEQ ID NO: | |
| | | | 494)(A02.01) | |
| | | | ASRTASCIL (SEQ ID NO: | |
| | | | 495)(B07.02) | |
| | | | EALPRPLLAL (SEQ ID | |
| | | | NO: 496)(B08.01) | |
| | | | HLGHPVASR (SEQ ID NO: | |
| | | | 497)(A03.01) | |
| | | | HPVASRTAS (SEQ ID NO: | |
| | | | 498)(B07.02) | |
| | | | HPVASRTASC (SEQ ID | |
| | | | NO: 499)(B07.02) | |
| | | | IIQLSLLSLL (SEQ ID NO: | |
| | | | 500)(A02.01) | |
| | | | IQLSLLSLL (SEQ ID NO: | |
| | | | 501)(A02.01) | |
| | | | IQLSLLSLLI (SEQ ID NO: | |
| | | | 502)(A02.01, A24.02) | |
| | | | LLALPPVLL (SEQ ID NO: | |
| | | | 503)(A02.01) | |
| | | | LLIPPLTCL (SEQ ID NO: | |
| | | | 504)(A02.01) | |
| | | | LLIPPLTCLA (SEQ ID NO: | |
| | | | 505)(A02.01) | |
| | | | LLSLLIPPL (SEQ ID NO: | |
| | | | 506)(A02.01) | |
| | | | LLSLLIPPLT (SEQ ID NO: | |
| | | | 507)(A02.01) | |
| | | | LPRPLLALPP (SEQ ID NO: | |
| | | | 508)(B07.02) | |
| | | | QLSLLSLLI (SEQ ID NO: | |
| | | | 509)(A02.01) | |
| | | | RLLQCQATR (SEQ ID NO: | |
| | | | 510)(A03.01) | |
| | | | RPLLALPPV (SEQ ID NO: | |
| | | | 511)(B07.02) | |
| | | | RPLLALPPVL (SEQ ID NO: | |
| | | | 512)(B07.02) | |
| | | | SLAQDHSRL (SEQ ID NO: | |
| | | | 513)(A02.01) | |
| | | | SLAQDHSRLL (SEQ ID | |
| | | | NO: 514)(A02.01) | |
| | | | SLLIPPLTCL (SEQ ID NO: | |
| | | | 515)(A02.01) | |
| | | | SLLSLLIPP (SEQ ID NO: | |
| | | | 516)(A02.01) | |
| | | | SLLSLLIPPL (SEQ ID NO: | |
| | | | 517)(A02.01, B08.01) | |
| ARID1 | G414fs | PILAATGTSVRTAARTWVP | AAATSAASTL (SEQ ID | STAD, UCEC, |
| A | Q473fs | RAAIRVPDPAAVPDDHAGP | NO: 518)(B07.02) | BLCA, BRCA, |
| | H477fs | GAECHGRPLLYTADSSLWT | AAIPASTSAV (SEQ ID NO: | LUSC, CESC, |
| | S499fs | TRPQRVWSTGPDSILQPAK | 519)(B07.02) | KIRC, UCS |
| | P504fs | SSPSAAAATLLPATTVPDPS | AIPASTSAV (SEQ ID NO: | |
| | Q548fs | CPTFVSAAATVSTTTAPVLS | 520)(A02.01) | |
| | P549fs | ASILPAAIPASTSAVPGSIPL | ALPAGCVSSA (SEQ ID | |
| | | PAVDDTAAPPEPAPLLTAT | NO: 521)(A02.01) | |
| | | GSVSLPAAATSAASTLDAL | APLLTATGSV (SEQ ID | |
| | | PAGCVSSAPVSAVPANCLF | NO: 522)(B07.02) | |
| | | PAALPSTAGAISRFIWVSGI | APVLSASIL (SEQ ID NO: | |
| | | LSPLNDLQ* (SEQ ID NO: | 523)(B07.02) | |
| | | 100) | ATLLPATTV (SEQ ID NO: | |
| | | | 524)(A02.01) | |
| | | | ATVSTTTAPV (SEQ ID | |
| | | | NO: 525)(A02.01) | |
| | | | AVPANCLFPA (SEQ ID | |
| | | | NO: 526)(A02.01) | |
| | | | CLFPAALPST (SEQ ID NO: | |
| | | | 527)(A02.01) | |
| | | | CPTFVSAAA (SEQ ID NO: | |
| | | | 528)(B07.02) | |
| | | | FPAALPSTA (SEQ ID NO: | |
| | | | 529)(B07.02) | |
| | | | FPAALPSTAG (SEQ ID | |
| | | | NO. 530)(B07.02) | |
| | | | GAECHGRPL (SEQ ID NO: | |
| | | | 531)(B07.02) | |
| | | | GAISRFIWV (SEQ ID NO: | |
| | | | 532)(A02.01) | |
| | | | ILPAAIPAST (SEQ ID NO: | |
| | | | 533)(A02.01) | |
| | | | IWVSGILSPL (SEQ ID NO: | |
| | | | 534)(A24.02) | |
| | | | LLTATGSVSL (SEQ ID | |
| | | | NO: 535)(A02.01) | |
| | | | LLYTADSSL (SEQ ID NO: | |
| | | | 536)(A02.01) | |
| | | | LPAAATSAA (SEQ ID NO: | |
| | | | 537)(B07.02) | |
| | | | LPAAATSAAS (SEQ ID | |
| | | | NO: 538)(B07.02) | |
| | | | LPAAIPAST (SEQ ID NO: | |
| | | | 539)(B07.02) | |
| | | | LPAGCVSSA (SEQ ID NO: | |
| | | | 540)(B07.02) | |
| | | | LPAGCVSSAP (SEQ ID | |
| | | | NO: 541)(B07.02) | |
| | | | LYTADSSLW (SEQ ID NO: | |
| | | | 542)(A24.02) | |
| | | | QPAKSSPSA (SEQ ID NO: | |
| | | | 543)(B07.02) | |
| | | | QPAKSSPSAA (SEQ ID | |
| | | | NO: 544)(B07.02) | |
| | | | RFIWVSGIL (SEQ ID NO: | |
| | | | 545)(A24.02) | |
| | | | RPQRVWSTG (SEQ ID NO: | |
| | | | 546)(B07.02) | |
| | | | RVWSTGPDSI (SEQ ID | |
| | | | NO: 547)(A02.01) | |
| | | | SAVPGSIPL (SEQ ID NO: | |
| | | | 548)(B07.02) | |
| | | | SILPAAIPA (SEQ ID NO: | |
| | | | 549)(A02.01) | |
| | | | SLPAAATSA (SEQ ID NO: | |
| | | | 550)(A02.01) | |
| | | | SLPAAATSAA (SEQ ID | |
| | | | NO: 551)(A02.01) | |
| | | | SLWTTRPQR (SEQ ID NO: | |
| | | | 552)(A03.01) | |
| | | | SLWTTRPQRV (SEQ ID | |
| | | | NO: 553)(A02.01) | |
| | | | SPSAAAATL (SEQ ID NO: | |
| | | | 554)(B07.02) | |
| | | | SPSAAAATLL (SEQ ID | |
| | | | NO: 555)(B07.02) | |
| | | | TLDALPAGCV (SEQ ID | |
| | | | NO: 556)(A02.01) | |
| | | | TVSTTTAPV (SEQ ID NO: | |
| | | | 557)(A02.01) | |
| | | | VLSASILPA (SEQ ID NO: | |
| | | | 558)(A02.01) | |
| | | | VLSASILPAA (SEQ ID NO: | |
| | | | 559)(A02.01) | |
| | | | VPANCLFPA (SEQ ID NO: | |
| | | | 560)(B07.02) | |
| | | | VPANCLFPAA (SEQ ID | |
| | | | NO: 561)(B07.02) | |
| | | | VPDPSCPTF (SEQ ID NO: | |
| | | | 562)(B07.02) | |
| | | | VPGSIPLPA (SEQ ID NO: | |
| | | | 563)(B07.02) | |
| | | | VPGSIPLPAV (SEQ ID NO: | |
| | | | 564)(B07.02) | |
| | | | WVSGILSPL (SEQ ID NO: | |
| | | | 565)(A02.01) | |
| | | | YTADSSLWTT (SEQ ID | |
| | | | NO: 566)(A02.01) | |
| ARID1 | T433fs | PCRAGRRVPWAASLIHSRF | APAGMVNRA (SEQ ID | STAD, UCEC, |
| A | A441fs | LLMDNKAPAGMVNRARLH | NO: 567)(B07.02) | BLCA, BRCA, |
| | Y447fs | ITTSKVLTLSSSSHPTPSNHR | ASLHRRSYL (SEQ ID NO: | LUSC, CESC, |
| | P483fs | PRPLMPNLRISSSHSLNHHS | 568)(B08.01) | KIRC, UCS |
| | P484fs | SSPLSLHTPSSHPSLHISSPR | ASLHRRSYLK (SEQ ID | |
| | P504fs | LHTPPSSRRHSSTPRASPPT | NO: 569)(A03.01) | |
| | S519fs | HSHRLSLLTSSSNLSSQHPR | FLLMDNKAPA (SEQ ID | |
| | H544fs | RSPSRLRILSPSLSSPSKLPIP | NO: 570)(A02.01) | |
| | P549fs | SSASLHRRSYLKIHLGLRHP | HPRRSPSRL (SEQ ID NO: | |
| | P554fs | QPPQ* (SEQ ID NO: 101) | 571)(B07.02, B08.01) | |
| | Q563fs | | HPSLHISSP (SEQ ID NO: | |
| | | | 572)(B07.02) | |
| | | | HRRSYLKIHL (SEQ ID | |
| | | | NO: 573)(B08.01) | |
| | | | HSRFLLMDNK (SEQ ID | |
| | | | NO: 574)(A03.01) | |
| | | | KLPIPSSASL (SEQ ID NO: | |
| | | | 575)(A02.01) | |
| | | | KVLTLSSSSH (SEQ ID NO: | |
| | | | 576)(A03.01) | |
| | | | LIHSRFLLM (SEQ ID NO: | |
| | | | 577)(B08.01) | |
| | | | LLMDNKAPA (SEQ ID | |
| | | | NO: 578)(A02.01) | |
| | | | LMDNKAPAGM (SEQ ID | |
| | | | NO: 579)(A02.01) | |
| | | | LPIPSSASL (SEQ ID NO: | |
| | | | 580)(B07.02) | |
| | | | MPNLRISSS (SEQ ID NO: | |
| | | | 581)(B07.02, B08.01) | |
| | | | MPNLRISSSH (SEQ ID NO: | |
| | | | 582)(B07.02) | |
| | | | NLRISSSHSL (SEQ ID NO: | |
| | | | 583)(B07.02, B08.01) | |
| | | | PPTHSHRLSL (SEQ ID NO: | |
| | | | 584)(B07.02) | |
| | | | RAGRRVPWAA (SEQ ID | |
| | | | NO: 585)(B08.01) | |
| | | | RARLHITTSK (SEQ ID NO: | |
| | | | 586)(A03.01) | |
| | | | RISSSHSLNH (SEQ ID NO: | |
| | | | 587)(A03.01) | |
| | | | RLHTPPSSR (SEQ ID NO: | |
| | | | 588)(A03.01) | |
| | | | RLHTPPSSRR (SEQ ID NO: | |
| | | | 589)(A03.01) | |
| | | | RLRILSPSL (SEQ ID NO: | |
| | | | 590)(A02.01, B07.02, | |
| | | | B08.01) | |
| | | | RPLMPNLRI (SEQ ID NO: | |
| | | | 591)(B07.02) | |
| | | | RPRPLMPNL (SEQ ID NO: | |
| | | | 592)(B07.02) | |
| | | | SASLHRRSYL (SEQ ID | |
| | | | NO: 593)(B07.02, B08.01) | |
| | | | SLHISSPRL (SEQ ID NO: | |
| | | | 594)(A02.01) | |
| | | | SLHRRSYLK (SEQ ID NO: | |
| | | | 595)(A03.01) | |
| | | | SLHRRSYLKI (SEQ ID NO: | |
| | | | 596)(B08.01) | |
| | | | SLIHSRFLL (SEQ ID NO: | |
| | | | 597)(A02.01) | |
| | | | SLIHSRFLLM (SEQ ID NO: | |
| | | | 598)(A02.01, B08.01) | |
| | | | SLLTSSSNL (SEQ ID NO: | |
| | | | 599)(A02.01) | |
| | | | SLNHHSSSPL (SEQ ID NO: | |
| | | | 600)(A02.01, B07.02, | |
| | | | B08.01) | |
| | | | SLSSPSKLPI (SEQ ID NO: | |
| | | | 601)(A02.01) | |
| | | | SPLSLHTPS (SEQ ID NO: | |
| | | | 602)(B07.02) | |
| | | | SPLSLHTPSS (SEQ ID NO: | |
| | | | 603)(B07.02) | |
| | | | SPPTHSHRL (SEQ ID NO: | |
| | | | 604)(B07.02) | |
| | | | SPRLHTPPS (SEQ ID NO: | |
| | | | 605)(B07.02) | |
| | | | SPRLHTPPSS (SEQ ID NO: | |
| | | | 606)(B07.02) | |
| | | | SPSLSSPSKL (SEQ ID NO: | |
| | | | 607)(B07.02) | |
| | | | SYLKIHLGL (SEQ ID NO: | |
| | | | 608)(A24.02) | |
| | | | TPSNHRPRPL (SEQ ID NO: | |
| | | | 609)(B07.02, B08.01) | |
| | | | TPSSHIPSLHI (SEQ ID NO: | |
| | | | 610)(B07.02) | |
| ARID1 | A2137fs | RTNPTVRMRPHCVPFWTG | CVPFWTGRIL (SEQ ID | STAD, UCEC, |
| A | P2139fs | RILLPSAASVCPIPFEACHLC | NO: 611)(B07.02) | BLCA, BRCA, |
| | L1970fs | QAMTLRCPNTQGCCSSWA | HCVPFWTGRIL (SEQ ID | LUSC, CESC, |
| | V1994fs | S* (SEQ ID NO: 102) | NO: 612)(B07.02) | KIRC, UCS |
| | | | ILLPSAASV (SEQ ID NO: | |
| | | | 613)(A02.01) | |
| | | | ILLPSAASVC (SEQ ID NO: | |
| | | | 614)(A02.01) | |
| | | | LLPSAASVCPI (SEQ ID | |
| | | | NO: 615)(A02.01) | |
| | | | LPSAASVCPI (SEQ ID NO: | |
| | | | 616)(B07.02) | |
| | | | MRPHCVPF (SEQ ID NO: | |
| | | | 617)(B08.01) | |
| | | | RILLPSAASV (SEQ ID NO: | |
| | | | 618)(A02.01) | |
| | | | RMRPHCVPF (SEQ ID NO: | |
| | | | 619)(A24.02, B07.02, | |
| | | | B08.01) | |
| | | | RMRPHCVPFW (SEQ ID | |
| | | | NO: 620)(A24.02) | |
| | | | RTNPTVRMR (SEQ ID NO: | |
| | | | 621)(A03.01) | |
| | | | SVCPIPFEA (SEQ ID NO: | |
| | | | 622)(A02.01) | |
| | | | TVRMRPHCV (SEQ ID NO: | |
| | | | 623)(B08.01) | |
| | | | TVRMRPHCVPF (SEQ ID | |
| | | | NO: 624)(B08.01) | |
| | | | VPFWTGRIL (SEQ ID NO: | |
| | | | 625)(B07.02) | |
| | | | VPFWTGRILL (SEQ ID | |
| | | | NO: 626)(B07.02) | |
| | | | VRMRPHCVPF (SEQ ID | |
| | | | NO: 627)(B08.01) | |
| ARID1 | N756fs | TNQALPKIEVICRGTPRCPS | AMVPRGVSM (SEQ ID | STAD, UCEC, |
| A | S764fs | TVPPSPAQPYLRVSLPEDRY | NO: 628)(B07.02, B08.01) | BLCA, BRCA, |
| | T783fs | TQAWAPTSRTPWGAMVPR | AMVPRGVSMA (SEQ ID | LUSC, CESC, |
| | Q799fs | GVSMAHKVATPGSQTIMPC | NO: 629)(A02.01) | KIRC, UCS |
| | A817fs | PMPTTPVQAWLEA* (SEQ | AWAPTSRTPW (SEQ ID | |
| | | ID NO: 103) | NO: 630)(A24.02) | |
| | | | CPMPTTPVQA (SEQ ID | |
| | | | NO: 631)(B07.02) | |
| | | | CPSTVPPSPA (SEQ ID NO: | |
| | | | 632)(B07.02) | |
| | | | GAMVPRGVSM (SEQ ID | |
| | | | NO: 633)(B07.02, B08.01) | |
| | | | MPCPMPTTPV (SEQ ID | |
| | | | NO: 634)(B07.02) | |
| | | | MPTTPVQAW (SEQ ID | |
| | | | NO: 635)(B07.02) | |
| | | | MPTTPVQAWL (SEQ ID | |
| | | | NO: 636)(B07.02) | |
| | | | SLPEDRYTQA (SEQ ID | |
| | | | NO: 637)(A02.01) | |
| | | | SPAQPYLRV (SEQ ID NO: | |
| | | | 638)(B07.02) | |
| | | | SPAQPYLRVS (SEQ ID | |
| | | | NO: 639)(B07.02) | |
| | | | TIMPCPMPT (SEQ ID NO: | |
| | | | 640)(A02.01) | |
| | | | TPVQAWLEA (SEQ ID NO: | |
| | | | 641)(B07.02) | |
| | | | TSRTPWGAM (SEQ ID | |
| | | | NO: 642)(B07.02) | |
| | | | VPPSPAQPYL (SEQ ID | |
| | | | NO: 643)(B07.02) | |
| | | | VPRGVSMAH (SEQ ID | |
| | | | NO: 644)(B07.02) | |
| β2M | N62fs | RMERELKKWSIQTCLSART | CLSARTGLSI (SEQ ID NO: | CRC, STAD, |
| | E67fs | GLSISCTTLNSPPLKKMSMP | 645)(B08.01) | SKCM, HNSC |
| | L74fs | AV* (SEQ ID NO: 104) | CTTLNSPPLK (SEQ ID NO: | |
| | F82fs | | 646)(A03.01) | |
| | T91fs | | GLSISCTTL (SEQ ID NO: | |
| | E94fs | | 647)(A02.01) | |
| | | | SPPLKKMSM (SEQ ID NO: | |
| | | | 648)(B07.02, B08.01) | |
| | | | TLNSPPLKK (SEQ ID NO: | |
| | | | 649)(A03.01) | |
| | | | TTLNSPPLK (SEQ ID NO: | |
| | | | 650)(A03.01) | |
| | | | TTLNSPPLKK (SEQ ID | |
| | | | NO: 651)(A03.01) | |
| β2M | L13fs | LCSRYSLFLAWRLSSVLQR | LQRFRFTHV (SEQ ID NO: | CRC, STAD, |
| | S14fs | FRFTHVIQQRMESQIS* | 652)(B08.01) | SKCM, HNSC |
| | | (SEQ ID NO: 105) | LQRFRFTHVI (SEQ ID NO: | |
| | | | 653)(B08.01) | |
| | | | RLSSVLQRF (SEQ ID NO: | |
| | | | 654)(A24.02) | |
| | | | RLSSVLQRFR (SEQ ID | |
| | | | NO: 655)(A03.01) | |
| | | | VLQRFRFTHV (SEQ ID | |
| | | | NO: 656)(A02.01, B08.01) | |
| CDH1 | A691fs | RSACVTVKGPLASVGRHSL | ASVGRHSLSK (SEQ ID | ILC LumA |
| | P708fs | SKQDCKFLPFWGFLEEFLL | NO: 657)(A03.01) | Breast Cancer |
| | L711fs | C* (SEQ ID NO: 106) | KFLPFWGFL (SEQ ID NO: | |
| | | | 658)(A24.02) | |
| | | | LASVGRHSL (SEQ ID NO: | |
| | | | 659)(B07.02) | |
| | | | LPFWGFLEEF (SEQ ID | |
| | | | NO: 660)(B07.02) | |
| | | | PFWGFLEEF (SEQ ID NO: | |
| | | | 661)(A24.02) | |
| | | | SVGRHSLSK (SEQ ID NO: | |
| | | | 662)(A03.01) | |
| CDH1 | H121fs | IQWGTTTAPRPIRPPFLESK | APRPIRPPF (SEQ ID NO: | ILC LumA |
| | P126fs | QNCSHFPTPLLASEDRRET | 663)(B07.02) | Breast Cancer |
| | H128fs | GLFLPSAAQKMKKAHFLK | APRPIRPPFL (SEQ ID NO: | |
| | N144fs | TWFRSNPTKTKKARFSTAS | 664)(B07.02) | |
| | V157fs | LAKELTHPLLVSLLLKEKQ | AQKMKKAHFL (SEQ ID | |
| | P159fs | DG* (SEQ ID NO: 107) | NO: 665)(B08.01) | |
| | N166fs | | FLPSAAQKM (SEQ ID NO: | |
| | N181fs | | 666)(A02.01) | |
| | F189fs | | GLFLPSAAQK (SEQ ID | |
| | P201fs | | NO: 667)(A03.01) | |
| | F205fs | | HPLLVSLLL (SEQ ID NO: | |
| | | | 668)(B07.02) | |
| | | | KAHFLKTWFR (SEQ ID | |
| | | | NO: 669)(A03.01) | |
| | | | KARFSTASL (SEQ ID NO: | |
| | | | 670)(B07.02) | |
| | | | KMKKAHFLK (SEQ ID | |
| | | | NO: 671)(A03.01) | |
| | | | KTWFRSNPTK (SEQ ID | |
| | | | NO: 672)(A03.01) | |
| | | | LAKELTHPL (SEQ ID NO: | |
| | | | 673)(B07.02, B08.01) | |
| | | | LAKELTHPLL (SEQ ID | |
| | | | NO: 674)(B08.01) | |
| | | | NPTKTKKARF (SEQ ID | |
| | | | NO: 675)(B07.02) | |
| | | | QKMKKAHFL (SEQ ID | |
| | | | NO: 676)(B08.01) | |
| | | | RFSTASLAK (SEQ ID NO: | |
| | | | 677)(A03.01) | |
| | | | RPIRPPFLES (SEQ ID NO: | |
| | | | 678)(B07.02) | |
| | | | RSNPTKTKK (SEQ ID NO: | |
| | | | 679)(A03.01) | |
| | | | SLAKELTHPL (SEQ ID | |
| | | | NO: 680)(A02.01, B08.01) | |
| | | | TKKARFSTA (SEQ ID NO: | |
| | | | 681)(B08.01) | |
| CDH1 | V114fs | PTDPFLGLRLGLHLQKVFH | GLRFWNPSR (SEQ ID NO: | ILC LumA |
| | P127fs | QSHAEYSGAPPPPPAPSGLR | 682)(A03.01) | Breast Cancer |
| | V132fs | FWNPSRIAHISQLLSWPQKT | ISQLLSWPQK (SEQ ID | |
| | P160fs | EERLGYSSHQLPRK* (SEQ | NO: 683)(A03.01) | |
| | | ID NO: 108) | RIAHISQLL (SEQ ID NO: | |
| | | | 684)(A02.01) | |
| | | | RLGYSSHQL (SEQ ID NO: | |
| | | | 685)(A02.01) | |
| | | | SQLLSWPQK (SEQ ID NO: | |
| | | | 686)(A03.01) | |
| | | | SRIAHISQL (SEQ ID NO: | |
| | | | 687)(B08.01) | |
| | | | WPQKTEERL (SEQ ID NO: | |
| | | | 688)(B07.02) | |
| | | | YSSHQLPRK (SEQ ID NO: | |
| | | | 689)(A03.01) | |
| CDH1 | L731fs | FCCSCCFFGGERWSKSPYC | CPQRMTPGTT (SEQ ID | ILC LumA |
| | R749fs | PQRMTPGTTFITMMKKEAE | NO: 690)(B07.02) | Breast Cancer |
| | E757fs | KRTRTLT* (SEQ ID NO: | EAEKRTRTL (SEQ ID NO: | |
| | G759fs | 109) | 691)(B08.01) | |
| | | | GTTFITMMK (SEQ ID NO: | |
| | | | 692)(A03.01) | |
| | | | GTTFITMMKK (SEQ ID | |
| | | | NO: 693)(A03.01) | |
| | | | ITMMKKEAEK (SEQ ID | |
| | | | NO: 694)(A03.01) | |
| | | | RMTPGTTFI (SEQ ID NO: | |
| | | | 695)(A02.01) | |
| | | | SPYCPQRMT (SEQ ID NO: | |
| | | | 696)(B07.02) | |
| | | | TMMKKEAEK (SEQ ID | |
| | | | NO: 697)(A03.01) | |
| | | | TPGTTFITM (SEQ ID NO: | |
| | | | 698)(B07.02) | |
| | | | TPGTTFITMM (SEQ ID | |
| | | | NO: 699)(B07.02) | |
| | | | TTFITMMKK (SEQ ID NO: | |
| | | | 700)(A03.01) | |
| CDH1 | S19fs | WRRNCKAPVSLRKSVQTP | CPGATWREA (SEQ ID NO: | ILC LumA |
| | E24fs | ARSSPARPDRTRRLPSLGVP | 701)(B07.02) | Breast Cancer |
| | S36fs | GQPWALGAAASRRCCCCC | CPGATWREAA (SEQ ID | |
| | | RSPLGSARSRSPATLALTPR | NO: 702)(B07.02) | |
| | | ATRSRCPGATWREAASWA | RSRCPGATWR (SEQ ID | |
| | | E* (SEQ ID NO: 110) | NO: 703)(A03.01) | |
| | | | TPRATRSRC (SEQ ID NO: | |
| | | | 704)(B07.02) | |
| GATA3 | P394fs | PGRPLQTHVLPEPHLALQP | HVLPEPHLAL (SEQ ID | Breast Cancer |
| | P387fs | LQPHADHAHADAPAIQPVL | NO: 705)(B07.02) | |
| | S398fs | WTTPPLQHGHRHGLEPCS | RPLQTHVLPE (SEQ ID | |
| | H400fs | MLTGPPARVPAVPFDLHFC | NO: 706)(B07.02) | |
| | M401fs | RSSIMKPKRDGYMFLKAES | VLWTTPPLQH (SEQ ID | |
| | S408fs | KIMFATLQRSSLWCLCSNH* | NO: 707)(A03.01) | |
| | P409fs | (SEQ ID NO: 111) | | |
| | S408fs | | | |
| | P409fs | | | |
| | T419fs | | | |
| | H424fs | | | |
| | P425fs | | | |
| | S427fs | | | |
| | F431fs | | | |
| | S430fs | | | |
| | H434fs | | | |
| | H435fs | | | |
| | S438fs | | | |
| | M443fs | | | |
| | G444fs | | | |
| | *445fs | | | |
| GATA3 | P426fs | PRPRRCTRHPACPLDHTTPP | APSESPCSPF (SEQ ID NO: | Breast Cancer |
| | H434fs | AWSPPWVRALLDAHRAPS | 708)(B07.02) | |
| | P433fs | ESPCSPFRLAFLQEQYHEA* | CPLDHTTPPA (SEQ ID | |
| | T441fs | (SEQ ID NO: 112) | NO: 709)(B07.02) | |
| | | | FLQEQYHEA (SEQ ID NO: | |
| | | | 710)(A02.01, B08.01) | |
| | | | RLAFLQEQYH (SEQ ID | |
| | | | NO: 711)(A03.01) | |
| | | | SPCSPFRLAF (SEQ ID NO: | |
| | | | 712)(B07.02) | |
| | | | SPPWVRALL (SEQ ID NO: | |
| | | | 713)(B07.02) | |
| | | | YPACPLDHTT (SEQ ID | |
| | | | NO: 714)(B07.02) | |
| MLL2 | P519fs | TRRCHCCPHLRSHPCPHHL | ALHLRSCPC (SEQ ID NO: | STAD, BLCA, |
| | E524fs | RNHPRPHHLRHHACHHHL | 715)(B08.01) | CRC, HNSC, |
| | P647fs | RNCPHPHFLRHCTCPGRWR | CLHHRRHLV (SEQ ID NO: | BRCA |
| | S654fs | NRPSLRRLRSLLCLPHLNH | 716)(B08.01) | |
| | L656fs | HLFLHWRSRPCLHRKSHPH | CLHHRRHLVC (SEQ ID | |
| | R755fs | LLHLRRLYPHHLKHRPCPH | NO: 717)(B08.01) | |
| | L761fs | HLKNLLCPRHLRNCPLPRH | CLHRKSHPHL (SEQ ID | |
| | Q773fs | LKHLACLHHLRSHPCPLHL | NO: 718)(B08.01) | |
| | | KSHPCLHHRRHLVCSHHLK | CLRSHACPP (SEQ ID NO: | |
| | | SLLCPLHLRSLPFPHHLRHH | 719)(B08.01) | |
| | | ACPHHLRTRLCPHHLKNHL | CLRSHTCPP (SEQ ID NO: | |
| | | CPPHLRYRAYPPCLWCHAC | 720)(B08.01) | |
| | | LHRLRNLPCPHRLRSLPRPL | CLWCHACLH (SEQ ID | |
| | | HLRLHASPHHLRTPPHPHH | NO. 721)(A03.01) | |
| | | LRTHLLPHHRRTRSCPCRW | CPHHLKNHL (SEQ ID NO: | |
| | | RSHPCCHYLRSRNSAPGPR | 722)(B07.02) | |
| | | GRTCHPGLRSRTCPPGLRS | CPHHLKNLL (SEQ ID NO: | |
| | | HTYLRRLRSHTCPPSLRSH | 723)(B07.02) | |
| | | AYALCLRSHTCPPRLRDHI | CPHHLRTRL (SEQ ID NO: | |
| | | CPLSLRNCTCPPRLRSRTCL | 724)(B07.02, B08.01) | |
| | | LCLRSHACPPNLRNHTCPPS | CPLHLRSLPF (SEQ ID NO: | |
| | | LRSHACPPGLRNRICPLSLR | 725)(B07.02, B08.01) | |
| | | SHPCPLGLKSPLRSQANAL | CPLPRHLKHL (SEQ ID | |
| | | HLRSCPCSLPLGNHPYLPCL | NO. 726)(B07.02, B08.01) | |
| | | ESQPCLSLGNHLCPLCPRSC | CPLSLRSHPC (SEQ ID NO: | |
| | | RCPHLGSHPCRLS* (SEQ ID | 727)(B07.02) | |
| | | NO: 113) | CPRHLRNCPL (SEQ ID | |
| | | | NO. 728)(B07.02, B08.01) | |
| | | | FPHHLRHHA (SEQ ID NO: | |
| | | | 729)(B07.02, B08.01) | |
| | | | FPHHLRHHAC (SEQ ID | |
| | | | NO: 730)(B07.02, B08.01) | |
| | | | GLRSRTCPP (SEQ ID NO: | |
| | | | 731)(B08.01) | |
| | | | HACLHRLRNL (SEQ ID | |
| | | | NO: 732)(B08.01) | |
| | | | HLACLHHLR (SEQ ID NO: | |
| | | | 733)(A03.01) | |
| | | | HLCPPHLRY (SEQ ID NO: | |
| | | | 734)(A03.01) | |
| | | | HLCPPHLRYR (SEQ ID | |
| | | | NO: 735)(A03.01) | |
| | | | HLKHLACLH (SEQ ID NO: | |
| | | | 736)(A03.01) | |
| | | | HLKHRPCPH (SEQ ID NO: | |
| | | | 737)(B08.01) | |
| | | | HLKNHLCPP (SEQ ID NO: | |
| | | | 738)(B08.01) | |
| | | | HLKSHPCLH (SEQ ID NO: | |
| | | | 739)(A03.01) | |
| | | | HLKSLLCPL (SEQ ID NO: | |
| | | | 740)(A02.01, B08.01) | |
| | | | HLLHLRRLY (SEQ ID NO: | |
| | | | 741)(A03.01) | |
| | | | HLRNCPLPR (SEQ ID NO: | |
| | | | 742)(A03.01) | |
| | | | HLRNCPLPRH (SEQ ID | |
| | | | NO: 743)(A03.01) | |
| | | | HLRRLYPHHL (SEQ ID | |
| | | | NO: 744)(B08.01) | |
| | | | HLRSHPCPL (SEQ ID NO: | |
| | | | 745)(B07.02, B08.01) | |
| | | | HLRSHPCPLH (SEQ ID | |
| | | | NO: 746)(A03.01) | |
| | | | HLRSLPFPH (SEQ ID NO: | |
| | | | 747)(A03.01) | |
| | | | HLRTRLCPH (SEQ ID NO: | |
| | | | 748)(A03.01, B08.01) | |
| | | | HLVCSHHLK (SEQ ID NO: | |
| | | | 749)(A03.01) | |
| | | | HPCLHHRRHL (SEQ ID | |
| | | | NO: 750)(B07.02, B08.01) | |
| | | | HPGLRSRTC (SEQ ID NO: | |
| | | | 751)(B07.02) | |
| | | | HPHLLHLRRL (SEQ ID | |
| | | | NO: 752)(B07.02, B08.01) | |
| | | | HRKSHPHLL (SEQ ID NO: | |
| | | | 753)(B08.01) | |
| | | | HRRTRSCPC (SEQ ID NO: | |
| | | | 754)(B08.01) | |
| | | | KSHPHLLHLR (SEQ ID | |
| | | | NO: 755)(A03.01) | |
| | | | KSLLCPLHLR (SEQ ID | |
| | | | NO: 756)(A03.01) | |
| | | | LLCPLHLRSL (SEQ ID NO: | |
| | | | 757)(A02.01, B08.01) | |
| | | | LLHLRRLYPH (SEQ ID | |
| | | | NO: 758)(B08.01) | |
| | | | LPRHLKHLA (SEQ ID NO: | |
| | | | 759)(B07.02) | |
| | | | LPRHLKHLAC (SEQ ID | |
| | | | NO: 760)(B07.02, B08.01) | |
| | | | LRRLRSHTC (SEQ ID NO: | |
| | | | 761)(B08.01) | |
| | | | LRRLYPHHL (SEQ ID NO: | |
| | | | 762)(B08.01) | |
| | | | LVCSHHLKSL (SEQ ID | |
| | | | NO: 763)(B08.01) | |
| | | | NLRNHTCPPS (SEQ ID | |
| | | | NO: 764)(B08.01) | |
| | | | PLHLRSLPF (SEQ ID NO: | |
| | | | 765)(B08.01) | |
| | | | RLCPHHLKNH (SEQ ID | |
| | | | NO: 766)(A03.01) | |
| | | | RLYPHHLKH (SEQ ID NO: | |
| | | | 767)(A03.01) | |
| | | | RLYPHHLKHR (SEQ ID | |
| | | | NO: 768)(A03.01) | |
| | | | RPCPHHLKNL (SEQ ID | |
| | | | NO: 769)(B07.02) | |
| | | | RSHPCPLHLK (SEQ ID | |
| | | | NO: 770)(A03.01) | |
| | | | RSLPFPHHLR (SEQ ID NO: | |
| | | | 771)(A03.01) | |
| | | | RTRLCPHHL (SEQ ID NO: | |
| | | | 772)(B07.02) | |
| | | | RTRLCPHHLK (SEQ ID | |
| | | | NO: 773)(A03.01) | |
| | | | SLLCPLHLR (SEQ ID NO: | |
| | | | 774)(A03.01) | |
| | | | SLRSHACPP (SEQ ID NO: | |
| | | | 775)(B08.01) | |
| | | | SPLRSQANA (SEQ ID NO: | |
| | | | 776)(B07.02) | |
| | | | YLRRLRSHT (SEQ ID NO: | |
| | | | 777)(B08.01) | |
| | | | YPHHLKHRPC (SEQ ID | |
| | | | NO: 778)(B07.02, B08.01) | |
| PTEN | I122fs | SWKGTNWCNDMCIFITSGQ | FITSGQIFK (SEQ ID NO: | UCEC, PRAD, |
| | I135fs | IFKGTRGPRFLWGSKDQRQ | 779)(A03.01) | SKCM, STAD, |
| | A148fs | KGSNYSQSEALCVLL* | IFITSGQIF (SEQ ID NO: | BRCA, LUSC, |
| | L152fs | (SEQ ID NO: 114) | 780)(A24.02) | KIRC, LIHC, |
| | D162fs | | SQSEALCVL (SEQ ID NO: | KIRP, GBM |
| | I168fs | | 781)(A02.01) | |
| | | | SQSEALCVLL (SEQ ID | |
| | | | NO: 782)(A02.01) | |
| PTEN | L265fs | KRTKCFTFG* (SEQ ID NO: | | UCEC, PRAD, |
| | K266fs | 115) | | SKCM, STAD, |
| | | | | BRCA, LUSC, |
| | | | | KIRC, LIHC, |
| | | | | KIRP, GBM |
| PTEN | A39fs | PIFIQTLLLWDFLQKDLKAY | AYTGTILMM (SEQ ID NO: | UCEC, PRAD, |
| | E40fs | TGTILMM* (SEQ ID NO: | 783)(A24.02) | SKCM, STAD, |
| | V45fs | 116) | DLKAYTGTIL (SEQ ID | BRCA, LUSC, |
| | R47fs | | NO: 784)(B08.01) | KIRC, LIHC, |
| | N48fs | | | KIRP, GBM |
| PTEN | T319fs | QKMILTKQIKTKPTDTFLQI | ILTKQIKTK (SEQ ID NO: | UCEC, PRAD, |
| | T321fs | LR* (SEQ ID NO: 117) | 785)(A03.01) | SKCM, STAD, |
| | K327fs | | KMILTKQIK (SEQ ID NO: | BRCA, LUSC, |
| | A328fs | | 786)(A03.01) | KIRC, LIHC, |
| | A333fs | | KPTDTFLQI (SEQ ID NO: | KIRP, GBM |
| | | | 787)(B07.02) | |
| | | | KPTDTFLQIL (SEQ ID NO: | |
| | | | 788)(B07.02) | |
| | | | MILTKQIKTK (SEQ ID | |
| | | | NO: 789)(A03.01) | |
| PTEN | N63fs | GFWIQSIKTITRYTIFVLKDI | ITRYTIFVLK (SEQ ID NO: | UCEC, PRAD, |
| | E73fs | MTPPNLIAELHNILLKTITH | 790)(A03.01) | SKCM, STAD, |
| | A86fs | HS* (SEQ ID NO: 118) | LIAELHNIL (SEQ ID NO: | BRCA, LUSC, |
| | N94fs | | 791)(A02.01) | KIRC, LIHC, |
| | | | LIAELHNILL (SEQ ID NO: | KIRP, GBM |
| | | | 792)(A02.01) | |
| | | | MTPPNLIAEL (SEQ ID NO: | |
| | | | 793)(A02.01) | |
| | | | NLIAELHNI (SEQ ID NO: | |
| | | | 794)(A02.01) | |
| | | | NLIAELHNIL (SEQ ID NO: | |
| | | | 795)(A02.01) | |
| | | | RYTIFVLKDI (SEQ ID NO: | |
| | | | 796)(A24.02) | |
| | | | TITRYTIFVL (SEQ ID NO: | |
| | | | 797)(A02.01) | |
| | | | TPPNLIAEL (SEQ ID NO: | |
| | | | 798)(B07.02) | |
| PTEN | T202fs | NYSNVQWRNLQSSVCGLP | FLQFRTHTT (SEQ ID NO: | UCEC, PRAD, |
| | G209fs | AKGEDIFLQFRTHTTGRQV | 799)(A02.01, B08.01) | SKCM, STAD, |
| | C211fs | HVL* (SEQ ID NO: 119) | LPAKGEDIFL (SEQ ID NO: | BRCA, LUSC, |
| | I224fs | | 800)(B07.02) | KIRC, LIHC, |
| | G230fs | | LQFRTHTTGR (SEQ ID | KIRP, GBM |
| | P231fs | | NO: 801)(A03.01) | |
| | R233fs | | NLQSSVCGL (SEQ ID NO: | |
| | D236fs | | 802)(A02.01) | |
| | | | SSVCGLPAK (SEQ ID NO: | |
| | | | 803)(A03.01) | |
| | | | VQWRNLQSSV (SEQ ID | |
| | | | NO: 804)(A02.01) | |
| PTEN | G251fs | YQSRVLPQTEQDAKKGQN | GQNVSLLGK (SEQ ID NO: | UCEC, PRAD, |
| | E256fs | VSLLGKYILHTRTRGNLRK | 805)(A03.01) | SKCM, STAD, |
| | K260fs | SRKWKSM* (SEQ ID NO: | HTRTRGNLRK (SEQ ID | BRCA, LUSC, |
| | Q261fs | 120) | NO: 806)(A03.01) | KIRC, LIHC, |
| | L265fs | | ILHTRTRGNL (SEQ ID | KIRP, GBM |
| | M270fs | | NO: 807)(B08.01) | |
| | H272fs | | KGQNVSLLGK (SEQ ID | |
| | T286fs | | NO: 808)(A03.01) | |
| | E288fs | | LLGKYILHT (SEQ ID NO: | |
| | | | 809)(A02.01) | |
| | | | LRKSRKWKSM (SEQ ID | |
| | | | NO: 810)(B08.01) | |
| | | | SLLGKYILH (SEQ ID NO: | |
| | | | 811)(A03.01) | |
| | | | SLLGKYILHT (SEQ ID NO: | |
| | | | 812)(A02.01) | |
| TP53 | A70fs | SSQNARGCSPRGPCTSSSYT | CTSPLLAPV (SEQ ID NO: | BRCA, CRC, |
| | P72fs | GGPCTSPLLAPVIFCPFPEN | 813)(A02.01) | LUAD, PRAD, |
| | A76fs | LPGQLRFPSGLLAFWDSQV | FPENLPGQL (SEQ ID NO: | HNSC, LUSC, |
| | A79fs | CDLHVLPCPQQDVLPTGQD | 814)(B07.02) | PAAD, STAD, |
| | P89fs | LPCAAVG* (SEQ ID NO: | GLLAFWDSQV (SEQ ID | BLCA, OV, |
| | W91fs | 121) | NO: 815)(A02.01) | LIHC, SKCM, |
| | S96fs | | IFCPFPENL (SEQ ID NO: | UCEC, LAML, |
| | V97fs | | 816)(A24.02) | UCS, KICH, |
| | V97fs | | LLAFWDSQV (SEQ ID NO: | GBM, ACC |
| | G108fs | | 817)(A02.01) | |
| | G117fs | | LLAPVIFCP (SEQ ID NO: | |
| | S121fs | | 818)(A02.01) | |
| | V122fs | | LLAPVIFCPF (SEQ ID NO: | |
| | C124fs | | 819)(A02.01, A24.02) | |
| | K139fs | | LPCPQQDVL (SEQ ID NO: | |
| | V143fs | | 820)(B07.02) | |
| | | | RFPSGLLAF (SEQ ID NO: | |
| | | | 821)(A24.02) | |
| | | | RFPSGLLAFW (SEQ ID | |
| | | | NO: 822)(A24.02) | |
| | | | SPLLAPVIF (SEQ ID NO: | |
| | | | 823)(B07.02) | |
| | | | SPRGPCTSS (SEQ ID NO: | |
| | | | 824)(B07.02) | |
| | | | SPRGPCTSSS (SEQ ID NO: | |
| | | | 825)(B07.02) | |
| | | | SQVCDLHVL (SEQ ID NO: | |
| | | | 826)(A02.01) | |
| | | | VIFCPFPENL (SEQ ID NO: | |
| | | | 827)(A02.01) | |
| TP53 | V173fs | GAAPTMSAAQIAMVWPLL | AMVWPLLSI (SEQ ID NO: | BRCA, CRC, |
| | H178fs | SILSEWKEICVWSIWMTET | 828)(A02.01) | LUAD, PRAD, |
| | D186fs | LFDIVWWCPMSRLRLALTV | AMVWPLLSIL (SEQ ID | HNSC, LUSC, |
| | H193fs | PPSTTTTCVTVPAWAA* | NO: 829)(A02.01) | PAAD, STAD, |
| | L194fs | (SEQ ID NO: 122) | AQIAMVWPL (SEQ ID NO: | BLCA, OV, |
| | E198fs | | 830)(A02.01, A24.02) | LIHC, SKCM, |
| | V203fs | | AQIAMVWPLL (SEQ ID | UCEC, LAML, |
| | E204fs | | NO: 831)(A02.01) | UCS, KICH, |
| | L206fs | | CPMSRLRLA (SEQ ID NO: | GBM, ACC |
| | D207fs | | 832)(B07.02, B08.01) | |
| | N210fs | | CPMSRLRLAL (SEQ ID | |
| | T211fs | | NO: 833)(B07.02, B08.01) | |
| | F212fs | | IAMVWPLLSI (SEQ ID | |
| | V225fs | | NO: 834)(A02.01, A24.02, | |
| | S241fs | | B08.01) | |
| | | | ILSEWKEICV (SEQ ID NO: | |
| | | | 835)(A02.01) | |
| | | | IVWWCPMSR (SEQ ID | |
| | | | NO: 836)(A03.01) | |
| | | | IVWWCPMSRL (SEQ ID | |
| | | | NO: 837)(A02.01) | |
| | | | IWMTETLFDI (SEQ ID NO: | |
| | | | 838)(A24.02) | |
| | | | LLSILSEWK (SEQ ID NO: | |
| | | | 839)(A03.01) | |
| | | | MSAAQIAMV (SEQ ID | |
| | | | NO: 840)(A02.01) | |
| | | | MSRLRLALT (SEQ ID NO: | |
| | | | 841)(B08.01) | |
| | | | MSRLRLALTV (SEQ ID | |
| | | | NO: 842)(B08.01) | |
| | | | MVWPLLSIL (SEQ ID NO: | |
| | | | 843)(A02.01) | |
| | | | RLALTVPPST (SEQ ID NO: | |
| | | | 844)(A02.01) | |
| | | | TLFDIVWWC (SEQ ID NO: | |
| | | | 845)(A02.01) | |
| | | | TLFDIVWWCP (SEQ ID | |
| | | | NO: 846)(A02.01) | |
| | | | TMSAAQIAMV (SEQ ID | |
| | | | NO: 847)(A02.01) | |
| | | | VWSIWMTETL (SEQ ID | |
| | | | NO: 848)(A24.02) | |
| | | | WMTETLFDI (SEQ ID NO: | |
| | | | 849)(A02.01, A24.02) | |
| | | | WMTETLFDIV (SEQ ID | |
| | | | NO: 850)(A01.01, A02.01) | |
| TP53 | R248fs | TGGPSSPSSHWKTPVVIYW | ALRCVFVPV (SEQ ID NO: | BRCA, CRC, |
| | P250fs | DGTALRCVFVPVLGETGAQ | 851)(A02.01, B08.01) | LUAD, PRAD, |
| | S260fs | RKRISARKGSLTTSCPQGAL | ALRCVFVPVL (SEQ ID | HNSC, LUSC, |
| | N263fs | SEHCPTTPAPLPSQRRNHW | NO: 852)(A02.01, B08.01) | PAAD, STAD, |
| | G266fs | MENISPFRSVGVSASRCSES* | ALSEHCPTT (SEQ ID NO: | BLCA, OV, |
| | N268fs | (SEQ ID NO: 123) | 853)(A02.01) | LIHC, SKCM, |
| | V272fs | | AQRKRISARK (SEQ ID | UCEC, LAML, |
| | V274fs | | NO: 854)(A03.01) | UCS, KICH, |
| | P278fs | | GAQRKRISA (SEQ ID NO: | GBM, ACC |
| | D281fs | | 855)(B08.01) | |
| | R282fs | | HWMENISPF (SEQ ID NO: | |
| | T284fs | | 856)(A24.02) | |
| | E285fs | | LPSQRRNHW (SEQ ID NO: | |
| | L289fs | | 857)(B07.02) | |
| | K292fs | | LPSQRRNHWM (SEQ ID | |
| | P301fs | | NO: 858)(B07.02, B08.01) | |
| | S303fs | | NISPFRSVGV (SEQ ID NO: | |
| | T312fs | | 859)(A02.01) | |
| | S314fs | | RISARKGSL (SEQ ID NO: | |
| | K319fs | | 860)(B07.02, B08.01) | |
| | K320fs | | SPFRSVGVSA (SEQ ID | |
| | P322fs | | NO: 861)(B07.02) | |
| | Y327fs | | SPSSHWKTPV (SEQ ID | |
| | F328fs | | NO: 862)(B07.02, B08.01) | |
| | L330fs | | TALRCVFVPV (SEQ ID | |
| | R333fs | | NO: 863)(A02.01) | |
| | R335fs | | VIYWDGTAL (SEQ ID NO: | |
| | R337fs | | 864)(A02.01) | |
| | E339fs | | VIYWDGTALR (SEQ ID | |
| | | | NO: 865)(A03.01) | |
| | | | VLGETGAQRK (SEQ ID | |
| | | | NO: 866)(A03.01) | |
| TP53 | S149fs | FHTPARHPRPRHGHLQAVT | HPRPRHGHL (SEQ ID NO: | BRCA, CRC, |
| | P151fs | AHDGGCEALPPP* (SEQ ID | 867)(B07.02, B08.01) | LUAD, PRAD, |
| | P152fs | NO: 124) | HPRPRHGHLQ (SEQ ID | HNSC, LUSC, |
| | V157fs | | NO: 868)(B07.02) | PAAD, STAD, |
| | Q165fs | | RPRHGHLQA (SEQ ID NO: | BLCA, OV, |
| | S166fs | | 869)(B07.02) | LIHC, SKCM, |
| | H168fs | | RPRHGHLQAV (SEQ ID | UCEC, LAML, |
| | V173fs | | NO: 870)(B07.02, B08.01) | UCS, KICH, |
| | | | | GBM, ACC |
| TP53 | P47fs | CCPRTILNNGSLKTQVQMK | GSLKTQVQMK (SEQ ID | BRCA, CRC, |
| | D48fs | LPECQRLLPPWPLHQQLLH | NO: 871)(A03.01) | LUAD, PRAD, |
| | D49fs | RRPLHQPPPGPCHLLSLPRK | PPGPCHLLSL (SEQ ID NO: | HNSC, LUSC, |
| | Q52fs | PTRAATVSVWASCILGQPS | 872)(B07.02) | PAAD, STAD, |
| | F54fs | L* (SEQ ID NO: 125) | RTILNNGSLK (SEQ ID | BLCA, OV, |
| | E56fs | | NO: 873)(A03.01) | LIHC, SKCM, |
| | P58fs | | SLKTQVQMK (SEQ ID | UCEC, LAML, |
| | P60fs | | NO: 874)(A03.01) | UCS, KICH, |
| | E62fs | | SLKTQVQMKL (SEQ ID | GBM, ACC |
| | M66fs | | NO: 875)(B08.01) | |
| | P72fs | | TILNNGSLK (SEQ ID NO: | |
| | V73fs | | 876)(A03.01) | |
| | P75fs | | | |
| | A78fs | | | |
| | P82fs | | | |
| | P85fs | | | |
| | S96fs | | | |
| | P98fs | | | |
| | T102fs | | | |
| | Y103fs | | | |
| | G108fs | | | |
| | F109fs | | | |
| | R110fs | | | |
| | G117fs | | | |
| TP53 | L26fs | VRKHFQTYGNYFLKTTFCP | CPPCRPKQWM (SEQ ID | BRCA, CRC, |
| | P27fs | PCRPKQWMI* (SEQ ID NO: | NO: 877)(B07.02) | LUAD, PRAD, |
| | P34fs | 126) | TTFCPPCRPK (SEQ ID NO: | HNSC, LUSC, |
| | P36fs | | 878)(A03.01) | PAAD, STAD, |
| | A39fs | | | BLCA, OV, |
| | Q38fs | | | LIHC, SKCM, |
| | | | | UCEC, LAML, |
| | | | | UCS, KICH, |
| | | | | GBM, ACC |
| TP53 | C124fs | LARTPLPSTRCFANWPRPA | CFANWFPRPAL (SEQ ID | BRCA, CRC, |
| | L130fs | LCSCGLIPHPRPAPASAPWP | NO: 879)(A24.02) | LUAD, PRAD, |
| | N131fs | STSSHST* (SEQ ID NO: 127) | FANWFPRPAL (SEQ ID NO: | HNSC, LUSC, |
| | C135fs | | 880)(B07.02, B08.01) | PAAD, STAD, |
| | K139fs | | GLIPHPRPA (SEQ ID NO: | BLCA, OV, |
| | A138fs | | 881)(A02.01) | LIHC, SKCM, |
| | T140fs | | HPRPAPASA (SEQ ID NO: | UCEC, LAML, |
| | V143fs | | 882)(B07.02, B08.01) | UCS, KICH, |
| | Q144fs | | HPRPAPASAP (SEQ ID | GBM, ACC |
| | V147fs | | NO: 883)(B07.02) | |
| | T150fs | | IPHPRPAPA (SEQ ID NO: | |
| | P151fs | | 884)(B07.02, B08.01) | |
| | P152fs | | IPHPRPAPAS (SEQ ID NO: | |
| | G154fs | | 885)(B07.02) | |
| | R156fs | | RPALCSCGL (SEQ ID NO: | |
| | R158fs | | 886)(B07.02) | |
| | A161fs | | RPALCSCGLI (SEQ ID NO: | |
| | | | 887)(B07.02) | |
| | | | TPLPSTRCF (SEQ ID NO: | |
| | | | 888)(B07.02) | |
| | | | WPRPALCSC (SEQ ID NO: | |
| | | | 889)(B07.02) | |
| | | | WPRPALCSCG (SEQ ID | |
| | | | NO: 890)(B07.02) | |
| VHL | L178fs | ELQETGHRQVALRRSGRPP | ALRRSGRPPK (SEQ ID | KIRC, KIRP |
| | D179fs | KCAERPGAADTGAHCTST | NO: 891)(A03.01) | |
| | L184fs | DGRLKISVETYTVSSQLLM | GLVPSLVSK (SEQ ID NO: | |
| | T202fs | VLMSLDLDTGLVPSLVSKC | 892)(A03.01) | |
| | R205fs | LILRVK* (SEQ ID NO: 128) | KISVETYTV (SEQ ID NO: | |
| | D213fs | | 893)(A02.01) | |
| | G212fs | | LLMVLMSLDL (SEQ ID | |
| | | | NO: 894)(A02.01, B08.01) | |
| | | | LMSLDLDTGL (SEQ ID | |
| | | | NO: 895)(A02.01) | |
| | | | LMVLMSLDL (SEQ ID NO: | |
| | | | 896)(A02.01) | |
| | | | LVSKCLILRV (SEQ ID NO: | |
| | | | 897)(A02.01) | |
| | | | QLLMVLMSL (SEQ ID NO: | |
| | | | 898)(A02.01, B08.01) | |
| | | | RPGAADTGA (SEQ ID NO: | |
| | | | 899)(B07.02) | |
| | | | RPGAADTGAH (SEQ ID | |
| | | | NO: 900)(B07.02) | |
| | | | SLDLDTGLV (SEQ ID NO: | |
| | | | 901)(A02.01) | |
| | | | SLVSKCLIL (SEQ ID NO: | |
| | | | 902)(A02.01, B08.01) | |
| | | | SQLLMVLMSL (SEQ ID | |
| | | | NO: 903)(A02.01) | |
| | | | TVSSQLLMV (SEQ ID NO: | |
| | | | 904)(A02.01) | |
| | | | TYTVSSQLL (SEQ ID NO: | |
| | | | 905)(A24.02) | |
| | | | TYTVSSQLLM (SEQ ID | |
| | | | NO: 906)(A24.02) | |
| | | | VLMSLDLDT (SEQ ID NO: | |
| | | | 907)(A02.01) | |
| | | | VPSLVSKCL (SEQ ID NO: | |
| | | | 908)(B07.02) | |
| | | | VSKCLILRVK (SEQ ID | |
| | | | NO: 909)(A03.01) | |
| | | | YTVSSQLLM (SEQ ID NO: | |
| | | | 910)(A01.01) | |
| | | | YTVSSQLLMV (SEQ ID | |
| | | | NO: 911)(A02.01) | |
| VHL | L158fs | KSDASRLSGA* (SEQ ID | | KIRC, KIRP |
| | K159fs | NO: 129) | | |
| | R161fs | | | |
| | Q164fs | | | |
| VHL | P146fs | RTAYFCQYHTASVYSERA | FCQYHTASV (SEQ ID NO: | KIRC, KIRP |
| | I147fs | MPPGCPEPSQA* (SEQ ID | 912)(B08.01) | |
| | F148fs | NO: 130) | | |
| | L158fs | | | |
| VHL | S68fs | TRASPPRSSSAIAVRASCCP | CPYGSTSTA (SEQ ID NO: | KIRC, KIRP |
| | S72fs | YGSTSTASRSPTQRCRLAR | 913)(B07.02) | |
| | I75fs | AAASTATEVTFGSSEMQGH | CPYGSTSTAS (SEQ ID | |
| | S80fs | TMGFWLTKLNYLCHLSML | NO: 914)(B07.02) | |
| | P86fs | TDSLFLPISHCQCIL* (SEQ | LARAAASTAT (SEQ ID | |
| | P97fs | ID NO: 131) | NO: 915)(B07.02) | |
| | I109fs | | MLTDSLFLP (SEQ ID NO: | |
| | H115fs | | 916)(A02.01) | |
| | L116fs | | PPRSSSAIAV (SEQ ID NO: | |
| | G123fs | | 917)(B07.02) | |
| | T124fs | | RAAASTATEV (SEQ ID | |
| | N131fs | | NO: 918)(B07.02) | |
| | L135fs | | SPPRSSSAI (SEQ ID NO: | |
| | V137fs | | 919)(B07.02) | |
| | G144fs | | SPPRSSSAIA (SEQ ID NO: | |
| | D143fs | | 920)(B07.02) | |
| | I147fs | | SPTQRCRLA (SEQ ID NO: | |
| | | | 921)(B07.02) | |
| | | | TQRCRLARA (SEQ ID NO: | |
| | | | 922)(B08.01) | |
| | | | TQRCRLARAA (SEQ ID | |
| | | | NO: 923)(B08.01) | |
| VHL | K171fs | SSLRITGDWTSSGRSTKIWK | KIWKTTQMCR (SEQ ID | KIRC, KIRP |
| | P172fs | TTQMCRKTWSG* (SEQ ID | NO: 924)(A03.01) | |
| | N174fs | NO: 132) | WTSSGRSTK (SEQ ID NO: | |
| | L178fs | | 925)(A03.01) | |
| | D179fs | | | |
| | L188fs | | | |
| VHL | V62fs | RRRRGGVGRRGVRPGRVR | ALGELARAL (SEQ ID NO: | KIRC, KIRP |
| | V66fs | PGGTGRRGGDGGRAAAAR | 926)(A02.01) | |
| | Q73fs | AALGELARALPGHLLQSQS | AQLRRRAAA (SEQ ID NO: | |
| | V84fs | ARRAARMAQLRRRAAALP | 927)(B08.01) | |
| | F91fs | NAAAWHGPPHPQLPRSPLA | AQLRRRAAAL (SEQ ID | |
| | T100fs | LQRCRDTRWASG* (SEQ ID | NO: 928)(B08.01) | |
| | P103fs | NO: 133) | ARRAARMAQL (SEQ ID | |
| | S111fs | | NO: 929)(B08.01) | |
| | L116fs | | HPQLPRSPL (SEQ ID NO: | |
| | H115fs | | 930)(B07.02, B08.01) | |
| | D126fs | | HPQLPRSPLA (SEQ ID | |
| | | | NO. 931)(B07.02) | |
| | | | LARALPGHL (SEQ ID NO: | |
| | | | 932)(B07.02) | |
| | | | LARALPGHLL (SEQ ID | |
| | | | NO: 933)(B07.02) | |
| | | | MAQLRRRAA (SEQ ID | |
| | | | NO: 934)(B07.02, B08.01) | |
| | | | MAQLRRRAAA (SEQ ID | |
| | | | NO: 935)(B07.02, B08.01) | |
| | | | QLRRRAAAL (SEQ ID NO: | |
| | | | 936)(B07.02, B08.01) | |
| | | | RAAALPNAAA (SEQ ID | |
| | | | NO: 937)(B07.02) | |
| | | | RMAQLRRRAA (SEQ ID | |
| | | | NO: 938)(B07.02, B08.01) | |
| | | | SQSARRAARM (SEQ ID | |
| | | | NO: 939)(B08.01) | |

TABLE 1D
CRYPTIC EXON

| | | | | |
|---|---|---|---|---|
| AR-v7 | cryptic | SCKVFFKRAAEGKQKYLC | GMTLGEKFRV (SEQ ID | Prostate Cancer, |
| | final | ASRNDCTIDKFRRKNCPSC | NO: 940) (A02:01) | Castration- |
| | exon | RLRKCYEAGMTLGEKFRV | RVGNCKEILK (SEQ ID | resistant |
| | | GNCKEILKMTRP* (SEQ ID | NO: 941) (A03.01) | Prostate Cancer |
| | | NO: 134) | | |

TABLE 1E
OUT OF FRAME FUSIONS

| | | | | |
|---|---|---|---|---|
| AC011997.1: | AC011997.1: | MAGAPPPASLPPCSLISDCC | GPSEPGNNI (SEQ ID NO: | LUSC, Breast |
| LRRC69 | LRRC69 | ASNQRDSVGVGPSEP:G: [symbol] | 942) (B07.02) | Cancer, Head |
| | *out-of- | [symbol] (SEQ ID | KICNESASRK (SEQ ID | and Neck |
| | frame | NO: 135) | NO: 943) (A03.01) | Cancer, LUAD |
| EEF1DP3 | EEF1DP3: | HGWRPFLPVRARSRWNRR | GIQVLNVSLK (SEQ ID | Breast Cancer |
| | FRY | LDVTVANGR:S: [symbol] | NO: 944) (A03.01) | |
| | *out-of- | [symbol] | IQVLNVSLK (SEQ ID NO: | |
| | | [symbol] | 945) (A03.01) | |
| | | (SEQ ID NO: 136) | KSSSNVISY (SEQ ID NO: | |
| | | | 946) (A01.01, A03.01) | |
| | | | KYGWSLLRV (SEQ ID | |
| | | | NO: 947) (A24.02) | |
| | | | RSWKYGWSL (SEQ ID | |
| | | | NO: 948) (A02.01) | |
| | | | SLKSSSNVI (SEQ ID NO: | |
| | | | 949) (B08.01) | |
| | | | SWKYGWSLL (SEQ ID | |
| | | | NO: 950) (A24.02) | |
| | | | TVANGRSWK (SEQ ID | |
| | | | NO: 951) (A03.01) | |
| | | | VPQVNGIQV (SEQ ID NO: | |
| | | | 952) (B07.02) | |
| | | | VPQVNGIQVL (SEQ ID | |
| | | | NO: 953) (B07.02) | |
| | | | VTVANGRSWK (SEQ ID | |
| | | | NO: 954) (A03.01) | |
| | | | WSLLRVPQV (SEQ ID NO: | |
| | | | 955) (B08.01) | |
| MAD1L1: | MAD1L1: | RLKEVFQTKIQEFRKACYT | HPGDCLIFKL (SEQ ID NO: | CLL |
| MAFK | MAFK | LTGYQIDITTENQYRLTSLY | 956) (B07.02) | |
| | | AEHPGDCLIFK:: [symbol] | KLRVPGSSV (SEQ ID NO: | |
| | | [symbol] (SEQ ID NO: | 957) (B07.02) | |
| | | 137) | KLRVPGSSVL (SEQ ID | |
| | | | NO: 958) (B07.02) | |
| | | | RVPGSSVLV (SEQ ID NO: | |
| | | | 959) (A02.01) | |
| | | | SVLVTVPGL (SEQ ID NO: | |
| | | | 960) (A02.01) | |
| | | | VPGSSVLVTV (SEQ ID | |
| | | | NO: 961) (B07.02) | |
| PPP1R1B: | PPP1R1B: | AEVLKVIRQSAGQKTTCGQ | ALLLRPRPPR (SEQ ID NO: | Breast Cancer |
| STARD3 | STARD3 | GLEGPWERPPPLDESERDG | 962) (A03.01) | |
| | | GSEDQVEDPALS:A: [symbol] | ALSALLLRPR (SEQ ID | |
| | | [symbol] | NO: 963) (A03.01) | |
| | | [symbol] | | |
| | | [symbol] | | |
| | | (SEQ ID NO: 138) | | |

TABLE 1F
IN-FRAME DELETIONS and FUSIONS

| | | | | |
|---|---|---|---|---|
| BCR:ABL | BCR:ABL | ERAEWRENIREQQKKCFRS | LTINKEEAL (SEQ ID NO: | CML, AML |
| | | FSLTSVELQMLTNSCVKLQ | 964) (A02.01, B08.01) | |
| | | TVHSIPLTINKE::EALQRPV | | |
| | | ASDFEPQGLSEAARWNSK | | |
| | | ENLLAGPSENDPNLFVAL | | |
| | | YDFVASG (SEQ ID NO: 139) | | |
| BCR:ABL | BCR:ABL | ELQMLTNSCVKLQTVHSIP | IVHSATGFK (SEQ ID NO: | CML, AML |
| | | LTINKEDDESPGLYGFLNVI | 965) (A03.01) | |
| | | VHSATGFKQSS:K:ALQRPV | ATGFKQSSK (SEQ ID NO: | |
| | | ASDFEPQGLSEAARWNSK | 966) (A03.01) | |
| | | ENLLAGPSENDPNLFVAL | | |
| | | YDFVASGD (SEQ ID NO: | | |
| | | 140) | | |
| C11orf95:RELA | C11orf95:RELA | ISNSWDAHLGLGACGEAEG | ELFPLIFPA (SEQ ID NO: | Supretentorial |
| | | LGVQGAEEEEEEEEEEEEE | 967) (A02.01, B08.01) | ependyomas |
| | | GAGVPACPPKGP:E:LFPLIF | KGPELFPLI (SEQ ID NO: | |
| | | PAEPAQASGPYVEIIEQPK | 968) (A02.01, A24.02) | |
| | | QRGMRFRYKCEGRSAGSI | KGPELFPLIF (SEQ ID NO: | |
| | | PGERSTD (SEQ ID NO: 141) | 969) (A24.02) | |
| CBFB:MYH11 | (variant | LQRLDGMGCLEFDEERAQ | | AML |
| | “type a”) | QEDALAQQAFEEARRRTRE | | |
| | | FEDRDRSHREEME::VHELE | | |
| | | KSKRALETQMEEMKTQL | | |
| | | EELEDELQATEDAKLRLE | | |
| | | VNMQALKGQF (SEQ ID | | |
| | | NO: 142) | | |
| CD74:ROS1 | (exon6:exon32) | KGSFPENLRHLKNTMETID | KPTDAPPKAGV (SEQ ID | NSCLC, |
| | | WKVFESWMEIHWILFEMS | NO: 970) (B07.02) | Crizotinib |
| | | RHSLEQKPTDAPPK::AGVP | | resistance |
| | | NKPGIPKLLEGSKNSIQW | | |
| | | EKAEDNGCRITYYILEIRK | | |
| | | STSNNLQNQ (SEQ ID NO: | | |
| | | 143) | | |
| EGFR | EGFRvIII | MRPSGTAGAALLALLAAL | ALEEKKGNYV (SEQ ID | GBM |
| | (internal | CPASRALEEKK:G:NYVVTD | NO: 971) (A02.01) | |
| | deletion) | HGSCVRACGADSYEMEED | | |
| | | GVRKCKKCEGPCRKVCNGI | | |
| | | GIGEFKD (SEQ ID NO: 144) | | |
| EGFR:SEPT14 | EGFR:SEPT14 | LPQPPICTIDVYMIMVKCW | IQLQDKFEHL (SEQ ID | GBM, Glioma, |
| | | MIDADSRPKFRELIIEFSKM | NO: 972) (A02.01, B08.01) | Head and Neck |
| | | ARDPQRYLVIQ::LQDKFEH | QLQDKFEHL (SEQ ID NO: | Cancer |
| | | LKMQQEEIRKLEEEKKQ | 973) (A02.01, B08.01) | |
| | | LEGEHDFYKMKAASEAL | QLQDKFEHLK (SEQ ID | |
| | | QTQLSTD (SEQ ID NO: 145) | NO: 974) (A03.01) | |
| | | | YLVIQLQDKF (SEQ ID | |
| | | | NO: 975) (A02.01, A24.02) | |
| EML4:ALK | EML4:ALK | SWENSDDSRNKLSKIPSTPK | QVYRRKHQEL (SEQ ID | NSCLC |
| | | LIPKVTKTADKHKDVIINQ | NO: 976) (B08.01) | |
| | | AKMSTREKNSQ:V:YRRKH | STREKNSQV (SEQ ID NO: | |
| | | QELQAMQMELQSPEYKL | 977) (B08.01) | |
| | | SKLRTSTIMTDYNPNYCF | VYRRKHQEL (SEQ ID NO: | |
| | | AGKTSSISDL (SEQ ID NO: | 978) (A24.02, B08.01) | |
| | | 146) | | |
| FGFR3:TACC3 | FGFR3:TACC3 | EGHRMDKPANCTHDLYMI | VLTVTSTDV (SEQ ID NO: | Bladder Cancer, |
| | | MRECWHAAPSQRPTFKQL | 979) (A02.01) | LUSC |
| | | VEDLDRVLTVTSTD::VKAT | VLTVTSTDVK (SEQ ID | |
| | | QEENRELRSRCEELHGKN | NO: 980) (A03.01) | |
| | | LELGKIMDRFEEVVYQA | | |
| | | MEEVQKQKELS (SEQ ID | | |
| | | NO: 147) | | |
| NAB:STAT6 | NAB:STAT6 “” | RDNTLLLRRVELFSLSRQV | IMSLWGLVS (SEQ ID NO: | Solitary fibrous |
| | | ARESTYLSSLKGSRLEIPEEL | 981) (A02.01) | tumors |
| | | GGPPLKKLKQE::ATSKSQI | IMSLWGLVSK (SEQ ID | |
| | | MSLWGLVSKMPPEKVQR | NO: 982) (A03.01) | |
| | | LYVDFPQHLRHLLGDWL | KLKQEATSK (SEQ ID NO: | |
| | | ESQPWEFLVGSDAFCC | 983) (A03.01) | |
| | | (SEQ ID NO: 148) | QIMSLWGLV (SEQ ID NO: | |
| | | | 984) (A02.01) | |
| | | | SQIMSLWGL (SEQ ID NO: | |
| | | | 985) (A02.01, A24.02, | |
| | | | B08.01) | |
| | | | SQIMSLWGLV (SEQ ID | |
| | | | NO: 986) (A02.01) | |
| | | | TSKSQIMSL (SEQ ID NO: | |
| | | | 987) (B08.01) | |
| NDRG1:ERG | NDRG1:ERG | MSREMQDVDLAEVKPLVE | LLQEFDVQEA (SEQ ID | Prostate Cancer |
| | | KGETITGLLQEFDVQ::EAL | NO: 988) (A02.01) | |
| | | SVVSEDQSLFECAYGTPH | LQEFDVQEAL (SEQ ID | |
| | | LAKTEMTASSSSDYGQTS | NO: 989) (A02.01) | |
| | | KMSPRVPQQDW (SEQ ID | | |
| | | NO: 149) | | |
| PML:RARA | PML:RARA | VLDMEIGFLRQALCRLRQE | | Acute |
| | (exon3:exon3) | EPQSLQAAVRTDGFDEFKV | | promyelocytic |
| | | RLQDLSSCITQGK:A:IETQS | | leukemia |
| | | SSSEEIVPSPPSPPPLPRIY | | |
| | | KPCFVCQDKSSGYHYGVS | | |
| | | ACEGCKG (SEQ ID NO: | | |
| | | 150) | | |
| PML:RARA | PML:RARA | RSSPEQPRPSTSKAVSPPEIL | | Acute |
| | (exon6:exon3) | DGPPSPRSPVIGSEVFLPNS | | promyelocytic |
| | | NHVASGAGEA:A:IETQSSS | | leukemia |
| | | SEEIVPSPPSPPPLPRIYKP | | |
| | | CFVCQDKSSGYHYGVSAC | | |
| | | EGCKG (SEQ ID NO: 151) | | |
| RUNX1 | RUNX1 | VARFNDLRFVGRSGRGKSF | GPREPRNRT (SEQ ID NO: | AML |
| | (ex5)- | TLTITVFTNPPQVATYHRAI | 990) (B07.02) | |
| | RUNX1 | KITVDGPREPR:N:RTEKHS | RNRTEKHSTM (SEQ ID | |
| | T1(ex2) | TMPDSPVDVKTQSRLTPP | NO: 991) (B08.01) | |
| | | TMPPPPTTQGAPRTSSFTP | | |
| | | TTLTNGT (SEQ ID NO: 152) | | |
| TMPRSS2:ERG | TMPRSS2:ERG | MALNS::EALSVVSEDQSLF | ALNSEALSV (SEQ ID NO: | Prostate Cancer |
| | | ECAYGTPHLAKTEMTASSS | 992) (A02.01) | |
| | | SDYGQTSKMSPRVPQQDW | ALNSEALSVV (SEQ ID | |
| | | (SEQ ID NO: 153) | NO: 993) (A02.01) | |
| | | | MALNSEALSV (SEQ ID | |
| | | | NO: 994) (A02.01, B08.01) | |

TABLE 2A

| | | | | |
|---|---|---|---|---|
| | Amino Acid | Mutation Sequence | | Exemplary |
| Gene | Alteration | Context | Peptides (HLA allele example(s)) | Diseases |

POINT MUTATIONS¹

| | | | | |
|---|---|---|---|---|
| AKT1 | E17K | MSDVAIVKEGWLH | KYIKTWRPRY (SEQ ID NO: | BRCA, CESC, |
| | | KRGKYIKTWRPRYF | 1005) (A24.02) | HNSC, LUSC, |
| | | LLKNDGTFIGYKERP | WLHKRGKYI (SEQ ID NO: | PRAD, SKCM, |
| | | QDVDQREAPLNNFS | 1006) (A02.01, B07.02, B08.01) | THCA |
| | | VAQCQLMKTER | WLHKRGKYIK (SEQ ID NO: | |
| | | (SEQ ID NO: 995) | 1007) (A03.01) | |
| ANAPC1 | T537A | TMLVLEGSGNLVLY | APKPLSKLL (SEQ ID NO: 1008) | GBM, LUSC, |
| | | TGVVRVGKVFIPGLP | (B07.02) | PAAD, PRAD, |
| | | APSLTMSNTMPRPST | GVSAPKPLSK (SEQ ID NO: | SKCM |
| | | PLDGVSAPKPLSKLL | 1009) (A03.01) | |
| | | GSLDEVVLLSPVPEL | VSAPKPLSK (SEQ ID NO: 1010) | |
| | | RDSSKLEIDSLYNED | (A03.01) | |
| | | CTFQQLGTYIHSI | | |
| | | (SEQ ID NO: 996) | | |
| FGFR3 | S249C | HRIGGIKLRHQQWS | CPHRPILQA (SEQ ID NO: 1011) | BLCA, HNSC, |
| | | LVMESVVPSDRGNY | (B07.02) | KIRP, LUSC |
| | | TCVVENKFGSIRQTY | | |
| | | TLDVLERCPHRPILQ | | |
| | | AGLPANQTAVLGSD | | |
| | | VEFHCKVYSDAQPH | | |
| | | IQWLKHVEVNGSKV | | |
| | | G (SEQ ID NO: 33) | | |
| FRG1B | I10T | MREPIYMHSTMVFL | KLSDSRTAL (SEQ ID NO: 1012) | KIRP, PRAD, |
| | | PWELHTKKGPSPPE | (A02.01, B07.02, B08.01) | SKCM |
| | | QFMAVKLSDSRTAL | KLSDSRTALK (SEQ ID NO: | |
| | | KSGYGKYLGINSDE | 1013) (A03.01) | |
| | | LVGHSDAIGPREQW | LSDSRTALK (SEQ ID NO: 1014) | |
| | | EPVFQNGKMALLAS | (A01.01, A03.01) | |
| | | NSCFIR (SEQ ID NO: | RTALKSGYGK (SEQ ID NO: | |
| | | 997) | 1015) (A03.01) | |
| | | | TALKSGYGK (SEQ ID NO: | |
| | | | 1016) (A03.01) | |
| FRG1B | L52S | AVKLSDSRIALKSGY | ALSASNSCF (SEQ ID NO: 1017) | GBM, KIRP, |
| | | GKYLGINSDELVGH | (A02.01, A24.02, B07.02) | PRAD, SKCM |
| | | SDAIGPREQWEPVF | ALSASNSCFI (SEQ ID NO: | |
| | | QNGKMALSASNSCF | 1018) (A02.01) | |
| | | IRCNEAGDIEAKSKT | FQNGKMALSA (SEQ ID NO: | |
| | | AGEEEMIKIRSCAEK | 1019) (A02.01, B08.01) | |
| | | ETKKKDDIPEEDKG | | |
| | | (SEQ ID NO: 34) | | |
| HER2 | L755S | AMPNQAQMRILKET | KVSRENTSPK (SEQ ID NO: | BRCA |
| | (Resistance) | ELRKVKVLGSGAFG | 1020) (A03.01) | |
| | | TVYKGIWIPDGENV | | |
| | | KIPVAIKVSRENTSP | | |
| | | KANKEILDEAYVMA | | |
| | | GVGSPYVSRLLGICL | | |
| | | TSTVQLVTQLMPYG | | |
| | | C (SEQ ID NO: 998) | | |
| IDH1 | R132G | RVEEFKLKQMWKSP | KPIIIGGHAY (SEQ ID NO: | BLCA, BRCA, |
| | | NGTIRNILGGTVFRE | 1021) (B07.02) | CRC, GBM, |
| | | AIICKNIPRLVSGWV | | HNSC, LUAD, |
| | | KPIIIGGHAYGDQYR | | PAAD, PRAD, |
| | | ATDFVVPGPGKVEIT | | UCEC |
| | | YTPSDGTQKVTYLV | | |
| | | HNFEEGGGVAMGM | | |
| | | (SEQ ID NO: 38) | | |
| KRAS | G12C | MTEYKLVVVGACG | KLVVVGACGV (SEQ ID NO: | BRCA, CESC, |
| | | VGKSALTIQLIQNHF | 154) (A02.01) | CRC, HNSC, |
| | | VDEYDPTIEDSYRK | LVVVGACGV (SEQ ID NO: | LUAD, PAAD, |
| | | QVVIDGETCLLDILD | 155) (A02.01) | UCEC |
| | | TAGQE (SEQ ID NO: | VVGACGVGK (SEQ ID NO: | |
| | | 8) | 156) (A03.01, A11.01) | |
| | | | VVVGACGVGK (SEQ ID NO: | |
| | | | 157) (A03.01) | |
| KRAS | G12D | MTEYKLVVVGADG | VVGADGVGK (SEQ ID NO: | BLCA, BRCA, |
| | | VGKSALTIQLIQNHF | 158) (A11.01) | CESC, CRC, |
| | | VDEYDPTIEDSYRK | VVVGADGVGK (SEQ ID NO: | GBM, HNSC, |
| | | QVVIDGETCLLDILD | 159) (A11.01) | KIRP, LIHC, |
| | | TAGQE (SEQ ID NO: | KLVVVGADGV (SEQ ID NO: | LUAD, PAAD, |
| | | 9) | 160) (A02.01) | SKCM, UCEC |
| | | | LVVVGADGV (SEQ ID NO: | |
| | | | 161) (A02.01) | |
| KRAS | G12V | MTEYKLVVVGAVG | KLVVVGAVGV (SEQ ID NO: | BRCA, CESC, |
| | | VGKSALTIQLIQNHF | 162) (A02.01) | CRC, LUAD, |
| | | VDEYDPTIEDSYRK | LVVVGAVGV (SEQ ID NO: | PAAD, THCA, |
| | | QVVIDGETCLLDILD | 163) (A02.01) | UCEC |
| | | TAGQE (SEQ ID NO: | VVGAVGVGK (SEQ ID NO: | |
| | | 10) | 164) (A03.01, A11.01) | |
| | | | VVVGAVGVGK (SEQ ID NO: 5) | |
| | | | (A03.01, A11.01) | |
| KRAS | Q61H | AGGVGKSALTIQLIQ | ILDTAGHEEY (SEQ ID NO: | CRC, LUSC, |
| | | NHFVDEYDPTIEDSY | 165) (A01.01) | PAAD, SKCM, |
| | | RKQVVIDGETCLLDI | | UCEC |
| | | LDTAGHEEYSAMRD | | |
| | | QYMRTGEGFLCVFA | | |
| | | INNTKSFEDIEHYRE | | |
| | | QIKRVKDSEDVPM | | |
| | | (SEQ ID NO: 11) | | |
| KRAS | Q61L | AGGVGKSALTIQLIQ | ILDTAGLEEY (SEQ ID NO: 166) | CRC, GBM, |
| | | NHFVDEYDPTIEDSY | (A01.01) | HNSC, LUAD, |
| | | RKQVVIDGETCLLDI | LLDILDTAGL (SEQ ID NO: 167) | SKCM, UCEC |
| | | LDTAGLEEYSAMRD | (A02.01) | |
| | | QYMRTGEGFLCVFA | | |
| | | INNTKSFEDIEHYRE | | |
| | | QIKRVKDSEDVPM | | |
| | | (SEQ ID NO: 12) | | |
| NRAS | Q61K | AGGVGKSALTIQLIQ | ILDTAGKEEY (SEQ ID NO: | BLCA, CRC, |
| | | NHFVDEYDPTIEDSY | 168) (A01.01) | LIHC, LUAD, |
| | | RKQVVIDGETCLLDI | | LUSC, SKCM, |
| | | LDTAGKEEYSAMRD | | THCA, UCEC |
| | | QYMRTGEGFLCVFA | | |
| | | INNSKSFADINLYRE | | |
| | | QIKRVKDSDDVPM | | |
| | | (SEQ ID NO: 13) | | |
| NRAS | Q61R | AGGVGKSALTIQLIQ | ILDTAGREEY (SEQ ID NO: | BLCA, CRC, |
| | | NHFVDEYDPTIEDSY | 169) (A01.01) | LUSC, PAAD, |
| | | RKQVVIDGETCLLDI | | PRAD, SKCM, |
| | | LDTAGREEYSAMRD | | THCA, UCEC |
| | | QYMRTGEGFLCVFA | | |
| | | INNSKSFADINLYRE | | |
| | | QIKRVKDSDDVPM | | |
| | | (SEQ ID NO: 14) | | |
| PIK3CA | E542K | IEEHANWSVSREAG | AISTRDPLSK (SEQ ID NO: | BLCA, BRCA, |
| | | FSYSHAGLSNRLAR | 1022) (A03.01) | CESC, CRC, |
| | | DNELRENDKEQLKA | | GBM, HNSC, |
| | | ISTRDPLSKITEQEKD | | KIRC, KIRP, |
| | | FLWSHRHYCVTIPEI | | LIHC, LUAD, |
| | | LPKLLLSVKWNSRD | | LUSC, PRAD, |
| | | EVAQMYCLVKDWP | | UCEC |
| | | P (SEQ ID NO: 48) | | |
| PTEN | R130Q | KFNCRVAQYPFEDH | QTGVMICAY (SEQ ID NO: | BRCA, CESC, |
| | | NPPQLELIKPFCEDL | 1023) (A01.01) | CRC, GBM, |
| | | DQWLSEDDNHVAAI | | KIRC, LUSC, |
| | | HCKAGKGQTGVMIC | | UCEC |
| | | AYLLHRGKFLKAQE | | |
| | | ALDFYGEVRTRDKK | | |
| | | GVTIPSQRRYVYYY | | |
| | | SY (SEQ ID NO: 52) | | |
| RAC1 | P29S | MQAIKCVVVGDGA | FSGEYIPTV (SEQ ID NO: 1024) | Melanoma |
| | | VGKTCLLISYTTNAF | (A02.01) | |
| | | SGEYIPTVFDNYSAN | TTNAFSGEY (SEQ ID NO: | |
| | | VMVDGKPVNLGLW | 1025) (A01.01) | |
| | | DTAGQEDYDRLRPL | YTTNAFSGEY (SEQ ID NO: | |
| | | SYPQTVGET (SEQ ID | 1026) (A01.01) | |
| | | NO: 53) | | |
| SF3B1 | K700E | AVCKSKKSWQARH | GLVDEQQEV (SEQ ID NO: | AML associated |
| | | TGIKIVQQIAILMGC | 1027) (A02.01) | with MDS; |
| | | AILPHLRSLVEIIEHG | | Chronic |
| | | LVDEQQEVRTISALA | | lymphocytic |
| | | IAALAEAATPYGIES | | leukaemia-small |
| | | FDSVLKPLWKGIRQ | | lymphocytic |
| | | HRGKGLAAFLKAI | | lymphoma; |
| | | (SEQ ID NO: 999) | | Myelodysplastic |
| | | | | syndrome; AML; |
| | | | | Luminal NS |
| | | | | carcinoma of |
| | | | | breast; Chronic |
| | | | | myeloid |
| | | | | leukaemia; Ductal |
| | | | | carcinoma of |
| | | | | pancreas; Chronic |
| | | | | myelomonocytic |
| | | | | leukaemia; |
| | | | | Chronic |
| | | | | lymphocytic |
| | | | | leukaemia-small |
| | | | | lymphocytic |
| | | | | lymphoma; |
| | | | | Myelofibrosis; |
| | | | | Myelodysplastic |
| | | | | syndrome; PRAD; |
| | | | | Essential |
| | | | | thrombocythaemia; |
| | | | | Medullomyoblastoma |
| SPOP | F133L | YLSLYLLLVSCPKSE | FVQGKDWGL (SEQ ID NO: | PRAD |
| | | VRAKFKFSILNAKGE | 1028) (A02.01, B08.01) | |
| | | ETKAMESQRAYRFV | | |
| | | QGKDWGLKKFIRRD | | |
| | | FLLDEANGLLPDDK | | |
| | | LTLFCEVSVVQDSV | | |
| | | NISGQNTMNMVKVP | | |
| | | E (SEQ ID NO: 1000) | | |
| SPOP | F133V | YLSLYLLLVSCPKSE | FVQGKDWGV (SEQ ID NO: | PRAD |
| | | VRAKFKFSILNAKGE | 1029) (A02.01) | |
| | | ETKAMESQRAYRFV | | |
| | | QGKDWGVKKFIRRD | | |
| | | FLLDEANGLLPDDK | | |
| | | LTLFCEVSVVQDSV | | |
| | | NISGQNTMNMVKVP | | |
| | | E (SEQ ID NO: 1001) | | |
| TP53 | G245S | IRVEGNLRVEYLDD | CMGSMNRRPI (SEQ ID NO: | BLCA, BRCA, |
| | | RNTFRHSVVVPYEPP | 1030) (A02.01, B08.01) | CRC, GBM, |
| | | EVGSDCTTIHYNYM | GSMNRRPIL (SEQ ID NO: 1031) | HNSC, LUSC, |
| | | CNSSCMGSMNRRPI | (B08.01) | PAAD, PRAD |
| | | LTIITLEDSSGNLLGR | MGSMNRRPI (SEQ ID NO: | |
| | | NSFEVRVCACPGRD | 1032) (B08.01) | |
| | | RRTEEENLRKKGEP | MGSMNRRPIL (SEQ ID NO: | |
| | | (SEQ ID NO: 54) | 1033) (B08.01) | |
| | | | SMNRRPILTI (SEQ ID NO: | |
| | | | 1034) (A02.01, A24.02, B08.01) | |
| TP53 | R248Q | EGNLRVEYLDDRNT | CMGGMNQRPI (SEQ ID NO: | BLCA, BRCA, |
| | | FRHSVVVPYEPPEV | 1035) (A02.01, B08.01) | CRC, GBM, |
| | | GSDCTTIHYNYMCN | GMNQRPILTI (SEQ ID NO: | HNSC, KIRC, |
| | | SSCMGGMNQRPILTI | 1036) (A02.01, B08.01) | LIHC, LUSC, |
| | | ITLEDSSGNLLGRNS | NQRPILTII (SEQ ID NO: 1037) | PAAD, PRAD, |
| | | FEVRVCACPGRDRR | (A02.01, B08.01) | UCEC |
| | | TEEENLRKKGEPHH | | |
| | | E (SEQ ID NO: 56) | | |
| TP53 | R248W | EGNLRVEYLDDRNT | CMGGMNWRPI (SEQ ID NO: | BLCA, BRCA, |
| | | FRHSVVVPYEPPEV | 1038) (A02.01, A24.02, B08.01) | CRC, GBM, |
| | | GSDCTTIHYNYMCN | GMNWRPILTI (SEQ ID NO: | HNSC, LIHC, |
| | | SSCMGGMNWRPILT | 1039) (A02.01, B08.01) | LUSC, PAAD, |
| | | IITLEDSSGNLLGRNS | MNWRPILTI (SEQ ID NO: 1040) | SKCM, UCEC |
| | | FEVRVCACPGRDRR | (A02.01, A24.02, B08.01) | |
| | | TEEENLRKKGEPHH | MNWRPILTII (SEQ ID NO: | |
| | | E (SEQ ID NO: 57) | 1041) (A02.01, A24.02) | |
| TP53 | R273C | PEVGSDCTTIHYNY | NSFEVCVCA (SEQ ID NO: | BLCA, BRCA, |
| | | MCNSSCMGGMNRR | 1042) (A02.01) | CRC, GBM, |
| | | PILTIITLEDSSGNLL | | HNSC, LUSC, |
| | | GRNSFEVCVCACPG | | PAAD, UCEC |
| | | RDRRTEEENLRKKG | | |
| | | EPHHELPPGSTKRAL | | |
| | | PNNTSSSPQPKKKPL | | |
| | | (SEQ ID NO: 58) | | |
| TP53 | R273H | PEVGSDCTTIHYNY | NSFEVHVCA (SEQ ID NO: | BRCA, CRC, |
| | | MCNSSCMGGMNRR | 1043) (A02.01) | GBM, HNSC, |
| | | PILTIITLEDSSGNLL | | LIHC, LUSC, |
| | | GRNSFEVHVCACPG | | PAAD, UCEC |
| | | RDRRTEEENLRKKG | | |
| | | EPHHELPPGSTKRAL | | |
| | | PNNTSSSPQPKKKPL | | |
| | | (SEQ ID NO: 1002) | | |
| TP53 | Y220C | TEVVRRCPEIHERCS | VVPCEPPEV (SEQ ID NO: 1044) | BLCA, BRCA, |
| | | DSDGLAPPQHLIRVE | (A02.01) | GBM, HNSC, |
| | | GNLRVEYLDDRNTF | VVVPCEPPEV (SEQ ID NO: | LIHC, LUAD, |
| | | RHSVVVPCEPPEVGS | 1045) (A02.01) | LUSC, PAAD, |
| | | DCTTIHYNYMCNSS | | SKCM, UCEC |
| | | CMGGMNRRPILTIIT | | |
| | | LEDSSGNLLGRNSF | | |
| | | (SEQ ID NO: 1003) | | |

TABLE 2B
MSI-ASSOCIATED FRAMESHIFTS¹

| | | | | |
|---|---|---|---|---|
| MSH6 | F1088fs; +1 | YNFDKNYKDWQSA | ILLPEDTPPL (SEQ ID NO: 1046) | MSI+ CRC, MSI+ |
| | | VECIAVLDVLLCLA | (A02.01) | Uterine/Endometrium |
| | | NYSRGGDGPMCRPV | LLPEDTPPL (SEQ ID NO: 1047) | Cancer, MSI+ |
| | | ILLPEDTPPLLRA | (A02.01) | Stomach Cancer, |
| | | (SEQ ID NO: 1004) | | Lynch syndrome |

TABLE 2C
FRAMESHIFT¹

| | Amino Acid | Mutation Sequence | | Exemplary |
|---|---|---|---|---|
| Gene | Alteration | Context | Peptides (HLA allele example(s)) | Diseases |
| APC | F1354fs | AKFQQCHSTLEPNP | APFRVNHAV (SEQ ID NO: | CRC, LUAD, |
| | | ADCRVLVYLQNQPG | 1048)(B07.02) | UCEC, STAD |
| | | TKLLNFLQERNLPPK | CLADVLLSV (SEQ ID NO: | |
| | | VVLRHPKVHLNTMF | 1049)(A02.01) | |
| | | RRPHSCLADVLLSV | FLQERNLPPK (SEQ ID NO: | |
| | | HLIVLRVVRLPAPFR | 1050)(A03.01) | |
| | | VNHAVEW* (SEQ ID | HLIVLRVVRL (SEQ ID NO: | |
| | | NO: 95) | 1051)(A02.01, B08.01) | |
| | | | HPKVHLNTM (SEQ ID NO: | |
| | | | 1052)(B07.02, B08.01) | |
| | | | HPKVHLNTMF (SEQ ID NO: | |
| | | | 1053)(B07.02, B08.01) | |
| | | | KVHLNTMFR (SEQ ID NO: | |
| | | | 1054)(A03.01) | |
| | | | KVHLNTMFRR (SEQ ID NO: | |
| | | | 1055)(A03.01) | |
| | | | LPAPFRVNHA (SEQ ID NO: | |
| | | | 1056)(B07.02) | |
| | | | MFRRPHSCL (SEQ ID NO: | |
| | | | 1057)(B07.02, B08.01) | |
| | | | MFRRPHSCLA (SEQ ID NO: | |
| | | | 1058)(B08.01) | |
| | | | NTMFRRPHSC (SEQ ID NO: | |
| | | | 1059)(B08.01) | |
| | | | RPHSCLADV (SEQ ID NO: | |
| | | | 1060)(B07.02) | |
| | | | RPHSCLADVL (SEQ ID NO: | |
| | | | 1061)(B07.02) | |
| | | | RVVRLPAPFR (SEQ ID NO: | |
| | | | 1062)(A03.01) | |
| | | | SVHLIVLRV (SEQ ID NO: 1063) | |
| | | | (A02.01) | |
| | | | TMFRRPHSC (SEQ ID NO: | |
| | | | 1064)(B08.01) | |
| | | | TMFRRPHSCL (SEQ ID NO: | |
| | | | 1065)(A02.01, B08.01) | |
| | | | VLLSVHLIV (SEQ ID NO: 1066) | |
| | | | (A02.01) | |
| | | | VLLSVHLIVL (SEQ ID NO: | |
| | | | 1067)(A02.01) | |
| | | | VLRVVRLPA (SEQ ID NO: | |
| | | | 1068)(B08.01) | |
| | | | VVRLPAPFR (SEQ ID NO: 1069) | |
| | | | (A03.01) | |

A subset of peptides from Table 1 (n=562) were synthesized and their affinity for their given HLA class I molecule was measured as described. The values are shown in Table 3. These data show a strong correlation between prediction and measurement (dotted line represents best fit, R²=0.45), demonstrating the value of the predictions. However, the outliers demonstrate the importance of these measurements. Thick vertical and horizontal lines are shown at 500 nM for the predicted affinity and observed affinity, respectively. 500 nM is commonly accepted in the field as the maximum affinity for an epitope that is a “weak binder” to HLA class I. Therefore, the points in the lower right quadrant (prediction greater than 500 nM, measurement less than 500 nM) are epitopes that were considered very weak binders but were observed to bind within an acceptable range. Epitopes in this quadrant (n=75) represent 30.5% of epitopes not considered to be binders by prediction (combination of bottom right and top right quadrants, n=246).

TABLE 3

| | | | | | Observed | |
|---|---|---|---|---|---|---|
| | | | | Predicted | Affinity | |
| | | | SEQ ID | affinity | (IC50; | Stability |
| Mutation | Allele | Peptide | NO: | (IC50; (nM)) | (nM)) | (T1/2 (h)) |
| ABL1, M351T | A02.01 | TQISSATEYL | 208 | 2921.0 | 2644.0 | 0 |
| ABL1, T315I | A02.01 | YIIIEFMTYG | 214 | 3502.0 | 186.0 | 0 |
| ABL1, T315I | A02.01 | IIIEFMTYG | 212 | 1991.0 | 779.0 | 0 |
| ABL1, T315I | A02.01 | IIIEFMTYGN | 213 | 16793.0 | 1551.0 | 0 |
| ABL1, T315I | A02.01 | IIEFMTYGNL | 211 | 2134.0 | 9702.0 | 0 |
| ABL1, Y253H | A02.01 | KLGGGQHGEV | 216 | 1705.0 | 387.0 | 0.4 |
| AKT1, E17K | B08.01 | WLHKRGKYI | 1006 | 47.0 | 417.0 | 1.3 |
| AKT1, E17K | A02.01 | WLHKRGKYI | 1006 | 4972.0 | 1250.0 | 1.2 |
| AKT1, E17K | B07.02 | WLHKRGKYI | 1006 | 7185.0 | 2648.0 | 0 |
| ALK, G1269A | A02.01 | RVAKIADFGM | 218 | 5258.0 | 125.0 | 0.5 |
| ALK, G1269A | B07.02 | RVAKIADFGM | 218 | 7260.0 | 9723.0 | 0.2 |
| ALK, L1196M | A02.01 | SLPRFILMEL | 226 | 94.0 | 26.0 | 0.5 |
| ALK, L1196M | A02.01 | ILMELMAGG | 220 | 192.0 | 223.0 | 0.5 |
| ALK, L1196M | A02.01 | LMELMAGGDL | 222 | 5617.0 | 311.0 | 8.9 |
| ALK, L1196M | A02.01 | LQSLPRFILM | 225 | 2519.0 | 413.0 | 0 |
| ALK, L1196M | B07.02 | SLPRFILMEL | 226 | 17.0 | 583.0 | 0.4 |
| ALK, L1196M | B08.01 | LQSLPRFILM | 225 | 1288.0 | 1547.0 | 0 |
| ALK, L1196M | A02.01 | FILMELMAGG | 219 | 189.0 | 1580.0 | 0 |
| ALK, L1196M | B08.01 | SLPRFILMEL | 226 | 686.0 | 1762.0 | 0 |
| ALK, L1196M | A24.02 | SLPRFILMEL | 226 | 5143.0 | 2774.0 | 0.2 |
| ALK, L1196M | A02.01 | ILMELMAGGD | 221 | 5761.0 | 3451.0 | 0 |
| APC, AVEW | A02.01 | VLLSVHLIV | 1066 | 36.0 | 72.0 | 11 |
| (SEQ ID NO: | | | | | | |
| 1130) | | | | | | |
| APC, AVEW | A02.01 | CLADVLLSV | 1049 | 5.0 | 219.0 | 24 |
| (SEQ ID NO: | | | | | | |
| 1130) | | | | | | |
| APC, VHPA | A02.01 | KVLQMDFLV | 479 | 25.0 | 11.0 | 6.4 |
| (SEQ ID NO: | | | | | | |
| 1131) | | | | | | |
| APC, VHPA | A02.01 | LQMDFLVEIPA | 481 | 26.0 | 68.0 | 1.5 |
| (SEQ ID NO: | | | | | | |
| 1131) | | | | | | |
| β2M, . . . MPAV | A03.01 | TTLNSPPLKK | 651 | 62.5 | 14.3 | not |
| (SEQ ID NO: | | | | | | measured |
| 1132) | | | | | | |
| β2M, . . . MPAV | A03.01 | TTLNSPPLK | 650 | 165.4 | 9.8 | not |
| (SEQ ID NO: | | | | | | measured |
| 1132) | | | | | | |
| β2M, . . . MPAV | A03.01 | TLNSPPLKK | 649 | 27.5 | 5.4 | not |
| (SEQ ID NO: | | | | | | measured |
| 1132) | | | | | | |
| β2M, . . . MPAV | A03.01 | CTTLNSPPLK | 646 | 225.3 | 63.6 | not |
| (SEQ ID NO: | | | | | | measured |
| 1132) | | | | | | |
| β2M, . . . MPAV | B08.01 | CLSARTGLSI | 645 | 1106.6 | 149.6 | not |
| (SEQ ID NO: | | | | | | measured |
| 1132) | | | | | | |
| β2M, . . . MPAV | A02.01 | GLSISCTTL | 647 | 669.0 | 114.5 | not |
| (SEQ ID NO: | | | | | | measured |
| 1132) | | | | | | |
| β2M, . . . SIRH | A03.01 | LTSSSREWK | 1070 | 413.9 | 117.8 | not |
| (SEQ ID NO: | | | | | | measured |
| 1133) | | | | | | |
| β2M, . . . SIRH | A03.01 | LLTSSSREWK | 1071 | 206.1 | 1769.8 | not |
| (SEQ ID NO: | | | | | | measured |
| 1133) | | | | | | |
| β2M, . . . SIRH | B07.02 | YPAYSKDSGL | 1072 | 41.1 | 79.5 | not |
| (SEQ ID NO: | | | | | | measured |
| 1133) | | | | | | |
| β2M, . . . SIRH | B08.01 | EWKVKFPEL | 1073 | 488.7 | 538.4 | not |
| (SEQ ID NO: | | | | | | measured |
| 1133) | | | | | | |
| β2M, . . . SIRH | A24.02 | KFPELLCVW | 1074 | 83.7 | 13.7 | not |
| (SEQ ID NO: | | | | | | measured |
| 1133) | | | | | | |
| β2M, . . . SQIS | B08.01 | LQRFRFTHV | 652 | 55.5 | 37.3 | not |
| (SEQ ID NO: | | | | | | measured |
| 1134) | | | | | | |
| β2M, . . . SQIS | A24.02 | RLSSVLQRF | 654 | 288.9 | 28.2 | not |
| (SEQ ID NO: | | | | | | measured |
| 1134) | | | | | | |
| β2M, . . . SQIS | A02.01 | VLQRFRFTHV | 656 | 163.4 | 106.7 | not |
| (SEQ ID NO: | | | | | | measured |
| 1134) | | | | | | |
| β2M, . . . SQIS | B08.01 | VLQRFRFTHV | 656 | 264.1 | 480.1 | not |
| (SEQ ID NO: | | | | | | measured |
| 1134) | | | | | | |
| β2M, . . . SQIS | A03.01 | RLSSVLQRFR | 655 | 168.1 | 12.5 | not |
| (SEQ ID NO: | | | | | | measured |
| 1134) | | | | | | |
| BCR: ABL | B08.01 | LTINKEEAL | 964 | 4972.0 | 895.0 | 0 |
| (e13a2, aka b2a2) | | | | | | |
| BCR: ABL | A02.01 | LTINKEEAL | 964 | 12671.0 | 4413.0 | 0 |
| (e13a2, aka b2a2) | | | | | | |
| BRAF, V600E | A02.01 | LATEKSRWSG | 228 | 39130.0 | 23337.0 | 0 |
| BRAF, V600E | B08.01 | LATEKSRWS | 227 | 24674.0 | 36995.0 | 0 |
| BRAF, V600E | B08.01 | LATEKSRWSG | 228 | 13368.0 | 46582.0 | 0 |
| BRAF, V600E | A02.01 | LATEKSRWS | 227 | 39109.0 | 60997.0 | 0 |
| BTK, C481S | A02.01 | SLLNYLREM | 173 | 48.0 | 87.0 | 3 |
| BTK, C481S | A02.01 | MANGSLLNYL | 172 | 2979.0 | 1082.0 | 0 |
| BTK, C481S | B07.02 | SLLNYLREM | 173 | 6544.0 | 1110.0 | 0 |
| BTK, C481S | B08.01 | SLLNYLREM | 173 | 1091.0 | 1230.0 | 0 |
| BTK, C481S | A02.01 | YMANGSLLN | 174 | 7856.0 | 4444.0 | 0 |
| BTK, C481S | B07.02 | MANGSLLNYL | 172 | 8921.0 | 17715.0 | 0 |
| BTK, C481S | B08.01 | MANGSLLNYL | 172 | 7639.0 | 19853.0 | 0 |
| BTK, C481S | A03.01 | MANGSLLNY | 171 | 1030.3 | 35.6 | not |
| | | | | | | measured |
| BTK, C481S | A01.01 | MANGSLLNY | 171 | 285.7 | 439.0 | not |
| | | | | | | measured |
| BTK, C481S | A24.02 | EYMANGSLL | 170 | 213.2 | 5.0 | not |
| | | | | | | measured |
| BTK, C481S | A01.01 | YMANGSLLNY | 175 | 95.7 | 13.2 | not |
| | | | | | | measured |
| BTK, C481S | A03.01 | YMANGSLLNY | 175 | 109.4 | 95.9 | not |
| | | | | | | measured |
| C11orf95: RELA | A02.01 | ELFPLIFPA | 967 | 13.0 | 13.0 | 5.1 |
| C11orf95: RELA | A24.02 | KGPELFPLI | 968 | 909.0 | 14.0 | 1.7 |
| C11orf95: RELA | A02.01 | KGPELFPLI | 968 | 6840.0 | 101.0 | 0.3 |
| C11orf95: RELA | B08.01 | ELFPLIFPA | 967 | 7316.0 | 449.0 | 0 |
| C15ORF40(+1) | A02.01 | KLFSCLSFL | 344 | 6.0 | 6.0 | 14.3 |
| C15ORF40(+1) | A03.01 | KLFSCLSFL | 344 | 1488.0 | 308.0 | 0.8 |
| C15ORF40(+1) | A03.01 | SLQPPPPGFK | 352 | 26.3 | 19.0 | not |
| | | | | | | measured |
| C15ORF40(+1) | A03.01 | LFFFFFETK | 347 | 658.4 | 413.3 | not |
| | | | | | | measured |
| C15ORF40(+1) | A02.01 | ALFFFFFET | 334 | 28.9 | 470.7 | not |
| | | | | | | measured |
| C15ORF40(+1) | A03.01 | ALFFFFFETK | 335 | 31.5 | 216.4 | not |
| | | | | | | measured |
| C15ORF40(+1) | A02.01 | FFFETKSCSV | 340 | 754.5 | 61.2 | not |
| | | | | | | measured |
| C15ORF40(+1) | B08.01 | FFETKSCSV | 339 | 807.6 | 7.6 | not |
| | | | | | | measured |
| C15ORF40(+1) | A01.01 | LSFLSSWDY | 348 | 211.1 | 52.9 | not |
| | | | | | | measured |
| C15ORF40(+1) | A02.01 | FLSSWDYRRM | 342 | 62.2 | 323.5 | not |
| | | | | | | measured |
| C15ORF40(+1) | A03.01 | LSFLSSWDYR | 349 | 508.7 | 100.9 | not |
| | | | | | | measured |
| C15ORF40(+1) | A02.01 | FKLFSCLSFL | 341 | 9.9 | 662.9 | not |
| | | | | | | measured |
| C15ORF40(+1) | A02.01 | VQWRSLGSL | 353 | 986.0 | 4733.2 | not |
| | | | | | | measured |
| C15ORF40(+1) | A02.01 | KLFSCLSFLS | 345 | 65.1 | 0.6 | not |
| | | | | | | measured |
| C15ORF40(+1) | A03.01 | KLFSCLSFLS | 345 | 805.1 | 104.0 | not |
| | | | | | | measured |
| C15ORF40(+1) | A02.01 | AQAGVQWRSL | 336 | 630.2 | 670.0 | not |
| | | | | | | measured |
| C15ORF40(+1) | A24.02 | RRMPPCLANF | 351 | 253.0 | 141.1 | not |
| | | | | | | measured |
| C15ORF40(+1) | A03.01 | CLSFLSSWDY | 338 | 890.5 | 2705.8 | not |
| | | | | | | measured |
| C15ORF40(+1) | A24.02 | GFKLFSCLSF | 343 | 387.4 | 643.0 | not |
| | | | | | | measured |
| C15ORF40(+1) | A24.02 | RMPPCLANF | 350 | 34.4 | 8.7 | not |
| | | | | | | measured |
| C15ORF40(+1) | A03.01 | CLANFCIFNR | 337 | 575.4 | 221.8 | not |
| | | | | | | measured |
| C15ORF40(+1) | A01.01 | CLSFLSSWDY | 338 | 538.7 | 987.3 | not |
| | | | | | | measured |
| CNOT1(+1) | A02.01 | SVCFFFFSV | 356 | 27.0 | 175.0 | 9.4 |
| CNOT1(+1) | B08.01 | SVCFFFFSV | 356 | 4940.0 | 10599.0 | 0 |
| CNOT1(+1) | A02.01 | MSVCFFFFSV | 355 | 131.0 | 1706.4 | not |
| | | | | | | measured |
| CNOT1(+1) | A02.01 | FFFSVIFST | 354 | 608.9 | 4556.0 | not |
| | | | | | | measured |
| CNOT1(−1) | A01.01 | MSVCFFFFCY | 359 | 310.4 | 4369.3 | not |
| | | | | | | measured |
| CNOT1(−1) | A02.01 | SVCFFFFCYI | 360 | 237.2 | 519.8 | not |
| | | | | | | measured |
| CNOT1(−1) | A24.02 | FFCYILNTMF | 358 | 583.4 | 73.4 | not |
| | | | | | | measured |
| EGFR, T790M | A02.01 | MQLMPFGCLL | 184 | 21.0 | 20.0 | 0.4 |
| EGFR, T790M | A02.01 | MQLMPFGCL | 183 | 842.0 | 166.0 | 0.4 |
| EGFR, T790M | A02.01 | LIMQLMPFGC | 181 | 1984.0 | 177.0 | 0.4 |
| EGFR, T790M | A02.01 | QLIMQLMPF | 185 | 2511.0 | 227.0 | 0.3 |
| EGFR, T790M | B08.01 | QLIMQLMPF | 185 | 891.0 | 388.0 | 0 |
| EGFR, T790M | B08.01 | IMQLMPFGCL | 179 | 1302.0 | 548.0 | 0 |
| EGFR, T790M | A02.01 | CLTSTVQLIM | 177 | 3465.0 | 716.0 | 0 |
| EGFR, T790M | A02.01 | IMQLMPFGCL | 179 | 143.0 | 837.0 | 0.5 |
| EGFR, T790M | A02.01 | IMQLMPFGC | 178 | 1123.0 | 1607.0 | 0.4 |
| EGFR, T790M | B07.02 | MQLMPFGCL | 183 | 10169.0 | 2270.0 | 0 |
| EGFR, T790M | A24.02 | QLIMQLMPF | 185 | 3209.0 | 2389.0 | 0.4 |
| EGFR, T790M | A02.01 | LIMQLMPFG | 180 | 4961.0 | 3513.0 | 0 |
| EGFR, T790M | A24.02 | VQLIMQLMPF | 188 | 1455.0 | 4559.0 | 0 |
| EGFR, T790M | A02.01 | VQLIMQLMPF | 188 | 4464.0 | 5492.0 | 0 |
| EGFR, T790M | A02.01 | QLIMQLMPFG | 186 | 5751.0 | 5926.0 | 0 |
| EGFR, T790M | B08.01 | MQLMPFGCL | 183 | 1105.0 | 7045.0 | 0 |
| EGFR, T790M | A02.01 | STVQLIMQL | 187 | 2151.0 | 8537.0 | 0 |
| EGFR, T790M | A01.01 | CLTSTVQLIM | 177 | 2998.0 | 11036.0 | 0 |
| EGFR, T790M | B08.01 | MQLMPFGCLL | 184 | 970.0 | 14056.0 | 0 |
| EGFR, T790M | B08.01 | VQLIMQLMPF | 188 | 3370.0 | 17898.0 | 0 |
| EGFR, T790M | A24.02 | IMQLMPFGCL | 179 | 4394.0 | 18102.0 | 0 |
| EGFR, T790M | A24.02 | MQLMPFGCLL | 184 | 4168.0 | 23572.0 | 0 |
| EGFR, T790M | A01.01 | LTSTVQLIM | 182 | 1000.7 | 2891.1 | not |
| | | | | | | measured |
| EGFR: SEPT14 | B08.01 | QLQDKFEHL | 973 | 917.0 | 989.0 | 0 |
| EGFR: SEPT14 | A02.01 | QLQDKFEHL | 973 | 422.0 | 1155.0 | 0.6 |
| EGFR: SEPT14 | A02.01 | YLVIQLQDKF | 975 | 9963.0 | 2057.0 | 0 |
| EGFR: SEPT14 | A24.02 | YLVIQLQDKF | 975 | 9508.0 | 2152.0 | 2.6 |
| EGFR: SEPT14 | A02.01 | IQLQDKFEHL | 972 | 820.0 | 4265.0 | 0.2 |
| EGFR: SEPT14 | B08.01 | IQLQDKFEHL | 972 | 4278.0 | 10247.0 | 0 |
| EGFRvIII | A02.01 | ALEEKKGNYV | 971 | 2445.0 | 141.0 | 0 |
| (internal deletion) | | | | | | |
| EIF2B3(−1) | A02.01 | KQWSSVTSL | 361 | 54.4 | 26.5 | not |
| | | | | | | measured |
| EML4: ALK | B08.01 | QVYRRKHQEL | 976 | 194.0 | 160.0 | 0 |
| EPHB2(−1) | A02.01 | ILIRKAMTV | 363 | 38.2 | 19.5 | not |
| | | | | | | measured |
| ESR1, D538G | A24.02 | PLYGLLLEML | 234 | 1519.0 | 444.0 | 6.3 |
| ESR1, D538G | A02.01 | GLLLEMLDA | 230 | 705.0 | 558.0 | 0.4 |
| ESR1, D538G | A02.01 | PLYGLLLEM | 233 | 349.0 | 640.0 | 0.7 |
| ESR1, D538G | A24.02 | VVPLYGLLL | 236 | 2965.0 | 658.0 | 0.8 |
| ESR1, D538G | A02.01 | PLYGLLLEML | 234 | 542.0 | 797.0 | 0 |
| ESR1, D538G | A02.01 | VVPLYGLLL | 236 | 4432.0 | 1039.0 | 0.6 |
| ESR1, D538G | A02.01 | NVVPLYGLL | 232 | 4835.0 | 10471.0 | 0 |
| ESR1, D538G | B07.02 | VPLYGLLLEM | 235 | 145.1 | 27.9 | not |
| | | | | | | measured |
| ESR1, D538G | A24.02 | LYGLLLEML | 231 | 218.3 | 0.8 | not |
| | | | | | | measured |
| ESR1, S463P | A02.01 | FLPSTLKSL | 237 | 71.0 | 21.0 | 2.1 |
| ESR1, S463P | A02.01 | GVYTFLPST | 238 | 307.0 | 779.0 | 1.3 |
| ESR1, S463P | A24.02 | FLPSTLKSL | 237 | 10723.0 | 995.0 | 1 |
| ESR1, S463P | A02.01 | GVYTFLPSTL | 239 | 248.0 | 1197.0 | 0.4 |
| ESR1, S463P | B08.01 | FLPSTLKSL | 237 | 2314.0 | 1968.0 | 0 |
| ESR1, S463P | A24.02 | GVYTFLPSTL | 239 | 954.0 | 7696.0 | 0 |
| ESR1, Y537C | A02.01 | PLCDLLLEM | 245 | 1067.0 | 602.0 | 0.8 |
| ESR1, Y537C | A02.01 | VVPLCDLLL | 248 | 5533.0 | 1200.0 | 0 |
| ESR1, Y537C | A02.01 | NVVPLCDLLL | 244 | 1964.0 | 1373.0 | 0 |
| ESR1, Y537C | A02.01 | PLCDLLLEML | 246 | 1320.0 | 2008.0 | 0.9 |
| ESR1, Y537C | A02.01 | NVVPLCDLL | 243 | 3473.0 | 3027.0 | 0 |
| ESR1, Y537C | A24.02 | VVPLCDLLL | 248 | 7992.0 | 3888.0 | 0.4 |
| ESR1, Y537N | A02.01 | PLNDLLLEM | 251 | 1062.0 | 151.0 | 4.2 |
| ESR1, Y537N | A02.01 | NVVPLNDLL | 249 | 4725.0 | 2900.0 | 0 |
| ESR1, Y537N | A02.01 | NVVPLNDLLL | 250 | 2606.0 | 4190.0 | 0 |
| ESR1, Y537N | A02.01 | PLNDLLLEML | 252 | 1741.0 | 11957.0 | 0 |
| ESR1, Y537S | A02.01 | PLSDLLLEM | 256 | 713.0 | 404.0 | 2.7 |
| ESR1, Y537S | A02.01 | NVVPLSDLLL | 255 | 2510.0 | 741.0 | 0 |
| ESR1, Y537S | A02.01 | NVVPLSDLL | 254 | 4259.0 | 916.0 | 0 |
| ESR1, Y537S | A02.01 | VVPLSDLLL | 259 | 8320.0 | 2551.0 | 0 |
| E5R1, Y537S | A02.01 | PLSDLLLEML | 257 | 1138.0 | 6469.0 | 0 |
| ESR1, Y537S | A24.02 | VVPLSDLLL | 259 | 8463.0 | 8252.0 | 0.5 |
| FAM111B(−1) | A03.01 | RMKVPLMK | 364 | 58.9 | 33.0 | not |
| | | | | | | measured |
| FGFR3, S249C | A02.01 | YTLDVLERC | 261 | 3309.0 | 1764.0 | 6.6 |
| FGFR3, S249C | B08.01 | VLERCPHRPI | 260 | 3629.0 | 7223.0 | 0 |
| FGFR3, S249C | A02.01 | VLERCPHRPI | 260 | 4505.0 | 15321.0 | 0 |
| FGFR3: TACC3 | A02.01 | VLTVTSTDV | 979 | 1255.0 | 295.0 | 1.1 |
| FRG1B, I1OT | B07.02 | KLSDSRTAL | 1012 | 225.0 | 9.0 | 6.8 |
| FRG1B, I1OT | A02.01 | KLSDSRTAL | 1012 | 275.0 | 111.0 | 2.8 |
| FRG1B, I1OT | B08.01 | KLSDSRTAL | 1012 | 3276.0 | 122.0 | 0 |
| FRG1B, L52S | A02.01 | ALSASNSCFI | 1018 | 327.0 | 226.0 | 0.8 |
| FRG1B, L52S | B08.01 | FQNGKMALSA | 1019 | 7796.0 | 425.0 | 0 |
| FRG1B, L52S | B07.02 | ALSASNSCF | 1017 | 13989.0 | 684.0 | 0 |
| FRG1B, L52S | A02.01 | ALSASNSCF | 1017 | 7913.0 | 728.0 | 0.3 |
| FRG1B, L52S | A02.01 | FQNGKMALS | 262 | 2305.0 | 3276.0 | 0 |
| FRG1B, L52S | A02.01 | FQNGKMALSA | 1019 | 1205.0 | 6158.0 | 0 |
| FRG1B, L52S | A24.02 | ALSASNSCF | 1017 | 9672.0 | 16338.0 | 0.2 |
| GATA3 . . . CSNH | B08.01 | FLKAESKIM | 1075 | 263.4 | 21.9 | not |
| (SEQ ID NO: | | | | | | measured |
| 1135) | | | | | | |
| GATA3 . . . CSNH | B08.01 | LQHGHRHGL | 1076 | 693.0 | 550.4 | not |
| (SEQ ID NO: | | | | | | measured |
| 1135) | | | | | | |
| GATA3 . . . CSNH | B07.02 | EPHLALQPL | 1077 | 106.6 | 17.0 | not |
| (SEQ ID NO: | | | | | | measured |
| 1135) | | | | | | |
| GATA3 . . . CSNH | B07.02 | RPLQTHVLPE | 706 | 968.0 | 2534.4 | not |
| (SEQ ID NO: | | | | | | measured |
| 1135) | | | | | | |
| GATA3 . . . CSNH | B08.01 | FATLQRSSL | 1078 | 138.0 | 26.6 | not |
| (SEQ ID NO: | | | | | | measured |
| 1135) | | | | | | |
| GATA3 . . . CSNH | B07.02 | MFATLQRSSL | 1079 | 1285.0 | 266.9 | not |
| (SEQ ID NO: | | | | | | measured |
| 1135) | | | | | | |
| GATA3 . . . CSNH | A24.02 | MFLKAESKI | 1080 | 1065.7 | 332.1 | not |
| (SEQ ID NO: | | | | | | measured |
| 1135) | | | | | | |
| GATA3 . . . CSNH | B07.02 | FATLQRSSL | 1078 | 261.9 | 14.0 | not |
| (SEQ ID NO: | | | | | | measured |
| 1135) | | | | | | |
| GATA3 . . . CSNH | A02.01 | MLTGPPARV | 1081 | 145.4 | 10.6 | not |
| (SEQ ID NO: | | | | | | measured |
| 1135) | | | | | | |
| GATA3 . . . CSNH | B08.01 | EPHLALQPL | 1077 | 1128.3 | 12.4 | not |
| (SEQ ID NO: | | | | | | measured |
| 1135) | | | | | | |
| GATA3 . . . CSNH | B07.02 | GPPARVPAV | 1082 | 297.6 | 221.2 | not |
| (SEQ ID NO: | | | | | | measured |
| 1135) | | | | | | |
| GATA3 . . . CSNH | B08.01 | MFATLQRSSL | 1079 | 220.5 | 53.4 | not |
| (SEQ ID NO: | | | | | | measured |
| 1135) | | | | | | |
| GATA3 . . . CSNH | A02.01 | ALQPLQPHA | 1083 | 644.4 | 603.9 | not |
| (SEQ ID NO: | | | | | | measured |
| 1135) | | | | | | |
| GATA3 . . . CSNH | A03.01 | VLWTTPPLQH | 707 | 962.3 | 16.0 | not |
| (SEQ ID NO: | | | | | | measured |
| 1135) | | | | | | |
| GATA3 . . . CSNH | A02.01 | VLPEPHLAL | 1084 | 140.7 | 16.0 | not |
| (SEQ ID NO: | | | | | | measured |
| 1135) | | | | | | |
| GATA3 . . . CSNH | B07.02 | HVLPEPHLAL | 705 | 1057.2 | 1332.6 | not |
| (SEQ ID NO: | | | | | | measured |
| 1135) | | | | | | |
| GATA3 . . . CSNH | A03.01 | YMFLKAESK | 1085 | 53.1 | 79.8 | not |
| (SEQ ID NO: | | | | | | measured |
| 1135) | | | | | | |
| GATA3 . . . CSNH | B07.02 | VPAVPFDLHF | 1086 | 1996.2 | 2114.2 | not |
| (SEQ ID NO: | | | | | | measured |
| 1135) | | | | | | |
| GATA3 . . . CSNH | A02.01 | AIQPVLWTT | 1087 | 229.3 | 8.1 | not |
| (SEQ ID NO: | | | | | | measured |
| 1135) | | | | | | |
| GATA3 . . . CSNH | A02.01 | TLQRSSLWCL | 1088 | 319.2 | 117.7 | not |
| (SEQ ID NO: | | | | | | measured |
| 1135) | | | | | | |
| GATA3 . . . CSNH | A03.01 | KIMFATLQR | 1089 | 62.5 | 2.5 | not |
| (SEQ ID NO: | | | | | | measured |
| 1135) | | | | | | |
| GATA3 . . . CSNH | B07.02 | QPVLWTTPPL | 1090 | 54.4 | 109.3 | not |
| (SEQ ID NO: | | | | | | measured |
| 1135) | | | | | | |
| GATA3 . . . CSNH | B08.01 | ESKIMFATL | 1091 | 253.7 | 17.7 | not |
| (SEQ ID NO: | | | | | | measured |
| 1135) | | | | | | |
| GATA3 . . . CSNH | B08.01 | IMKPKRDGYM | 1092 | 342.1 | 33.2 | not |
| (SEQ ID NO: | | | | | | measured |
| 1135) | | | | | | |
| GATA3 . . . CSNH | B07.02 | KPKRDGYMF | 1093 | 109.7 | 28.2 | not |
| (SEQ ID NO: | | | | | | measured |
| 1135) | | | | | | |
| GATA3 . . . CSNH | B08.01 | FLKAESKIMF | 1094 | 1539.9 | 82.3 | not |
| (SEQ ID NO: | | | | | | measured |
| 1135) | | | | | | |
| GATA3 . . . CSNH | B07.02 | KPKRDGYMFL | 1095 | 32.5 | 98.1 | not |
| (SEQ ID NO: | | | | | | measured |
| 1135) | | | | | | |
| GATA3 . . . CSNH | B08.01 | LHFCRSSIM | 1096 | 2141.2 | 118.7 | not |
| (SEQ ID NO: | | | | | | measured |
| 1135) | | | | | | |
| GATA3 . . . CSNH | A02.01 | SMLTGPPARV | 6 | 57.0 | 15.0 | 21.7 |
| (SEQ ID NO: | | | | | | |
| 1135) | | | | | | |
| GATA3 . . . CSNH | B08.01 | YMFLKAESKI | 1097 | 606.0 | 32.0 | 0.4 |
| (SEQ ID NO: | | | | | | |
| 1135) | | | | | | |
| GATA3 . . . CSNH | A02.01 | YMFLKAESKI | 1097 | 163.0 | 166.0 | 0.6 |
| (SEQ ID NO: | | | | | | |
| 1135) | | | | | | |
| GATA3 . . . CSNH | A03.01 | YMFLKAESKI | 1097 | 1338.0 | 21111.0 | 0 |
| (SEQ ID NO: | | | | | | |
| 1135) | | | | | | |
| GATA3 YHEA | A02.01 | FLQEQYHEA | 710 | 7.0 | 11.0 | 9.5 |
| (SEQ ID NO: | | | | | | |
| 1136) | | | | | | |
| GATA3 YHEA | B08.01 | FLQEQYHEA | 710 | 1222.0 | 2285.0 | 0 |
| (SEQ ID NO: | | | | | | |
| 1136) | | | | | | |
| GBP3(−1) | B08.01 | TLKKKPRDI | 365 | 286.3 | 3.3 | not |
| | | | | | | measured |
| HER2, | A02.01 | VMAYVMAGV | 1098 | 6.0 | 2.0 | 16.5 |
| G776insYVMA | | | | | | |
| (SEQ ID NO: | | | | | | |
| 1137) | | | | | | |
| HER2, | A02.01 | YVMAYVMAGV | 1099 | 5.0 | 57.0 | 20.9 |
| G776insYVMA | | | | | | |
| (SEQ ID NO: | | | | | | |
| 1137) | | | | | | |
| HER2, | B07.02 | YVMAYVMAG | 1100 | 6910.0 | 170.0 | 0 |
| G776insYVMA | | | | | | |
| (SEQ ID NO: | | | | | | |
| 1137) | | | | | | |
| HER2, | B08.01 | YVMAYVMAGV | 1099 | 721.0 | 353.0 | 0 |
| G776insYVMA | | | | | | |
| (SEQ ID NO: | | | | | | |
| 1137) | | | | | | |
| HER2, | A02.01 | YVMAYVMAG | 1100 | 841.0 | 11535.0 | 2.5 |
| G776insYVMA | | | | | | |
| (SEQ ID NO: | | | | | | |
| 1137) | | | | | | |
| HER2, | B08.01 | YVMAYVMAG | 1100 | 836.0 | 19413.0 | 1.2 |
| G776insYVMA | | | | | | |
| (SEQ ID NO: | | | | | | |
| 1137) | | | | | | |
| HER2, | B07.02 | YVMAYVMAGV | 1099 | 11445.0 | 52630.0 | 0 |
| G776insYVMA | | | | | | |
| (SEQ ID NO: | | | | | | |
| 1137) | | | | | | |
| HER2, L7555 | A03.01 | KVSRENTSPK | 1020 | 66.0 | 7.0 | 13.5 |
| HER2, V777L | A02.01 | VMAGLGSPYV | 263 | 20.0 | 102.0 | 2.9 |
| HER2, V777L | A03.01 | VMAGLGSPYV | 263 | 3951.0 | 11222.0 | 0 |
| JAK1(−1) | A02.01 | SLMPAHWSI | 1101 | 4.0 | 48.0 | 5 |
| JAK1(−1) | A02.01 | LSLMPAHWSI | 1102 | 21.0 | 164.0 | 0.4 |
| JAK1(−1) | B08.01 | SLMPAHWSI | 1101 | 282.0 | 177.0 | 0 |
| JAK1(−1) | A02.01 | FQMQPLSLM | 1103 | 33.0 | 553.0 | 0.3 |
| JAK1(−1) | A24.02 | SLMPAHWSI | 1101 | 194.0 | 633.0 | 0.4 |
| JAK1(−1) | B08.01 | LSLMPAHWSI | 1102 | 1914.0 | 860.0 | 0 |
| JAK1(−1) | B07.02 | SLMPAHWSI | 1101 | 3907.0 | 1040.0 | 0 |
| JAK1(−1) | B08.01 | FQMQPLSLM | 1103 | 2261.0 | 6714.0 | 0 |
| JAK1(−1) | B07.02 | FQMQPLSLM | 1103 | 3458.0 | 10207.0 | 0 |
| JAK1(−1) | A24.02 | LSLMPAHWSI | 1102 | 2125.0 | 12398.0 | 0 |
| JAK1(−1) | A24.02 | FQMQPLSLM | 1103 | 4021.0 | 14612.0 | 0 |
| KIT, T670I | A02.01 | VIIEYCCYG | 270 | 4225.0 | 191.0 | 0.5 |
| KIT, T670I | A02.01 | IIEYCCYGDL | 268 | 3918.0 | 7310.0 | 0 |
| KIT, T670I | A02.01 | TIGGPTLVII | 269 | 5425.0 | 10685.0 | 0 |
| KIT, V654A | A02.01 | YLGNHMNIA | 274 | 92.0 | 117.0 | 0.6 |
| KIT, V654A | A02.01 | MNIANLLGA | 273 | 4522.0 | 128.0 | 0.3 |
| KIT, V654A | A02.01 | HMNIANLLGA | 271 | 294.0 | 430.0 | 0 |
| KIT, V654A | B08.01 | YLGNHMNIA | 274 | 2480.0 | 872.0 | 0 |
| KIT, V654A | A02.01 | YLGNHMNIAN | 275 | 7103.0 | 1342.0 | 0 |
| KIT, V654A | A02.01 | IANLLGACTI | 272 | 11214.0 | 6417.0 | 0 |
| KRAS, G12C | A02.01 | KLVVVGACGV | 154 | 204.0 | 150.0 | 1 |
| KRAS, G12C | A02.01 | LVVVGACGV | 155 | 658.0 | 1213.0 | 0.6 |
| KRAS, G12C | A03.01 | VVVGACGVGK | 157 | 300.7 | 1.6 | not |
| | | | | | | measured |
| KRAS, G12C | A03.01 | VVGACGVGK | 156 | 182.0 | 4.1 | not |
| | | | | | | measured |
| KRAS, G12D | A02.01 | KLVVVGADGV | 160 | 361.0 | 184.0 | 0.9 |
| KRAS, G12D | A02.01 | LVVVGADGV | 161 | 2120.0 | 1192.0 | 0 |
| KRAS, G12V | A02.01 | KLVVVGAVGV | 162 | 163.0 | 96.0 | 0.9 |
| KRAS, G12V | A02.01 | LVVVGAVGV | 163 | 453.0 | 975.0 | 0.6 |
| KRAS, G12V | A03.01 | VVGAVGVGK | 164 | 168.9 | 1.9 | not |
| | | | | | | measured |
| KRAS, Q61H | A01.01 | ILDTAGHEEY | 165 | 131.8 | 64.2 | not |
| | | | | | | measured |
| KRAS, Q61L | A01.01 | ILDTAGLEEY | 166 | 65.9 | 8.6 | not |
| | | | | | | measured |
| KRAS, Q61L | A02.01 | LLDILDTAGL | 167 | 113.4 | 715.7 | not |
| | | | | | | measured |
| LMAN1(+1) | B07.02 | GPPRPPRAAC | 373 | 69.3 | 48.6 | not |
| | | | | | | measured |
| LMAN1(+1) | B07.02 | PPRPPRAAC | 374 | 263.7 | 32.8 | not |
| | | | | | | measured |
| LMAN1(−1) | B08.01 | SLRRKYLRV | 375 | 28.0 | 0.4 | not |
| | | | | | | measured |
| MEK, C121S | A02.01 | VLHESNSPYI | 277 | 189.0 | 131.0 | 1.9 |
| MEK, P124L | A02.01 | VLHECNSLYI | 285 | 67.0 | 10.0 | 5.1 |
| MEK, P124L | A02.01 | SLYIVGFYGA | 283 | 104.0 | 390.0 | 0.4 |
| MEK, P124L | A02.01 | SLYIVGFYG | 282 | 2987.0 | 1063.0 | 0 |
| MEK, P124L | A02.01 | LQVLHECNSL | 278 | 5803.0 | 4723.0 | 0 |
| MEK, P124L | A02.01 | QVLHECNSL | 281 | 8695.0 | 7774.0 | 0.5 |
| MEK, P124L | A03.01 | VLHECNSLYI | 285 | 4733.0 | 10500.0 | 0 |
| MEK, P124L | B08.01 | QVLHECNSL | 281 | 6854.0 | 14532.0 | 0 |
| MEK, P124L | B08.01 | LQVLHECNSL | 278 | 2782.0 | 19316.0 | 0 |
| MLL2, . . . LSPH | A02.01 | LLQVTQTSFA | 1104 | 1935.0 | 676.9 | not |
| (SEQ ID NO: | | | | | | measured |
| 1138) | | | | | | |
| MLL2, . . . LSPH | A02.01 | RLWHLLLQV | 1105 | 8.3 | 1.3 | not |
| (SEQ ID NO: | | | | | | measured |
| 1138) | | | | | | |
| MLL2, . . . LSPH | A02.01 | LQVTQTSFAL | 1106 | 1147.4 | 718.3 | not |
| (SEQ ID NO: | | | | | | measured |
| 1138) | | | | | | |
| MLL2, . . . LSPH | A02.01 | RLWHLLLQVT | 1107 | 140.8 | 50.9 | not |
| (SEQ ID NO: | | | | | | measured |
| 1138) | | | | | | |
| MLL2, . . . LSPH | A02.01 | ALAPTLTHM | 1108 | 98.4 | 59.0 | not |
| (SEQ ID NO: | | | | | | measured |
| 1138) | | | | | | |
| MLL2, . . . LSPH | A02.01 | ALAPTLTHML | 1109 | 66.4 | 39.0 | not |
| (SEQ ID NO: | | | | | | measured |
| 1138) | | | | | | |
| MLL2, CRLS | B08.01 | SLGNHLCPL | 1110 | 136.0 | 6.0 | 0.5 |
| (SEQ ID NO: | | | | | | |
| 1139) | | | | | | |
| MLL2, CRLS | A02.01 | SLGNHLCPL | 1110 | 28.0 | 18.0 | 3.5 |
| (SEQ ID NO: | | | | | | |
| 1139) | | | | | | |
| MLL2, CRLS | B07.02 | SLGNHLCPL | 1110 | 3967.0 | 2590.0 | 0 |
| (SEQ ID NO: | | | | | | |
| 1139) | | | | | | |
| MSH3(−1) | A02.01 | LLALWECSL | 385 | 46.0 | 15.0 | 4 |
| MSH3(−1) | A02.01 | FLLALWECSL | 381 | 17.0 | 114.0 | 10.8 |
| MSH3(−1) | B08.01 | LLALWECSL | 385 | 1454.0 | 154.0 | 0 |
| MSH3(−1) | B08.01 | FLLALWECSL | 381 | 671.0 | 13100.0 | 0 |
| MSH3(−1) | A02.01 | LIVSRTLLL | 383 | 755.0 | 173.5 | not |
| | | | | | | measured |
| MSH3(−1) | A02.01 | LIVSRTLLLV | 384 | 146.6 | 10920.6 | not |
| | | | | | | measured |
| MSH3(−1) | B08.01 | LIVSRTLLL | 383 | 270.7 | 881.7 | not |
| | | | | | | measured |
| MSH3(−1) | A02.01 | IVSRTLLLV | 382 | 166.2 | 12.7 | not |
| | | | | | | measured |
| MSH3(−1) | B08.01 | SLPQARLCLI | 389 | 632.3 | 4313.9 | not |
| | | | | | | measured |
| MSH3(−1) | B08.01 | CLIVSRTLLL | 379 | 835.7 | 1100.4 | not |
| | | | | | | measured |
| MSH3(−1) | B08.01 | LPQARLCLI | 386 | 136.5 | 15.0 | not |
| | | | | | | measured |
| MSH3(−1) | A02.01 | SLPQARLCLI | 389 | 782.4 | 112.9 | not |
| | | | | | | measured |
| MSH3(−1) | A02.01 | CLIVSRTLLL | 379 | 560.5 | 2005.1 | not |
| | | | | | | measured |
| MSH3(−1) | A02.01 | FLLALWECS | 380 | 686.6 | 93.2 | not |
| | | | | | | measured |
| MSH3(−1) | A02.01 | FLLALWECSL | 381 | 16.6 | 0.9 | not |
| | | | | | | measured |
| MSH3(−1) | A02.01 | LLALWECSL | 385 | 46.1 | 12.5 | not |
| | | | | | | measured |
| MSH3(−1) | B07.02 | LPQARLCLI | 386 | 134.6 | 72.6 | not |
| | | | | | | measured |
| MSH3(−1) | B08.01 | CLIVSRTLL | 378 | 915.0 | 126.7 | not |
| | | | | | | measured |
| MSH3(−1) | A02.01 | ALWECSLPQA | 377 | 24.6 | 9.0 | not |
| | | | | | | measured |
| MSH3(−1) | B08.01 | LPQARLCLIV | 387 | 591.4 | 152.1 | not |
| | | | | | | measured |
| MSH6(+1) | A02.01 | LLPEDTPPL | 1047 | 8.9 | 2.8 | not |
| | | | | | | measured |
| MSH6(+1) | A02.01 | ILLPEDTPPL | 1046 | 16.3 | 6.7 | not |
| | | | | | | measured |
| MYC, E39D | A02.01 | QQSDLQPPA | 288 | 4930.0 | 70.0 | 0 |
| MYC, E39D | A02.01 | QQQSDLQPPA | 287 | 11835.0 | 646.0 | 0 |
| MYC, E39D | A02.01 | YQQQQQSDL | 289 | 8842.0 | 799.0 | 0.4 |
| MYC, E39D | B08.01 | YQQQQQSDL | 289 | 5259.0 | 18868.0 | 0 |
| MYC, P57S | A02.01 | FELLSTPPL | 290 | 2509.0 | 225.0 | 0 |
| MYC, P57S | A02.01 | LLSTPPLSPS | 291 | 5226.0 | 1770.0 | 0 |
| MYC, P57S | B08.01 | FELLSTPPL | 290 | 4208.0 | 3179.0 | 0 |
| MYC, T58I | A02.01 | LLPIPPLSPS | 294 | 2071.0 | 449.0 | 0 |
| MYC, T58I | A02.01 | FELLPIPPL | 292 | 2472.0 | 553.0 | 0 |
| NAB: STAT6 | A02.01 | SQIMSLWGL | 985 | 14.0 | 62.0 | 1 |
| (“variant 1” of | | | | | | |
| Chmielecki et al.) | | | | | | |
| NAB: STAT6 | A02.01 | IMSLWGLVS | 981 | 3630.0 | 7321.0 | 0 |
| (“variant 1” of | | | | | | |
| Chmielecki et al.) | | | | | | |
| NAB: STAT6 | A24.02 | SQIMSLWGL | 985 | 1604.0 | 8516.0 | 0 |
| (“variant 1” of | | | | | | |
| Chmielecki et al.) | | | | | | |
| NAB: STAT6 | B08.01 | SQIMSLWGL | 985 | 4587.0 | 15997.0 | 0 |
| (“variant 1” of | | | | | | |
| Chmielecki et al.) | | | | | | |
| NDRG1: ERG | A02.01 | LLQEFDVQEA | 988 | 200.0 | 45.0 | 2.5 |
| NDRG1: ERG | A02.01 | LQEFDVQEAL | 989 | 2229.0 | 50280.0 | 0 |
| NDUFC2 | A02.01 | ITAFFFCWI | 392 | 437.7 | 7490.4 | not |
| (-KCDT14)(+1) | | | | | | measured |
| NDUFC2 | A24.02 | LYITAFFFCW | 393 | 46.6 | 45.3 | not |
| (-KCDT14)(+1) | | | | | | measured |
| NDUFC2 | A03.01 | FFFCWILSCK | 391 | 325.9 | 597.1 | not |
| (-KCDT14)(+1) | | | | | | measured |
| NDUFC2 | A03.01 | FFCWILSCK | 390 | 985.7 | 184.9 | not |
| (-KCDT14)(+1) | | | | | | measured |
| NDUFC2 | A02.01 | LLYITAFFL | 396 | 24.0 | 713.0 | 17 |
| (-KCDT14)(−1) | | | | | | |
| NDUFC2 | B08.01 | LLYITAFFL | 396 | 3588.0 | 9592.0 | 0 |
| (-KCDT14)(−1) | | | | | | |
| NDUFC2 | A02.01 | ITAFFLLDI | 395 | 699.0 | 78.7 | not |
| (-KCDT14)(−1) | | | | | | measured |
| NDUFC2 | A02.01 | YITAFFLLDI | 400 | 157.0 | 64.5 | not |
| (-KCDT14)(−1) | | | | | | measured |
| NDUFC2 | A24.02 | LYITAFFLL | 398 | 15.6 | 0.1 | not |
| (-KCDT14)(−1) | | | | | | measured |
| NDUFC2 | A02.01 | LLYITAFFLL | 397 | 43.7 | 323.2 | not |
| (-KCDT14)(−1) | | | | | | measured |
| NDUFC2 | A24.02 | LLYITAFFLL | 397 | 59.7 | 60.1 | not |
| (-KCDT14)(−1) | | | | | | measured |
| NDUFC2 | A24.02 | LYITAFFLLD | 399 | 414.3 | 0.4 | not |
| (-KCDT14)(−1) | | | | | | measured |
| NRAS, Q61K | A01.01 | ILDTAGKEEY | 168 | 272.6 | 14.3 | not |
| | | | | | | measured |
| NRAS, Q61R | A01.01 | ILDTAGREEY | 169 | 255.8 | 7.0 | not |
| | | | | | | measured |
| PDGFRa, T674I | A02.01 | IIIEYCFYG | 297 | 693.0 | 16.0 | 1.2 |
| PDGFRa, T674I | A02.01 | YIIIEYCFYG | 300 | 1529.0 | 113.0 | 0 |
| PDGFRa, T674I | A02.01 | IIEYCFYGDL | 296 | 3049.0 | 1090.0 | 0 |
| PIK3CA, E542K | A02.01 | KITEQEKDFL | 301 | 12548.0 | 1397.0 | 0 |
| PIK3CA, E542K | A03.01 | AISTRDPLSK | 1022 | 41.3 | 57.5 | not |
| | | | | | | measured |
| PTEN, R130Q | A02.01 | QTGVMICAYL | 305 | 3786.0 | 9760.0 | 0 |
| RAC1, P29S | A02.01 | FSGEYIPTV | 1024 | 21.0 | 3.0 | 6.8 |
| RAC1, P29S | A02.01 | AFSGEYIPTV | 306 | 1008.0 | 781.0 | 0 |
| RAC1, P29S | A01.01 | TTNAFSGEY | 1025 | 23.0 | 4.4 | not |
| | | | | | | measured |
| RAC1, P29S | A01.01 | YTTNAFSGEY | 1026 | 20.0 | 10.5 | not |
| | | | | | | measured |
| RBM27(+1) | B07.02 | MPKDVNIQV | 402 | 291.6 | 12.5 | not |
| | | | | | | measured |
| RBM27(+1) | A01.01 | TGSNEVTTRY | 403 | 343.9 | 15545.2 | not |
| | | | | | | measured |
| RBM27(+1) | A01.01 | GSNEVTTRY | 401 | 151.6 | 605.5 | not |
| | | | | | | measured |
| RNF43, RHTP | A02.01 | TQLARFFPI | 1111 | 17.0 | 19.0 | 0 |
| (SEQ ID NO: | | | | | | |
| 1140) | | | | | | |
| RNF43, RHTP | A24.02 | TQLARFFPI | 1111 | 268.0 | 52.0 | 0 |
| (SEQ ID NO: | | | | | | |
| 1140) | | | | | | |
| RNF43, RHTP | B08.01 | TQLARFFPI | 1111 | 41.0 | 9150.0 | 0 |
| (SEQ ID NO: | | | | | | |
| 1140) | | | | | | |
| SEC31A(−1) | A02.01 | KLMLLRLNL | 405 | 58.0 | 17.0 | 16.9 |
| SEC31A(−1) | B08.01 | KLMLLRLNL | 405 | 421.0 | 29.0 | 0 |
| SEC31A(−1) | B07.02 | KLMLLRLNL | 405 | 2969.0 | 133.0 | 1.5 |
| SEC31A(−1) | A03.01 | KLMLLRLNL | 405 | 4664.0 | 210.0 | 0 |
| SEC31A(−1) | B08.01 | LLRLNLRKM | 407 | 185.1 | 68.2 | not |
| | | | | | | measured |
| SEC31A(−1) | A03.01 | MLLRLNLRKM | 412 | 171.4 | 116.9 | not |
| | | | | | | measured |
| SEC31A(−1) | A03.01 | KLMLLRLNLR | 406 | 95.4 | 48.4 | not |
| | | | | | | measured |
| SEC31A(−1) | A03.01 | MLLRLNLRK | 411 | 14.6 | 1.3 | not |
| | | | | | | measured |
| SEC31A(−1) | A03.01 | LMLLRLNLRK | 409 | 23.9 | 6.0 | not |
| | | | | | | measured |
| SEC31A(−1) | A02.01 | MLLRLNLRKM | 412 | 508.5 | 2507.4 | not |
| | | | | | | measured |
| SEC31A(−1) | B08.01 | MLLRLNLRKM | 412 | 565.9 | 95.3 | not |
| | | | | | | measured |
| SEC31A(−1) | A02.01 | KLMLLRLNL | 405 | 57.6 | 2.6 | not |
| | | | | | | measured |
| SEC31A(−1) | B08.01 | LMLLRLNL | 408 | 116.0 | 9.3 | not |
| | | | | | | measured |
| SEC31A(−1) | B08.01 | KLMLLRLNL | 405 | 420.6 | 58.4 | not |
| | | | | | | measured |
| SEC31A(−1) | A02.01 | KKLMLLRLNL | 404 | 275.4 | 288.9 | not |
| | | | | | | measured |
| SEC31A(−1) | B 08. 01 | NLRKMCGPF | 413 | 163.5 | 35.9 | not |
| | | | | | | measured |
| SEC31A(−1) | B08.01 | YCQKKLMLL | 415 | 203.1 | 222.1 | not |
| | | | | | | measured |
| SEC31A(−1) | B 08. 01 | LNLRKMCGPF | 410 | 782.2 | 438.7 | not |
| | | | | | | measured |
| SEC63 (+1) | A03.01 | YTCAITTVK | 424 | 279.0 | 122.4 | not |
| | | | | | | measured |
| SEC63 (+1) | A03.01 | TYTCAITTVK | 423 | 556.4 | 2362.2 | not |
| | | | | | | measured |
| SEC63 (+1) | A03.01 | ITTVKATETK | 417 | 795.8 | 1245.3 | not |
| | | | | | | measured |
| SEC63 (+1) | A03.01 | KSKKKETFKK | 419 | 744.0 | 39.6 | not |
| | | | | | | measured |
| SEC63 (+1) | B08.01 | TFKKKTYTC | 421 | 648.2 | 77.9 | not |
| | | | | | | measured |
| SEC63 (+1) | A03.01 | KSKKKETFK | 418 | 411.0 | 74.3 | not |
| | | | | | | measured |
| SEC63 (+1) | B08.01 | FKKKTYTCAI | 416 | 562.8 | 384.9 | not |
| | | | | | | measured |
| SEC63 (−1) | B08.01 | TAKSKKRNL | 425 | 213.8 | 30.6 | not |
| | | | | | | measured |
| SF3B1, K700E | A02.01 | GLVDEQQEV | 1027 | 50.0 | 44.0 | 7.4 |
| SLC35F5(−1) | A02.01 | FALCGFWQI | 426 | 10.5 | 0.4 | not |
| | | | | | | measured |
| SMAP1(−1) | A03.01 | KSRQNHLQLK | 1112 | 88.1 | 4.7 | not |
| | | | | | | measured |
| SMAP1(−1) | B07.02 | KSRQNHLQL | 1113 | 329.5 | 78.0 | not |
| | | | | | | measured |
| SMAP1(−1) | A24.02 | KLRSPLWIF | 1114 | 504.5 | 828.2 | not |
| | | | | | | measured |
| SMAP1(−1) | A03.01 | KISNWSLKK | 1115 | 11.5 | 8.8 | not |
| | | | | | | measured |
| SMAP1(−1) | A11.01 | KISNWSLKK | 1115 | 15.3 | 9.8 | not |
| | | | | | | measured |
| SMAP1(−1) | A11.01 | SLKKVPALK | 1116 | 117.6 | 129.1 | not |
| | | | | | | measured |
| SMAP1(−1) | B08.01 | SLKKVPAL | 428 | 66.8 | 7.9 | not |
| | | | | | | measured |
| SMAP1(−1) | A03.01 | WSLKKVPALK | 1117 | 148.9 | 94.9 | not |
| | | | | | | measured |
| SMAP1(−1) | A03.01 | KISNWSLKKV | 1118 | 168.3 | 114.6 | not |
| | | | | | | measured |
| SMAP1(−1) | A03.01 | RKISNWSLKK | 429 | 20.8 | 130.6 | not |
| | | | | | | measured |
| SMAP1(−1) | A03.01 | SLKKVPALK | 1116 | 29.6 | 4.4 | not |
| | | | | | | measured |
| SMAP1(−1) | B08.01 | SQKSRQNHL | 1119 | 305.0 | 44.6 | not |
| | | | | | | measured |
| SMAP1(−1) | B07.02 | ALKKLRSPL | 1120 | 355.5 | 223.2 | not |
| | | | | | | measured |
| SMAP1(−1) | B08.01 | ALKKLRSPL | 1120 | 58.9 | 0.5 | not |
| | | | | | | measured |
| SMAP1(−1) | B08.01 | WSLKKVPAL | 1121 | 110.7 | 12.5 | not |
| | | | | | | measured |
| SMAP1(−1) | A03.01 | HLQLKSCRRK | 1122 | 216.7 | 96.9 | not |
| | | | | | | measured |
| SMAP1(−1) | B08.01 | LKKLRSPL | 427 | 139.6 | 0.6 | not |
| | | | | | | measured |
| SMAP1(−1) | A03.01 | SLKKVPALKK | 1123 | 43.1 | 9.6 | not |
| | | | | | | measured |
| SPOP, F133L | A02.01 | FVQGKDWGL | 1028 | 121.0 | 34.0 | 2.1 |
| SPOP, F133L | B08.01 | FVQGKDWGL | 1028 | 1401.0 | 207.0 | 0 |
| TFAM(+1) | A03.01 | RVNTAWKTK | 433 | 136.4 | 8.6 | not |
| | | | | | | measured |
| TFAM(+1) | A03.01 | RVNTAWKTKK | 434 | 70.6 | 2.3 | not |
| | | | | | | measured |
| TFAM(+1) | B08.01 | TKKKRVNTA | 435 | 312.4 | 159.4 | not |
| | | | | | | measured |
| TFAM(+1) | A03.01 | KRVNTAWKTK | 431 | 304.1 | 331.6 | not |
| | | | | | | measured |
| TFAM(+1) | B08.01 | WKTKKTSFSL | 436 | 930.6 | 112.2 | not |
| | | | | | | measured |
| TFAM(+1) | B08.01 | MTKKKRVNTA | 432 | 534.2 | 186.9 | not |
| | | | | | | measured |
| TGFBR2(−1) | A02.01 | RLSSCVPVA | 446 | 83.0 | 4.0 | 18.7 |
| TGFBR2(−1) | A03.01 | RLSSCVPVA | 446 | 4264.0 | 439.0 | 0 |
| TGFBR2(−1) | A03.01 | AMTTSSSQK | 438 | 48.5 | 8.3 | not |
| | | | | | | measured |
| TGFBR2(−1) | A03.01 | AMTTSSSQKN | 439 | 887.2 | 2336.5 | not |
| | | | | | | measured |
| TGFBR2(−1) | B08.01 | IMKEKKSL | 442 | 69.8 | 14.1 | not |
| | | | | | | measured |
| TGFBR2(−1) | A02.01 | KSLVRLSSCV | 444 | 903.1 | 279.8 | not |
| | | | | | | measured |
| TGFBR2(−1) | A02.01 | SLVRLSSCV | 449 | 177.3 | 29.9 | not |
| | | | | | | measured |
| TGFBR2(−1) | A11.01 | SAMTTSSSQK | 448 | 36.4 | 15.8 | not |
| | | | | | | measured |
| TGFBR2(−1) | B08.01 | IMKEKKSLV | 443 | 80.8 | 16.8 | not |
| | | | | | | measured |
| TGFBR2(−1) | A11.01 | AMTTSSSQK | 438 | 89.9 | 161.6 | not |
| | | | | | | measured |
| TGFBR2(−1) | A03.01 | SAMTTSSSQK | 448 | 96.7 | 15.7 | not |
| | | | | | | measured |
| TGFBR2(−1) | A02.01 | RLSSCVPVAL | 447 | 84.5 | 54.2 | not |
| | | | | | | measured |
| TGFBR2(−1) | A02.01 | VRLSSCVPVA | 451 | 640.6 | 1206.8 | not |
| | | | | | | measured |
| TGFBR2(−1) | A02.01 | RLSSCVPVA | 446 | 82.7 | 49.5 | not |
| | | | | | | measured |
| TGFBR2(−1) | B08.01 | CIMKEKKSL | 440 | 218.5 | 7.5 | not |
| | | | | | | measured |
| TGFBR2(−1) | A02.01 | ALMSAMTTS | 437 | 320.4 | 139.1 | not |
| | | | | | | measured |
| TGFBR2(−1) | A02.01 | LVRLSSCVPV | 445 | 132.7 | 1237.6 | not |
| | | | | | | measured |
| THAP5(−1) | A03.01 | KMRKKYAQK | 452 | 23.7 | 5.7 | not |
| | | | | | | measured |
| TMPRSS2: ERG | A02.01 | ALNSEALSV | 992 | 66.0 | 14.0 | 9.1 |
| TMPRSS2: ERG | A02.01 | ALNSEALSVV | 993 | 84.0 | 15.0 | 2.9 |
| TMPRSS2: ERG | A02.01 | MALNSEALSV | 994 | 198.0 | 129.0 | 0.7 |
| TMPRSS2: ERG | B08.01 | MALNSEALSV | 994 | 8512.0 | 13457.0 | 0 |
| TP53, AAVG | A02.01 | GLLAFWDSQV | 815 | 57.0 | 10.0 | 14.2 |
| (SEQ ID NO: | | | | | | |
| 1141) | | | | | | |
| TP53, AAVG | A02.01 | LLAFWDSQV | 817 | 13.0 | 68.0 | 12.8 |
| (SEQ ID NO: | | | | | | |
| 1141) | | | | | | |
| TP53, AWAA | A02.01 | WMTETLFDI | 849 | 7.0 | 14.0 | 4 |
| (SEQ ID NO: | | | | | | |
| 1142) | | | | | | |
| TP53, AWAA | A02.01 | WMTETLFDIV | 850 | 15.0 | 40.0 | 0.4 |
| (SEQ ID NO: | | | | | | |
| 1142) | | | | | | |
| TP53, AWAA | A24.02 | WMTETLFDI | 849 | 4936.0 | 713.0 | 0 |
| (SEQ ID NO: | | | | | | |
| 1142) | | | | | | |
| TP53, AWAA | A01.01 | WMTETLFDIV | 850 | 4046.0 | 14394.0 | 0 |
| (SEQ ID NO: | | | | | | |
| 1142) | | | | | | |
| TP53, CSES | B07.02 | LPSQRRNHWM | 858 | 89.0 | 10.0 | 6.5 |
| (SEQ ID NO: | | | | | | |
| 1143) | | | | | | |
| TP53, CSES | B08.01 | LPSQRRNHWM | 858 | 325.0 | 47.0 | 0.7 |
| (SEQ ID NO: | | | | | | |
| 1143) | | | | | | |
| TP53, CSES | A02.01 | ALSEHCPTT | 853 | 208.0 | 79.0 | 27.3 |
| (SEQ ID NO: | | | | | | |
| 1143) | | | | | | |
| TP53, G245S | B08.01 | CMGSMNRRPI | 1030 | 1204.0 | 80.0 | 0 |
| TP53, G245S | A02.01 | YMCNSSCMGS | 308 | 2485.0 | 81.0 | 0.8 |
| TP53, G245S | B08.01 | SMNRRPILTI | 1034 | 260.0 | 337.0 | 0 |
| TP53, G245S | A02.01 | SMNRRPILTI | 1034 | 1644.0 | 1198.0 | 0.3 |
| TP53, G245S | B08.01 | SMNRRPILT | 307 | 2536.0 | 1282.0 | 0 |
| TP53, G245S | A02.01 | CMGSMNRRPI | 1030 | 7822.0 | 1989.0 | 0 |
| TP53, G245S | A02.01 | SMNRRPILT | 307 | 7251.0 | 3839.0 | 0 |
| TP53, G245S | A24.02 | SMNRRPILTI | 1034 | 10308.0 | 16292.0 | 0 |
| TP53, G245S | B08.01 | GSMNRRPIL | 1031 | 636.7 | 15.5 | not |
| | | | | | | measured |
| TP53, G245S | B08.01 | MGSMNRRPIL | 1033 | 89.1 | 6.3 | not |
| | | | | | | measured |
| TP53, G245S | B08.01 | MGSMNRRPI | 1032 | 324.2 | 29.1 | not |
| | | | | | | measured |
| TP53, QPSL | B07.02 | LPRKPTRAAT | 1124 | 47.0 | 3.0 | 3.7 |
| (SEQ ID NO: | | | | | | |
| 1144) | | | | | | |
| TP53, QPSL | B07.02 | LPRKPTRAA | 1125 | 8.0 | 8.0 | 5.5 |
| (SEQ ID NO: | | | | | | |
| 1144) | | | | | | |
| TP53, QPSL | B07.02 | KPTRAATVSV | 1126 | 12.0 | 8.0 | 3 |
| (SEQ ID NO: | | | | | | |
| 1144) | | | | | | |
| TP53, QPSL | B08.01 | LPRKPTRAA | 1125 | 873.0 | 1158.0 | 0 |
| (SEQ ID NO: | | | | | | |
| 1144) | | | | | | |
| TP53, R248Q | B08.01 | NQRPILTII | 1037 | 3433.0 | 20.0 | 0 |
| TP53, R248Q | A02.01 | GMNQRPILTI | 1036 | 1787.0 | 709.0 | 0.4 |
| TP53, R248Q | A02.01 | GMNQRPILT | 309 | 8115.0 | 3029.0 | 0 |
| TP53, R248Q | A02.01 | CMGGMNQRPI | 1035 | 3025.0 | 3673.0 | 0 |
| TP53, R248Q | A02.01 | NQRPILTII | 1037 | 10855.0 | 9606.0 | 0 |
| TP53, R248Q | B08.01 | CMGGMNQRPI | 1035 | 6364.0 | 18766.0 | 0 |
| TP53, R248Q | B08.01 | GMNQRPILTI | 1036 | 3266.0 | 29251.0 | 0 |
| TP53, R248W | B08.01 | MNWRPILTI | 1040 | 6447.0 | 1.0 | 0 |
| TP53, R248W | A02.01 | GMNWRPILTI | 1039 | 189.0 | 282.0 | 0.5 |
| TP53, R248W | A02.01 | CMGGMNWRPI | 1038 | 354.0 | 346.0 | 0.4 |
| TP53, R248W | A02.01 | MNWRPILTI | 1040 | 5834.0 | 516.0 | 3.8 |
| TP53, R248W | A02.01 | MNWRPILTII | 1041 | 8158.0 | 1026.0 | 0.4 |
| TP53, R248W | B08.01 | GMNWRPILTI | 1039 | 3990.0 | 1045.0 | 0 |
| TP53, R248W | A02.01 | GMNWRPILT | 310 | 3416.0 | 1130.0 | 0 |
| TP53, R248W | B08.01 | CMGGMNWRPI | 1038 | 3218.0 | 2248.0 | 0 |
| TP53, R248W | A24.02 | CMGGMNWRPI | 1038 | 9521.0 | 4453.0 | 0 |
| TP53, R248W | A24.02 | MNWRPILTI | 1040 | 3634.0 | 6977.0 | 0.2 |
| TP53, R248W | A24.02 | MNWRPILTII | 1041 | 1517.0 | 44901.0 | 0 |
| TP53, R273C | A02.01 | LLGRNSFEVC | 311 | 1272.0 | 2081.0 | 0 |
| TP53, R273C | A02.01 | NSFEVCVCA | 1042 | 4239.0 | 2200.0 | 0 |
| TP53, R273H | A02.01 | NSFEVHVCA | 1043 | 6768.0 | 503.0 | 0 |
| TP53, SHST | B07.02 | HPRPAPASA | 882 | 13.0 | 11.0 | 4.9 |
| (SEQ ID NO: | | | | | | |
| 1145) | | | | | | |
| TP53, SHST | B08.01 | HPRPAPASA | 882 | 1718.0 | 25.0 | 0 |
| (SEQ ID NO: | | | | | | |
| 1145) | | | | | | |
| TP53, Y220C | A02.01 | VVPCEPPEV | 1044 | 1268.0 | 187.0 | 0.9 |
| TTK(−1) | A02.01 | VMSDTTYKI | 458 | 15.8 | 19.6 | not |
| | | | | | | measured |
| TTK(−1) | A03.01 | LFVMSDTTYK | 456 | 57.9 | 749.4 | not |
| | | | | | | measured |
| TTK(−1) | A02.01 | FVMSDTTYKI | 454 | 16.0 | 62.4 | not |
| | | | | | | measured |
| TTK(−1) | A03.01 | FVMSDTTYK | 453 | 63.1 | 66.9 | not |
| | | | | | | measured |
| TTK(−1) | A03.01 | KTFEKKGEK | 455 | 81.3 | 32.2 | not |
| | | | | | | measured |
| TTK(−1) | A01.01 | VMSDTTYKIY | 459 | 245.1 | 375.8 | not |
| | | | | | | measured |
| TTK(−1) | A01.01 | MSDTTYKIY | 457 | 18.9 | 10.2 | not |
| | | | | | | measured |
| UBR5(−1) | B07.02 | RVQNQGHLL | 1127 | 429.1 | 826.5 | not |
| | | | | | | measured |
| VHL, QCIL | A02.01 | MLTDSLFLPI | 1128 | 8.0 | 16.0 | 1 |
| (SEQ ID NO: | | | | | | |
| 1146) | | | | | | |
| VHL, QCIL | A02.01 | SMLTDSLFL | 1129 | 14.0 | 31.0 | 9.8 |
| (SEQ ID NO: | | | | | | |
| 1146) | | | | | | |
| VHL, QCIL | B08.01 | MLTDSLFLPI | 1128 | 2581.0 | 110.0 | 0 |
| (SEQ ID NO: | | | | | | |
| 1146) | | | | | | |
| VHL, QCIL | A01.01 | MLTDSLFLPI | 1128 | 429.0 | 7673.0 | 0 |
| (SEQ ID NO: | | | | | | |
| 1146) | | | | | | |
| XPOT(−1) | A02.01 | YLTKWPKFFL | 460 | 10.7 | 42.9 | not |
| | | | | | | measured |

TABLE 4A

| | | | SEQ ID | Peptide | Measured | Measured |
|---|---|---|---|---|---|---|
| Gene | HLA Allele | Peptide Sequence | NO: | Length | Affinity (nM) | stability (hr.) |
| KRAS, G12C | A02.01 | LVVVGACGV | 155 | 9 | 667.1 | 0.6 |
| KRAS, G12C | A02.01 | KLVVVGACGV | 154 | 10 | 70.3 | 1.0 |
| KRAS, G12D | A02.01 | LVVVGADGV | 161 | 9 | 977.4 | 0.0 |
| KRAS, G12D | A02.01 | KLVVVGADGV | 160 | 10 | 137.7 | 0.9 |
| KRAS, G12V | A02.01 | LVVVGAVGV | 163 | 9 | 682.5 | 0.6 |
| KRAS, G12V | A02.01 | KLVVVGAVGV | 162 | 10 | 57.6 | 0.9 |
| KRAS, G12C | A03.01 | VVGACGVGK | 156 | 9 | 4.1 | 5.0 |
| KRAS, G12C | A03.01 | VVVGACGVGK | 157 | 10 | 1.6 | 2.5 |
| KRAS, G12D | A03.01 | VVGADGVGK | 158 | 9 | 518.7 | NB |
| KRAS, G12D | A03.01 | VVVGADGVGK | 159 | 10 | 314.9 | 2.3 |
| KRAS, G12V | A03.01 | VVGAVGVGK | 164 | 9 | 1.9 | 1.2 |
| KRAS, G12V | A03.01 | VVVGAVGVGK | 5 | 10 | 44.2 | 6.7 |
| KRAS, G12C | A11.01 | VVGACGVGK | 156 | 9 | 43.2 | 10.0 |
| KRAS, G12C | A11.01 | VVVGACGVGK | 157 | 10 | 69.3 | 15.7 |
| KRAS, G12D | A11.01 | VVGADGVGK | 158 | 9 | 203.9 | 3.4 |
| KRAS, G12D | A11.01 | VVVGADGVGK | 159 | 10 | 33.1 | 13.0 |
| KRAS, G12V | A11.01 | VVGAVGVGK | 164 | 9 | 7.7 | 16.9 |
| KRAS, G12V | A11.01 | VVVGAVGVGK | 5 | 10 | 26.1 | 24.3 |
| KRAS, G12D | B08: 01 | DGVGKSAL | 1147 | 8 | | |
| KRAS, G12V | B08: 01 | VGVGKSAL | 1148 | 8 | | |
| KRAS, G12C | B08: 01 | CGVGKSAL | 1149 | 8 | | |

Table 4B-4M show peptide sequences comprising RAS mutations, corresponding HLA allele to which it binds, and corresponding predicted binding affinity score with the lowest number (e.g., 1) having the highest affinity and vice-versa.

TABLE 4B
RAS Q61H Mutation

| | SEQ | | |
|---|---|---|---|
| | ID | | Rank of |
| Peptide | NO: | Allele | Binding Potential |
| ILDTAGHEEY | 165 | HLA-A36: 01 | 1 |
| ILDTAGHEEY | 165 | HLA-A01: 01 | 2 |
| DTAGHEEYSAM | 1150 | HLA-A26: 01 | 3 |
| DTAGHEEYSAM | 1150 | HLA-A25: 01 | 4 |
| GHEEYSAM | 1151 | HLA-B15: 09 | 4 |
| DTAGHEEY | 1152 | HLA-A26: 01 | 5 |
| ILDTAGHEE | 1153 | HLA-C08: 02 | 5 |
| AGHEEYSAM | 1154 | HLA-C01: 02 | 6 |
| AGHEEYSAM | 1154 | HLA-B46: 01 | 6 |
| DTAGHEEY | 1152 | HLA-A25: 01 | 6 |
| DTAGHEEY | 1152 | HLA-A01: 01 | 6 |
| DTAGHEEY | 1152 | HLA-B18: 01 | 7 |
| DTAGHEEY | 1152 | HLA-A36: 01 | 7 |
| ILDTAGHEE | 1153 | HLA-C05: 01 | 7 |
| ILDTAGHEE | 1153 | HLA-A02: 07 | 7 |
| ILDTAGHEEY | 165 | HLA-A29: 02 | 7 |
| ILDTAGHEEY | 165 | HLA-C08: 02 | 7 |
| HEEYSAMRD | 1155 | HLA-B49: 01 | 8 |
| TAGHEEYSA | 1156 | HLA-B35: 03 | 8 |
| DTAGHEEYS | 1157 | HLA-A68: 02 | 9 |
| DTAGHEEYSAMR | 1158 | HLA-A68: 01 | 9 |
| GHEEYSAM | 1151 | HLA-B39: 01 | 9 |
| ILDTAGHEE | 1153 | HLA-A01: 01 | 9 |
| LDTAGHEEY | 1159 | HLA-B53: 01 | 9 |
| HEEYSAMRD | 1155 | HLA-B41: 01 | 10 |
| ILDTAGHEE | 1153 | HLA-A36: 01 | 10 |
| DTAGHEEY | 1152 | HLA-B58: 01 | 11 |
| LLDILDTAGH | 1160 | HLA-A01: 01 | 12 |
| TAGHEEYSAM | 1161 | HLA-B35: 03 | 12 |
| LDTAGHEEY | 1159 | HLA-B35: 01 | 13 |
| DILDTAGHE | 1162 | HLA-A26: 01 | 14 |
| DTAGHEEY | 1152 | HLA-C12: 03 | 14 |
| ILDTAGHEEY | 165 | HLA-C05: 01 | 14 |
| AGHEEYSAM | 1154 | HLA-A30: 02 | 15 |
| DILDTAGHEEY | 1163 | HLA-A25: 01 | 15 |
| DTAGHEEY | 1152 | HLA-C02: 02 | 15 |
| ILDTAGHEE | 1153 | HLA-C04: 01 | 15 |
| DILDTAGH | 1164 | HLA-A26: 01 | 16 |
| ILDTAGHEE | 1153 | HLA-A02: 01 | 16 |
| LDTAGHEEY | 1159 | HLA-A29: 02 | 16 |
| ILDTAGHE | 1165 | HLA-A01: 01 | 17 |
| LDTAGHEEY | 1159 | HLA-B18: 01 | 17 |
| AGHEEYSAM | 1154 | HLA-C14: 03 | 18 |
| DILDTAGHEEY | 1163 | HLA-A29: 02 | 18 |
| DTAGHEEYS | 1157 | HLA-A26: 01 | 18 |
| ILDTAGHEEY | 165 | HLA-B15: 01 | 18 |
| DTAGHEEYSA | 1166 | HLA-A68: 02 | 19 |
| ILDTAGHE | 1165 | HLA-C05: 01 | 19 |
| ILDTAGHEEY | 165 | HLA-A02: 07 | 19 |
| ILDTAGHEEY | 165 | HLA-A30: 02 | 19 |
| LDTAGHEEY | 1159 | HLA-A36: 01 | 19 |
| AGHEEYSAM | 1154 | HLA-C14: 02 | 20 |
| AGHEEYSAM | 1154 | HLA-B15: 03 | 20 |
| LLDILDTAGH | 1160 | HLA-A02: 07 | 20 |

TABLE 4C
RAS Q61R Mutation

| | SEQ | | |
|---|---|---|---|
| | ID | | Rank of |
| Peptide | NO: | Allele | Binding Potential |
| ILDTAGREEY | 169 | HLA-A36: 01 | 1 |
| ILDTAGREEY | 169 | HLA-A01: 01 | 2 |
| DTAGREEYSAM | 1167 | HLA-A26: 01 | 3 |
| DILDTAGR | 1168 | HLA-A33: 03 | 4 |
| DILDTAGR | 1168 | HLA-A68: 01 | 5 |
| DTAGREEY | 1169 | HLA-A26: 01 | 6 |
| DTAGREEYSAM | 1167 | HLA-A25: 01 | 6 |
| CLLDILDTAGR | 1170 | HLA-A74: 01 | 7 |
| DTAGREEY | 1169 | HLA-A01: 01 | 7 |
| REEYSAMRD | 1171 | HLA-B41: 01 | 7 |
| GREEYSAMR | 1172 | HLA-B27: 05 | 8 |
| ILDTAGREE | 1173 | HLA-C08: 02 | 8 |
| ILDTAGREEY | 169 | HLA-A29: 02 | 8 |
| REEYSAMRD | 1171 | HLA-B49: 01 | 8 |
| AGREEYSAM | 1174 | HLA-B46: 01 | 9 |
| DTAGREEY | 1169 | HLA-B18: 01 | 9 |
| DTAGREEY | 1169 | HLA-A25: 01 | 9 |
| DTAGREEY | 1169 | HLA-A36: 01 | 9 |
| DILDTAGR | 1168 | HLA-A74: 01 | 10 |
| DILDTAGRE | 1175 | HLA-A26: 01 | 10 |
| ILDTAGREE | 1173 | HLA-C05: 01 | 10 |
| DILDTAGR | 1168 | HLA-A26: 01 | 11 |
| GREEYSAM | 1176 | HLA-B39: 01 | 11 |
| AGREEYSAM | 1174 | HLA-B15: 03 | 12 |
| GREEYSAM | 1176 | HLA-C07: 02 | 12 |
| ILDTAGREE | 1173 | HLA-A01: 01 | 12 |
| TAGREEYSA | 1177 | HLA-B35: 03 | 12 |
| ILDTAGREEY | 169 | HLA-A30: 02 | 13 |
| DTAGREEYS | 1178 | HLA-A68: 02 | 14 |
| ILDTAGRE | 1179 | HLA-A01: 01 | 14 |
| CLLDILDTAGR | 1170 | HLA-A31: 01 | 15 |
| DTAGREEYSAMR | 1180 | HLA-A68: 01 | 15 |
| LLDILDTAGR | 1181 | HLA-A01: 01 | 15 |
| DTAGREEY | 1169 | HLA-B58: 01 | 16 |
| ILDTAGREEY | 169 | HLA-C08: 02 | 16 |
| DILDTAGR | 1168 | HLA-A31: 01 | 17 |
| ILDTAGREE | 1173 | HLA-C04: 01 | 17 |
| ILDTAGREEY | 169 | HLA-A32: 01 | 17 |
| LLDILDTAGR | 1181 | HLA-A74: 01 | 17 |
| TAGREEYSAM | 1182 | HLA-B35: 03 | 17 |
| DILDTAGREEY | 1183 | HLA-A32: 01 | 18 |
| ILDTAGRE | 1179 | HLA-C05: 01 | 18 |
| ILDTAGREE | 1173 | HLA-A02: 07 | 18 |
| REEYSAMRD | 1171 | HLA-B40: 01 | 18 |
| AGREEYSAM | 1174 | HLA-B15: 01 | 19 |
| AGREEYSAMR | 1184 | HLA-A31: 01 | 19 |
| ILDTAGRE | 1179 | HLA-A36: 01 | 19 |
| LDILDTAGR | 1185 | HLA-A68: 01 | 19 |
| LDTAGREEY | 1186 | HLA-A29: 02 | 19 |
| LDTAGREEY | 1186 | HLA-B35: 01 | 19 |
| REEYSAMRD | 1171 | HLA-B45: 01 | 19 |
| REEYSAMRDQY | 1187 | HLA-A36: 01 | 19 |
| DTAGREEY | 1169 | HLA-C02: 02 | 20 |

TABLE 4D
RAS Q61K Mutation

| | | | Rank of |
|---|---|---|---|
| | | | Binding |
| Peptide | SEQ ID NO: | Allele | Potential |
| ILDTAGKEEY | 168 | HLA-A36:01 | 1 |
| ILDTAGKEEY | 168 | HLA-A01:01 | 2 |
| DTAGKEEYSAM | 1188 | HLA-A26:01 | 3 |
| CLLDILDTAGK | 1189 | HLA-A03:01 | 4 |
| DTAGKEEY | 1190 | HLA-A01:01 | 5 |
| DTAGKEEY | 1190 | HLA-A26:01 | 5 |
| DTAGKEEYSAM | 1188 | HLA-A25:01 | 5 |
| AGKEEYSAM | 1191 | HLA-B46:01 | 6 |
| DILDTAGKE | 1192 | HLA-A26:01 | 7 |
| KEEYSAMRD | 1193 | HLA-B41:01 | 7 |
| DTAGKEEY | 1190 | HLA-B18:01 | 8 |
| GKEEYSAM | 1194 | HLA-B15:03 | 8 |
| ILDTAGKEE | 1195 | HLA-C08:02 | 8 |
| ILDTAGKEEY | 168 | HLA-A29:02 | 8 |
| DTAGKEEYS | 1196 | HLA-A68:02 | 9 |
| LDTAGKEEY | 1197 | HLA-B53:01 | 9 |
| TAGKEEYSA | 1198 | HLA-B35:03 | 9 |
| DILDTAGK | 1199 | HLA-A68:01 | 10 |
| DTAGKEEY | 1190 | HLA-A36:01 | 10 |
| KEEYSAMRD | 1193 | HLA-B49:01 | 10 |
| LDTAGKEEY | 1197 | HLA-C07:01 | 10 |
| DTAGKEEYSAMR | 1200 | HLA-A68:01 | 11 |
| ILDTAGKEE | 1195 | HLA-C05:01 | 11 |
| ILDTAGKEEY | 168 | HLA-C08:02 | 11 |
| LLDILDTAGK | 1201 | HLA-A01:01 | 12 |
| AGKEEYSAM | 1191 | HLA-A30:02 | 13 |
| DTAGKEEY | 1190 | HLA-A25:01 | 13 |
| DTAGKEEYS | 1196 | HLA-A26:01 | 13 |
| ILDTAGKE | 1202 | HLA-C05:01 | 13 |
| LDTAGKEEY | 1197 | HLA-B35:01 | 13 |
| AGKEEYSAMR | 1203 | HLA-A31:01 | 14 |
| DILDTAGK | 1199 | HLA-A33:03 | 14 |
| ILDTAGKE | 1202 | HLA-A01:01 | 14 |
| ILDTAGKEE | 1195 | HLA-A01:01 | 14 |
| ILDTAGKEE | 1195 | HLA-A02:07 | 14 |
| TAGKEEYSAM | 1204 | HLA-B35:03 | 14 |
| AGKEEYSAM | 1191 | HLA-B15:01 | 15 |
| ILDTAGKEEY | 168 | HLA-A30:02 | 15 |
| LDTAGKEEY | 1197 | HLA-B46:01 | 15 |
| DTAGKEEY | 1190 | HLA-B58:01 | 16 |
| ILDTAGKEEY | 168 | HLA-C05:01 | 17 |
| AGKEEYSAM | 1191 | HLA-A30:01 | 18 |
| AGKEEYSAM | 1191 | HLA-B15:03 | 18 |
| DTAGKEEY | 1190 | HLA-C02:02 | 18 |
| LDTAGKEEY | 1197 | HLA-A29:02 | 18 |

TABLE 4E
RAS Q61L Mutation

| | | | Rank of |
|---|---|---|---|
| | | | Binding |
| Peptide | SEQ ID NO: | Allele | Potential |
| ILDTAGLEEY | 166 | HLA-A36:01 | 1 |
| ILDTAGLEEY | 166 | HLA-A01:01 | 2 |
| LLDILDTAGL | 167 | HLA-A02:07 | 3 |
| GLEEYSAMRDQY | 1205 | HLA-A36:01 | 4 |
| DTAGLEEY | 1206 | HLA-A25:01 | 5 |
| DTAGLEEY | 1206 | HLA-A26:01 | 5 |
| DTAGLEEYSAM | 1207 | HLA-A26:01 | 5 |
| DTAGLEEY | 1206 | HLA-A01:01 | 6 |
| ILDTAGLEE | 1208 | HLA-C08:02 | 6 |
| ILDTAGLEE | 1208 | HLA-A01:01 | 6 |
| CLLDILDTAGL | 1209 | HLA-A02:04 | 7 |
| ILDTAGLEE | 1208 | HLA-A36:01 | 7 |
| LLDILDTAGL | 167 | HLA-A01:01 | 7 |
| DILDTAGL | 1210 | HLA-B14:02 | 8 |
| DILDTAGLEEY | 1211 | HLA-A25:01 | 8 |
| DTAGLEEYS | 1212 | HLA-A68:02 | 8 |
| DTAGLEEYSAM | 1207 | HLA-A25:01 | 8 |
| GLEEYSAMR | 1213 | HLA-A74:01 | 8 |
| ILDTAGLE | 1214 | HLA-A01:01 | 8 |
| DILDTAGLEEY | 1211 | HLA-A26:01 | 9 |
| DTAGLEEY | 1206 | HLA-A36:01 | 9 |
| ILDTAGLEEY | 166 | HLA-A29:02 | 9 |
| DILDTAGL | 1210 | HLA-B08:01 | 10 |
| DTAGLEEY | 1206 | HLA-B18:01 | 10 |
| ILDTAGLEE | 1208 | HLA-A02:07 | 10 |
| LDTAGLEEY | 1215 | HLA-B35:01 | 10 |
| CLLDILDTAGL | 1209 | HLA-A02:01 | 11 |
| DTAGLEEY | 1206 | HLA-C02:02 | 11 |
| ILDTAGLEE | 1208 | HLA-C05:01 | 11 |
| ILDTAGLEEY | 166 | HLA-C08:02 | 11 |
| ILDTAGLEEY | 166 | HLA-A02:07 | 11 |
| LLDILDTAGL | 167 | HLA-C08:02 | 11 |
| DILDTAGL | 1210 | HLA-A26:01 | 12 |
| LDTAGLEEY | 1215 | HLA-B53:01 | 12 |
| DTAGLEEY | 1206 | HLA-C03:02 | 13 |
| DTAGLEEY | 1206 | HLA-B58:01 | 13 |
| ILDTAGLEEY | 166 | HLA-A30:02 | 13 |
| LLDILDTAGL | 167 | HLA-C05:01 | 13 |
| LLDILDTAGL | 167 | HLA-C04:01 | 13 |
| DTAGLEEYSAMR | 1216 | HLA-A68:01 | 14 |
| ILDTAGLE | 1214 | HLA-A36:01 | 15 |
| LLDILDTAGL | 167 | HLA-A02:01 | 15 |
| AGLEEYSAM | 1217 | HLA-B15:03 | 16 |
| DTAGLEEYSA | 1218 | HLA-A68:02 | 16 |
| GLEEYSAMRDQY | 1205 | HLA-A01:01 | 16 |
| ILDTAGLE | 1214 | HLA-C04:01 | 16 |
| ILDTAGLEEY | 166 | HLA-B15:01 | 16 |
| LDILDTAGL | 1219 | HLA-B37:01 | 16 |
| AGLEEYSAM | 1217 | HLA-A30:02 | 17 |
| AGLEEYSAM | 1217 | HLA-B48:01 | 17 |
| AGLEEYSAMR | 1220 | HLA-A31:01 | 17 |
| ILDTAGLEE | 1208 | HLA-C04:01 | 17 |
| LDTAGLEEY | 1215 | HLA-C03:02 | 17 |
| AGLEEYSAM | 1217 | HLA-C14:02 | 18 |
| GLEEYSAMR | 1213 | HLA-A31:01 | 18 |
| LEEYSAMRD | 1221 | HLA-B41:01 | 18 |
| LLDILDTAGLE | 1222 | HLA-A01:01 | 18 |
| AGLEEYSAM | 1217 | HLA-C14:03 | 19 |
| LDILDTAGL | 1219 | HLA-B40:02 | 19 |
| LDTAGLEEY | 1215 | HLA-A29:02 | 19 |
| DILDTAGLE | 1223 | HLA-A26:01 | 20 |
| DTAGLEEY | 1206 | HLA-B15:01 | 20 |
| ILDTAGLEEY | 166 | HLA-A02:01 | 20 |
| LDTAGLEEY | 1215 | HLA-A36:01 | 20 |
| LDTAGLEEY | 1215 | HLA-B46:01 | 20 |
| DTAGLEEY | 1206 | HLA-A68:02 | 21 |
| DTAGLEEY | 1206 | HLA-C12:03 | 21 |
| ILDTAGLE | 1214 | HLA-C05:01 | 21 |
| LDTAGLEEY | 1215 | HLA-B18:01 | 21 |
| LEEYSAMRD | 1221 | HLA-B49:01 | 21 |
| TAGLEEYSA | 1224 | HLA-B54:01 | 21 |
| DILDTAGLEEY | 1211 | HLA-A29:02 | 22 |
| GLEEYSAM | 1225 | HLA-C05:01 | 22 |

TABLE 4F
RAS G12A Mutation

| | | | Rank of |
|---|---|---|---|
| | | | Binding |
| Peptide | SEQ ID NO: | Allele | Potential |
| AAGVGKSAL | 1226 | HLA-C03:04 | 1 |
| VVVGAAGVGK | 1227 | HLA-A11:01 | 1 |
| VVGAAGVGK | 1228 | HLA-A11:01 | 2 |
| TEYKLVVVGAA | 1229 | HLA-B50:01 | 3 |
| VVGAAGVGK | 1228 | HLA-A03:01 | 3 |
| VVVGAAGVGK | 1227 | HLA-A68:01 | 3 |
| AAGVGKSAL | 1226 | HLA-C08:02 | 4 |
| AAGVGKSAL | 1226 | HLA-C08:01 | 4 |
| AAGVGKSAL | 1226 | HLA-B46:01 | 4 |
| AAGVGKSAL | 1226 | HLA-B81:01 | 5 |
| GAAGVGKSAL | 1230 | HLA-B48:01 | 5 |
| LVVVGAAGV | 1231 | HLA-A68:02 | 5 |
| AAGVGKSAL | 1226 | HLA-C03:04 | 1 |
| VVVGAAGVGK | 1227 | HLA-A11:01 | 1 |
| VVGAAGVGK | 1228 | HLA-A11:01 | 2 |
| TEYKLVVVGAA | 1229 | HLA-B50:01 | 3 |
| VVGAAGVGK | 1228 | HLA-A03:01 | 3 |
| VVVGAAGVGK | 1227 | HLA-A68:01 | 3 |
| AAGVGKSAL | 1226 | HLA-C08:02 | 4 |
| AAGVGKSAL | 1226 | HLA-C08:01 | 4 |
| AAGVGKSAL | 1226 | HLA-B46:01 | 4 |
| AAGVGKSAL | 1226 | HLA-B81:01 | 5 |
| AAGVGKSAL | 1226 | HLA-C03:02 | 5 |
| AAGVGKSAL | 1226 | HLA-C01:02 | 5 |
| GAAGVGKSAL | 1230 | HLA-B48:01 | 5 |
| LVVVGAAGV | 1231 | HLA-A68:02 | 5 |
| AAGVGKSAL | 1226 | HLA-C03:03 | 6 |
| VVGAAGVGK | 1228 | HLA-A68:01 | 6 |
| GAAGVGKSAL | 1230 | HLA-B81:01 | 7 |
| VVVGAAGVGK | 1227 | HLA-A03:01 | 7 |
| AAGVGKSAL | 1226 | HLA-C05:01 | 8 |
| AAGVGKSAL | 1226 | HLA-C12:03 | 8 |
| GAAGVGKSA | 1232 | HLA-B46:01 | 8 |
| VVGAAGVGK | 1228 | HLA-A30:01 | 8 |
| GAAGVGKSA | 1232 | HLA-B55:01 | 9 |
| KLVVVGAAGV | 1233 | HLA-A02:01 | 9 |
| AGVGKSAL | 1234 | HLA-B08:01 | 10 |
| GAAGVGKSAL | 1230 | HLA-C03:04 | 10 |
| AAGVGKSAL | 1226 | HLA-C17:01 | 11 |
| GAAGVGKSAL | 1230 | HLA-C03:03 | 11 |
| VVVGAAGV | 1235 | HLA-A68:02 | 11 |
| YKLVVVGAA | 1236 | HLA-B54:01 | 11 |
| AAGVGKSAL | 1226 | HLA-B48:01 | 12 |
| AGVGKSAL | 1234 | HLA-C03:04 | 12 |
| AGVGKSAL | 1234 | HLA-C07:01 | 12 |
| VVVGAAGVGK | 1227 | HLA-A30:01 | 12 |
| AAGVGKSA | 1237 | HLA-B46:01 | 13 |
| KLVVVGAAGV | 1233 | HLA-A02:07 | 13 |
| YKLVVVGAA | 1236 | HLA-B50:01 | 13 |
| AAGVGKSAL | 1226 | HLA-B07:02 | 14 |
| GAAGVGKSAL | 1230 | HLA-A68:02 | 14 |
| VVGAAGVGK | 1228 | HLA-A74:01 | 14 |
| AGVGKSAL | 1234 | HLA-C08:01 | 15 |
| GAAGVGKSAL | 1230 | HLA-C17:01 | 15 |
| GAAGVGKSAL | 1230 | HLA-C08:01 | 16 |
| GAAGVGKSAL | 1230 | HLA-B35:03 | 16 |
| AAGVGKSAL | 1226 | HLA-C02:02 | 17 |
| AAGVGKSAL | 1226 | HLA-B35:03 | 17 |
| AAGVGKSAL | 1226 | HLA-C12:02 | 17 |
| AAGVGKSAL | 1226 | HLA-C14:03 | 17 |
| GAAGVGKSA | 1232 | HLA-B50:01 | 17 |
| AGVGKSAL | 1234 | HLA-C03:02 | 18 |
| GAAGVGKSA | 1232 | HLA-C03:04 | 18 |
| LVVVGAAGV | 1231 | HLA-B55:01 | 18 |
| TEYKLVVVGAA | 1229 | HLA-B41:01 | 18 |
| AGVGKSAL | 1234 | HLA-C01:02 | 19 |
| GAAGVGKSA | 1232 | HLA-B54:01 | 19 |
| GAAGVGKSAL | 1230 | HLA-B07:02 | 19 |
| VGAAGVGKSA | 1238 | HLA-B55:01 | 19 |
| AGVGKSAL | 1234 | HLA-B48:01 | 20 |
| AGVGKSALTI | 1239 | HLA-B49:01 | 20 |
| VVVGAAGV | 1235 | HLA-B55:01 | 20 |

TABLE 4G
RAS G12C Mutation

| | | | Rank of |
|---|---|---|---|
| | | | Binding |
| Peptide | SEQ ID NO: | Allele | Potential |
| VVVGACGVGK | 157 | HLA-A11:01 | 1 |
| VVGACGVGK | 156 | HLA-A03:01 | 2 |
| VVGACGVGK | 156 | HLA-A11:01 | 3 |
| VVVGACGVGK | 157 | HLA-A68:01 | 4 |
| VVGACGVGK | 156 | HLA-A68:01 | 5 |
| VVVGACGVGK | 157 | HLA-A03:01 | 5 |
| VVGACGVGK | 156 | HLA-A30:01 | 6 |
| ACGVGKSAL | 1240 | HLA-B81:01 | 7 |
| ACGVGKSAL | 1240 | HLA-C01:02 | 7 |
| ACGVGKSAL | 1240 | HLA-C14:03 | 8 |
| ACGVGKSAL | 1240 | HLA-C03:04 | 9 |
| VVVGACGVGK | 157 | HLA-A30:01 | 9 |
| ACGVGKSAL | 1240 | HLA-C14:02 | 10 |
| CGVGKSAL | 1149 | HLA-B08:01 | 10 |
| KLVVVGACGV | 154 | HLA-A02:01 | 10 |
| ACGVGKSAL | 1240 | HLA-B07:02 | 11 |
| GACGVGKSAL | 1241 | HLA-B48:01 | 12 |
| GACGVGKSAL | 1241 | HLA-C03:03 | 13 |
| ACGVGKSAL | 1240 | HLA-B48:01 | 14 |
| ACGVGKSAL | 1240 | HLA-B40:01 | 14 |
| YKLVVVGAC | 1242 | HLA-B48:01 | 14 |
| YKLVVVGAC | 1242 | HLA-B15:03 | 14 |
| GACGVGKSA | 1243 | HLA-B46:01 | 15 |
| GACGVGKSAL | 1241 | HLA-C03:04 | 15 |
| GACGVGKSAL | 1241 | HLA-C01:02 | 15 |
| LVVVGACGV | 155 | HLA-A68:02 | 15 |
| CGVGKSAL | 1149 | HLA-C03:04 | 16 |
| GACGVGKSAL | 1241 | HLA-C08:02 | 16 |
| VVGACGVGK | 156 | HLA-A74:01 | 16 |

TABLE 4H
RAS G12D Mutation

| | | | Rank of |
|---|---|---|---|
| | | | Binding |
| Peptide | SEQ ID NO: | Allele | Potential |
| GADGVGKSAL | 1244 | HLA-C08:02 | 1 |
| GADGVGKSAL | 1244 | HLA-C05:01 | 2 |
| VVVGADGVGK | 159 | HLA-A11:01 | 3 |
| DGVGKSAL | 1147 | HLA-B14:02 | 4 |
| VVGADGVGK | 158 | HLA-A11:01 | 4 |
| VVGADGVGK | 158 | HLA-A03:01 | 5 |
| DGVGKSAL | 1147 | HLA-B08:01 | 6 |
| VVVGADGVGK | 159 | HLA-A68:01 | 6 |
| GADGVGKSAL | 1244 | HLA-C03:03 | 7 |
| VVGADGVGK | 158 | HLA-A30:01 | 7 |
| ADGVGKSAL | 1245 | HLA-B37:01 | 8 |
| GADGVGKSAL | 1244 | HLA-C08:01 | 8 |
| VVGADGVGK | 158 | HLA-A68:01 | 8 |
| GADGVGKSA | 1246 | HLA-C08:02 | 9 |
| GADGVGKSAL | 1244 | HLA-B35:03 | 9 |
| GADGVGKS | 1247 | HLA-C05:01 | 10 |
| GADGVGKSA | 1246 | HLA-C05:01 | 10 |
| ADGVGKSAL | 1245 | HLA-C07:01 | 11 |
| VVVGADGVGK | 159 | HLA-A03:01 | 11 |
| ADGVGKSAL | 1245 | HLA-B40:02 | 12 |
| ADGVGKSAL | 1245 | HLA-B46:01 | 13 |
| GADGVGKSAL | 1244 | HLA-C03:04 | 13 |
| ADGVGKSAL | 1245 | HLA-B81:01 | 14 |
| GADGVGKSAL | 1244 | HLA-C17:01 | 14 |
| VVVGADGVGK | 159 | HLA-A30:01 | 14 |
| GADGVGKSA | 1246 | HLA-B35:03 | 15 |
| GADGVGKSA | 1246 | HLA-B46:01 | 15 |
| GADGVGKSAL | 1244 | HLA-B48:01 | 15 |
| KLVVVGADGV | 160 | HLA-A02:01 | 15 |
| LVVVGADGV | 161 | HLA-A68:02 | 15 |
| VGADGVGKSA | 1248 | HLA-B55:01 | 15 |
| VVGADGVGK | 158 | HLA-A74:01 | 16 |
| GADGVGKSA | 1246 | HLA-B53:01 | 17 |
| KLVVVGADGV | 160 | HLA-A02:07 | 17 |
| VGADGVGK | 1249 | HLA-A68:01 | 17 |
| YKLVVVGAD | 1250 | HLA-B48:01 | 17 |
| ADGVGKSAL | 1245 | HLA-C14:03 | 18 |
| DGVGKSALTI | 1251 | HLA-B51:01 | 18 |
| VGADGVGK | 1249 | HLA-A11:01 | 18 |
| GADGVGKSAL | 1244 | HLA-B07:02 | 19 |
| KLVVVGADGVGK | 1252 | HLA-A03:01 | 20 |

TABLE 4I
RAS G12R Mutation

| | | | Rank of |
|---|---|---|---|
| | | | Binding |
| Peptide | SEQ ID NO: | Allele | Potential |
| VVGARGVGK | 1 | HLA-A03:01 | 1 |
| EYKLVVVGAR | 2 | HLA-A33:03 | 2 |
| VVVGARGVGK | 3 | HLA-A11:01 | 3 |
| ARGVGKSAL | 4 | HLA-C07:02 | 4 |
| ARGVGKSAL | 4 | HLA-B39:01 | 5 |
| ARGVGKSAL | 4 | HLA-C07:01 | 5 |
| VVGARGVGK | 1 | HLA-A11:01 | 5 |
| VVVGARGVGK | 3 | HLA-A68:01 | 5 |
| GARGVGKSA | 1253 | HLA-B46:01 | 6 |
| ARGVGKSAL | 4 | HLA-B27:05 | 7 |
| GARGVGKSA | 1253 | HLA-B55:01 | 7 |
| RGVGKSAL | 1254 | HLA-C07:01 | 8 |
| VVGARGVGK | 1 | HLA-A30:01 | 9 |
| ARGVGKSAL | 4 | HLA-B38:01 | 10 |
| ARGVGKSAL | 4 | HLA-B14:02 | 10 |
| VVGARGVGK | 1 | HLA-A68:01 | 10 |
| VVVGARGVGK | 3 | HLA-A03:01 | 11 |
| GARGVGKSAL | 1255 | HLA-B48:01 | 12 |
| RGVGKSAL | 1254 | HLA-B48:01 | 12 |
| RGVGKSALTI | 1256 | HLA-A23:01 | 12 |
| ARGVGKSAL | 4 | HLA-C06:02 | 13 |
| GARGVGKSA | 1253 | HLA-A30:01 | 13 |
| GARGVGKSAL | 1255 | HLA-B81:01 | 13 |
| VVVGARGVGK | 3 | HLA-A30:01 | 13 |
| GARGVGKSAL | 1255 | HLA-B07:02 | 14 |
| LVVVGARGV | 1257 | HLA-C06:02 | 14 |
| RGVGKSAL | 1254 | HLA-B81:01 | 14 |
| VVGARGVGK | 1 | HLA-A74:01 | 15 |
| KLVVVGARGV | 1258 | HLA-A02:01 | 16 |
| LVVVGARGV | 1257 | HLA-B55:01 | 16 |
| YKLVVVGAR | 1259 | HLA-A33:03 | 16 |
| KLVVVGAR | 1260 | HLA-A74:01 | 17 |
| KLVVVGARGV | 1258 | HLA-B13:02 | 17 |
| RGVGKSAL | 1254 | HLA-C01:02 | 17 |
| LVVVGARGV | 1257 | HLA-A68:02 | 18 |
| VVVGARGV | 1261 | HLA-B55:01 | 18 |
| ARGVGKSAL | 4 | HLA-B15:09 | 19 |
| ARGVGKSAL | 4 | HLA-C14:03 | 20 |
| GARGVGKSA | 1253 | HLA-B54:01 | 20 |
| VVVGARGV | 1261 | HLA-B52:01 | 20 |
| KLVVVGARGVGK | 1262 | HLA-A03:01 | 21 |

TABLE 4J
RAS G12S Mutation

| | | | Rank of |
|---|---|---|---|
| | | | Binding |
| Peptide | SEQ ID NO: | Allele | Potential |
| VVVGASGVGK | 1263 | HLA-A11:01 | 1 |
| VVGASGVGK | 1264 | HLA-A11:01 | 2 |
| VVGASGVGK | 1264 | HLA-A03:01 | 3 |
| VVVGASGVGK | 1263 | HLA-A68:01 | 4 |
| ASGVGKSAL | 1265 | HLA-C03:04 | 5 |
| ASGVGKSAL | 1265 | HLA-B46:01 | 5 |
| VVGASGVGK | 1264 | HLA-A68:01 | 6 |
| VVVGASGVGK | 1263 | HLA-A03:01 | 6 |
| ASGVGKSAL | 1265 | HLA-C01:02 | 7 |
| GASGVGKSAL | 1266 | HLA-B48:01 | 7 |
| ASGVGKSAL | 1265 | HLA-C07:01 | 8 |
| ASGVGKSAL | 1265 | HLA-C08:02 | 9 |
| GASGVGKSAL | 1266 | HLA-B81:01 | 9 |
| SGVGKSAL | 1267 | HLA-B08:01 | 9 |
| ASGVGKSAL | 1265 | HLA-C03:03 | 10 |
| ASGVGKSAL | 1265 | HLA-C03:02 | 10 |
| SGVGKSAL | 1267 | HLA-B14:02 | 10 |
| VVGASGVGK | 1264 | HLA-A30:01 | 10 |
| ASGVGKSAL | 1265 | HLA-C08:01 | 11 |
| VVVGASGVGK | 1263 | HLA-A30:01 | 11 |
| GASGVGKSAL | 1266 | HLA-B35:03 | 12 |
| SGVGKSAL | 1267 | HLA-C07:01 | 12 |
| ASGVGKSAL | 1265 | HLA-B81:01 | 13 |
| GASGVGKSA | 1268 | HLA-B55:01 | 13 |
| GASGVGKSAL | 1266 | HLA-C03:03 | 13 |
| KLVVVGASGV | 1269 | HLA-A02:01 | 13 |
| LVVVGASGV | 1270 | HLA-A68:02 | 13 |
| SGVGKSAL | 1267 | HLA-C01:02 | 13 |
| ASGVGKSA | 1271 | HLA-B46:01 | 14 |
| ASGVGKSAL | 1265 | HLA-C15:02 | 14 |
| GASGVGKSAL | 1266 | HLA-C08:01 | 15 |
| SGVGKSAL | 1267 | HLA-C03:04 | 15 |
| ASGVGKSAL | 1265 | HLA-C05:01 | 16 |
| GASGVGKSAL | 1266 | HLA-C03:04 | 16 |
| VVGASGVGK | 1264 | HLA-A74:01 | 16 |
| ASGVGKSAL | 1265 | HLA-B48:01 | 17 |
| GASGVGKSAL | 1266 | HLA-C01:02 | 17 |
| SGVGKSAL | 1267 | HLA-C03:02 | 17 |
| SGVGKSALTI | 1272 | HLA-A23:01 | 17 |
| VGASGVGKSA | 1273 | HLA-B55:01 | 18 |
| ASGVGKSAL | 1265 | HLA-C12:03 | 19 |
| ASGVGKSAL | 1265 | HLA-B57:03 | 19 |
| KLVVVGASGV | 1269 | HLA-A02:07 | 19 |
| SGVGKSAL | 1267 | HLA-B81:01 | 19 |
| ASGVGKSAL | 1265 | HLA-C17:01 | 20 |
| KLVVVGASG | 1274 | HLA-A32:01 | 20 |

TABLE 4K
RAS G12V Mutation

| | | | Rank of |
|---|---|---|---|
| | | | Binding |
| Peptide | SEQ ID NO: | Allele | Potential |
| VVGAVGVGK | 164 | HLA-A03:01 | 1 |
| VVGAVGVGK | 164 | HLA-A11:01 | 2 |
| VVVGAVGVGK | 5 | HLA-A11:01 | 2 |
| VVVGAVGVGK | 5 | HLA-A68:01 | 3 |
| VVGAVGVGK | 164 | HLA-A68:01 | 4 |
| LVVVGAVGV | 163 | HLA-A68:02 | 5 |
| VVGAVGVGK | 164 | HLA-A30:01 | 5 |
| AVGVGKSAL | 1275 | HLA-B81:01 | 6 |
| KLVVVGAVGV | 162 | HLA-A02:01 | 6 |
| AVGVGKSAL | 1275 | HLA-B46:01 | 7 |
| GAVGVGKSAL | 1276 | HLA-C03:03 | 7 |
| GAVGVGKSAL | 1276 | HLA-B48:01 | 7 |
| VVVGAVGVGK | 5 | HLA-A03:01 | 7 |
| AVGVGKSAL | 1275 | HLA-C03:04 | 8 |
| GAVGVGKSAL | 1276 | HLA-C03:04 | 8 |
| KLVVVGAVGV | 162 | HLA-A02:07 | 9 |
| VGVGKSAL | 1148 | HLA-B08:01 | 9 |
| VVVGAVGV | 1277 | HLA-A68:02 | 9 |
| AVGVGKSAL | 1275 | HLA-C08:02 | 10 |
| AVGVGKSAL | 1275 | HLA-B07:02 | 10 |
| GAVGVGKSAL | 1276 | HLA-B35:03 | 10 |
| AVGVGKSAL | 1275 | HLA-C08:01 | 11 |
| AVGVGKSAL | 1275 | HLA-C01:02 | 11 |
| GAVGVGKSA | 1278 | HLA-B55:01 | 11 |
| GAVGVGKSAL | 1276 | HLA-B81:01 | 11 |
| GAVGVGKSAL | 1276 | HLA-C08:01 | 11 |
| KLVVVGAVGV | 162 | HLA-B13:02 | 11 |
| VGVGKSAL | 1148 | HLA-C03:04 | 11 |
| AVGVGKSAL | 1275 | HLA-A32:01 | 12 |
| GAVGVGKSA | 1278 | HLA-B46:01 | 12 |
| VGVGKSAL | 1148 | HLA-C03:02 | 12 |
| VGVGKSALTI | 1279 | HLA-A23:01 | 12 |
| GAVGVGKSA | 1278 | HLA-B54:01 | 13 |
| VGVGKSAL | 1148 | HLA-C01:02 | .3 |
| AVGVGKSAL | 1275 | HLA-B48:01 | 14 |
| AVGVGKSAL | 1275 | HLA-C03:03 | 14 |
| AVGVGKSAL | 1275 | HLA-B42:01 | 14 |
| LVVVGAVGV | 163 | HLA-B55:01 | 14 |
| VGVGKSAL | 1148 | HLA-C08:01 | 14 |
| VVGAVGVGK | 164 | HLA-A74:01 | 14 |
| AVGVGKSAL | 1275 | HLA-C05:01 | 15 |
| AVGVGKSAL | 1275 | HLA-C03:02 | 15 |
| GAVGVGKSA | 1278 | HLA-C03:04 | 15 |
| KLVVVGAVGV | 162 | HLA-A02:04 | 15 |
| LVVVGAVGV | 163 | HLA-A02:07 | 15 |
| VGVGKSAL | 1148 | HLA-B14:02 | 15 |
| VVVGAVGVGK | 5 | HLA-A30:01 | 15 |
| VVGAVGVGK | 164 | HLA-B81:01 | 16 |
| VVVGAVGV | 1277 | HLA-B55:01 | 16 |
| AVGVGKSAL | 1275 | HLA-C14:03 | 17 |
| AVGVGKSAL | 1275 | HLA-B15:01 | 17 |
| LVVVGAVGV | 163 | HLA-B54:01 | 17 |
| AVGVGKSA | 1280 | HLA-B55:01 | 18 |
| AVGVGKSAL | 1275 | HLA-C17:01 | 18 |
| GAVGVGKSA | 1278 | HLA-B50:01 | 19 |
| GAVGVGKSAL | 1276 | HLA-C17:01 | 19 |
| YKLVVVGAV | 1281 | HLA-A02:04 | 19 |
| GAVGVGKSAL | 1276 | HLA-B35:01 | 20 |
| VVGAVGVGK | 164 | HLA-A31:01 | 20 |
| YKLVVVGAV | 1281 | HLA-B51:01 | 20 |
| LVVVGAVGVGK | 1282 | HLA-A03:01 | 21 |
| KLVVVGAVGVGK | 1283 | HLA-A03:01 | 22 |

TABLE 4L
RAS G13C Mutation

| | | | Rank of |
|---|---|---|---|
| | | | Binding |
| Peptide | SEQ ID NO: | Allele | Potential |
| VVVGAGCVGK | 1284 | HLA-A11:01 | 1 |
| VVGAGCVGK | 1285 | HLA-A11:01 | 2 |
| AGCVGKSAL | 1286 | HLA-C01:02 | 3 |
| VVGAGCVGK | 1285 | HLA-A03:01 | 4 |
| VVVGAGCVGK | 1284 | HLA-A68:01 | 4 |
| CVGKSALTI | 1287 | HLA-B13:02 | 5 |
| VVGAGCVGK | 1285 | HLA-A68:01 | 5 |
| VVGAGCVGK | 1285 | HLA-A30:01 | 6 |
| AGCVGKSAL | 1286 | HLA-B48:01 | 7 |
| AGCVGKSAL | 1286 | HLA-C03:04 | 8 |
| GCVGKSALTI | 1288 | HLA-B49:01 | 8 |
| AGCVGKSAL | 1286 | HLA-C08:02 | 9 |
| VVVGAGCVGK | 1284 | HLA-A03:01 | 9 |
| KLVVVGAGC | 1289 | HLA-A30:02 | 10 |
| GCVGKSAL | 1290 | HLA-C07:01 | 11 |
| VVGAGCVGK | 1285 | HLA-A74:01 | 12 |
| AGCVGKSAL | 1286 | HLA-C14:03 | 13 |
| KLVVVGAGC | 1289 | HLA-B15:01 | 14 |

TABLE 4M
RAS G13D Mutation

| | | | Rank of |
|---|---|---|---|
| | | | Binding |
| Peptide | SEQ ID NO: | Allele | Potential |
| AGDVGKSAL | 1291 | HLA-C08:02 | 1 |
| AGDVGKSAL | 1291 | HLA-C05:01 | 2 |
| VVGAGDVGK | 1292 | HLA-A1L01 | 3 |
| VVVGAGDVGK | 1293 | HLA-A1L01 | 3 |
| VVVGAGDVGK | 1293 | HLA-A68:01 | 4 |
| GAGDVGKSA | 1294 | HLA-B46:01 | 5 |
| GAGDVGKSAL | 1295 | HLA-B48:01 | 5 |
| VVGAGDVGK | 1292 | HLA-A68:01 | 5 |
| VVGAGDVGK | 1292 | HLA-A03:01 | 5 |
| AGDVGKSAL | 1291 | HLA-C03:04 | 6 |
| AGDVGKSAL | 1291 | HLA-C04:01 | 6 |
| AGDVGKSAL | 1291 | HLA-C0L02 | 6 |
| DVGKSALTI | 1296 | HLA-B13:02 | 6 |
| DVGKSALTI | 1296 | HLA-A25:01 | 6 |
| GDVGKSAL | 1297 | HLA-C07:01 | 6 |
| GDVGKSAL | 1297 | HLA-B40:02 | 7 |
| GDVGKSAL | 1297 | HLA-B37:01 | 8 |
| AGDVGKSAL | 1291 | HLA-B48:01 | 9 |
| DVGKSALTI | 1296 | HLA-B51:01 | 10 |
| VVGAGDVGK | 1292 | HLA-A30:01 | 10 |
| GAGDVGKSAL | 1295 | HLA-C08:01 | 11 |
| GAGDVGKSAL | 1295 | HLA-B81:01 | 11 |
| AGDVGKSAL | 1291 | HLA-C08:01 | 12 |
| GAGDVGKSAL | 1295 | HLA-C03:04 | 12 |
| DVGKSALTI | 1296 | HLA-B53:01 | 13 |
| AGDVGKSAL | 1291 | HLA-B07:02 | 14 |
| AGDVGKSAL | 1291 | HLA-B46:01 | 14 |
| DVGKSALTI | 1296 | HLA-A26:01 | 14 |
| VVGAGDVGK | 1292 | HLA-A74:01 | 14 |
| GAGDVGKSA | 1294 | HLA-B54:01 | 15 |
| DVGKSALTI | 1296 | HLA-B38:01 | 16 |
| GAGDVGKSAL | 1295 | HLA-C03:03 | 16 |
| VVVGAGDVGK | 1293 | HLA-A03:01 | 16 |

Also provided herein is a method of treating cancer in a subject comprising administering to the subject (i) a polypeptide comprising a G12R RAS epitope, or (ii) a polynucleotide encoding the polypeptide; wherein: (a) the G12R RAS epitope is vvgaRgvgk (SEQ ID NO: 1) and the subject expresses a protein encoded by an HLA-A03:01 allele; (b) the G12R RAS epitope is eyklvvvgaR (SEQ ID NO: 2) and the subject expresses a protein encoded by an HLA-A33:03 allele; (c) the G12R RAS epitope is vvvgaRgvgk (SEQ ID NO: 3) and the subject expresses a protein encoded by an HLA-A11:01 allele; or (d) the G12R RAS epitope is aRgvgksal (SEQ ID NO: 4) and the subject expresses a protein encoded by an HLA-allele selected from the group consisting of HLA-C07:02, HLA-B39:01 and HLA-C07:01.

Table 5 shows GATA peptides and their HLA binding partners.

TABLE 5

| | | | | |
|---|---|---|---|---|
| | Exemplary | | | |
| | Protein | Mutation Sequence | Peptides (HLA allele | Exemplary |
| Gene | Change | Context | example(s)) | Diseases |

FRAMESHIFT¹

| | | | | |
|---|---|---|---|---|
| GATA3 | L328fs | AQAKAVCSQESRDV | CLQCLWALL (SEQ ID NO: | Breast Cancer |
| | N334fs | LCELSDHHNHTLEEE | 1299)(A02.01) | |
| | | CQWGPCLQCLWALL | CQWGPCLQCL (SEQ ID NO: | |
| | | QASQY* (SEQ ID NO: | 1300)(A02.01) | |
| | | 1298) | QWGPCLQCL (SEQ ID NO: | |
| | | | 1301)(A24.02) | |
| | | | QWGPCLQCLW (SEQ ID NO: | |
| | | | 1302)(A24.02) | |
| GATA3 | H400fs | PGRPLQTHVLPEPHL | AIQPVLWTT (SEQ ID NO: | Breast Cancer |
| | S408fs | ALQPLQPHADHAHA | 1303)(A02.01) | |
| | S408fs | DAPAIQPVLWTTPPL | ALQPLQPHA (SEQ ID NO: | |
| | S430fs | QHGHRHGLEPCSML | 1304)(A02.01) | |
| | H434fs | TGPPARVPAVPFDLH | DLHFCRSSIM (SEQ ID NO: | |
| | H435fs | FCRSSIMKPKRDGYM | 1305)(B08.01) | |
| | | FLKAESKIMFATLQR | EPHLALQPL (SEQ ID NO: | |
| | | SSLWCLCSNH* (SEQ | 1306)(B07.02, B08.01) | |
| | | ID NO: 111) | ESKIMFATL (SEQ ID NO: 1307) | |
| | | | (B08.01) | |
| | | | FATLQRSSL (SEQ ID NO: 1308) | |
| | | | (B07.02, B08.01) | |
| | | | FLKAESKIM (SEQ ID NO: | |
| | | | 1309)(B08.01) | |
| | | | FLKAESKIMF (SEQ ID NO: | |
| | | | 1310)(B08.01) | |
| | | | GPPARVPAV (SEQ ID NO: | |
| | | | 1311)(B07.02) | |
| | | | IMKPKRDGYM (SEQ ID NO: | |
| | | | 1312)(B08.01) | |
| | | | KIMFATLQR (SEQ ID NO: | |
| | | | 1313)(A03.01) | |
| | | | KPKRDGYMF (SEQ ID NO: | |
| | | | 1314)(B07.02) | |
| | | | KPKRDGYMFL (SEQ ID NO: | |
| | | | 1315)(B07.02) | |
| | | | LHFCRSSIM (SEQ ID NO: 1316) | |
| | | | (B08.01) | |
| | | | LQHGHRHGL (SEQ ID NO: | |
| | | | 1317)(B08.01) | |
| | | | MFATLQRSSL (SEQ ID NO: | |
| | | | 1318)(B07.02, B08.01) | |
| | | | MFLKAESKI (SEQ ID NO: | |
| | | | 1319)(A24.02) | |
| | | | MLTGPPARV (SEQ ID NO: | |
| | | | 1320)(A02.01) | |
| | | | QPVLWTTPPL (SEQ ID NO: | |
| | | | 1321)(B07.02) | |
| | | | SMLTGPPARV (SEQ ID NO: | |
| | | | 1322)(A02.01) | |
| | | | TLQRSSLWCL (SEQ ID NO: | |
| | | | 1323)(A02.01) | |
| | | | VLPEPHLAL (SEQ ID NO: | |
| | | | 1324)(A02.01) | |
| | | | VPAVPFDLHF (SEQ ID NO: | |
| | | | 1325)(B07.02) | |
| | | | YMFLKAESK (SEQ ID NO: | |
| | | | 1326)(A03.01) | |
| | | | YMFLKAESKI (SEQ ID NO: | |
| | | | 1327)(A02.01, A03.01, A24.02, | |
| | | | B08.01) | |

Table 6 shows HLA affinity and stability of selected BTK peptides:

TABLE 6

| | | | SEQ | | Stability |
|---|---|---|---|---|---|
| | HLA | Peptide | ID | Affinity | (half-life |
| Gene | Allele | Sequence | NO: | (nM) | (hr.)) |
| BTK, C481S | A01.01 | YMANGSLLNY | 175 | 13.24495 | 0.866167 |
| BTK, C481S | A01.01 | MANGSLLNY | 171 | 439.029 | 0.216408 |
| BTK, C481S | A03.01 | MANGSLLNY | 171 | 35.62463 | 0.237963 |
| BTK, C481S | A03.01 | YMANGSLLNY | 175 | 95.93212 | 0.279088 |
| BTK, C481S | A11.01 | MANGSLLNY | 171 | 535.6333 | NB |
| BTK, C481S | A11.01 | YMANGSLLNY | 175 | 974.2881 | NB |
| BTK, C481S | A24.02 | EYMANGSLL | 170 | 4.961145 | 5.716141 |
| BTK_C481S | A02.01 | SLLNYLREM | 173 | 67.69132 | 3.043604 |
| BTK_C481S | A02.01 | MANGSLLNYL | 172 | 1006.566 | 0 |
| BTK_C481S | A02.01 | YMANGSLLN | 174 | 3999.442 | 0 |
| BTK_C481S | B07.02 | SLLNYLREM | 173 | 865.8805 | 0 |
| BTK_C481S | B07.02 | MANGSLLNYL | 172 | 16474.59 | 0 |
| BTK_C481S | B08.01 | SLLNYLREM | 173 | 959.6542 | 0 |
| BTK_C481S | B08.01 | MANGSLLNYL | 172 | 18463.09 | 0 |

Table 7 shows HLA affinity and stability of selected EGFR peptides:

TABLE 7

| | | | SEQ | | Stability |
|---|---|---|---|---|---|
| | HLA | Peptide | ID | Affinity | (half-life |
| Gene | Allele | Sequence | NO: | (nM) | (hr.)) |
| EGFR, T790M | A01.01 | LTSTVQLIM | 182 | 2891.111 | 0.103721 |
| EGFR_T790M | A01.01 | CLTSTVQLIM | 177 | 8276.876 | 0 |
| EGFR_T790M | A02.01 | MQLMPFGCLL | 184 | 16.26147 | 0.381118 |
| EGFR_T790M | A02.01 | MQLMPFGCL | 183 | 116.3352 | 0.368273 |
| EGFR_T790M | A02.01 | LIMQLMPFGC | 181 | 132.4766 | 0.381284 |
| EGFR_T790M | A02.01 | QLIMQLMPF | 185 | 192.8406 | 0.34067 |
| EGFR_T790M | A02.01 | CLTSTVQLIM | 177 | 537.1391 | 0 |
| EGFR_T790M | A02.01 | IMQLMPFGCL | 179 | 653.1065 | 0.515559 |
| EGFR_T790M | A02.01 | IMQLMPFGC | 178 | 1205.368 | 0.370112 |
| EGFR_T790M | A02.01 | LIMQLMPFG | 180 | 3337.708 | 0 |
| EGFR_T790M | A02.01 | VQLIMQLMPF | 188 | 4942.892 | 0 |
| EGFR_T790M | A02.01 | QLIMQLMPFG | 186 | 5214.668 | 0 |
| EGFR_T790M | A02.01 | STVQLIMQL | 187 | 7256.773 | 0 |
| EGFR_T790M | A24.02 | QLIMQLMPF | 185 | 2030.807 | 0.368673 |
| EGFR_T790M | A24.02 | VQLIMQLMPF | 188 | 4103.131 | 0 |
| EGFR_T790M | A24.02 | IMQLMPFGCL | 179 | 14119.38 | 0 |
| EGFR_T790M | A24.02 | MQLMPFGCLL | 184 | 18857.47 | 0 |
| EGFR_T790M | B07.02 | MQLMPFGCL | 183 | 1589.188 | 0 |
| EGFR_T790M | B08.01 | QLIMQLMPF | 185 | 330.1933 | 0 |
| EGFR_T790M | B08.01 | IMQLMPFGCL | 179 | 427.3913 | 0 |
| EGFR_T790M | B08.01 | MQLMPFGCL | 183 | 4931.727 | 0 |
| EGFR_T790M | B08.01 | MQLMPFGCLL | 184 | 11244.9 | 0 |
| EGFR_T790M | B08.01 | VQLIMQLMPF | 188 | 16108.18 | 0 |
| EGFR_T790M | B08.02 | QLIMQLMPF | 185 | 5590.3 | ND |

Tumor Antigens Associated with Tumor Microenvironment

In many cases, predominant antigens are expressed by cells in the tumor microenvironment that not only serve as excellent biomarkers for the disease, but also can be important vaccine candidates for immunotherapy. Such tumor associated antigens (TAAs) are not necessarily presented on the surface of tumor cells, but on cells that are juxtaposed to the tumor, which could be the stromal cells, connective tissue cells, fibroblasts etc. These are cells that often contribute to the structural integrity of the tumor, feed the tumor and support growth of the tumor. In most cases, TAAs are overexpressed antigens in the tumor microenvironment, however some antigens in the tumor microenvironment may also be unique in the tumor associated cells. As an example, telomerase reverse transcriptase (TERT) is a TAA that is not present in most normal tissues but is activated in most human tumors. Tissue kallikrein-related peptidases, or kallikreins (KLKs), on the other hand are overexpressed in various cancers and comprise a large family of secreted trypsin- or chymotrypsin-like serine proteases. Kallikreins are upregulated in prostrate ovarian and breast cancers. Some TAAs are specific to certain cancers, some are expressed in a large variety of cancers. Carcinoembryonic antigen (CEA) is overexpressed in breast, colon, lung and pancreatic carcinomas, whereas MUC-1 is breast, lung, prostate, colon cancers. Some TAAs are differentiation or tissue specific, for example, MART-1/melan-A and gp100 are expressed in normal melanocytes and melanoma, and prostate specific membrane antigen (PSMA) and prostate-specific antigen (PSA) are expressed by prostate epithelial cells as well as prostate carcinoma.

In some embodiments, T cells are developed for adoptive therapy that are directed to overexpressed tissue specific or tumor associated antigens, such as prostrate specific kallikrein proteins KLK2, KLK3, KLK4 in case of prostate cancer therapy, or transglutamase protein 4, TGM4 for adenocarcinoma.

In some embodiments, the antigenic peptides that are targeted for the adoptive therapy in the methods disclosed herein are effective in modulating the tumor microenvironment. T cells are primed with antigens expressed by cells in the TME, so that the therapy is directed towards weakening and/or breaking down the tumor facilitating TME, oftentimes, in addition to directly targeting the tumor cells for T cell mediated lysis.

Tumor microenvironment comprises fibroblasts, stromal cells, endothelial cells and connective tissue cells which make up a large proportion of cells that induce or influence tumor growth. Just as T cells can be stimulated and directed attack the tumor cells in a immunosuppressive tumor environment, certain peptides and antigens can be utilized to direct the T cells against cells in the tumor vicinity that help in tumor propagation CD8+ and CD4+ T cells can be generated ex vivo that are directed against antigens on the surface of non-tumor cells in the tumor microenvironment that promote tumor sustenance and propagation. Cancer/tumor associated fibroblasts (CAFs) are hallmark feature of pancreatic cancers, such as pancreatic adenocarcinoma (PDACs). CAFs express Col10a1 antigen. CAFs are cells that may help perpetuate a tumor. Col10A1 often confers negative prognosis for the tumor. In some embodiments Col10A1 may be considered as a biomarker for tumor sustenance and progression. It is a 680 amino acid long heterodimer protein associated with poor prognosis in breast cancer and colorectal cancers.

Activation of Col10a1 specific CD8+ T cells and CD4+ T cells may help attack and destruction of Col10A1 specific fibroblasts and help break down the tissue matrix of solid tumors.

T cells can be generated ex vivo using the method described herein, so that the T cells are activated against cancer-associated fibroblasts (CAFs). For this, Col10a1 peptides comprising epitopes that can specifically activate T cells were generated, and the HLA binding partner determined, using the highly reliable data generated from the in-house generated machine learning epitope presentation software described previously as described in Table 8.

TABLE 8

| | SEQ | | Rank on |
|---|---|---|---|
| Peptide | ID NO: | HLA Allele | HLA allele |
| FTCQIPGIYY | 1328 | HLA-A01:01 | 1 |
| GSDGKPGY | 1329 | HLA-A01:01 | 2 |
| NAESNGLY | 1330 | HLA-A01:01 | 3 |
| LTENDQVWL | 1331 | HLA-A01:01 | 4 |
| GTHVWVGLY | 1332 | HLA-A01:01 | 5 |
| TYDEYTKGY | 1333 | HLA-A01:01 | 6 |
| YTYDEYTKGY | 1334 | HLA-A01:01 | 7 |
| FTCQIPGIY | 1335 | HLA-A01:01 | 8 |
| NAESNGLYSSEY | 1336 | HLA-A01:01 | 9 |
| YLDQASGSA | 1337 | HLA-A01:01 | 10 |
| FLLLVSLNL | 1338 | HLA-A02:01 | 1 |
| FLLLVSLNLV | 1339 | HLA-A02:01 | 2 |
| GLYKNGTPV | 1340 | HLA-A02:01 | 3 |
| GLDGPKGNPGL | 1341 | HLA-A02:01 | 4 |
| LLLVSLNLV | 1342 | HLA-A02:01 | 5 |
| SLSGTPLVSA | 1343 | HLA-A02:01 | 6 |
| GLYSSEYV | 1344 | HLA-A02:01 | 7 |
| SLSGTPLV | 1345 | HLA-A02:01 | 8 |
| MLPQIPFLL | 1346 | HLA-A02:01 | 9 |
| GLPGPPGPSA | 1347 | HLA-A02:01 | 10 |
| SAFTVILSK | 1348 | HLA-A03:01 | 1 |
| AVMPEGFIK | 1349 | HLA-A03:01 | 2 |
| GLYKNGTPVMY | 1350 | HLA-A03:01 | 3 |
| AIGTPIPFDK | 1351 | HLA-A03:01 | 4 |
| GLPGGPGAK | 1352 | HLA-A03:01 | 5 |
| ILYNRQQHY | 1353 | HLA-A03:01 | 6 |
| AGPPGPPGFGK | 1354 | HLA-A03:01 | 7 |
| GIPGFPGSK | 1355 | HLA-A03:01 | 8 |
| GTHVWVGLYK | 1356 | HLA-A03:01 | 9 |
| GVPGQPGIK | 1357 | HLA-A03:01 | 10 |
| AVMPEGFIK | 1349 | HLA-A11:01 | 1 |
| SAFTVILSK | 1348 | HLA-A11:01 | 2 |
| VSAFTVILSK | 1358 | HLA-A11:01 | 3 |
| GTHVWVGLYK | 1356 | HLA-A11:01 | 4 |
| AIGTPIPFDK | 1351 | HLA-A11:01 | 5 |
| AVMPEGFIKA | 1359 | HLA-A11:01 | 6 |
| SSFSGFLVA | 1360 | HLA-A11:01 | 7 |
| PVSAFTVILSK | 1361 | HLA-A11:01 | 8 |
| GIPGFPGSK | 1355 | HLA-A11:01 | 9 |
| GVPGMNGQK | 1362 | HLA-A11:01 | 10 |
| AYPAIGTPIPF | 1363 | HLA-A24:02 | 1 |
| IGPPGIPGF | 1364 | HLA-A24:02 | 2 |
| HYDPRTGIF | 1365 | HLA-A24:02 | 3 |
| EYVHSSFSGF | 1366 | HLA-A24:02 | 4 |
| AGPPGPPGF | 1367 | HLA-A24:02 | 5 |
| YYFSYHVHV | 1368 | HLA-A24:02 | 6 |
| AYPAIGTPI | 1369 | HLA-A24:02 | 7 |
| PLPNTKTQF | 1370 | HLA-A24:02 | 8 |
| MLPQIPFLL | 1346 | HLA-A24:02 | 9 |
| CQIPGIYYF | 1371 | HLA-A24:02 | 10 |
| RPSLSGTPL | 1372 | HLA-B07:02 | 1 |
| LPQIPFLLL | 1373 | HLA-B07:02 | 2 |
| IPFLLLVSL | 1374 | HLA-B07:02 | 3 |
| LPGPPGPSAV | 1375 | HLA-B07:02 | 4 |
| GPIGPPGIPGF | 1376 | HLA-B07:02 | 5 |
| IPGPAGISV | 1377 | HLA-B07:02 | 6 |
| YPAIGTPIPF | 1378 | HLA-B07:02 | 7 |
| SPGPPGPAGI | 1379 | HLA-B07:02 | 8 |
| LPGPPGPSA | 1380 | HLA-B07:02 | 9 |
| SPGPPGPAG | 1381 | HLA-B07:02 | 10 |
| TIKSKGIAV | 1382 | HLA-B08:01 | 1 |
| IPFLLLVSL | 1374 | HLA-B08:01 | 2 |
| HVHVKGTHV | 1383 | HLA-B08:01 | 3 |
| LPNTKTQF | 1384 | HLA-B08:01 | 4 |
| LPQIPFLL | 1385 | HLA-B08:01 | 5 |
| PFLLLVSL | 1386 | HLA-B08:01 | 6 |
| SLNLVHGV | 1387 | HLA-B08:01 | 7 |
| LPQIPFLLL | 1373 | HLA-B08:01 | 8 |
| TGMPVSAF | 1388 | HLA-B08:01 | 9 |
| TPIPFDKIL | 1389 | HLA-B08:01 | 10 |

Neoantigenic peptides provided herein are prevalidated for HLA binding immunogenicity (Tables 1-8 and 11-14). In some embodiments the neoantigenic peptides, prepared and stored earlier, are used to contact an antigen presenting cell (APC) to then allow presentation to a T cell in vitro for preparation of neoantigen-specific activated T cell. In some embodiments, between 2-80 or more neoantigenic peptides are used to stimulate T cells from a patient at a time.

In some embodiments the APC is an autologous APC. In some embodiments the APC is a non-autologous APC. In some embodiments the APC is a synthetic cell designed to function as an APC. In some embodiments the T cell is an autologous cell. In some embodiments, an antigen presenting cell is a cell that expresses an antigen. For example, an antigen presenting cell may be a phagocytic cell such as a dendritic cell or myeloid cell, which process an antigen after cellular uptake and presents the antigen in association with an MEC for T cell activation. For certain purposes, an APC as used herein is a cell that normally presents an antigen on its surface. In a non-binding or non-limiting example, relevant to certain cytotoxicity assays as described herein, a tumor cell is an antigen presenting cell, that the T cell can recognize an antigen presenting cell (tumor cell). Similarly, a cell or cell line expressing an antigen can be, for certain purposes as used herein, an antigen presenting cell.

In some embodiments, one or more polynucleotides encoding one or more neoantigenic peptides may be used to express in a cell to present to a T cell for activation in vitro. The one or more polynucleotides encoding one or more of the neoantigenic peptides are encoded in a vector. In some embodiments, the composition comprises from about 2 to about 80 neoantigenic polynucleotides. In embodiments, at least one of the additional neoantigenic peptide is specific for an individual subject's tumor. In embodiments, the subject specific neoantigenic peptide is selected by identifying sequence differences between the genome, exome, and/or transcriptome of the subject's tumor sample and the genome, exome, and/or transcriptome of a non-tumor sample. In embodiments, the samples are fresh or formalin-fixed paraffin embedded tumor tissues, freshly isolated cells, or circulating tumor cells. In embodiments, the sequence differences are determined by Next Generation Sequencing.

In some embodiments the method and compositions provided herein can be used to identify or isolate a T cell receptor (TCR) capable of binding at least one neoantigenic peptide described herein or an MEC-peptide complex comprising at least one neoantigenic peptide described herein. In embodiments, the MHC of the MHC-peptide is MHC class I or class II. In embodiments, TCR is a bispecific TCR further comprising a domain comprising an antibody or antibody fragment capable of binding an antigen. In embodiments, the antigen is a T cell-specific antigen. In embodiments, the antigen is CD3. In embodiments, the antibody or antibody fragment is an anti-CD3 scFv.

In some embodiments the method and compositions provided herein can be used to prepare a chimeric antigen receptor comprising: (i) a T cell activation molecule; (ii) a transmembrane region; and (iii) an antigen recognition moiety capable of binding at least one neoantigenic peptide described herein or an MEC-peptide complex comprising at least one neoantigenic peptide described herein. In embodiments, CD3-zeta is the T cell activation molecule. In embodiments, the chimeric antigen receptor further comprises at least one costimulatory signaling domain. The In embodiments, the signaling domain is CD28, 4-1BB, ICOS, OX40, ITAM, or Fc epsilon RI-gamma. In embodiments, the antigen recognition moiety is capable of binding the isolated neoantigenic peptide in the context of MEW class I or class II. In embodiments, the CD3-zeta, CD28, CTLA-4, ICOS, BTLA, KIR, LAG3, CD137, OX40, CD27, CD40L, Tim-3, A2aR, or PD-1 transmembrane region. In embodiments, the neoantigenic peptide is located in the extracellular domain of a tumor associated polypeptide. In embodiments, the MHC of the MHC-peptide is MHC class I or class II.

Provided herein is a T cell comprising the T cell receptor or chimeric antigen receptor described herein, optionally wherein the T cell is a helper or cytotoxic T cell. In embodiments, the T cell is a T cell of a subject.

Provided herein is a T cell comprising a T cell receptor (TCR) capable of binding at least one neoantigenic peptide described herein or an MHC-peptide complex comprising at least one neoantigenic peptide described herein, wherein the T cell is a T cell isolated from a population of T cells from a subject that has been incubated with antigen presenting cells and one or more of the at least one neoantigenic peptide described herein for a sufficient time to activate the T cells. In embodiments, the T cell is a CD8+ T cell, a helper T cell or cytotoxic T cell. In embodiments, the population of T cells from a subject is a population of CD8+ T cells from the subject. In embodiments, the one or more of the at least one neoantigenic peptide described herein is a subject-specific neoantigenic peptide. In embodiments, the subject-specific neoantigenic peptide has a different tumor neo-epitope that is an epitope specific to a tumor of the subject. In embodiments, the subject-specific neoantigenic peptide is an expression product of a tumor-specific non-silent mutation that is not present in a non-tumor sample of the subject. In embodiments, the subject-specific neoantigenic peptide binds to a HLA protein of the subject. In embodiments, the subject-specific neoantigenic peptide binds to a HLA protein of the subject with an IC50 less than 500 nM. In embodiments, the activated CD8+ T cells are separated from the antigen presenting cells. In embodiments, the antigen presenting cells are dendritic cells or CD40L-expanded B cells. In embodiments, the antigen presenting cells are non-transformed cells. In embodiments, the antigen presenting cells are non-infected cells. In embodiments, the antigen presenting cells are autologous. In embodiments, the antigen presenting cells have been treated to strip endogenous MEC-associated peptides from their surface. In embodiments, the treatment to strip the endogenous MHC-associated peptides comprises culturing the cells at about 26° C. In embodiments, the treatment to strip the endogenous MEC-associated peptides comprises treating the cells with a mild acid solution. In embodiments, the antigen presenting cells have been pulsed with at least one neoantigenic peptide described herein. In embodiments, pulsing comprises incubating the antigen presenting cells in the presence of at least about 2 μg/mL of each of the at least one neoantigenic peptide described herein. In embodiments, ratio of isolated T cells to antigen presenting cells is between about 30:1 and 300:1. In embodiments, the incubating the isolated population of T cells is in the presence of IL-2 and IL-7. In embodiments, the MHC of the MHC-peptide is MHC class I or class II.

Provided herein is a method for activating tumor specific T cells comprising: isolating a population of T cells from a subject; and incubating the isolated population of T cells with antigen presenting cells and at least one neoantigenic peptide described herein for a sufficient time to activate the T cells. In embodiments, the T cell is a CD8+ T cell, a helper T cell or cytotoxic T cell. In embodiments, the population of T cells from a subject is a population of CD8+ T cells from the subject. In embodiments, the one or more of the at least one neoantigenic peptide described herein is a subject-specific neoantigenic peptide. In embodiments, the subject-specific neoantigenic peptide has a different tumor neo-epitope that is an epitope specific to a tumor of the subject. In embodiments, the subject-specific neoantigenic peptide is an expression product of a tumor-specific non-silent mutation that is not present in a non-tumor sample of the subject. In embodiments, the subject-specific neoantigenic peptide binds to a HLA protein of the subject. In embodiments, the subject-specific neoantigenic peptide binds to a HLA protein of the subject with an IC50 less than 500 nM. In embodiments, the method further comprises separating the activated T cells from the antigen presenting cells. In embodiments, the method further comprises testing the activated T cells for evidence of reactivity against at least one of neoantigenic peptide of described herein. In embodiments, the antigen presenting cells are dendritic cells or CD40L-expanded B cells. In embodiments, the antigen presenting cells are non-transformed cells. In embodiments, the antigen presenting cells are non-infected cells. In embodiments, the antigen presenting cells are autologous. In embodiments, the antigen presenting cells have been treated to strip endogenous MEC-associated peptides from their surface. In embodiments, the treatment to strip the endogenous MHC-associated peptides comprises culturing the cells at about 26° C. In embodiments, the treatment to strip the endogenous MEC-associated peptides comprises treating the cells with a mild acid solution. In embodiments, the antigen presenting cells have been pulsed with at least one neoantigenic peptide described herein. In embodiments, pulsing comprises incubating the antigen presenting cells in the presence of at least about 2 μg/ml of each of at least one neoantigenic peptide described herein. In embodiments, ratio of isolated T cells to antigen presenting cells is between about 30:1 and 300:1. In embodiments, the incubating the isolated population of T cells is in the presence of IL-2 and IL-7. In embodiments, the MHC of the MHC-peptide is MHC class I or class II.

Provided herein is a composition comprising activated tumor specific T cells produced by a method described herein.

Provided herein is a method of treating cancer in a subject comprising administering to the subject a therapeutically effective amount of activated tumor specific T cell described herein, or produced by a method described herein. In embodiments, the administering comprises administering from about 10\hat{ }6 to 10\hat{ }12, from about 10\hat{ }8 to 10\hat{ }11, or from about 10\hat{ }9 to 10\hat{ }10 of the activated tumor specific T cells.

Provided herein is a nucleic acid comprising a promoter operably linked to a polynucleotide encoding the T cell receptor described herein. In embodiments, the TCR is capable of binding the at least one neoantigenic peptide in the context of major histocompatibility complex (MHC) class I or class II.

Provided herein is a nucleic acid comprising a promoter operably linked to a polynucleotide encoding the chimeric antigen receptor described herein. In embodiments, the antigen recognition moiety is capable of binding the at least one neoantigenic peptide in the context of major histocompatibility complex (MHC) class I or class II. In embodiments, the neoantigenic peptide is located in the extracellular domain of a tumor associated polypeptide. In embodiments, the nucleic acid comprises the CD3-zeta, CD28, CTLA-4, ICOS, BTLA, KIR, LAG3, CD137, OX40, CD27, CD40L, Tim-3, A2aR, or PD-1 transmembrane region.

In some embodiments the autologous immune cells from the peripheral blood of the patient constitute peripheral blood mononuclear cells (PBMC). In some embodiments the autologous immune cells from the peripheral blood of the patient are collected via an apheresis procedure. In some embodiments, the PBMCs are collected from more than one apheresis procedures, or more than one draw of peripheral blood.

In some embodiments, both CD25+ cells and the CD14+ cells are depleted prior to addition of peptides. In some embodiments, either of CD25+ cells or the CD14+ cells are depleted prior to addition of peptides. In some embodiments, CD25+ cells and not the CD14+ cells are depleted prior to addition of peptides.

In some embodiments, the depletion procedure is followed by the addition of FMS-like tyrosine kinase 3 receptor ligand (FLT3L) to stimulate the antigen presenting cells (APCs), constituted by the monocytes, macrophages or dendritic cells (DCs) prior to addition of the peptides. In some embodiments, the depletion procedure is followed by selection of DC as suitable PACs for peptide presentation to the T cells, and mature macrophages and other antigen presenting cells are removed from the autologous immune cells from the patient. In some embodiments, the depletion procedure is followed by selection of immature DC as suitable PACs for peptide presentation to the T cells.

In some embodiments, a selection of ‘n’ number of neoantigenic peptides is contacted with the APCs for stimulation of the APCs for antigen presentation to the T cells.

In some embodiments, a first level selection of ‘n’ number of neoantigenic peptides is based on the binding ability of each of the peptides to at least on HLA haplotype that is predetermined to be present in the recipient patient. In order to determine HLA haplotype that is predetermined to be present in the recipient patient, as is known to one of skill in the art, a patient is subjected to HLA haplotyping assay form a blood sample prior to the commencement of the treatment procedure. In some embodiments, a first level selection of ‘n’ number of neoantigenic peptides is followed by a second level selection based on the determination of whether the mutation present in the neoantigenic peptide(s) match the neoantigens (or mutations leading to) known to be found in at least 5% of patients known to have the cancer. In some embodiments, the second level of the selection involves further determination of whether the mutation is evident in the patient.

In some embodiments, a first and the second level selection of ‘n’ number of neoantigenic peptides for contacting the APCs is followed by a third level of selection, based on the binding affinity of the peptide with the HLA that the peptide is capable of binding to and is at least less than 500 nM, with the determination that higher the binding affinity, the better the choice of the peptide to be selected. In some embodiments, the finally selected ‘n’ number of peptides can range from 1-200 peptides which are in a mix, for exposing APCs to the peptides in the culture media, and contacting with APCs.

In some embodiments the ‘n’ number of peptides can range from 10-190 neoantigenic peptides. In some embodiments the ‘n’ number of peptides can range from 20-180 neoantigenic peptides. In some embodiments the ‘n’ number of peptides can range from 30-170 neoantigenic peptides. In some embodiments the ‘n’ number of peptides can range from 40-160 neoantigenic peptides. In some embodiments the ‘n’ number of peptides can range from 50-150 neoantigenic peptides. In some embodiments the ‘n’ number of peptides can range from 60-140 neoantigenic peptides. In some embodiments the ‘n’ number of peptides can range from 70-130 neoantigenic peptides. In some embodiments the ‘n’ number of peptides can range from 80-120 neoantigenic peptides. In some embodiments the ‘n’ number of peptides can range from 50-100 neoantigenic peptides. In some embodiments the ‘n’ number of peptides can range from 50-90 neoantigenic peptides. In some embodiments the ‘n’ number of peptides can range from 50-80 neoantigenic peptides. In some embodiments the ‘n’ number of peptides comprise at least 60 neoantigenic peptides. In some embodiments the ‘n’ number of peptides comprise a mixture of (a) neoantigenic peptides that are short, 8-15 amino acids long, comprising the mutated amino acid as described previously, following the formula AxByCz; these peptides are interchangeably called shortmers or short peptides for the purpose of this application; and (b) long peptides that are 15, 30, 50, 60, 80, 100-300 amino acids long and any length in between, which are subject to endogenous processing by dendritic cells for better antigen presentation; these peptides are interchangeably called longmers or long peptides for the purpose of this application. In some embodiments the at least 60 neoantigenic peptides comprise at least 30 shortmers and at least 30 longmers or variations of the same. Exemplary variations of the same include, but are not limited to the following: in some embodiments the at least 60 neoantigenic peptides comprise at least 32 shortmers and at least 32 longmers or variations of the same. In some embodiments the at least 60 neoantigenic peptides comprise at least 34 shortmers and at least 30 longmers or variations of the same. In some embodiments the at least 60 neoantigenic peptides comprise at least 28 shortmers and at least 34 longmers or variations of the same.

In some embodiments, the ‘n’ number of peptides are incubated in the medium comprising APCs in culture, where the APCs (DCs) have been isolated from the PBMCs, and previously stimulated with FLT3L. In some embodiments, the ‘n’ number of peptides are incubated with APCs in presence of FLT3L. In some embodiments, following the step of incubation of the APCs with FLT3L, the cells are added with fresh media containing FL3TL for incubation with peptides. In some embodiments, the maturation of APCs to mature peptide loaded DCs may comprise several steps of culturing the DCs towards maturation, examining the state of maturation by analysis of one or more released substances, (e.g. cytokines, chemokines) in the culture media or obtaining an aliquot of the DCs in culture form time to time. In some embodiments, the maturation of DCs take at least 5 days in culture from onset of the culture. In some embodiments, the maturation of DCs take at least 7 days in culture from onset of the culture. In some embodiments, the maturation of DCs take at least 11 days in culture from onset of the culture, or any number of days in between.

In some embodiments, the DCs are contacted with T cells after being verified for presence of or absence of maturation factors and peptide tetramer assay for verifying the repertoire of antigens presented.

In some embodiments, the DCs are contacted with T cells in a T cell media for about 2 days for the first induction. In some embodiments, the DCs are contacted with T cells in a T cell media for about 3 days for the first induction. In some embodiments, the DCs are contacted with T cells in a T cell media for about 4 days for the first induction. In some embodiments, the DCs are contacted with T cells in a T cell media for at least about 2 days for the second induction. In some embodiments, the DCs are contacted with T cells in a T cell media for at least about 3 days for the second induction. In some embodiments, the DCs are contacted with T cells in a T cell media for at least about 4 days for the second induction. In some embodiments, the DCs are contacted with T cells in a T cell media for 5 days for the second induction. In some embodiments, the DCs are contacted with T cells in a T cell media for about 6 days for the second induction. In some embodiments, the DCs are contacted with T cells in a T cell media for about 7, 8, 9 or 10 days for the second induction. In some embodiments, the DCs are contacted with T cells in a T cell media for about less than 1 days for the third induction. In some embodiments, the DCs are contacted with T cells in a T cell media for at least about 2 or 3 days for the third induction. In some embodiments, the DCs are contacted with T cells in a T cell media for at least about 4 days for the third induction. In some embodiments, the DCs are contacted with T cells in a T cell media for 5 days for the third induction. In some embodiments, the DCs are contacted with T cells in a T cell media for about 6 days for the third induction. In some embodiments, the DCs are contacted with T cells in a T cell media for about 7, 8, 9 or 10 days for the second induction.

In some embodiments, the T cells are further contacted with one or more shortmer peptides during incubation with DCs (and in addition to the DCs) at either the first induction phase, the second induction phase or the third induction phase. In some embodiments, the T cells are further contacted with one or more shortmer peptides during incubation with DCs at the first induction phase and the second induction phase. In some embodiments, the T cells are further contacted with one or more shortmer peptides during incubation with DCs at the second induction phase and the third induction phase. In some embodiments, the T cells are further contacted with one or more shortmer peptides in all the three induction phases.

In some embodiments, the APCs and the T cells are comprised in the same autologous immune cells from the peripheral blood of the patient drawn at the first step from the patient. The T cells are isolated and preserved for the time of activation with the DCs at the end of the DC maturation phase. In some embodiments the T cells are cocultured in the presence of a suitable media for activation for the time of activation with the DCs at the end of the DC maturation phase. In some embodiments the T cells are prior cyropreserved cells from the patient, which are thawed and cultured for at least 4 hours to up to about 48 hours for induction at the time of activation with the DCs at the end of the DC maturation phase.

In some embodiments, the APCs and the T cells are comprised in the same autologous immune cells from the peripheral blood of the patient drawn at the different time periods from the patient, e.g. at different apheresis procedures. In some embodiments the time from apheresis of the patient to the time of harvest, takes between about 20 days to about less than 26 days. In some embodiments the time from apheresis of the patient to the time of harvest, takes between about 21 days to about less than 25 days. In some embodiments the time from apheresis of the patient to the time of harvest, takes between about 21 days to about less than 24 days. In some embodiments the time from apheresis of the patient to the time of harvest, takes between about 21 days to about less than 23 days. In some embodiments the time from apheresis of the patient to the time of harvest, takes about 21 days. In some embodiments the time from apheresis of the patient to the time of harvest, takes about less than 21 days.

In some embodiments the release criteria for the activated T cells (the drug substance) comprises any one or more of sterility, endotoxin, cell phenotype, TNC Count, viability, cell concentration, potency. In some embodiments the release criteria for the activated T cells (the drug substance) comprises each one of sterility, endotoxin, cell phenotype, TNC Count, viability, cell concentration, potency.

In some embodiments the total number of cells is 2×10\hat{ }10. In some embodiments the total number of cells is 2×10\hat{ }9. In some embodiments the total number of cells is 5×10\hat{ }8. In some embodiments the total number of cells is 2×10\hat{ }8. In some embodiments the final concentration of the resuspended T cells is 2×10\hat{ }5 cells/ml or more. In some embodiments the final concentration of the resuspended T cells is 1×10\hat{ }6 cells/ml or more. In some embodiments the final concentration of the resuspended T cells is 2×10\hat{ }6 cells/ml or more.

The following criteria of released cells are described as exemplary non-limiting conditions, particularly because of the reason that the criteria for the cell population and subpopulations in Drug substance (DS) can vary based on the cancer, the state of the cancer, the state of the patient, the availability of the matched HLA haplotype and the growth potential of the APCs and T cells in the presence of the peptide. In some embodiments the activated T cells (the drug substance) comprises at least 2% or at least 3% or at least 4% or at least 5% of CD8+ T cells reactive to a particular neoantigen by tetramer assay. In some embodiments, the activated T cells (the drug substance) comprises at least 2% or at least 3% or at least 4% or at least 5% of CD4+ T cells reactive to a particular neoantigen by tetramer assay. In some embodiments, the activated T cells (the drug substance) comprise at least 5% or at least 6% or at least 7% or at least 8% or at least 9% or at least 10% of cells that are positive for memory T cell phenotype.

In some embodiments, the activated T cells (the drug substance) are selected based on one or more markers. In some embodiments, the activated T cells (the drug substance) are not selected based on one or more markers. In some embodiments, an aliquot of the activated T cells (the drug substance) are tested for the presence or absence of one or more of the following markers, and the proportions of cells thereof exhibiting each of the tested markers, the one or more markers are selected from a group consisting of: CD19, CD20, CD21, CD22, CD24, CD27, CD38, CD40, CD72, CD3, CD79a, CD79b, IGKC, IGHD, MZB1, TNFRSF17, MS4A1, CD138, TNFRSR13B, GUSPB11, BAFFR, AID, IGHM, IGHE, IGHA1, IGHA2, IGHA3, IGHA4, BCL6, FCRLA CCR7, CD27, CD45RO, FLT3LG, GRAP2, IL16, IL7R, LTB, S1PR1, SELL, TCF7, CD62L, PLACE, SORL1, MGAT4A, FAM65B, PXN, A2M, ATM, C20orf112, GPR183, EPB41, ADD3, GRAP2, KLRG1, GIMAP5, TC2N, TXNIP, GIMAP2, TNFAIP8, LMNA, NR4A3, CDKN1A, KDM6B, ELL2, TIPARP, SC5D, PLK3, CD55, NR4A1, REL, PBX4, RGCC, FOSL2, SIK1, CSRNP1, GPR132, GLUL, KIAA1683, RALGAPA1, PRNP, PRMT10, FAM177A1, CHMP1B, ZC3H12A, TSC22D2, P2RY8, NEU1, ZNF683, MYADM, ATP2B1, CREM, OAT, NFE2L2, DNAJB9, SKIL, DENND4A, SERTAD1, YPEL5, BCL6, EGR1, PDE4B, ANXA1, SOD2, RNF125, GADD45B, SELK, RORA, MXD1, IFRD1, PIK3R1, TUBB4B, HECA, MPZL3, USP36, INSIG1, NR4A2, SLC2A3, PER1, S100A10, AIM1, CDC42EP3, NDEL1, IDI1, EIF4A3, BIRC3, TSPYL2, DCTN6, HSPH1, CDK17, DDX21, PPP1R15B, ZNF331, BTG2, AMD1, SLC7A5 POLR3E, JMJD6, CHD1, TAF13, VPS37B, GTF2B, PAF1, BCAS2, RGPD6, TUBA4A, TUBA1A, RASA3, GPCPD1, RASGEF1B, DNAJA1, FAM46C, PTP4A1, KPNA2, ZFAND5, SLC38A2, PLIN2, HEXIM1, TMEM123, JUND, MTRNR2L1, GABARAPL1, STAT4, ALG13, FOSB, GPR65, SDCBP, HBP1, MAP3K8, RANBP2, FAM129A, FOS, DDIT3, CCNH, RGPD5, TUBA1C, ATP1B3, GLIPR1, PRDM2, EMD, HSPD1, MORF4L2, IL21R, NFKBIA, LYAR, DNAJB6, TMBIM1, PFKFB3, MED29, B4GALT1, NXF1, BIRC2, ARHGAP26, SYAP1, DNTTIP2, ETF1, BTG1, PBXIP1, MKNK2, DEDD2, AKIRIN1, HLA-DMA, HLA-DNB, HLA-DOA, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, CCL18, CCL19, CCL21, CXCL13, LAMP3, LTB, IL7R, MS4A1, CCL2, CCL3, CCL4, CCL5, CCL8, CXCL10, CXCL11, CXCL9, CD3, LTA, IL17, IL23, IL21, IL7, CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, TIGIT, CD56, CCL2, CCL3, CCL4, CCL5, CXCL8, IFN, IL-2, IL-12, IL-15, IL-18, NCR1, XCL1, XCL2, IL21R, KIR2DL3, KIR3DL1, KIR3DL2, NCAM1, HLA-DMA, HLA-DNB, HLA-DOA, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, and HLA-DRB5.

In some embodiments, at least 0.01% of naive T cells which were obtained from the obtaining of autologous immune cells from the peripheral blood of the patient were stimulated in response to a neoantigen, and was amplified at the end of the procedure and was harvested. In some embodiments, greater than 0.01% of naive T cells which were obtained from the obtaining of autologous immune cells from the peripheral blood of the patient were stimulated in response to a neoantigen, and was amplified at the end of the procedure and was harvested. In some embodiments, greater than 0.1% of naive T cells which were obtained from the obtaining of autologous immune cells from the peripheral blood of the patient were stimulated in response to a neoantigen, and was amplified at the end of the procedure and was harvested. In some embodiments, greater than 1% of naive T cells which were obtained from the obtaining of autologous immune cells from the peripheral blood of the patient were stimulated in response to a neoantigen, and was amplified at the end of the procedure and was harvested.

In some embodiments the total number of cells is harvested from 1, 2, or 3 cycles of the process of DC maturation and T cell activation.

In some embodiments the harvested cells are cryopreserved in vapor phase of liquid nitrogen in bags.

As is known to one of skill in the art, all applications described in the preceding paragraphs of this section from obtaining of autologous immune cells from the peripheral blood of the patient to the harvesting of cells is performed in an aseptic closed system, except the steps where aliquots of media or cells are taken out for examination by flow cytometry, mass spectroscopy, cell count, cell sorting or any functional assays, that are terminal to the cells or materials taken out as aliquots. In some embodiments the closed system for aseptic culture of up to the harvesting is proprietary to the applicant's process.

In some embodiments the T cells are method for culturing and expansion of activated T cells including the steps delineated above, starting from obtaining of autologous immune cells from the peripheral blood of the patient to harvesting, is scalable in an aseptic procedure. In some embodiments, at least 1 Liter of DC cell culture is performed at a time. In some embodiments, at least 1-2 Liters of T cell culture is performed at a time. In some embodiments, at least 5 Liters of DC cell culture is performed at a time. In some embodiments, at least 5-10 Liters of T cell culture is performed at a time. In some embodiments, at least 10 Liter of DC cell culture is performed at a time. In some embodiments, at least 10-40 Liters of T cell culture is performed at a time. In some embodiments, at least 10 Liter of DC cell culture is performed at a time. In some embodiments, at least 10-50 Liters of T cell culture is performed at a time. In some embodiments, simultaneous batch cultures are performed and tested in a system that is a closed system, and that can be manipulated and intervened from outside without introducing non-aseptic means. In some embodiments, a closed system described herein is fully automated.

When administration is by injection, the active agent can be formulated in aqueous solutions, specifically in physiologically compatible buffers such as Hanks solution, Ringer's solution, or physiological saline buffer. The solution can contain formulation agents such as suspending, stabilizing and/or dispersing agents. Alternatively, the active compound can be in powder form for constitution with a suitable vehicle, e.g., sterile pyrogen-free water, before use. In another embodiment, the drug product comprises a substance that further activates or inhibits a component of the host's immune response, for example, a substance to reduce or eliminate the host's immune response to the peptide.

The disclosure provided herein demonstrates that shared neoantigens can be used for ready therapeutic administration of a patient, thereby reducing the bench-to-bedside time lag considerably. The composition and methods described herein provide innovative advancements in the field of cancer therapeutics.

### EXAMPLES

### Example 1. Precision NEOSTIM Clinical Process

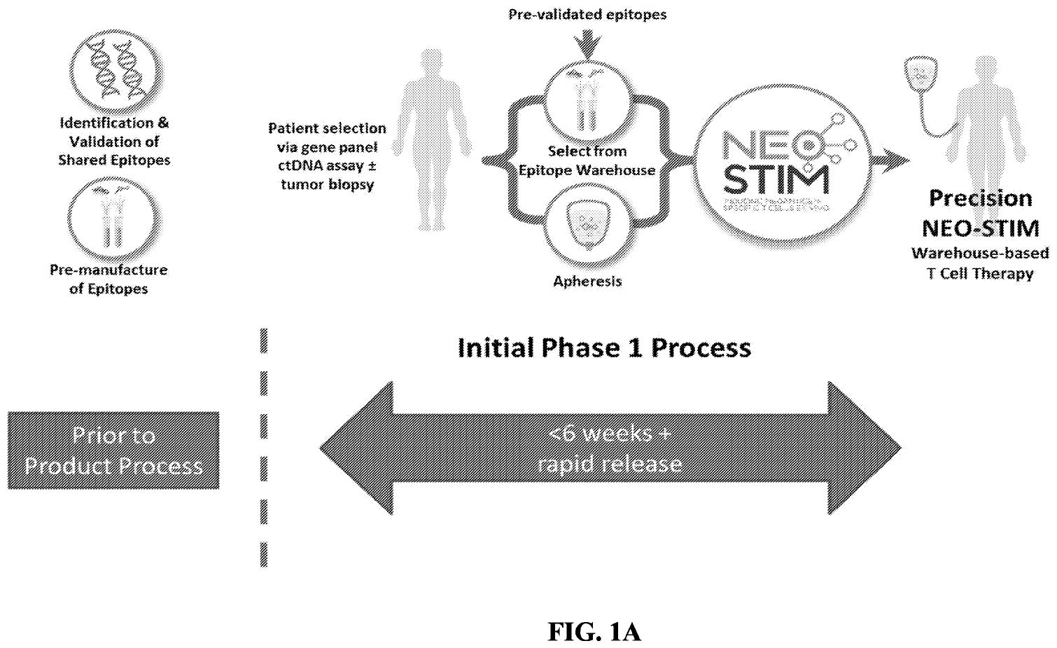

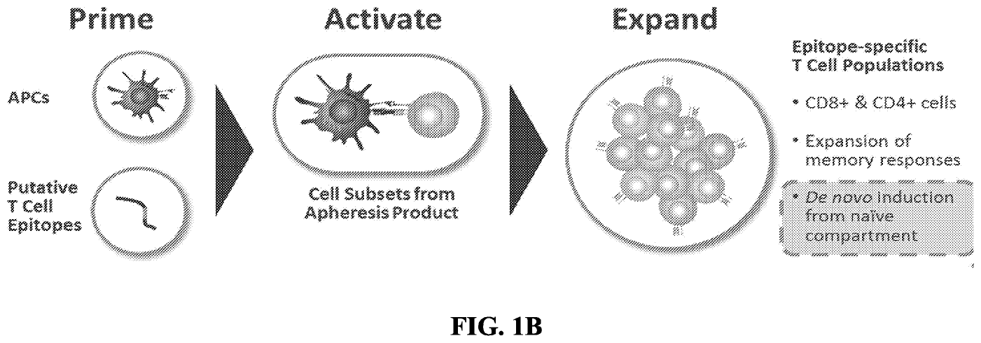

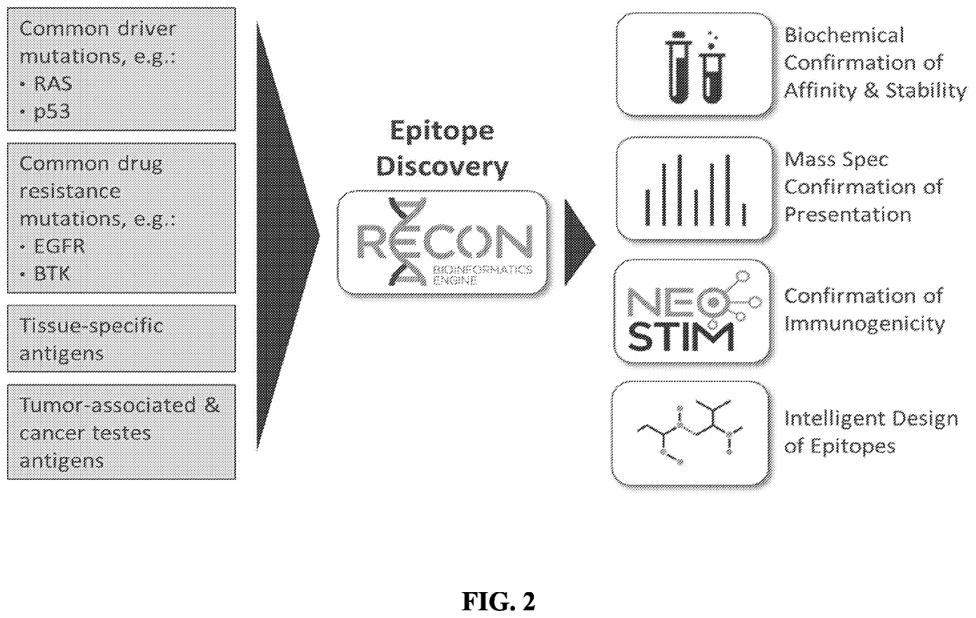

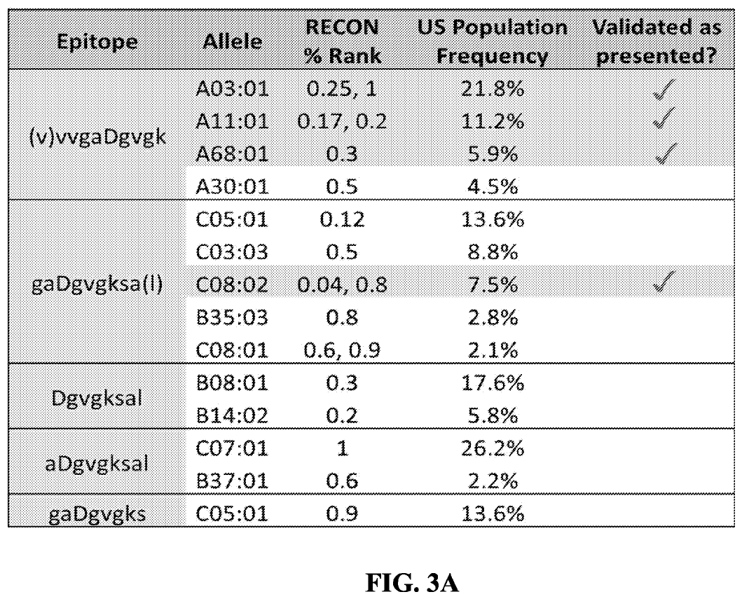

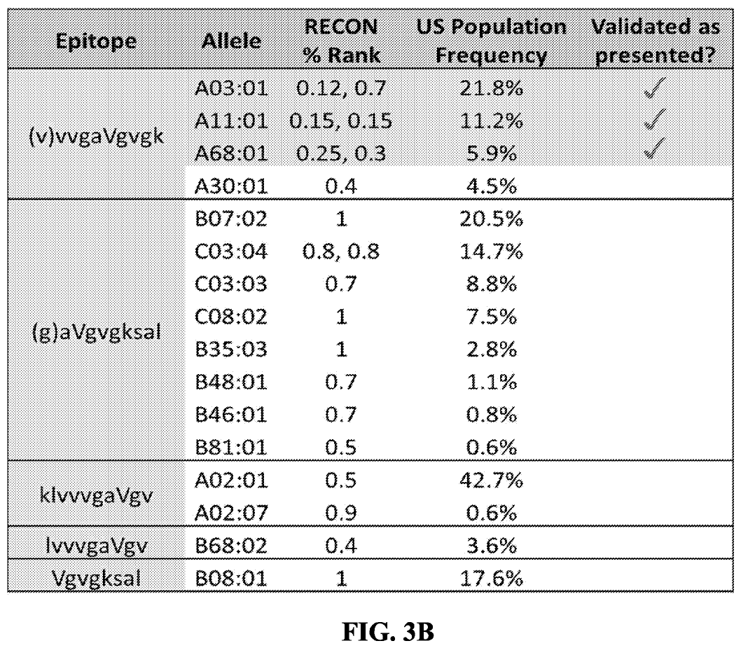

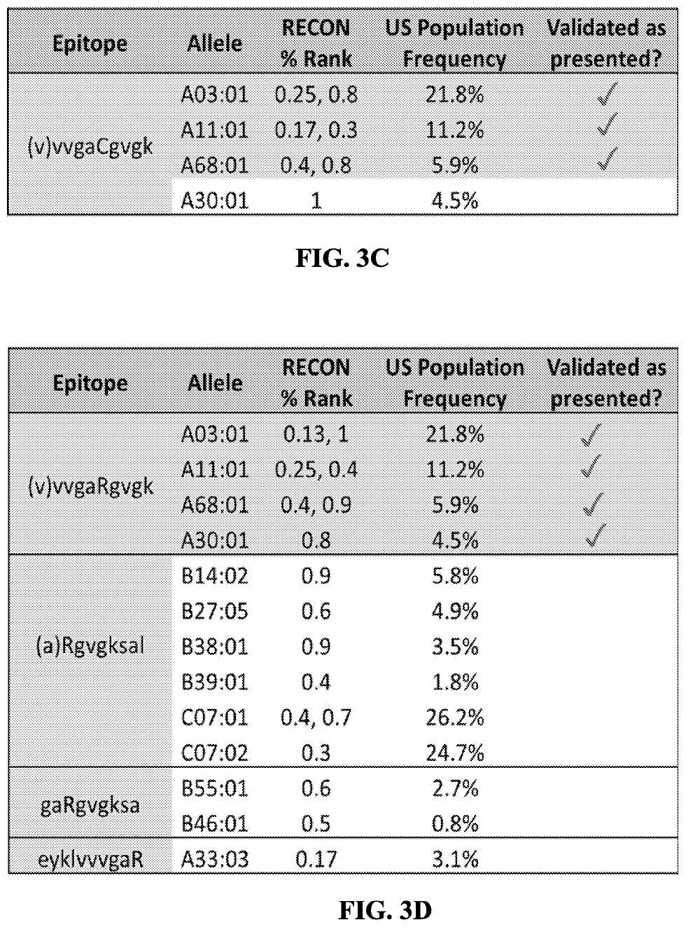

Provided herein is an adoptive T cell therapy where T cells primed and responsive against curated pre-validated, shelved, antigenic peptides specific for a subject's cancer is administered to the subject. Provided in this example is a method of bypassing lengthy sequencing, identification and manufacture of subject specific neoantigen peptides and thereafter generating T cells having the subject specific TCRs for cancer immunotherapy, at least for the time when a subject undergoes a process of such evaluation and preparations for the personalized therapy. Advantage of this process is that it is fast, targeted and robust. As shown in FIG. 1A, patient identified with a cancer or tumor can be administered T cells that are activated ex vivo with warehouse curated peptides having selected, prevalidated collection of epitopes generated from a library of shared antigens known for the identified cancer. The process from patient selection to the T cell therapy may require less than 6 weeks. FIG. 1B illustrates the method of generating cancer target specific T cells ex vivo by priming T cells with antigen presenting cells (APCs) expressing putative T cell epitopes and expanding the activated T cells to obtain epitope-specific CD8+ and CD4+ including a population of these cells exhibiting memory phenotype (see, e.g., WO2019094642, incorporated by reference in its entirety). A library of prevalidated epitopes is generated in advance. Such epitopes are collected from prior knowledge in the field, common driver mutations, common drug resistant mutations, tissue specific antigens, and tumor associated antigens. With the help of an efficient computer-based program for epitope prediction, HLA binding and presentation characteristics, pre-validated peptides are generated for storage and stocking as shown in a diagram in FIG. 2. Exemplary predictions for common RAS G12 mutations are shown in FIG. 3A-3D. Validations are performed using a systematic process as outlined in Examples 2-5. Target tumor cell antigen responsive T cells are generated ex vivo and immunogenicity is validated using an in vitro antigen-specific T cell assay (Example 2). Mass spectrometry is used to validate that cells that express the antigen of interest can process and present the peptides on the relevant HLA molecules (Example 3). Additionally, the ability of these T cells to kill cells presenting the peptide is confirmed using a cytotoxicity assay (Example 4). Exemplary data provided herein demonstrate this validation process for RAS and GATA3 neoantigens, and can be readily applied to other antigens.

### Example 2. Generation of Target Tumor Cell Antigen Responsive T Cells Ex Vivo

Materials:

AIM V media (Invitrogen)
Human FLT3L, preclinical CellGenix #1415-050 Stock 50 ng/μL
TNF-α, preclinical CellGenix #1406-050 Stock 10 ng/μL
IL-1β, preclinical CellGenix #1411-050 Stock 10 ng/μL
PGE1 or Alprostadil—Cayman from Czech republic Stock 0.5 μg/μL
R10 media—RPMI 1640 glutamax+10% Human serum+1% PenStrep
20/80 Media—18% AIM V+72% RPMI 1640 glutamax+10% Human Serum+1% PenStrep
IL7 Stock 5 ng/μL
IL15 Stock 5 ng/μL

### Procedure:

Step 1: Plate 5 million PBMCs (or cells of interest) in each well of 24 well plate with FLT3L in 2 mL AIM V media
Step 2: Peptide loading and maturation—in AIMV
1. Mix peptide pool of interest (except for no peptide condition) with PBMCs (or cells of interest) in respective wells.

### 2. Incubate for 1 hr.

3. Mix Maturation cocktail (including TNF-α, IL-1β, PGE1, and IL-7) to each well after incubation.
Step 3: Add human serum to each well at a final concentration of 10% by volume and mix.
Step 4: Replace the media with fresh RPMI+10% HS media supplemented with IL7+IL15.
Step 5: Replace the media with fresh 20/80 media supplemented with IL7+IL15 during the period of incubation every 1-6 days.
Step 6: Plate 5 million PBMCs (or cells of interest) in each well of new 6-well plate with FLT3L in 2 ml AIM V media
Step 7: Peptide loading and maturation for re-stimulation—(new plates)
1. Mix peptide pool of interest (except for no peptide condition) with PBMCs (or cells of interest) in respective wells

### 2. Incubate for 1 hr.

3. Mix Maturation cocktail to each well after incubation

### Step 8: Re-stimulation:

1. Count first stimulation FLT3L cultures and add 5 million cultured cells to the new Re-stimulation plates.
2. Bring the culture volume to 5 mL (AIM V) and add 500 μL of Human serum (10% by volume)
Step 9: Remove 3 ml of the media and add 6 ml of RPMI+10% HS media supplemented with IL7+IL15.
Step 10: Replace 75% of the media with fresh 20/80 media supplemented with IL7+IL15.
Step 11: Repeat re-stimulation if needed.

### Analysis of Antigen-Specific Induction

MHC tetramers are purchased or manufactured on-site according to methods known by one of ordinary skill, and are used to measure peptide-specific T cell expansion in the immunogenicity assays. For the assessment, tetramer is added to 1×10⁵ cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4° C. for 20 minutes. Cells are washed with cold FACS buffer and resuspended in buffer containing 1% formaldehyde. Cells are acquired on a LSR Fortessa (Becton Dickinson) instrument, and are analyzed by use of FlowJo software (Becton Dickinson). For analysis of tetramer positive cells, the lymphocyte gate is taken from the forward and side-scatter plots. Data are reported as the percentage of cells that were CD8⁺/tetramer⁺.

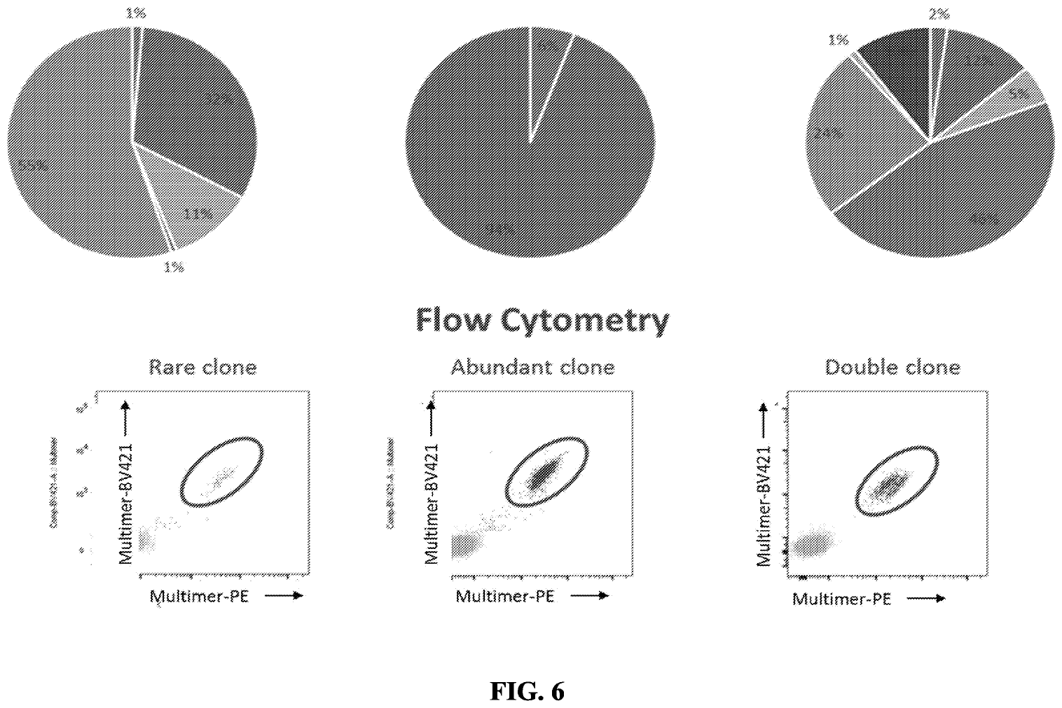

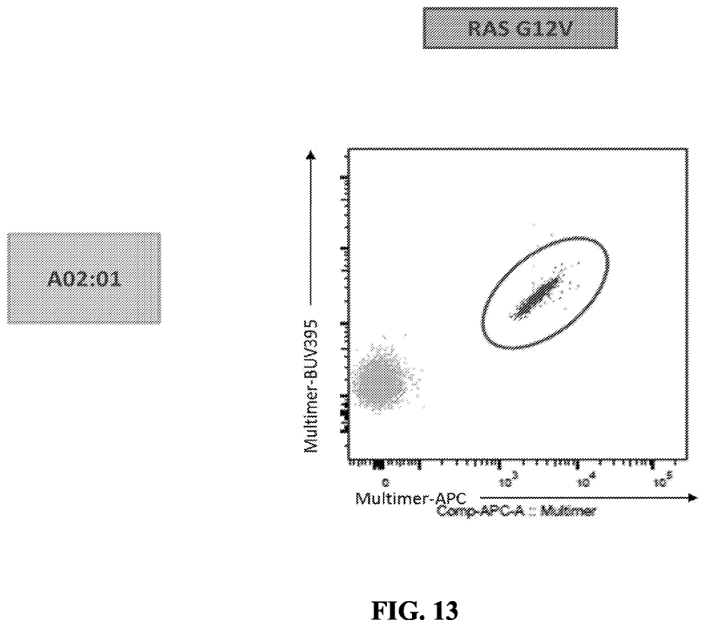

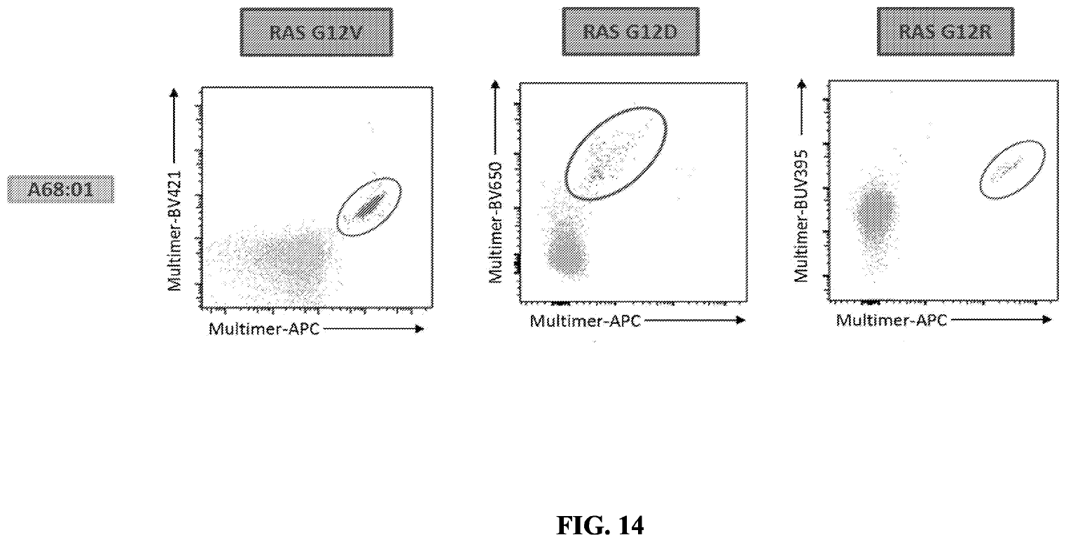

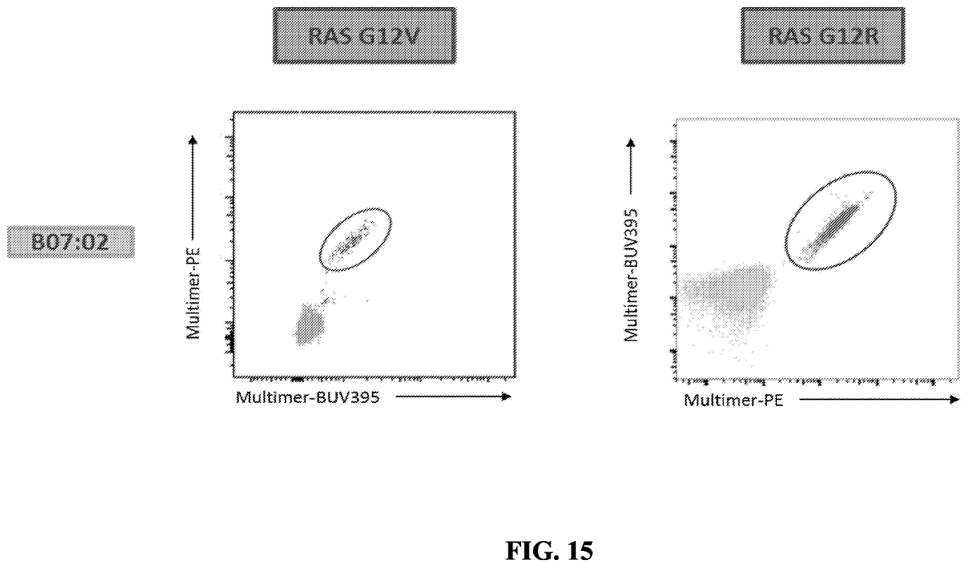

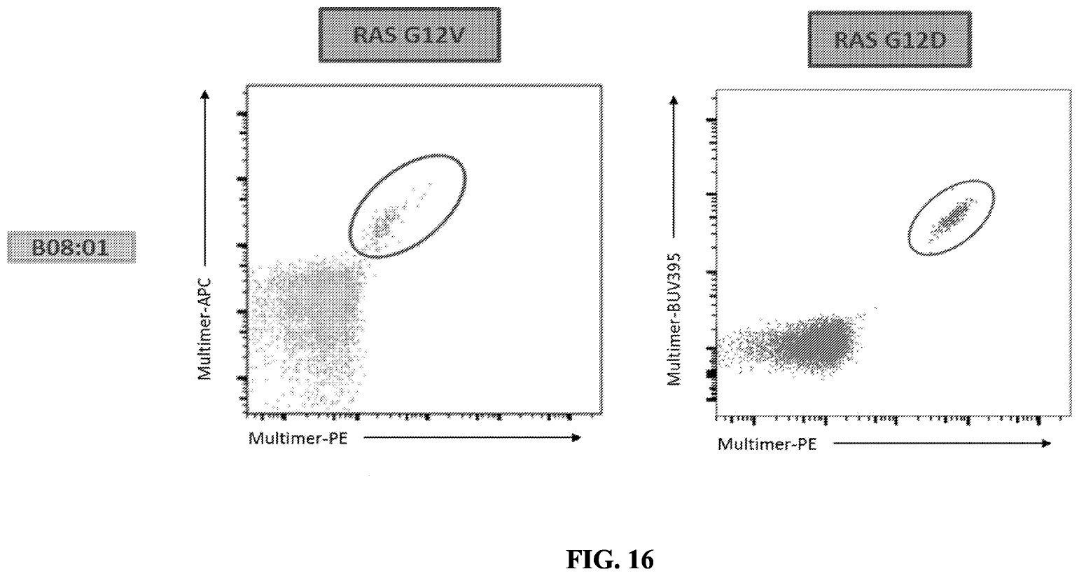

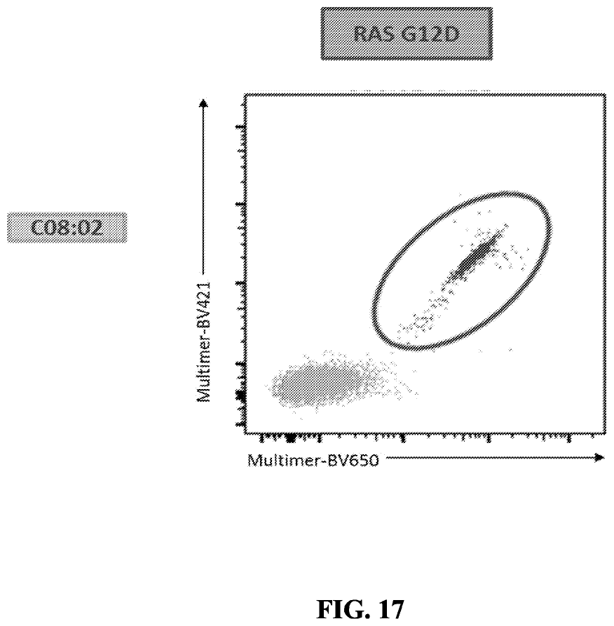

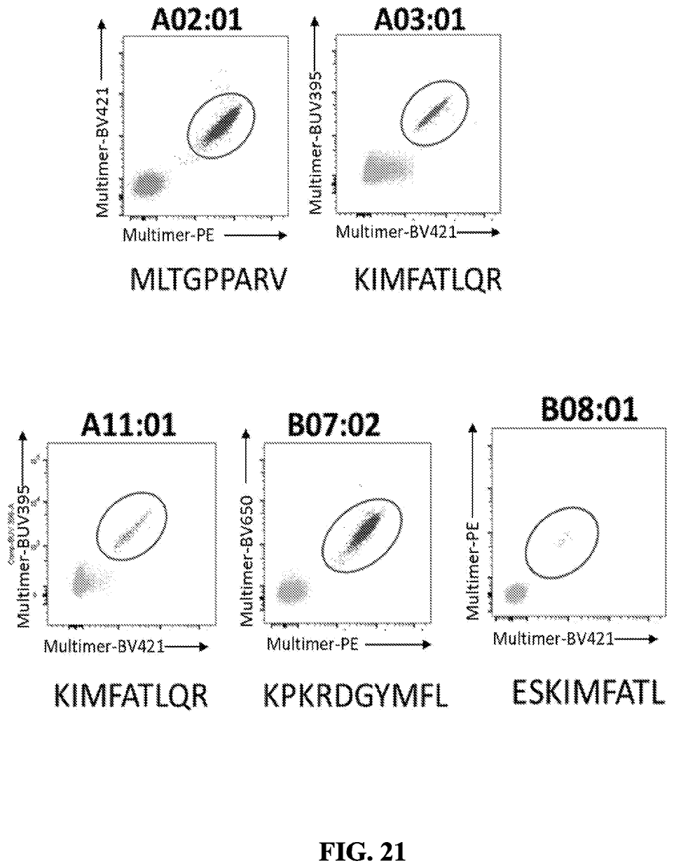

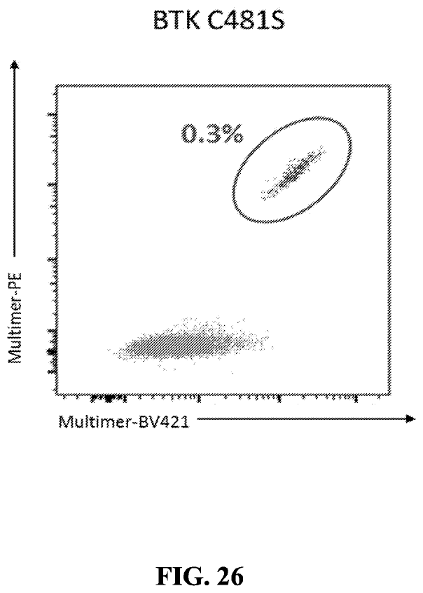

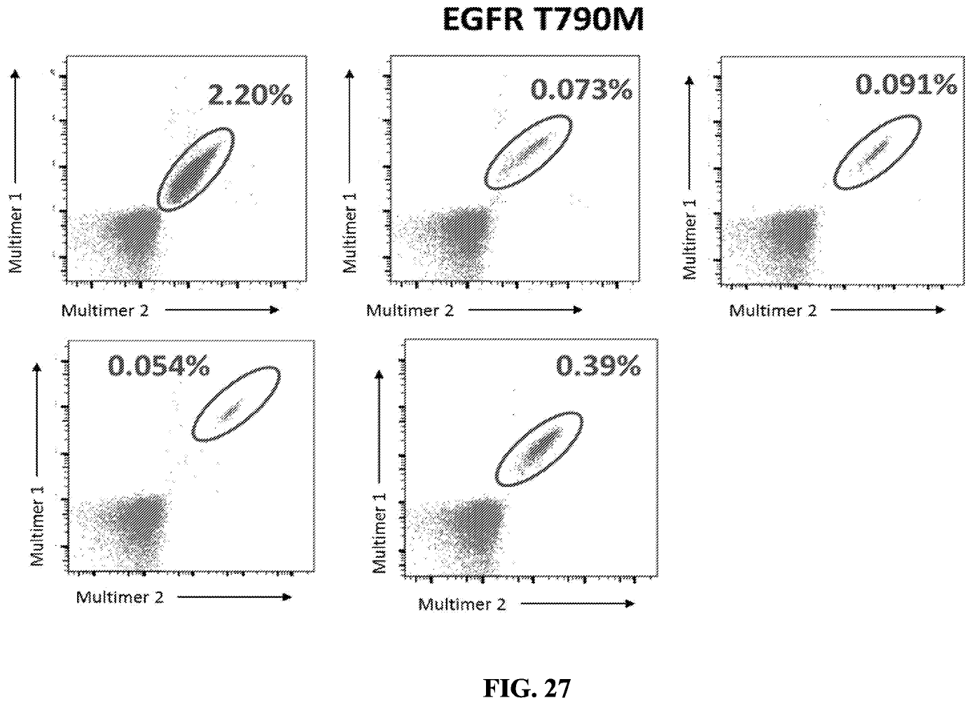

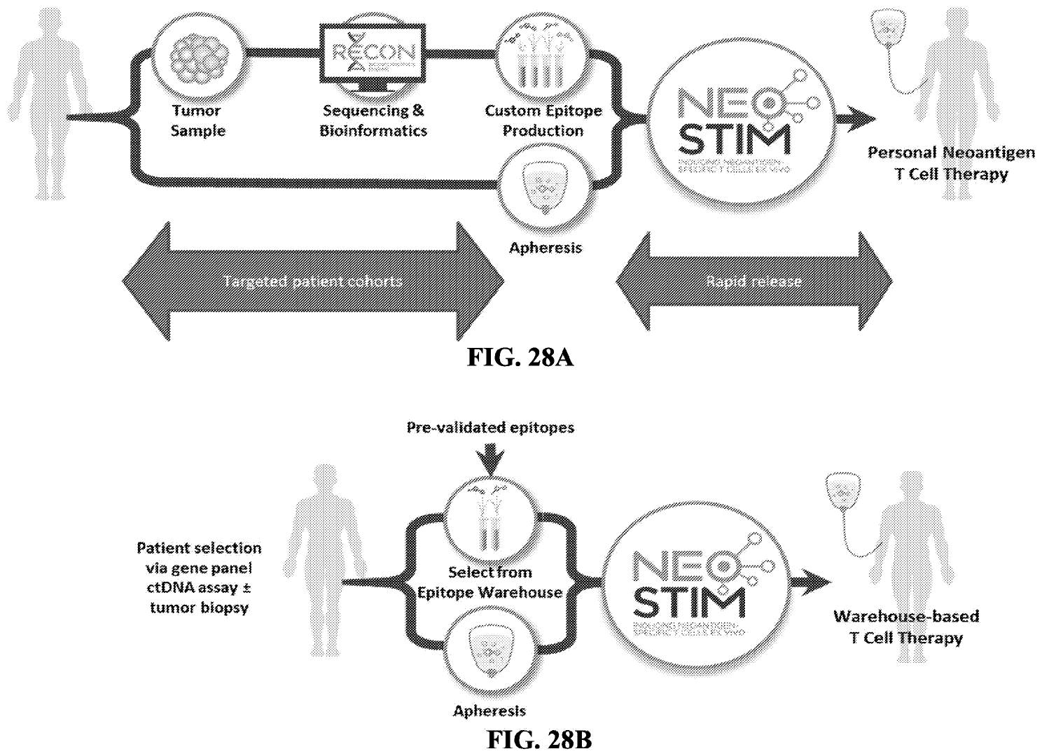

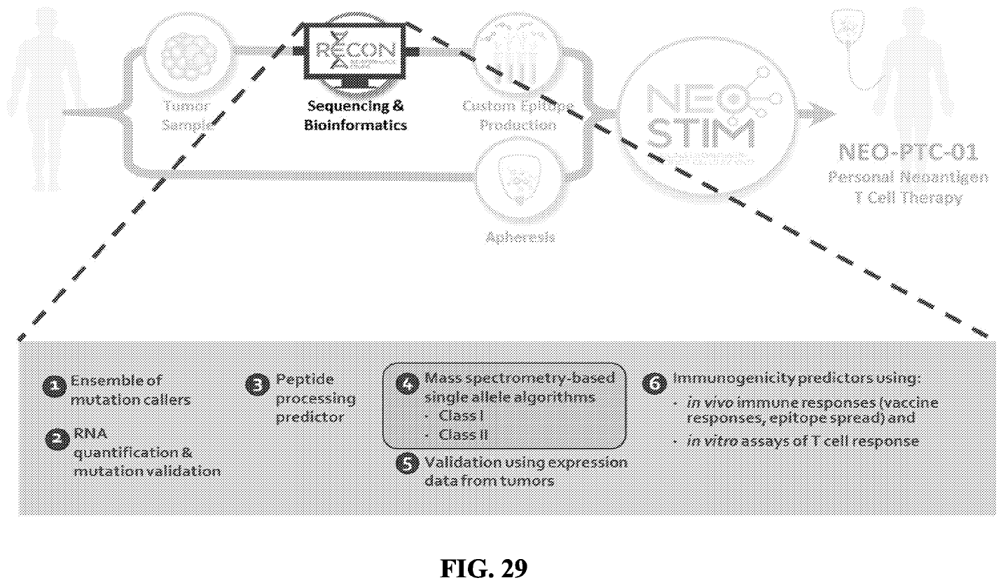

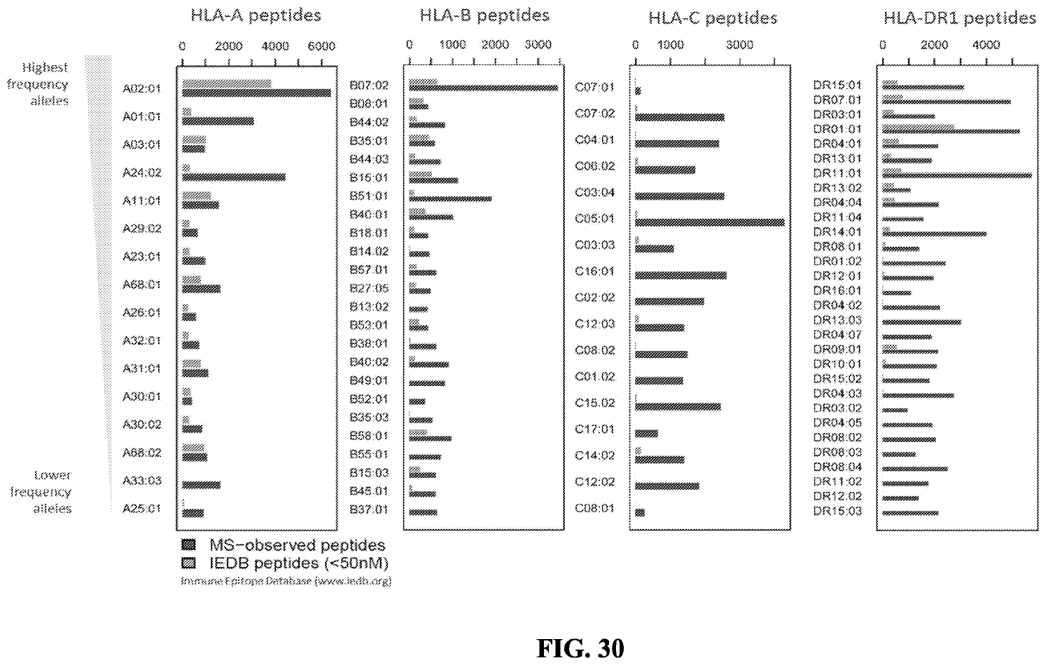

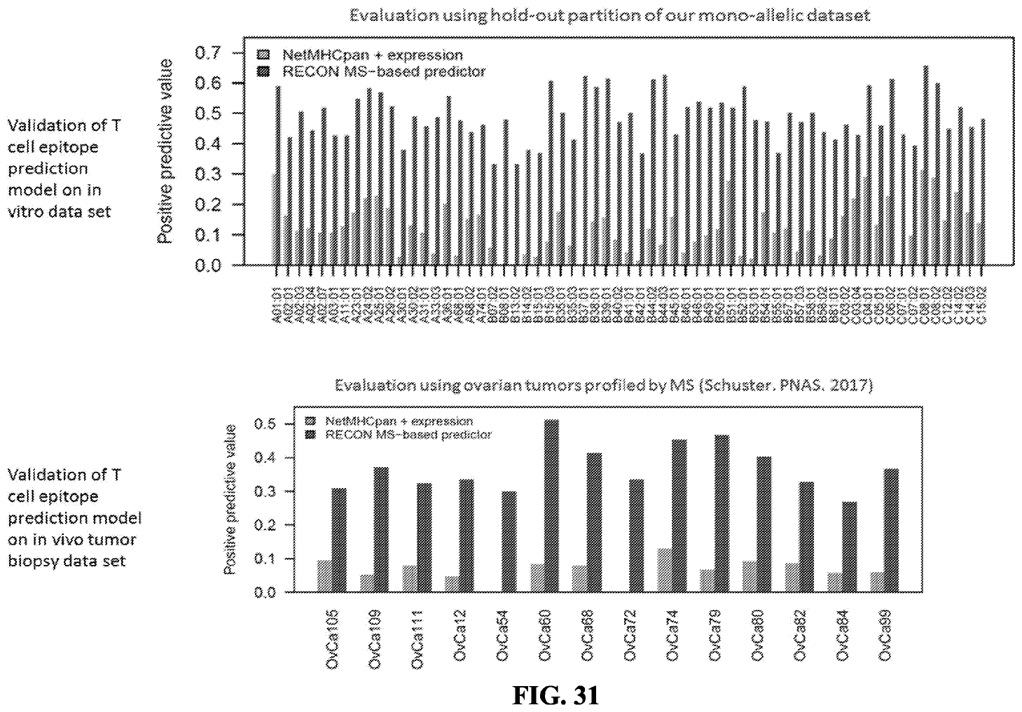

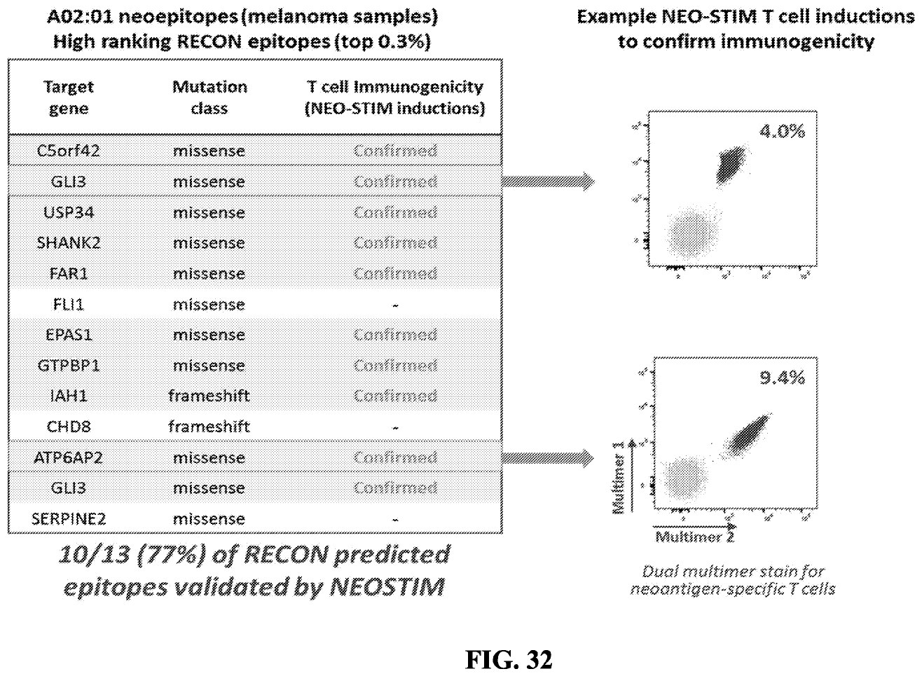

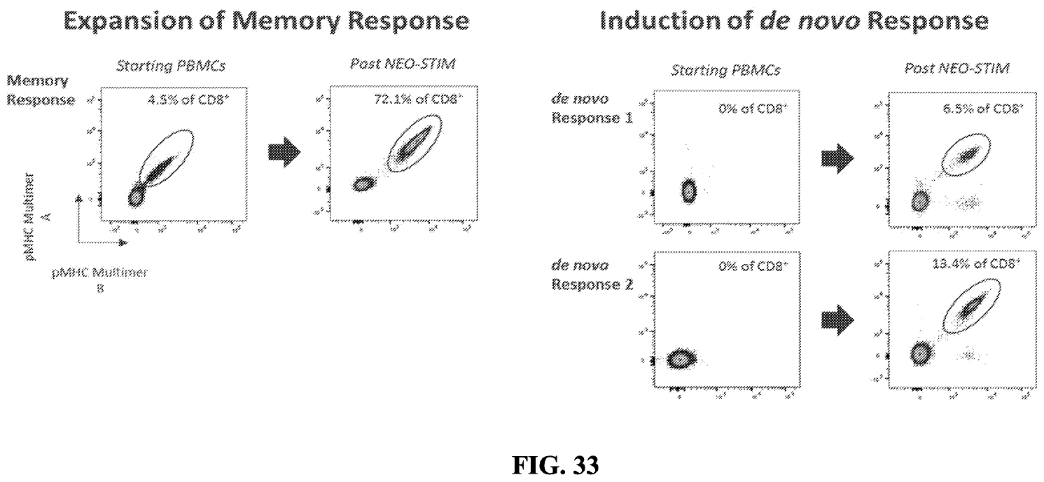

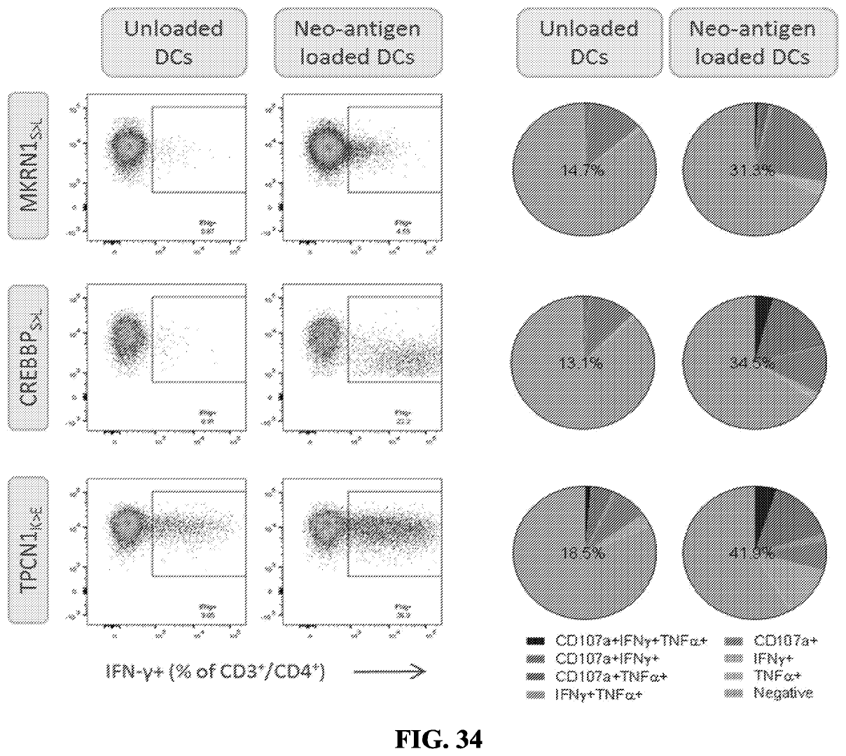

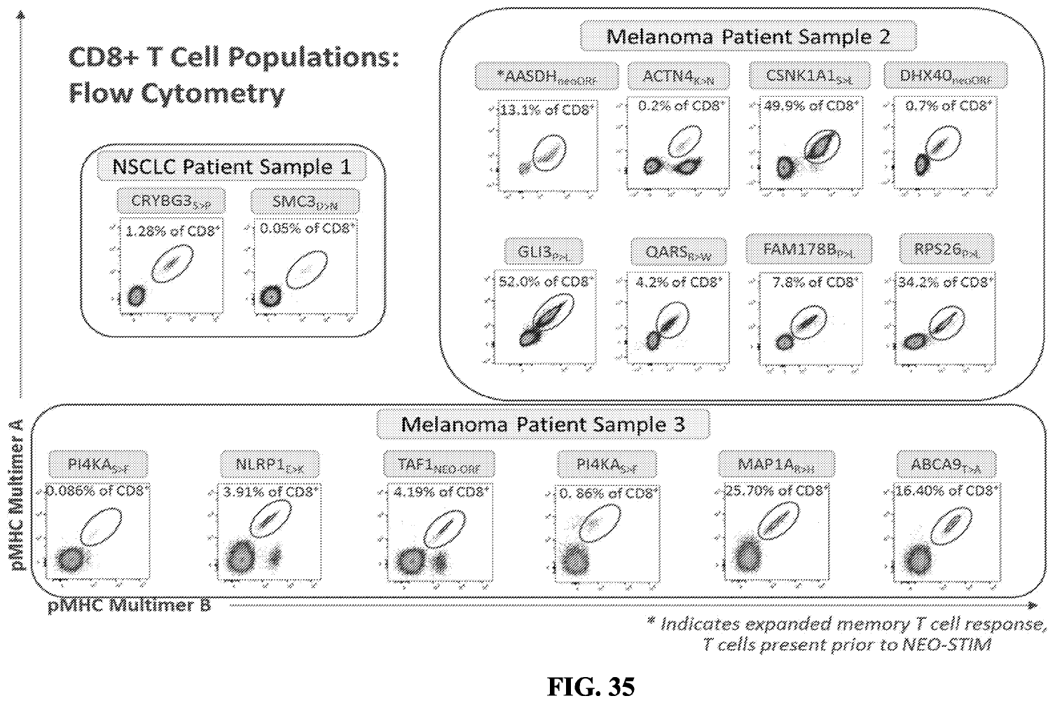

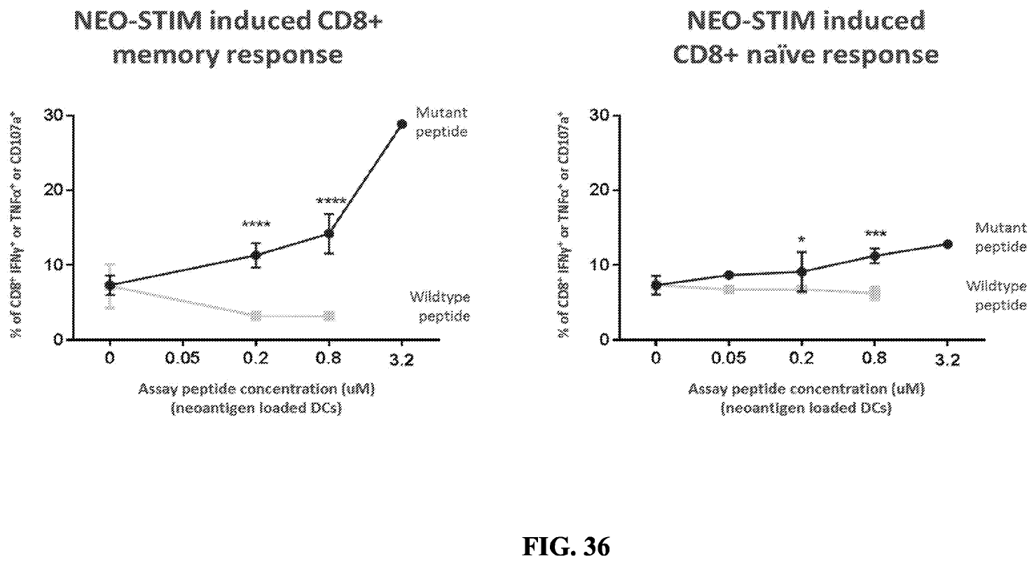

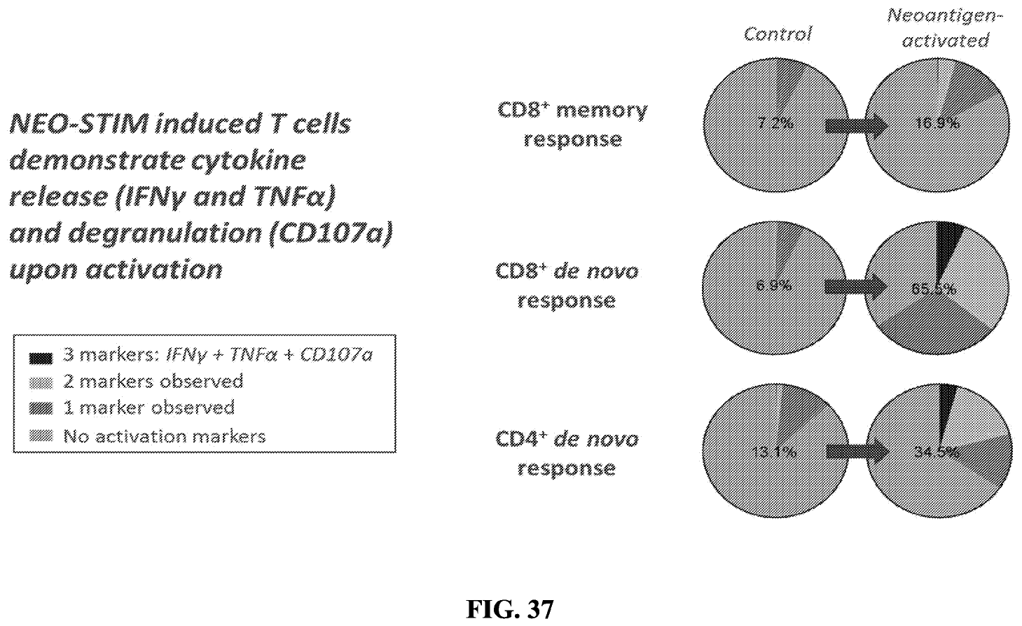

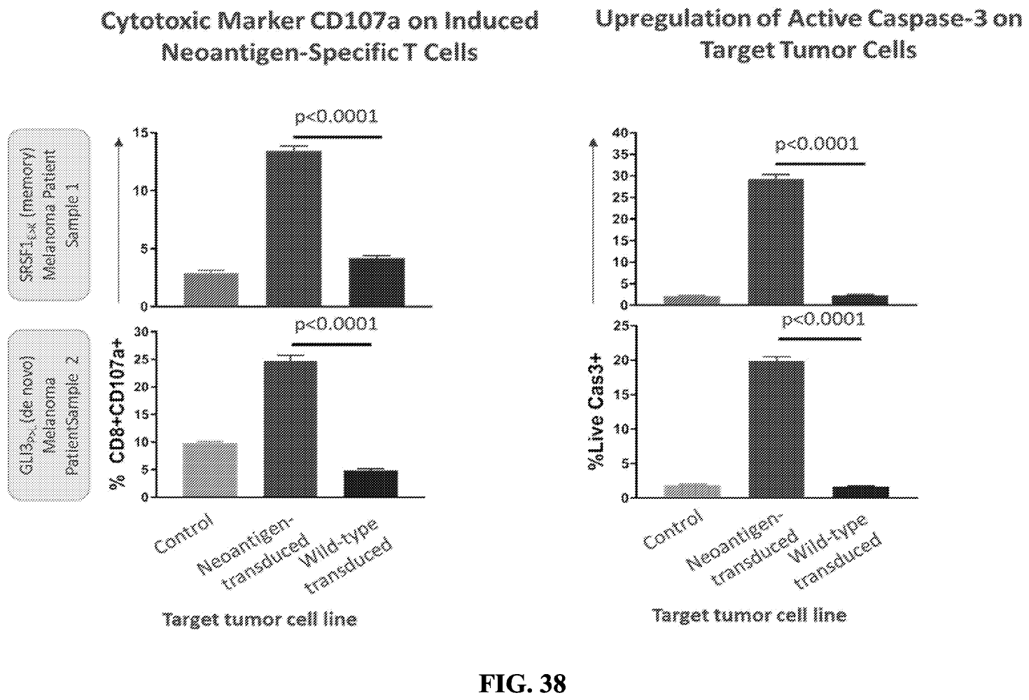

Exemplary data for RAS neoantigens on HLA-A03:01 and HLA-A11:01 are shown in FIG. 5. Exemplary data across multiple healthy donors for RAS G12V neoantigens on HLA-A11:01 are shown in FIG. 6. Exemplary data for RAS G12V neoantigens on HLA-A02:01 are shown in FIG. 13. Exemplary data for RAS neoantigens on HLA-A68:01 are shown in FIG. 14. Exemplary data for RAS neoantigens on HLA-B07:02 are shown in FIG. 15. Exemplary data for RAS neoantigens on HLA-B08:01 are shown in FIG. 16. Exemplary data for a RAS G12D neoantigens on HLA-008:02 are shown in FIG. 17. Exemplary data for GATA3 neoantigens on HLA-A02:01, HLA-A03:01, HLA-A11:01, HLA-B07:02, and HLA-B08:01 are shown in FIG. 21. Exemplary data for a BTK C481S neoantigen on HLA-A02:01 are shown in FIG. 26. Exemplary data for EGFR T790M neoantigens on HLA-A02:01 are shown in FIG. 27.

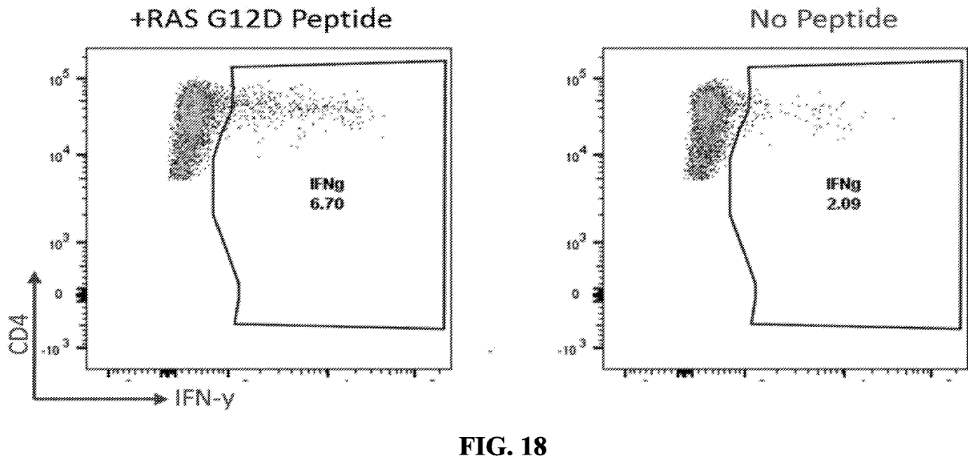

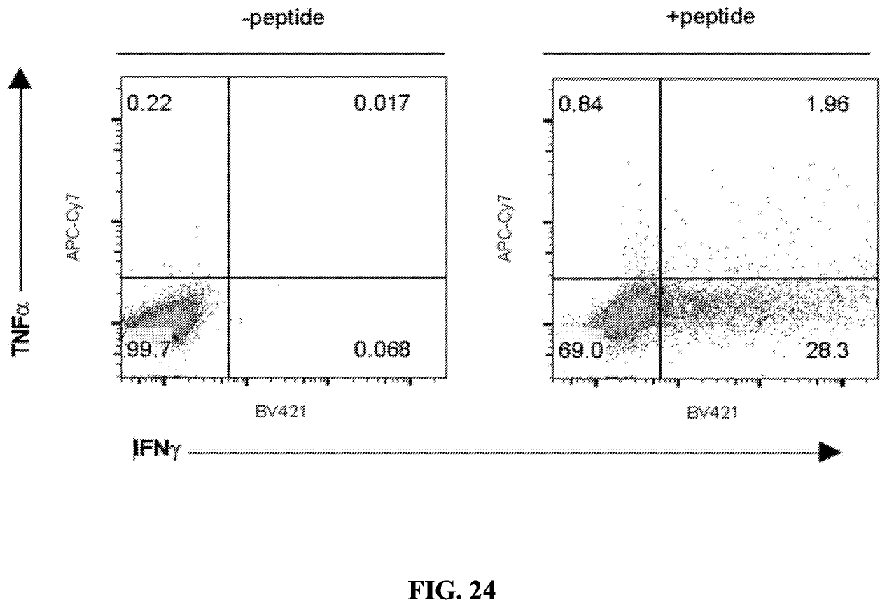

CD4⁺ T cell responses towards neoantigens can be induced using the ex vivo induction protocol. In this example, CD4⁺ T cell responses were identified by monitoring IFNγ and/or TNFα production in an antigen specific manner. FIG. 18 shows representative examples of such flow cytometric analysis for CD4+ T cells reactive to a RAS G12D neoantigen. FIG. 24 shows representative examples of such flow cytometric analysis for CD4+ T cells reactive to a GATA3 neoantigen.

### Example 3. Evaluation of Presentation of Antigens

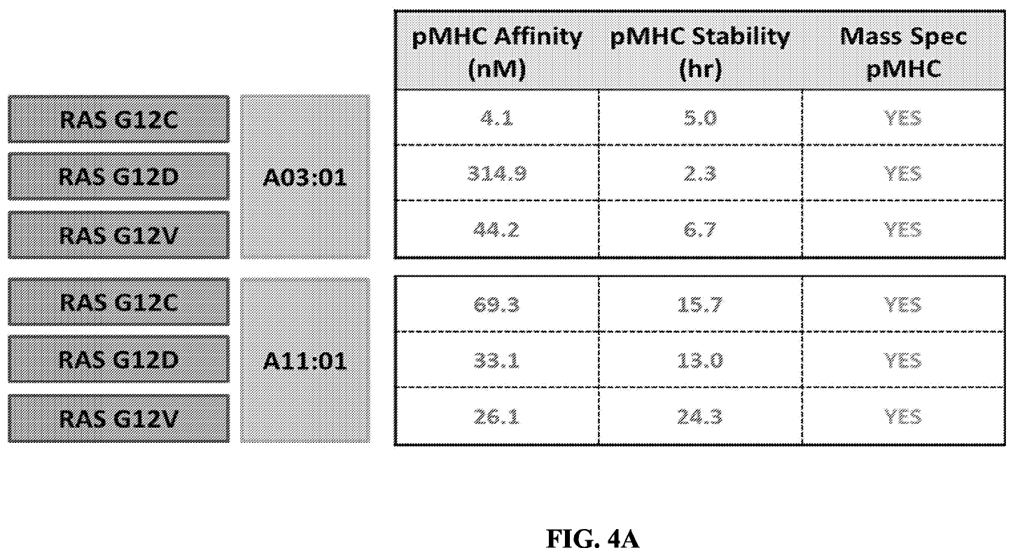

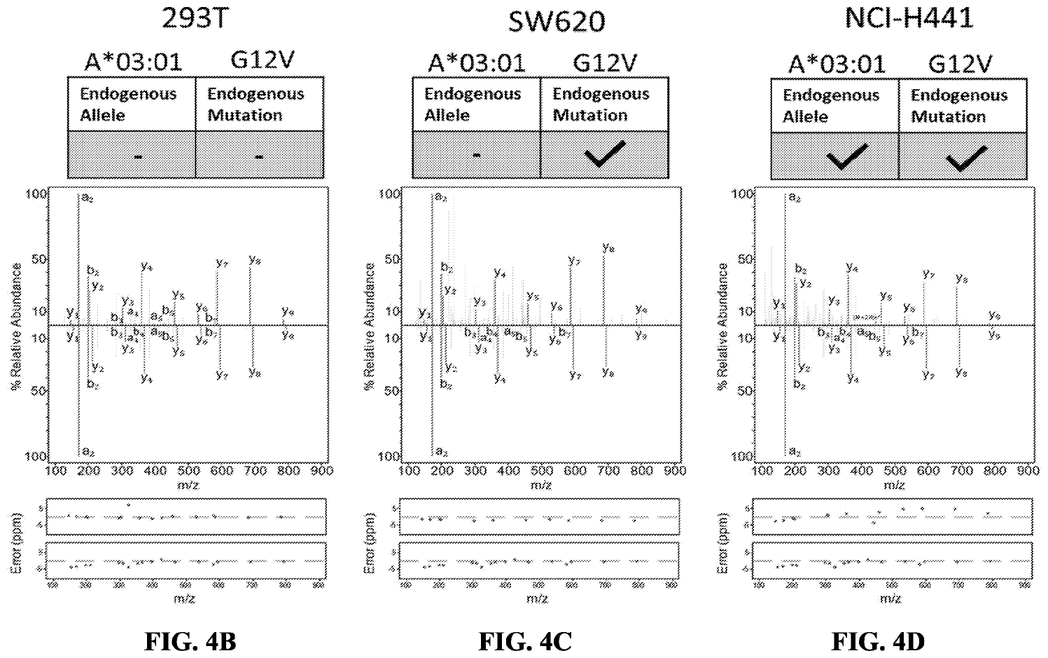

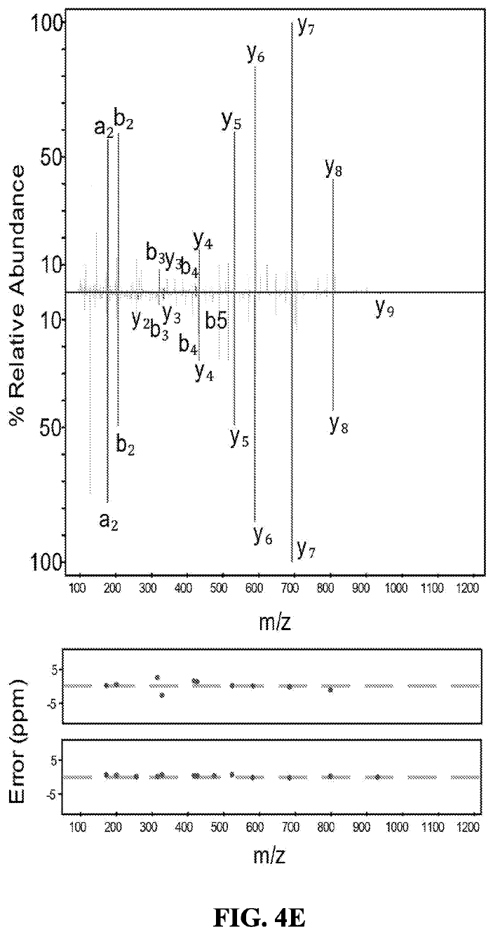

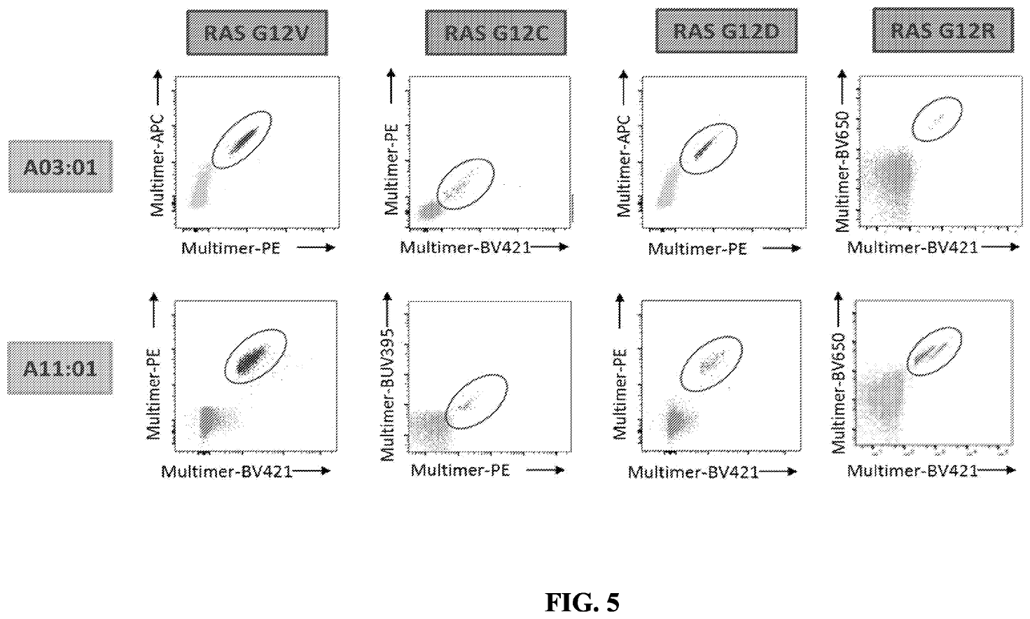

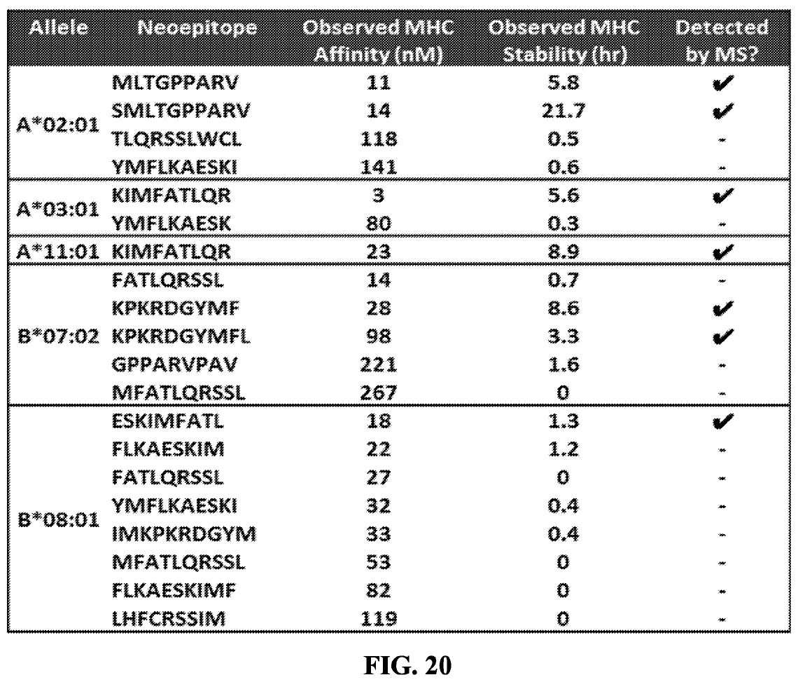

For a subset of predicted antigens, the affinity of the neoepitopes for the indicated HLA alleles and stability of the neoepitopes with the HLA alleles was determined. Exemplary data for a subset of RAS neoantigens and GATA3 neoantigens are shown in FIGS. 4A and 20, respectively.

An exemplary detailed description of the protocol utilized to measure the binding affinity of peptides to Class I MHC has been published (Sette et al, Mol. Immunol. 31(11):813-22, 1994). In brief, MHCI complexes were prepared and bound to radiolabeled reference peptides. Peptides were incubated at varying concentrations with these complexes for 2 days, and the amount of remaining radiolabeled peptide bound to WWI was measured using size exclusion gel-filtration. The lower the concentration of test peptide needed to displace the reference radiolabeled peptide demonstrates a stronger affinity of the test peptide for MHCI. Peptides with affinities to MHCI<50 nM are generally considered strong binders while those with affinities <150 nM are considered intermediate binders and those <500 nM are considered weak binders (Fritsch et al, 2014).

An exemplary detailed description of the protocol utilized to measure the binding stability of peptides to Class I MHC has been published (Harndahl et al. J Immunol Methods. 374:5-12, 2011). Briefly, synthetic genes encoding biotinylated MHC-I heavy and light chains are expressed in E. coli and purified from inclusion bodies using standard methods. The light chain (β2m) is radio-labeled with iodine (125I), and combined with the purified MHC-I heavy chain and peptide of interest at 18° C. to initiate pMHC-I complex formation. These reactions are carried out in streptavidin coated microplates to bind the biotinylated MHC-I heavy chains to the surface and allow measurement of radiolabeled light chain to monitor complex formation. Dissociation is initiated by addition of higher concentrations of unlabeled light-chain and incubation at 37° C. Stability is defined as the length of time in hours it takes for half of the complexes to dissociate, as measured by scintillation counts.

To assess whether antigens could be processed and presented from the larger polypeptide context, peptides eluted from HLA molecules isolated from cells expressing the genes of interest were analyzed by tandem mass spectrometry (MS/MS).

For analysis of presentation of RAS neoantigens, cell lines were utilized that have RAS mutations naturally or were lentivirally transduced to express the mutated RAS gene. HLA molecules were either isolated based on the natural expression of the cell lines or the cell lines were lentivirally transduced or transiently transfected to express the HLA of interest. 293T cells were transduced with a lentiviral vector encoding various regions of a mutant RAS peptide. Greater than 50 million cells expressing peptides encoded by a mutant RAS peptide were cultured and peptides were eluted from HLA-peptide complexes using an acid wash. Eluted peptides were then analyzed by targeted MS/MS with parallel reaction monitoring (PRM). For 293T cells lentivirally transduced with both a RAS^{G12V} mutation and an HLA-A*03:01 gene, the peptide with amino acid sequence vvvgaVgvgk (SEQ ID NO: 5) was detected by mass spectrometry. Spectral comparison to its corresponding stable heavy-isotope labeled synthetic peptide (FIG. 4B) showed mass accuracy of the detected peptide to be less than 5 parts per million (ppm). Endogenous peptide spectra are shown in the top panels and corresponding stable heavy-isotope labeled spectra are shown in the bottom panels. For SW620 cells naturally expressing a RAS^{G12V} mutation and lentivirally transduced with the HLA-A*03:01 gene, the peptide with amino sequence vvvgaVgvgk (SEQ ID NO: 5) was detected by mass spectrometry. Spectral comparison to its corresponding stable heavy-isotope labeled synthetic peptide showed mass accuracy of the detected peptide to be less than 5 ppm (FIG. 4C). Endogenous peptide spectra are shown in the top panels and corresponding stable heavy-isotope labeled spectra are shown in the bottom panels. For NCI-H441 cells naturally expressing both the RAS^{G12V} mutation and the HLA-A*03:01 gene, the peptide with amino acid sequence vvvgaVgvgk (SEQ ID NO: 5) was detected by mass spectrometry. Spectral comparison to its corresponding stable heavy-isotope labeled synthetic peptide showed mass accuracy of the detected peptide to be less than 5 ppm (FIG. 4D). Endogenous peptide spectra are shown in the top panels and corresponding stable heavy-isotope labeled spectra are shown in the bottom panels. A similar procedure was performed to analyze peptides derived from multiple RAS^{G12} mutations on HLA-A*03:01, HLA-A*11:01, HLA-A*30:01, HLA-A*68:01 and HLA-B*07:02 and Table 13 lists those peptides that were detected by mass spectrometry.

TABLE 13

| | | | SEQ | |
|---|---|---|---|---|
| Allele | Mutation | Neoantigen | ID NO: | Length |
| A*03:01 | G12C | vvvgaCgvgk | 157 | 10 |
| | G12D | vvvgaDgvgk | 159 | 10 |
| | G12D | klvvvgaDgvgk | 1252 | 12 |
| | G12R | vvvgaRgvgk | 3 | 10 |
| | G12R | klvvvgaRgvgk | 1262 | 12 |
| | G12V | vvvgaVgvgk | 5 | 10 |
| | G12V | vvgaVgvgk | 164 | 9 |
| | G12V | klvvvgaVgvgk | 1283 | 12 |
| A*11:01 | G12C | vvvgaCgvgk | 157 | 10 |
| | G12D | vvvgaDgvgk | 159 | 10 |
| | G12R | vvvgaRgvgk | 3 | 10 |
| | G12V | vvvgaVgvgk | 5 | 10 |
| | G12V | vvgaVgvgk | 164 | 9 |
| A*30:01 | G12R | vvvgaRgvgk | 3 | 10 |
| A*68:01 | G12C | vvvgaCgvgk | 157 | 10 |
| | G12D | vvvgaDgvgk | 159 | 10 |
| | G12R | vvvgaRgvgk | 3 | 10 |
| | G12V | vvvgaVgvgk | 5 | 10 |
| | G12V | vvgaVgvgk | 164 | 9 |
| | G12V | lvvvgaVgvgk | 1282 | 11 |
| B*07:02 | G12D | gaDgvgksal | 1244 | 10 |
| | G12R | gaRgvgksal | 1255 | 10 |

For analysis of presentation of GATA3 neoantigens, 293T cells were transduced with a lentiviral vector encoding various regions of peptides encoded by the GATA3 neoORF. Between 50 and 700 million of the transduced cells expressing peptides encoded by the GATA3 neoORF sequence were cultured and peptides were eluted from HLA-peptide complexes using an acid wash. Eluted peptides were then analyzed by targeted MS/MS using PRM. Spectral comparison between peptides derived from GATA3 neoORF and corresponding synthetic peptides were performed to confirm each detection. For 293T cells expressing an HLA-A*02:01 protein, the peptides VLPEPHLAL (SEQ ID NO: 1084), SMLTGPPARV (SEQ ID NO: 6) and MLTGPPARV (SEQ ID NO: 1081) were detected by mass spectrometry (Table 14 and FIG. 20). Spectral comparison to corresponding synthetic peptides showed mass accuracy of the detected peptide (SMLTGPPARV (SEQ ID NO: 6)) to be less than 5 ppm (FIG. 4E). For 293T cells expressing an HLA-A*03:01 or HLA-A*11:01 protein, the peptide KIMFATLQR (SEQ ID NO: 1089) was detected by mass spectrometry (FIG. 20). For 293T cells expressing an HLA-A*30:02 protein, the peptides IMKPKRDGY (SEQ ID NO: 1390) and SIMKPKRDGY (SEQ ID NO: 1391) were detected by mass spectrometry (Table 14). For 293T cells expressing an HLA-B*07:02 protein, the peptides KPKRDGYMF (SEQ ID NO: 1093) and KPKRDGYMFL (SEQ ID NO: 1095) were detected by mass spectrometry (Table 14 and FIG. 20). For 293T cells expressing an HLA-B*08:01 protein, the peptide ESKIMFATL (SEQ ID NO: 1091) was detected by mass spectrometry (Table 14 and FIG. 20). For 293T cells expressing an HLA-B*40:02 protein, the peptides AESKIMFATL (SEQ ID NO: 1392) and AESKIMFAT (SEQ ID NO: 1393) were detected by mass spectrometry (Table 14). For 293T cells expressing an HLA-C*03:03 protein, the peptide FATLQRSSL (SEQ ID NO: 1078) was detected by mass spectrometry (Table 14).

TABLE 14

| | | | SEQ ID | |
|---|---|---|---|---|
| Allele | Mutation | Neoantigen | NO: | Length |
| A*02:01 | GATA3 neoORF | VLPEPHLAL | 1084 | 9 |
| | GATA3 neoORF | SMLTGPPARV | 6 | 10 |
| | GATA3 neoORF | MLTGPPARV | 1081 | 9 |
| A*03:01 | GATA3 neoORF | KIMFATLQR | 1089 | 9 |
| A*11:01 | GATA3 neoORF | KIMFATLQR | 1089 | 9 |
| A*30:02 | GATA3 neoORF | EVIKPKRDGY | 1390 | 9 |
| | GATA3 neoORF | SIMKPKRDGY | 1391 | 10 |
| B*07:02 | GATA3 neoORF | KPKRDGYMF | 1093 | 9 |
| | GATA3 neoORF | KPKRDGYMFL | 1095 | 10 |
| B*08:01 | GATA3 neoORF | ESKIMFATL | 1091 | 9 |
| B*40:02 | GATA3 neoORF | AESKIMFATL | 1392 | 10 |
| | GATA3 neoORF | AESKIMFAT | 1393 | 9 |
| C*03:03 | GATA3 neoORF | FATLQRSSL | 1078 | 9 |

### HLA Class I Binding and Stability

A subset of the peptides used for affinity measurements were also used for stability measurements using the assay described (n=275). These data are shown in Table 3. Less than 50 nM was considered by the field as a strong binder, 50-150 nM was considered an intermediate binder, 150-500 nM was considered a weak binder, and greater than 500 nM was considered a very weak binder. The connection between the observed stability and observed affinity was evident by the decreasing median stability across these binned stability intervals. However, there is considerable overlap between the bins, and importantly there are epitopes in all bins with observed stability in the multiple hour range, including the very weak binders.

Immunogenicity assays are used to test the ability of each test peptide to expand T cells. Mature professional APCs are prepared for these assays in the following way. Monocytes are enriched from healthy human donor PBMCs using a bead-based kit (Miltenyi). Enriched cells are plated in GM-CSF and IL-4 to induce immature DCs. After 5 days, immature DCs are incubated at 37° C. with each peptide for 1 hour before addition of a cytokine maturation cocktail (GM-CSF, IL-1β, IL-4, IL-6, TNFα, PGE1β). Cells are incubated at 37° C. to mature DCs. In some embodiments the peptides, when administered into a patient is required to elicit an immune response.

Table 4A shows peptide sequences comprising RAS mutations, corresponding HLA allele to which it binds, and measured stability and affinity.

### Example 4. Assessment of Cytotoxic Capacity of Antigen-Specific T Cells In Vitro

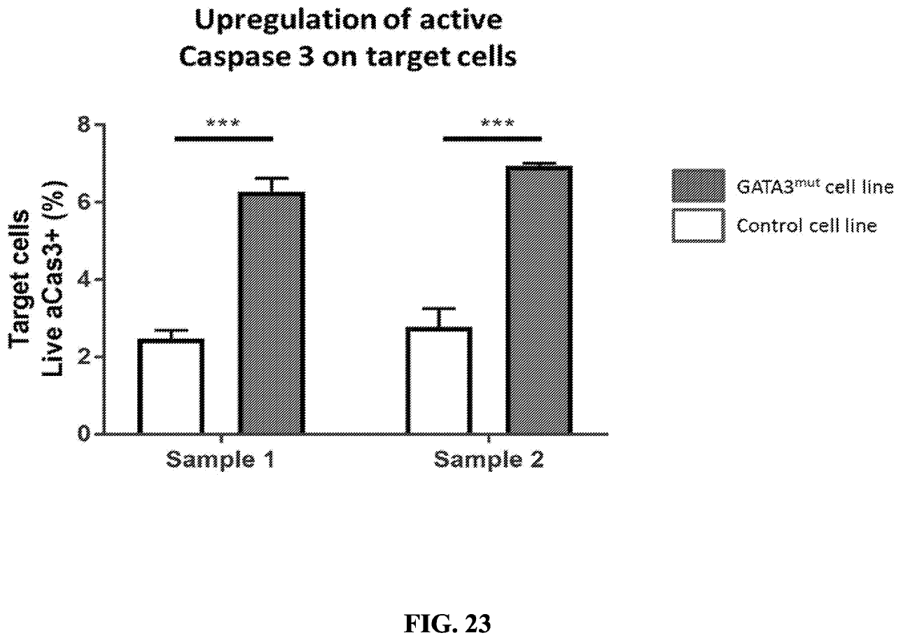

Cytotoxicity activity can be measured with the detection of cleaved Caspase 3 in target cells by Flow cytometry. Target cancer cells are engineered to express the mutant peptide along and the proper MHC-I allele. Mock-transduced target cells (i.e. not expressing the mutant peptide) are used as a negative control. The cells are labeled with CFSE to distinguish them from the stimulated PBMCs used as effector cells. The target and effector cells are co-cultured for 6 hours before being harvested. Intracellular staining is performed to detect the cleaved form of Caspase 3 in the CFSE-positive target cancer cells. The percentage of specific lysis is calculated as: Experimental cleavage of Caspase 3/spontaneous cleavage of Caspase 3 (measured in the absence of mutant peptide expression)×100. Exemplary data showing that T cells induced against GATA3 neoantigens can kill target cells expressing the GATA3 neoORF is shown in FIG. 23.

In some examples, cytotoxicity activity is assessed by co-culturing induced T cells with a population of antigen-specific T cells with target cells expressing the corresponding HLA, and by determining the relative growth of the target cells, along with measuring the apoptotic marker Annexin V in the target cancer cells specifically. Target cancer cells are engineered to express the mutant peptide or the peptide is exogenously loaded. Mock-transduced target cells (i.e. not expressing the mutant peptide), target cells loaded with wild-type peptides, or target cells with no peptide loaded are used as a negative control. The cells are also transduced to stably express GFP allowing the tracking of target cell growth. The GFP signal or Annexin-V signal are measured over time with an IncuCyte S3 apparatus. Annexin V signal originating from effector cells is filtered out by size exclusion. Target cell growth and death is expressed as GFP and Annexin-V area (mm²) over time, respectively.

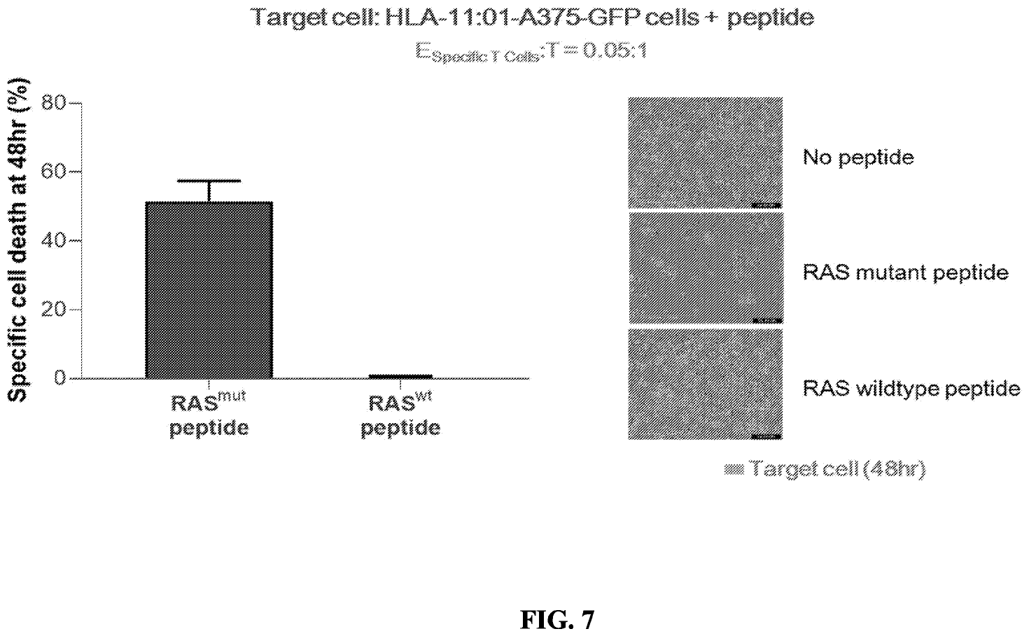

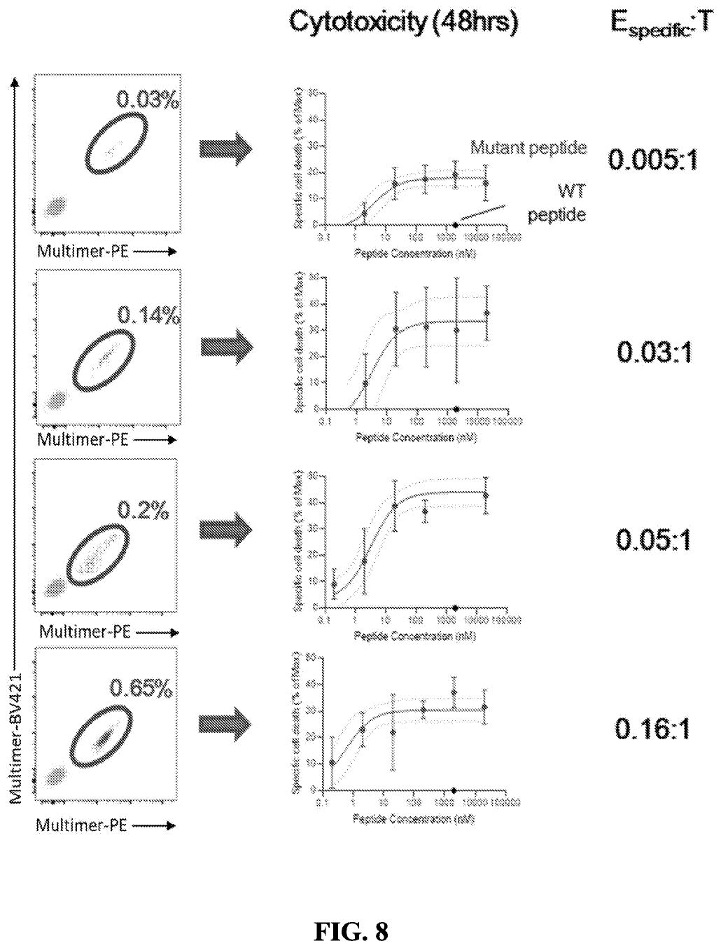

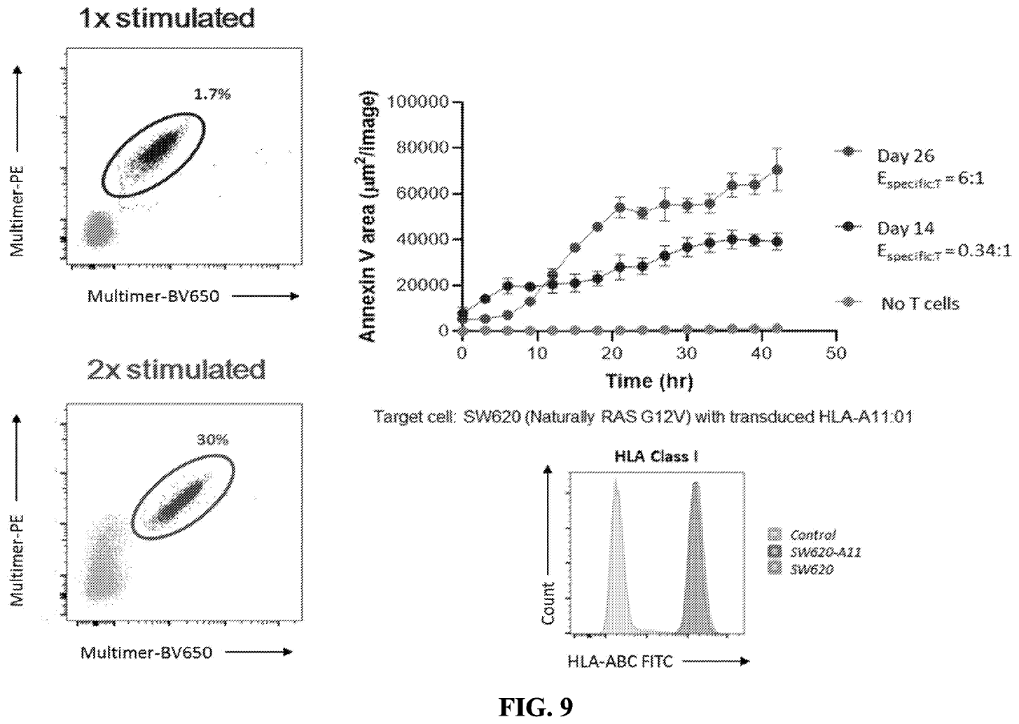

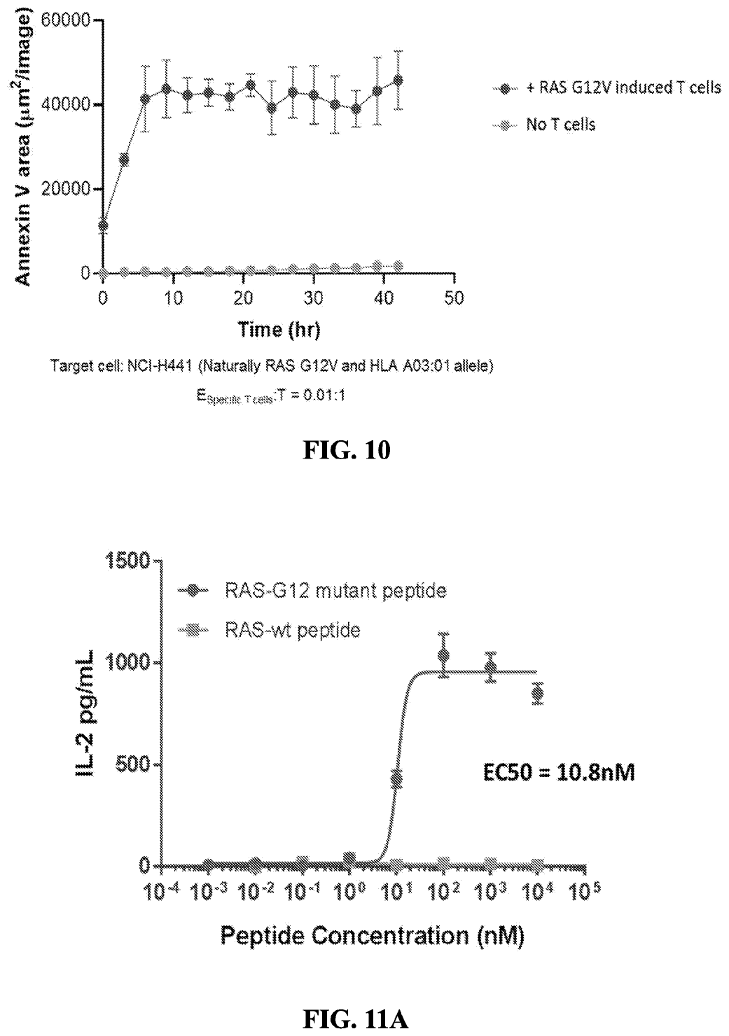

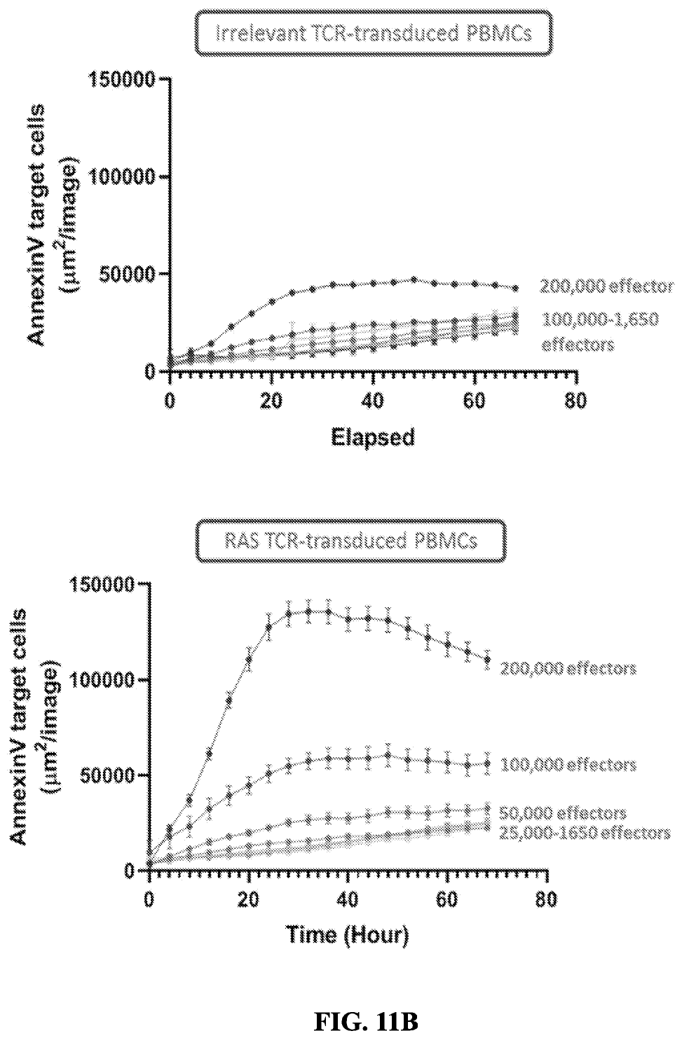

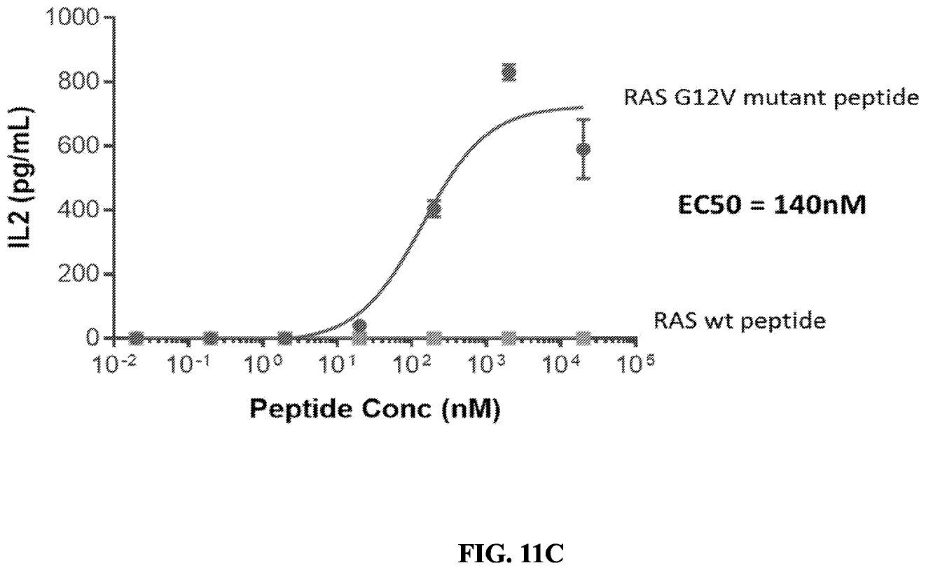

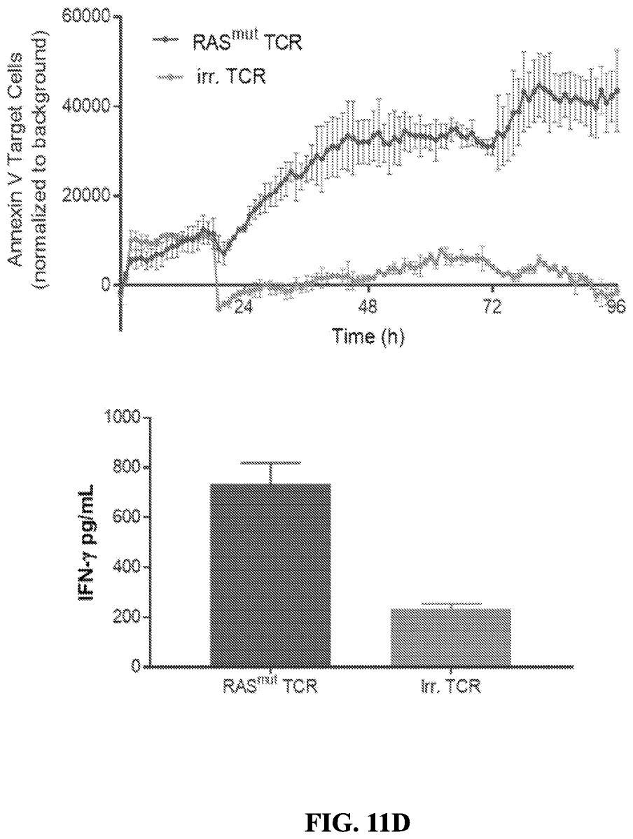

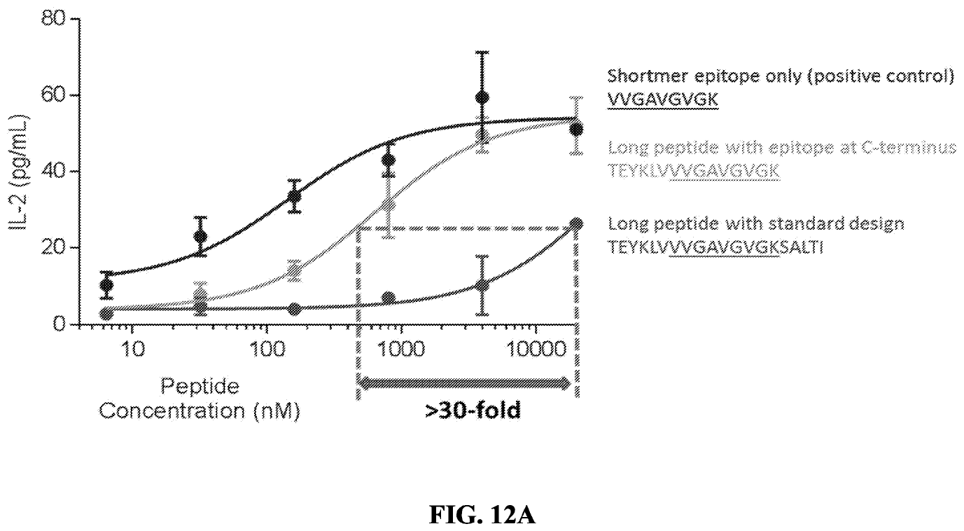

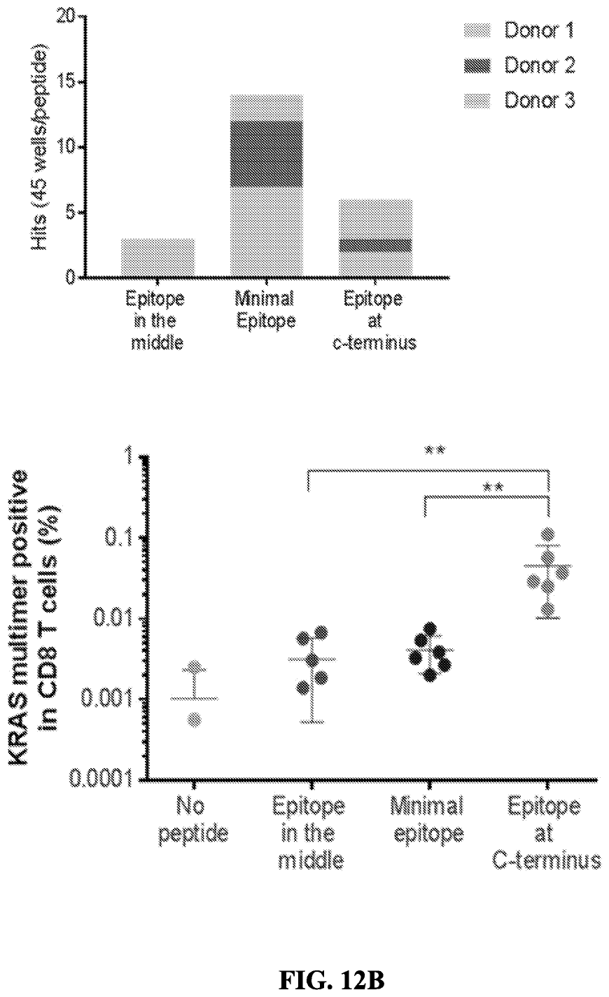

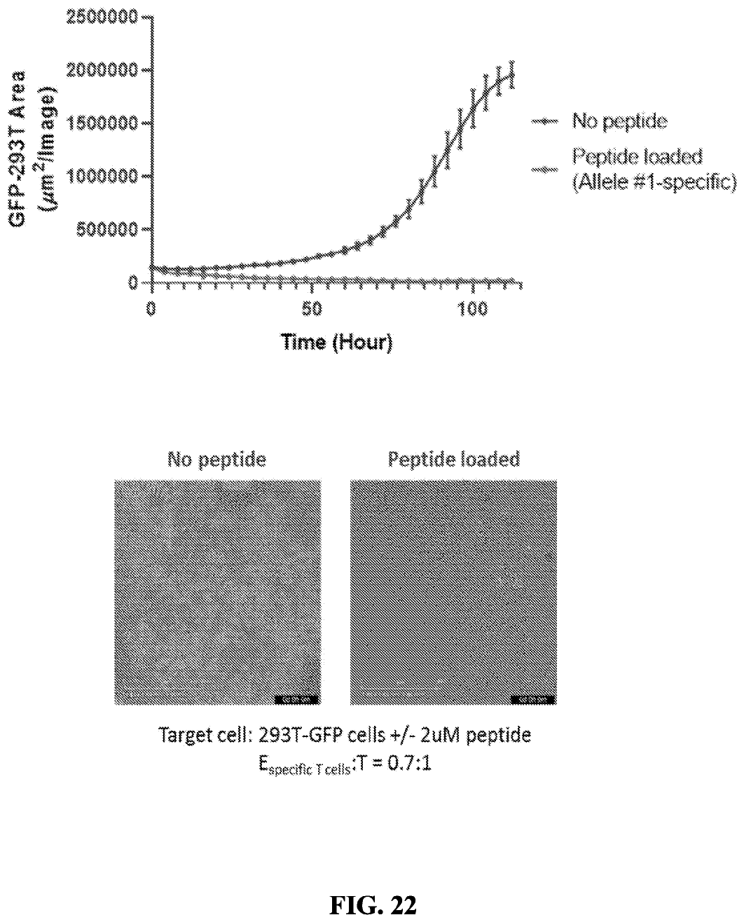

Exemplary data demonstrating that T cells stimulated to recognize a RAS^{G12V} neoantigen on HLA-A11:01 specifically recognize and kill target cells loaded with the mutant peptide but not the wild-type peptide is shown in FIG. 7. Exemplary data demonstrating that T cells stimulated to recognize a RAS^{G12V} neoantigen on HLA-A11:01 kill target cells loaded with nanomolar amounts of peptide at E:T ratios of <0.2:1 are shown in FIG. 8. Exemplary data demonstrating that T cells stimulated to recognize a RAS^{G12V} neoantigen on HLA-A11:01 kill SW620 cells that naturally have the RASG12V mutation and are transduced with HLA-A11:01 are shown in FIG. 9. Exemplary data demonstrating that T cells stimulated to recognize a RAS^{G12V} neoantigen on HLA-A03:01 kill NCI-H441 cells that naturally have the RASG12V mutation and HLA-A03:01 are shown in FIG. 10. Exemplary data demonstrating that T cells stimulated to recognize a GATA3 neoantigen on HLA-A02:01 kill 293T cells that naturally have HLA-A02:01 and are transduced with the GATA3 neoORF are shown in FIGS. 22 and 23.

### Example 5. Precision NEO-STIM Process Applied to Tissue-Specific Antigens

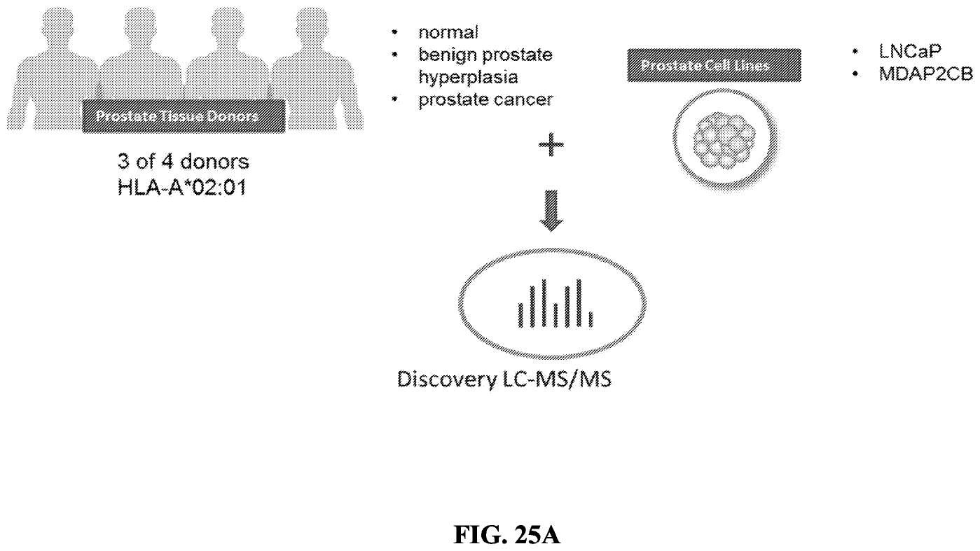

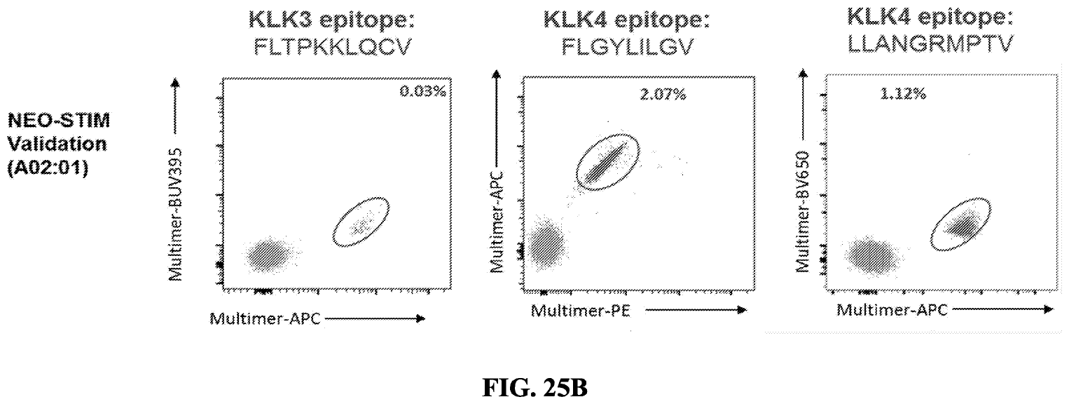

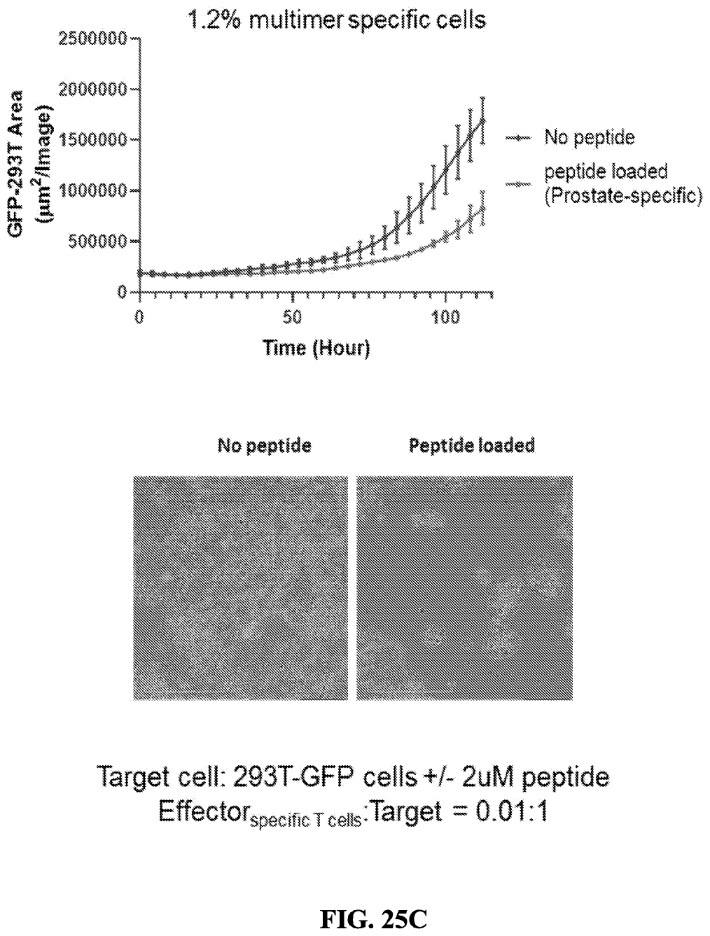

Antigens that are specifically expressed in a non-essential tissue can be targeted if a tumor arises in such a tissue. For example, antigens specifically expressed in prostate tissues can be targeted in the context of metastatic prostate cancer in which the primary tumor was resected, because the only cells expressing these antigens are metastatic cancer cells. There are multiple such non-essential tissues. As an example, prostate cells were evaluated using two methodologies to discover potential prostate-specific antigens. In one approach, prostate tissue or prostate cancer cell lines were evaluated using HLA-MS as outlined in Example 3. This approach can lead to identification of antigens that are validated to be processed and presented. Exemplary data from this approach is shown in FIG. 25A. In another approach, genes known to be expressed specifically in prostate cells can be evaluated through one or more MHC binding and presentation prediction software. A peptide-MHC prediction algorithm was generated and was used for these studies. As in Examples 2, 3 and 4, mass spectrometry, cellular and immunological assays further help validate a predicted peptide-HLA pair. Exemplary results from this analysis on 4 genes known to be specifically expressed in the prostate (KLK2, KLK3, KLK4, TGM4) are shown in the table below. These epitopes were further subjected to immunogenicity studies as in Example 2. The epitopes that are prefixed with ‘*’, were shown to induce positive CD8+ T cell response in either one or both the donors (marked as 1 or 2 in column 6 respectively) and also demonstrated in FIG. 25B.

TABLE 12

| | | | | Predicted | RECON | |
|---|---|---|---|---|---|---|
| | SEQ ID | | | Affinity | Percent | Immunogenicity |
| Peptide | NO: | Allele | Gene | (nM) | Rank | (#donors/2) |
| SLQCVSLHL | 1394 | HLA-A02:01 | KLK2 | 39.4 | 0.4 | |
| LVLSIALSV | 1395 | HLA-A02:01 | KLK2 | 54.9 | 1.1 | |
| VILGVHLSV | 1396 | HLA-A02:01 | KLK2 | 62.1 | 0.4 | |
| VLAPQESSV | 1397 | HLA-A02:01 | KLK2 | 65.7 | 0.08 | |
| SLQCVSLHLL | 1398 | HLA-A02:01 | KLK2 | 90.3 | 0.4 | |
| MLLRLSEPA | 1399 | HLA-A02:01 | KLK2; KLK3 | 56 | 2.5 | |
| LTMPALPMV | 1400 | HLA-A02:01 | KLK3 | 14.3 | 1.1 | |
| FLTLSVTWIA | 1401 | HLA-A02:01 | KLK3 | 16.9 | 3.5 | |
| KLQCVDLHV | 1402 | HLA-A02:01 | KLK3 | 21.2 | 0.3 | |
| *FLTPKKLQCV | 1403 | HLA-A02:01 | KLK3 | 126.4 | 0.17 | 1 |
| FLRPGDDSTL | 1404 | HLA-A02:01 | KLK3 | 982.7 | 0.4 | |
| *FLGYLILGV | 1405 | HLA-A02:01 | KLK4 | 6.3 | 0.05 | 1 |
| *LLANDLMLI | 1406 | HLA-A02:01 | KLK4 | 10.7 | 0.4 | 2 |
| *FQNSYTIGL | 1407 | HLA-A02:01 | KLK4 | 15.1 | 1.6 | 2 |
| MLIKLDESV | 1408 | HLA-A02:01 | KLK4 | 17.6 | 0.25 | |
| VLQCVNVSV | 1409 | HLA-A02:01 | KLK4 | 19.2 | 0.1 | |
| *LLANGRMPTV | 7 | HLA-A02:01 | KLK4 | 25.9 | 0.25 | 2 |
| *ILNDTGCHYV | 1410 | HLA-A02:01 | TGM4 | 17.2 | 0.1 | 1 |
| *FQYPEFSIEL | 1411 | HLA-A02:01 | TGM4 | 21.2 | 1 | 1 |
| ILGKYQLNV | 1412 | HLA-A02:01 | TGM4 | 22 | 0.3 | |
| LLGNSVNFTV | 1413 | HLA-A02:01 | TGM4 | 27.8 | 0.7 | |
| *VLDCCISLL | 1414 | HLA-A02:01 | TGM4 | 30.6 | 0.4 | 1 |
| ILGSFELQL | 1415 | HLA-A02:01 | TGM4 | 31.2 | 0.25 | |
| *RLIWLVKMV | 1416 | HLA-A02:01 | TGM4 | 64.4 | 0.17 | 1 |
| VLLGNSVNFTV | 1417 | HLA-A02:01 | TGM4 | 83.7 | 0.6 | |
| TLAIPLTDV | 1418 | HLA-A02:01 | TGM4 | 149.2 | 0.25 | |

In a further assay, T cells that are specific for the peptides indicated in the table were tested for ability to kill target cells as described in Example 4. An exemplary data is presented in FIG. 25C, where KLK4 prostate specific epitope were co-cultured with 293T-GFP cells either loaded with 2 uM of peptide or not loaded. Peptide loaded target cells were killed to a much greater extent (right image) compared to the no peptide control (left image).

### Example 6. Enrichment of Target Antigen Activated T Cells

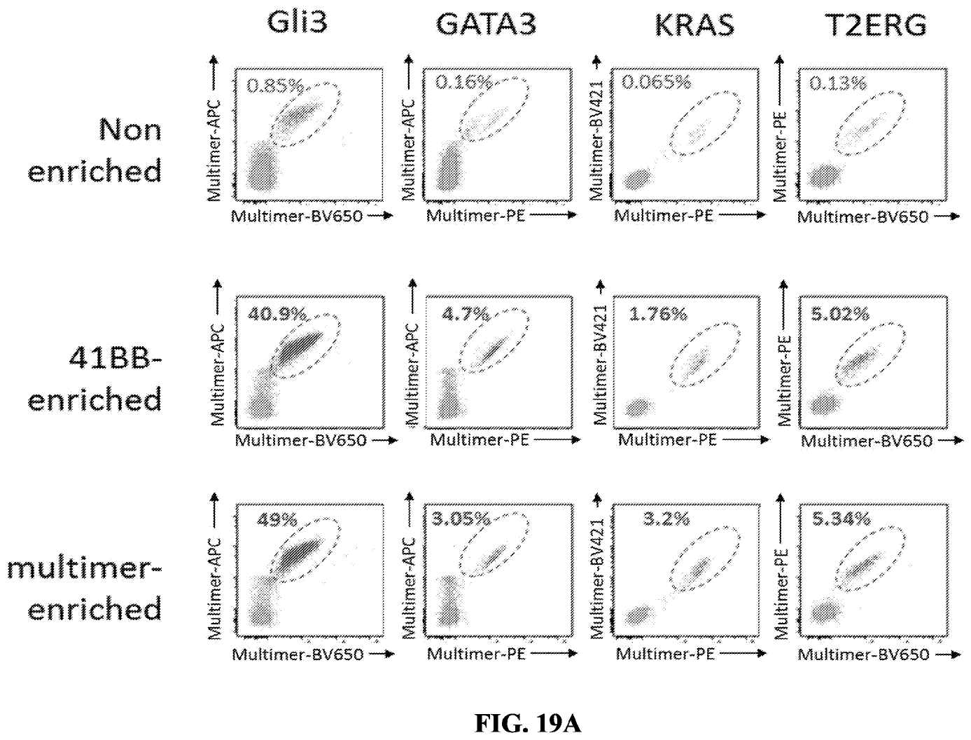

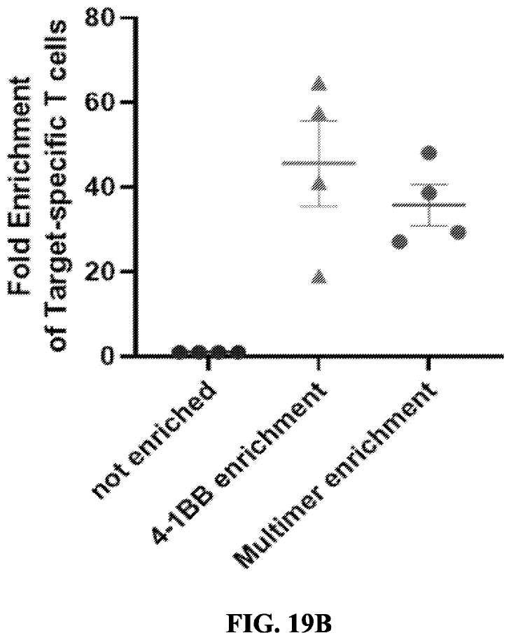

Tumor antigen responsive T cells may be further enriched. In this example, multiple avenues for enrichment of antigen responsive T cells are explored and results presented. After the initial stimulation of antigen-specific T cells (Example 2, Steps 1-5), an enrichment procedure can be used prior to further expansion of these cells. As an example, stimulated cultures and pulsed with the same peptides used for the initial stimulation on day 13, and cells upregulating 4-1BB are enriched using Magnetic-Assisted Cell Separation (MACS; Miltenyi). These cells can then be further expanded, for example, using anti-CD3 and anti-CD28 microbeads and low-dose IL-2. As shown in FIG. 19A (middle row) and FIG. 19B (middle column), this approach can enrich for multiple antigen-specific T cell populations. As another example, T cells that are stained by multimers can be enriched by MACS on day 14 of stimulation and further expanded, for example, using anti-CD3 and anti-CD28 microbeads and low-dose IL-2. As shown in FIG. 19A (bottom row) and FIG. 19B (right column), this approach can enrich for multiple antigen-specific T cell populations.

### Example 7. Immunogenicity Assays for Selected Peptides

After maturation of DCs, PBMCs (either bulk or enriched for T cells) are added to mature dendritic cells with proliferation cytokines. Cultures are monitored for peptide-specific T cells using a combination of functional assays and/or tetramer staining. Parallel immunogenicity assays with the modified and parent peptides allowed for comparisons of the relative efficiency with which the peptides expanded peptide-specific T cells. In some embodiments, the peptides elicit an immune response in the T cell culture comprises detecting an expression of a FAS ligand, granzyme, perforins, IFN, TNF, or a combination thereof in the T cell culture.

Immunogenicity can be measured by a tetramer assay. MHC tetramers are purchased or manufactured on-site, and are used to measure peptide-specific T cell expansion in the immunogenicity assays. For the assessment, tetramer is added to 1×10\hat{ }5 cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4 degrees Celsius for 20 minutes. Cells are washed with cold FACS buffer and resuspended in buffer containing 1% formaldehyde. Cells are acquired on a FACS Calibur (Becton Dickinson) instrument, and are analyzed by use of Cellquest software (Becton Dickinson). For analysis of tetramer positive cells, the lymphocyte gate is taken from the forward and side-scatter plots. Data are reported as the percentage of cells that were CD8⁺/Tetramer⁺.

Immunogenicity can be measured by intracellular cytokine staining. In the absence of well-established tetramer staining to identify antigen-specific T cell populations, antigen-specificity can be estimated using assessment of cytokine production using well-established flow cytometry assays. Briefly, T cells are stimulated with the peptide of interest and compared to a control. After stimulation, production of cytokines by CD4+ T cells (e.g., IFNγ and TNFα) are assessed by intracellular staining. These cytokines, especially IFNγ, used to identify stimulated cells.

In some embodiments the immunogenicity is measured by measuring a protein or peptide expressed by the T cell, using ELISpot assay. Peptide-specific T cells are functionally enumerated using the ELISpot assay (BD Biosciences), which measures the release of IFNγ from T cells on a single cell basis. Target cells (T2 or HLA-A0201 transfected C1Rs) were pulsed with 10 μM peptide for one hour at 37 degrees C., and washed three times. 1×10\hat{ }5 peptide-pulsed targets are co-cultured in the ELISPOT plate wells with varying concentrations of T cells (5×10\hat{ }2 to 2×10\hat{ }3) taken from the immunogenicity culture. Plates are developed according to the manufacturer's protocol, and analyzed on an ELISPOT reader (Cellular Technology Ltd.) with accompanying software. Spots corresponding to the number of IFN gamma-producing T cells are reported as the absolute number of spots per number of T cells plated. T cells expanded on modified peptides are tested not only for their ability to recognize targets pulsed with the modified peptide, but also for their ability to recognize targets pulsed with the parent peptide.

CD107a and b are expressed on the cell surface of CD8+ T cells following activation with cognate peptide. The lytic granules of T cells have a lipid bilayer that contains lysosomal-associated membrane glycoproteins (“LAMPs”), which include the molecules CD107a and b. When cytotoxic T cells are activated through the T cell receptor, the membranes of these lytic granules mobilize and fuse with the plasma membrane of the T cell. The granule contents are released, and this leads to the death of the target cell. As the granule membrane fuses with the plasma membrane, C107a and b are exposed on the cell surface, and therefore are markers of degranulation. Because degranulation as measured by CD107a and b staining is reported on a single cell basis, the assay is used to functionally enumerate peptide-specific T cells. To perform the assay, peptide is added to HLA-A0201-transfected cells C1R to a final concentration of 20 μM, the cells were incubated for 1 hour at 37 degrees C., and washed three times. 1×10\hat{ }5 of the peptide-pulsed C1R cells were aliquoted into tubes, and antibodies specific for CD107a and b are added to a final concentration suggested by the manufacturer (Becton Dickinson). Antibodies are added prior to the addition of T cells in order to “capture” the CD107 molecules as they transiently appear on the surface during the course of the assay. 1×10\hat{ }5 T cells from the immunogenicity culture are added next, and the samples were incubated for 4 hours at 37 degrees C. The T cells are further stained for additional cell surface molecules such as CD8 and acquired on a FACS Calibur instrument (Becton Dickinson). Data is analyzed using the accompanying Cellquest software, and results were reported as the percentage of CD8+ CD107 a and b+ cells.

Cytotoxic activity is measured using a chromium release assay. Target T2 cells are labeled for 1 hour at 37 degrees C. with Na51Cr and washed 5×10\hat{ }3 target T2 cells were then added to varying numbers of T cells from the immunogenicity culture. Chromium release is measured in supernatant harvested after 4 hours of incubation at 37 degrees C. The percentage of specific lysis is calculated as:

Experimental release−spontaneous release/Total release−spontaneous release×100

Immunogenicity assays were carried out to assess whether each peptide can elicit a T cell response by antigen-specific expansion. Though current methods are imperfect, and therefore negative results do not imply a peptide is incapable of inducing a response, a positive result demonstrates that a peptide can induce a T cell response. Several peptides from Table 3 were tested for their capacity to elicit CD8+ T cell responses with multimer readouts as described. Each positive result was measured with a second multimer preparation to avoid any preparation biases. In an exemplary assay, HLA-A02:01+ T cells were co-cultured with monocyte-derived dendritic cells loaded with TMPRSS2::ERG fusion neoepitope (ALNSEALSV (SEQ ID NO: 992); HLA-A02:01) for 10 days. CD8+ T cells were analyzed for antigen-specificity for TMPRSS2::ERG fusion neoepitope using multimers (initial: BV421 and PE; validation: APC and BUV396).

While antigen-specific CD8+ T cell responses are readily assessed using well-established HLA Class I multimer technology, CD4+ T cell responses require a separate assay to evaluate because HLA Class II multimer technology is not well-established. In order to assess CD4+ T cell responses, T cells were re-stimulated with the peptide of interest and compared to a control. In the case of a completely novel sequence (e.g., arising from a frame-shift or fusion), the control was no peptide. In the case of a point-mutation, the control was the WT peptide. After stimulation, production of cytokines by CD4+ T cells (e.g., IFNγ and TNFα) were assessed by intracellular staining. These cytokines, especially IFNγ, used to identify stimulated cells. Antigen-specific CD4+ T cell responses showed increased cytokine production relative to control.

### Example 8. Cell Expansion and Preparation

To prepare APCs, the following method was employed (a) obtain of autologous immune cells from the peripheral blood of the patient; enrich monocytes and dendritic cells in culture; load peptides and mature DCs.

### T Cell Induction (Protocol 1)

First induction: (a) Obtaining autologous T cells from an apheresis bag; (b) Depleting CD25+ cells and CD14+ cells, alternatively, depleting only CD25+ cells; (c) Washing the peptide loaded and mature DC cells, resuspending in the T cell culture media; (d) Incubating T cells with the matured DC.

Second induction: (a) Washing T cells, and resuspending in T cell media, and optionally evaluating a small aliquot from the cell culture to determine the cell growth, comparative growth and induction of T cell subtypes and antigen specificity and monitoring loss of cell population; (b) Incubating T cells with mature DC.

Third induction: (a) Washing T cells, and resuspending in T cell media, and optionally evaluating a small aliquot from the cell culture to determine the cell growth, comparative growth and induction of T cell subtypes and antigen specificity and monitoring loss of cell population; (b) Incubating T cells with mature DC.

To harvest peptide activated t cells and cryopreserve the T cells, the following method was employed (a) Washing and resuspension of the final formulation comprising the activated T cells which are at an optimum cell number and proportion of cell types that constitutes the desired characteristics of the Drug Substance (DS). The release criteria testing include inter alia, Sterility, Endotoxin, Cell Phenotype, TNC Count, Viability, Cell Concentration, Potency; (b) Filling drug substance in suitable enclosed infusion bags; (c) Preservation until time of use.

### Example 9. Methods of Functional Characterization of the CD4+ and CD8+ Neoantigen-Specific T Cells

Neoantigens, which arise in cancer cells from somatic mutations that alter protein-coding gene sequences, are emerging as an attractive target for immunotherapy. They are uniquely expressed on tumor cells as opposed to healthy tissue and may be recognized as foreign antigens by the immune system, increasing immunogenicity. T cell manufacturing processes were developed to raise memory and de novo CD4+ and CD8+ T cell responses to patient-specific neoantigens through multiple rounds of ex-vivo T cell stimulation, generating a neoantigen-reactive T cell product for use in adoptive cell therapy. Detailed characterization of the stimulated T cell product can be used to test the many potential variables these processes utilize.

To probe T cell functionality and/or specificity, an assay was developed to simultaneously detect antigen-specific T cell responses and characterize their magnitude and function. This assay employs the following steps. First T cell-APC co-cultures were used to elicit reactivity in antigen-specific T cells. Optionally, sample multiplexing using fluorescent cell barcoding is employed. To identify antigen-specific CD8+ T cells and to examine T cell functionality, staining of peptide-MHC multimers and multiparameter intracellular and/or cell surface cell marker staining were probed simultaneously using FACS analysis. The results of this streamlined assay demonstrated its application to study T cell responses induced from a healthy donor. Neoantigen-specific T cell responses induced toward peptides were identified in a healthy donor. The magnitude, specificity and functionality of the induced T cell responses were also compared. Briefly, different T cell samples were barcoded with different fluorescent dyes at different concentrations (see, e.g., Example 19). Each sample received a different concentration of fluorescent dye or combination of multiple dyes at different concentrations. Samples were resuspended in phosphate-buffered saline (PBS) and then fluorophores dissolved in DMSO (typically at 1:50 dilution) were added to a maximum final concentration of 5 μM After labeling for 5 min at 37° C., excess fluorescent dye was quenched by the addition of protein-containing medium (e.g. RPMI medium containing 10% pooled human type AB serum). Uniquely barcoded T cell cultures were challenged with autologous APC pulsed with the antigen peptides as described above.

The differentially labeled samples were combined into one FACS tube or well, and pelleted again if the resulting volume is greater than 100 μL. The combined, barcoded sample (typically 100 μL) was stained with surface marker antibodies including fluorochrome conjugated peptide-MHC multimers. After fixation and permeabilization, the sample was additionally stained intracellularly with antibodies targeting TNF-α and IFN-γ.

The cell marker profile and MEC tetramer staining of the combined, barcoded T cell sample were then analyzed simultaneously by flow cytometry on flow cytometer. Unlike other methods that analyze cell marker profiles and MEC tetramer staining of a T cell sample separately, the simultaneous analysis of the cell marker profile and MEC tetramer staining of a T cell sample described in this example provides information about the percentage of T cells that are both antigen specific and that have increased cell marker staining. Other methods that analyze cell marker profiles and MEC tetramer staining of a T cell sample, separately determine the percentage of T cells of a sample that are antigen specific, and separately determine the percentage of T cells that have increased cell marker staining, only allowing correlation of these frequencies.

The simultaneous analysis of the cell marker profile and MEC tetramer staining of a T cell sample described in this example does not rely on correlation of the frequency of antigen specific T cells and the frequency of T cells that have increased cell marker staining; rather, it provides a frequency of T cells that are both antigen specific and that have increased cell marker staining. The simultaneous analysis of the cell marker profile and MEC tetramer staining of a T cell sample described in this example allows for determination on a single cell level, those cells that are both antigen specific and that have increased cell marker staining.

To evaluate the success of a given induction process, a recall response assay was used followed by a multiplexed, multiparameter flow cytometry panel analysis. A sample taken from an induction culture was labeled with a unique two-color fluorescent cell barcode. The labeled cells were incubated on antigen-loaded DCs or unloaded DCs overnight to stimulate a functional response in the antigen-specific cells. The next day, uniquely labeled cells were combined prior to antibody and multimer staining according to Table 9 below.

TABLE 9

| Marker | Fluorochrome | Purpose |
|---|---|---|
| CD19/CD16/CD14 | BUV395 | Cell exclusion |
| Live/Dead | Near-IR | Dead cell exclusion |
| CD3 | BUV805 | Lineage gating |
| CD4 | Alexa Fluor 700 | Lineage gating |
| CD8 | PerCP-Cy5.5 | Lineage gating |
| Barcode 1 | CFSE | Sample multiplexing |
| Barcode 2 | TagIT Violet | Sample multiplexing |
| Multimer 1 | PE | CD8+ antigen specificity |
| Multimer 2 | BV650 | CD8+ antigen specificity |
| IFNγ | APC | Functionality |
| TNFα | BV711 | Functionality |
| CD107a | BV786 | Cytotoxicity |
| 4-1BB | PE/Dazzle 594 | Activation |

Patient-specific neoantigens were predicted using bioinformatics engine. Synthetic long peptides covering the predicted neoantigens were used as immunogens in the stimulation protocol to assess the immunogenic capacity. The stimulation protocol involves feeding these neoantigen-encoding peptides to patient-derived APCs, which are then co-cultured with patient-derived T cells to prime neoantigen specific T cells.

Multiple rounds of stimulations are incorporated in the stimulation protocol to prime, activate and expand memory and de novo T cell responses. The specificity, phenotype and functionality of these neoantigen-specific T cells was analyzed by characterizing these responses with the following assays: Combinatorial coding analysis using pMHC multimers was used to detect multiple neoantigen-specific CD8+ T cell responses. A recall response assay using multiplexed, multiparameter flow cytometry was used to identify and validate CD4+ T cell responses. The functionality of CD8+ and CD4+ T cell responses was assessed by measuring production of pro-inflammatory cytokines including IFN-γ and TNFα, and upregulation of the CD107a as a marker of degranulation. A cytotoxicity assay using neoantigen-expressing tumor lines was used to understand the ability of CD8+ T cell responses to recognize and kill target cells in response to naturally processed and presented antigen. The cytotoxicity was measured by the cell surface upregulation of CD107a on the T cells and upregulation of active Caspase3 on neoantigen-expressing tumor cells. The stimulation protocol was successful in the expansion of pre-existing CD8+ T cell responses, as well as the induction of de novo CD8+ T cell responses (Table 10).

TABLE 10
(“DEAH” disclosed as SEQ ID NO: 1419)

| | HUGO | | |
|---|---|---|---|
| Patient | Symbol | Full Gene Name | Type |
| NV10 | SRSF1E>K | Serine and Arginine Rich Splicing Factor 1 | CD8 |
| | ARAP1Y>H | Ankyrin Repeat And PH Domain | |
| | PKDREJG>R | Polycystin Family Receptor For Egg Jelly | |
| | MKRN1_{S>L} | Makorin Ring Finger Protein 1 | CD4 |
| | CREBBP_{S>L} | CRREB Binding Protein | |
| | TPCN1_{K>E} | Two Pore Segment Channel 1 | |
| NV6 | AASDH_{neoORF} | Aminoadipate-Semialdehyde Dehydrogenase | CD8 |
| | ACTN4_{K>N} | Actinin Alpha 4 | |
| | CSNK1A1_{S>L} | Casein Kinase 1 Alpha 1 | |
| | DHX40_{neoORF} | DEAH-Box Helicase 40 | |
| | GLI3_{P>L} | GLI Family Zinc Finger 3 | |
| | QARS_{R>W} | Glutamyl-tRNA Synthetase | |
| | FAM178B_{P>L} | Family With Sequence Similarity 178 Member 8 | |
| | RPS26_{P>L} | Ribosomal Protein S26 | |

Using PBMCs from a melanoma patient a clinical study performed by Applicant's group, expansion of a pre-existing CD8+ T cell response was observed from 4.5% of CD8+ T cells to 72.1% of CD8+ T cells (SRSF1E_{>K}). Moreover, the stimulation protocol was effective in inducing two presumed de novo CD8+ T cell responses towards patient-specific neoantigens (exemplary de novo CD8+ T cell responses: ARAP1_{Y>H}: 6.5% of CD8+ T cells and PKDREJ_{G>R}: 13.4% of CD8+ T cells; no cells were detectable prior to the stimulation process). The stimulation protocol successfully induced seven de novo CD8+ T cell responses towards both previously described and novel model neoantigens using PBMCs from another melanoma patient, NV6, up to varying magnitudes (ACTN4_{K>N} CSNK1A1_{S>L}, DHX40neoORF 7, GLI3_{P>L}, QARS_{R>W}, FAM178B_{P>L}, and RPS26_{P>L}, range: 0.2% of CD8+ T cells up to 52% of CD8+ T cells). Additionally, a CD8+ memory T cell response towards a patient-specific neoantigen was expanded (AASDHneoORF, up to 13% of CD8+ T cells post stimulation).

The induced CD8+ T cells from the patient was characterized in more detail. Upon re-challenge with mutant peptide loaded DCs, neoantigen-specific CD8+ T cells exhibited one, two and/or all three functions (16.9% and 65.5% functional CD8+ pMHC+ T cells for SRSF1E>K and ARAP1Y>H, respectively. When re-challenged with different concentrations of neoantigen peptides, the induced CD8+ T cells responded significantly to mutant neoantigen peptide but not to the wildtype peptide. In said patient, CD4+ T cell responses were identified using a recall response assay with mutant neoantigen loaded DCs. Three CD4+ T cell responses were identified (MKRN1S>L, CREBBPS>L and TPCN1K>E) based on the reactivity to DCs loaded with mutant neoantigen peptide. These CD4+ T cell responses also showed a polyfunctional profile when re-challenged with mutant neoantigen peptide. 31.3%, 34.5% & 41.9% of CD4+ T cells exhibited one, two and/or three functions; MKRN1S>L, CREBBPS>L and TPCN1K>E responses, respectively.

The cytotoxic capacity of the induced CD8+ responses from said patient was also assessed. Both SRSF1E>K and ARAP1Y>H responses showed a significant upregulation of CD107a on the CD8+ T cells and active Caspase3 on the tumor cells transduced with the mutant construct after co-culture.

Using the stimulation protocol, predicted patient-specific neoantigens, as well as model neoantigens, were confirmed to be immunogenic by the induction of multiple neoantigen-specific CD8+ and CD4+ T cell responses in patient material. The ability to induce polyfunctional and mutant-specific CD8+ and CD4+ T cell responses proves the capability of predicting high-quality neoantigens and generating potent T cell responses. The presence of multiple enriched neoantigen-specific T cell populations (memory and de novo) at the end of the stimulation process demonstrates the ability to raise new T cell responses and generate effective cancer immunotherapies to treat cancer patients.

Exemplary materials for T cell culture are provided below: Materials: AIM V media (Invitrogen)Human FLT3L; preclinical CellGenix #1415-050 Stock 50 ng/μL TNFα; preclinical CellGenix #1406-050 Stock 10 ng/μL; IL-1β, preclinical CellGenix #1411-050 Stock 10 ng/μL; PGE1 or Alprostadil—Cayman from Czech republic Stock 0.5 μg/μL; R10 media—RPMI 1640 glutamax+10% Human serum+1% PenStrep; 20/80 Media—18% AIM V+72% RPMI 1640 glutamax+10% Human Serum+1% PenStrep; IL7 Stock 5 ng/μL; IL15 Stock 5 ng/μL; DC media (Cellgenix); CD14 microbeads, human, Miltenyi #130-050-201, Cytokines and/or growth factors, T cell media (AIM V+RPMI 1640 glutamax+serum+PenStrep), Peptide stocks—1 mM per peptide (HIV A02—5-10 peptides, HIV B07—5-10 peptides, DOM—4-8 peptides, PIN—6-12 peptides).

1-76. (canceled)

77. A cell population comprising antigen-specific T cells, wherein the antigen-specific T cells comprise a T cell receptor (TCR) that binds to a peptide-MHC complex of antigen presenting cells (APCs), wherein the APCs comprise one or more peptides containing at least one selected epitope sequence, wherein the at least one selected epitope sequence is selected from a library of epitope sequences, wherein each epitope sequence in the library is matched to a protein encoded by an HLA allele, wherein the peptide-MHC complex comprises the at least one selected epitope sequence and the matched protein encoded by an HLA allele, and wherein each of the at least one selected epitope sequence satisfies at least two or three of the following criteria:
(i) binds to a protein encoded by the HLA allele,
(ii) is immunogenic according to an immunogenicity assay,
(iii) is presented by APCs according to a mass spectrometry assay, and
(iv) stimulates T cells to be cytotoxic according to a cytotoxicity assay.

78. The cell population of claim 77, wherein the at least one selected epitope sequence comprises a mutation expressed by cancer cells and not expressed by non-cancer cells.

79. The cell population of claim 77, wherein the at least one selected epitope sequence is within a protein overexpressed by cancer cells; or is within a protein expressed by a cell in a tumor microenvironment.

80. The cell population of claim 77, wherein the at least one selected epitope sequence is selected from one or more epitope sequences of Table 1A-1F, Table 2A-2C, Table 3, Table 4A-4M, Table 5, Table 6, Table 7, Table 8, Table 11, Table 12, Table 13 and Table 14.

81. The cell population of claim 77, wherein one or more of the at least one selected epitope sequence is from a protein overexpressed by a cancer cell of the subject, is from a tissue-specific protein, is from a cancer testes protein, comprises a driver mutation, comprises a drug resistance mutation, comprises a tumor specific mutation, is a viral epitope, is a minor histocompatibility epitope, is from a RAS protein, is from a GATA3 protein, is from an EGFR protein, is from a BTK protein, is from a p53 protein, is from a TMPRSS2::ERG fusion polypeptide or is from a Myc protein.

82. The cell population of claim 77, wherein at least one of the at least one selected epitope sequence is from a protein encoded by a gene selected from the group consisting of ANKRD30A, COL10A1, CTCFL, PPIAL4G, POTEE, DLL3, MMP13, SSX1, DCAF4L2, MAGEA4, MAGEA11, MAGEC2, MAGEA12, PRAME, CLDN6, EPYC, KLK3, KLK2, KLK4, TGM4, POTEG, RLN1, POTEH, SLC45A2, TSPAN10, PAGES, CSAG1, PRDM7, TG, TSHR, RSPH6A, SCXB, HIST1H4K, ALPPL2, PRM2, PRM1, TNP1, LELP1, HMGB4, AKAP4, CETN1, UBQLN3, ACTL7A, ACTL9, ACTRT2, PGK2, C2orf53, KIF2B, ADAD1, SPATA8, CCDC70, TPD52L3, ACTL7B, DMRTB1, SYCN CELA2A, CELA2B, PNLIPRP1, CTRC, AMY2A, SERPINI2, RBPJL, AQP12A, IAPP, KIRREL2, G6PC2, AQP12B, CYP11B1, CYP11B2, STAR, CYP11A1, and MC2R.

83. The cell population of claim 77, wherein the protein encoded by an HLA allele is a protein encoded by an HLA allele selected from the group consisting of HLA-A01:01, HLA-A02:01, HLA-A03:01, HLA-A11:01, HLA-A24:01, HLA-A30:01, HLA-A31:01, HLA-A32:01, HLA-A33:01, HLA-A68:01, HLA-B07:02, HLA-B08:01, HLA-B15:01, HLA-B44:03, HLA-007:01 and HLA-007:02.

84. The cell population of claim 77, wherein the at least one selected epitope sequence:
(i) binds to the matched protein encoded by an HLA allele with an affinity of 500 nM or less according to a binding assay, or
(ii) is predicted to bind to the matched protein encoded by the HLA allele with an affinity of 500 nM or less according to an MHC epitope prediction program implemented on a computer.

85. The cell population of claim 77, wherein the mass spectrometry assay comprises detecting the at least one selected epitope sequence by mass spectrometry after elution from the APCs with a mass accuracy of the detected peptide to be less than 15 Da or less than 10,000 parts per million (ppm).

86. The cell population of claim 77, wherein the immunogenicity assay is a multimer assay and the multimer assay comprises detecting T cells bound to a peptide-MHC multimer by flow cytometry, wherein the peptide-MHC multimer comprises the at least one selected epitope sequence and the matched protein encoded by an HLA allele, and wherein the T cells have been stimulated with APCs comprising a peptide containing the at least one selected epitope sequence.

87. The cell population of claim 77, wherein the at least one selected epitope sequence is immunogenic according to the multimer assay when (i) at least 10 T cells that have been stimulated with APCs comprising a peptide containing the at least one selected epitope sequence are detectable in at least one out of six stimulations from the same starting sample, (ii) the detectable T cells make up at least 0.005% of the CD8⁺ cells analyzed, and (iii) the percentage of detectable T cells of CD8+ T cells is higher than the percentage of detectable T cells of CD8+ T cells detectable in a control sample.

88. The cell population of claim 87, wherein the control sample comprises T cells that have been stimulated with APCs that (i) do not comprise a peptide containing the at least one selected epitope sequence, (ii) comprise a peptide derived from a different protein than the at least one selected epitope sequence, or (iii) comprise a peptide with a random sequence.

89. The cell population of claim 77, wherein the immunogenicity assay is a functional assay, wherein the functional assay comprises detecting T cells with intracellular staining of IFNγ or TNFα by an immunoassay or detecting T cells with cell surface expression of CD107a and/or CD107b by an immunoassay, wherein the T cells have been stimulated with APCs comprising a peptide containing the at least one selected epitope sequence.

90. The cell population of claim 89, wherein the at least one selected epitope sequence is immunogenic according to the functional assay when (i) at least 10 T cells that have been stimulated with APCs comprising a peptide containing the at least one selected epitope sequence are detected, (ii) the detected T cells make up at least 0.005% of the CD8⁺ or the CD4⁺ cells analyzed, and (iii) the percentage of detected T cells of CD8+ or CD4⁺ T cells is higher than the percentage of detected T cells of CD8+ or CD4⁺ T cells detected in a control sample.

91. The cell population of any one of claim 77, wherein the T cells stimulated to be cytotoxic according to the cytotoxicity assay are T cells that have been stimulated with APCs comprising a peptide containing the at least one selected epitope sequence that kill cells presenting the at least one selected epitope sequence, wherein a number of cells presenting the at least one selected epitope sequence that are killed by the T cells is at least 2 fold higher than (a) a number of cells that do not present the at least one selected epitope sequence that are killed by the T cells or (b) a number of cells presenting the at least one selected epitope sequence killed by T cells that have been stimulated with APCs that (i) do not comprise a peptide containing the at least one selected epitope sequence, (ii) comprise a peptide derived from a different protein than the at least one selected epitope sequence, or (iii) comprise a peptide with a random sequence.

92. The cell population of claim 77, wherein the T cells stimulated to be cytotoxic according to the cytotoxicity assay are T cells that produce a cytokine or IL2, wherein the cytokine is Interferon gamma (IFN-γ), Tumor Necrosis Factor (TNF) alpha (α) and/or TNF beta (β) or a combination thereof.

93. The cell population of claim 77, wherein at least 0.1% of the CD8+ T cells in the cell population are CD8+ tumor antigen-specific T cells derived from naïve CD8+ T cells.

94. The cell population of claim 77, wherein at least 0.1% of the CD4+ T cells in the cell population are CD4+ tumor antigen-specific T cells derived from naïve CD4+ T cells

95. The cell population of claim 77, wherein each of the at least one selected epitope sequence binds to a protein encoded by the HLA allele, is immunogenic according to an immunogenicity assay, is presented by APCs according to a mass spectrometry assay, and stimulates T cells to be cytotoxic according to a cytotoxicity assay.

96. A pharmaceutical composition comprising the cell population of claim 77 and a pharmaceutically acceptable excipient.

97. A cell population according to claim 77.

98. A method of preparing the cell population of claim 77, comprising contacting a cell population comprising T cells with antigen presenting cells (APCs) comprising one or more peptides containing at least one selected epitope sequence, wherein the at least one selected epitope sequence is selected from a library of epitope sequences, wherein each epitope sequence in the library is matched to a protein encoded by an HLA allele, and wherein each of the at least one selected epitope sequence satisfies at least two or three or each of the following criteria:
(i) binds to a protein encoded by the HLA allele,
(ii) is immunogenic according to an immunogenicity assay,
(iii) is presented by APCs according to a mass spectrometry assay, and
(iv) stimulates T cells to be cytotoxic according to a cytotoxicity assay;
thereby producing antigen-specific T cells comprising a T cell receptor (TCR) that binds to a peptide-MHC complex, the peptide-MHC complex comprising the at least one selected epitope sequence and the matched protein encoded by an HLA allele.

99. The method of claim 98, wherein the method further comprises depleting CD14+ cells and/or CD25+ cells from a population of immune cells comprising APCs and T cells prior to contacting the cell population comprising T cells with the APCs comprising the one or more peptides containing the at least one selected epitope sequence.

100. The method of claim 99, wherein the method further comprises incubating the CD14+ and/or CD25+ depleted cell population in the presence of
(i) FMS-like tyrosine kinase 3 receptor ligand (FLT3L), and
(ii) (A) a polypeptide comprising the at least one selected epitope sequence, or
(B) a polynucleotide encoding the polypeptide; to form a cell population of cells comprising stimulated T cells.
